SEQUENCE LISTING (289 sequences)

SEQ ID NO: 1 - protein, 7096 aa, unidentified [sequence omitted]

SEQ ID NO: 2 - protein, 9 aa, Homo sapiens
VMVELVAEL

SEQ ID NO: 3 - protein, 9 aa, Homo sapiens
YLATALLTL

SEQ ID NO: 4 - protein, 9 aa, Homo sapiens
CLEASFNYL

SEQ ID NO: 5 - protein, 9 aa, Homo sapiens
WLMWLIINL

SEQ ID NO: 6 - protein, 9 aa, Homo sapiens
SLPGVFCGV

SEQ ID NO: 7 - protein, 9 aa, Homo sapiens
FLLNKEMYL

SEQ ID NO: 8 - protein, 9 aa, Homo sapiens
SMWALIISV

SEQ ID NO: 9 - protein, 9 aa, Homo sapiens
YLASGGQPI

SEQ ID NO: 10 - protein, 9 aa, Homo sapiens
KLSYGIATV

SEQ ID NO: 11 - protein, 9 aa, Homo sapiens
YLDAYNMMI

SEQ ID NO: 12 - protein, 9 aa, Homo sapiens
LLLDDFVEI

SEQ ID NO: 13 - protein, 9 aa, Homo sapiens
FVFLVLLPL

SEQ ID NO: 14 - protein, 9 aa, Homo sapiens
SIIAYTMSL

SEQ ID NO: 15 - protein, 9 aa, Homo sapiens
ALNTLVKQL

SEQ ID NO: 16 - protein, 9 aa, Homo sapiens
VLNDILSRL

SEQ ID NO: 17 - protein, 9 aa, Homo sapiens
RLQSLQTYV

SEQ ID NO: 18 - protein, 9 aa, Homo sapiens
FIAGLIAIV

SEQ ID NO: 19 - protein, 9 aa, Homo sapiens
FLAFVVFLL

SEQ ID NO: 20 - protein, 9 aa, Homo sapiens
FLLVTLAIL

SEQ ID NO: 21 - protein, 9 aa, Homo sapiens
FLLNKEMYL

SEQ ID NO: 22 - protein, 9 aa, Homo sapiens
IFLWLLWPV

SEQ ID NO: 23 - protein, 9 aa, Homo sapiens
GLMWLSYFI

SEQ ID NO: 24 - protein, 9 aa, Homo sapiens
HLVDFQVTI

SEQ ID NO: 25 - protein, 9 aa, Homo sapiens
IIFWFSLEL

SEQ ID NO: 26 - protein, 9 aa, Homo sapiens
YVVDDPCPI

SEQ ID NO: 27 - protein, 9 aa, Homo sapiens
YIDIGNYTV

SEQ ID NO: 28 - protein, 9 aa, Homo sapiens
YINVFAFPF

SEQ ID NO: 29 - protein, 9 aa, Homo sapiens
NVFAFPFTI

SEQ ID NO: 30 - protein, 8 aa, Homo sapiens
VMVELVAE

SEQ ID NO: 31 - protein, 8 aa, Homo sapiens
LATALLTL

SEQ ID NO: 32 - protein, 8 aa, Homo sapiens
CLEASFNY

SEQ ID NO: 33 - protein, 8 aa, Homo sapiens
LMWLIINL

SEQ ID NO: 34 - protein, 8 aa, Homo sapiens
SLPGVFCG

SEQ ID NO: 35 - protein, 8 aa, Homo sapiens
LLNKEMYL

SEQ ID NO: 36 - protein, 8 aa, Homo sapiens
SMWALIIS

SEQ ID NO: 37 - protein, 8 aa, Homo sapiens
LASGGQPI

SEQ ID NO: 38 - protein, 8 aa, Homo sapiens
KLSYGIAT

SEQ ID NO: 39 - protein, 8 aa, Homo sapiens
LDAYNMMI

SEQ ID NO: 40 - protein, 8 aa, Homo sapiens
LLLDDFVE

SEQ ID NO: 41 - protein, 8 aa, Homo sapiens
VFLVLLPL

SEQ ID NO: 42 - protein, 8 aa, Homo sapiens
SIIAYTMS

SEQ ID NO: 43 - protein, 8 aa, Homo sapiens
LNTLVKQL

SEQ ID NO: 44 - protein, 8 aa, Homo sapiens
VLNDILSR

SEQ ID NO: 45 - protein, 8 aa, Homo sapiens
LQSLQTYV

SEQ ID NO: 46 - protein, 8 aa, Homo sapiens
FIAGLIAI

SEQ ID NO: 47 - protein, 8 aa, Homo sapiens
LAFVVFLL

SEQ ID NO: 48 - protein, 8 aa, Homo sapiens
FLLVTLAI

SEQ ID NO: 49 - protein, 8 aa, Homo sapiens
LLNKEMYL

SEQ ID NO: 50 - protein, 8 aa, Homo sapiens
IFLWLLWP

SEQ ID NO: 51 - protein, 8 aa, Homo sapiens
LMWLSYFI

SEQ ID NO: 52 - protein, 8 aa, Homo sapiens
HLVDFQVT

SEQ ID NO: 53 - protein, 8 aa, Homo sapiens
IFWFSLEL

SEQ ID NO: 54 - protein, 8 aa, Homo sapiens
YVVDDPCP

SEQ ID NO: 55 - protein, 8 aa, Homo sapiens
IDIGNYTV

SEQ ID NO: 56 - protein, 8 aa, Homo sapiens
YINVFAFP

SEQ ID NO: 57 - protein, 8 aa, Homo sapiens
VFAFPFTI

SEQ ID NO: 58 - protein, 15 aa, Homo sapiens
KSAFYILPSIISNEK

SEQ ID NO: 59 - protein, 15 aa, Homo sapiens
ESPFVMMSAPPAQYE

SEQ ID NO: 60 - protein, 15 aa, Homo sapiens
PNMLRIMASLVLARK

SEQ ID NO: 61 - protein, 14 aa, Homo sapiens
RIKVQMLSDTLKNL

SEQ ID NO: 62 - protein, 15 aa, Homo sapiens
LDAYNMMISAGFSLW

SEQ ID NO: 63 - protein, 13 aa, Homo sapiens
MFVFLVLLPLVSS

SEQ ID NO: 64 - protein, 15 aa, Homo sapiens
FLAFVVFLLVTLAIL

SEQ ID NO: 65 - protein, 15 aa, Homo sapiens
FLLVTLAILTALRLC

SEQ ID NO: 66 - protein, 15 aa, Homo sapiens
LSYYKLGASQRVAGD

SEQ ID NO: 67 - protein, 15 aa, Homo sapiens
AEILLIIMRTFKVSI

SEQ ID NO: 68 - protein, 15 aa, Homo sapiens
MKIILFLALITLATC

SEQ ID NO: 69 - protein, 14 aa, Homo sapiens
IIFLALITLATCEL

SEQ ID NO: 70 - protein, 15 aa, Homo sapiens
SPIFLIVAAIVFITL

SEQ ID NO: 71 - protein, 15 aa, Homo sapiens
DFYLCFLAFLLFLVL

SEQ ID NO: 72 - protein, 15 aa, Homo sapiens
MKFLVFLGIITTVAA

SEQ ID NO: 73 - protein, 16 aa, Homo sapiens
KQQTVTLLPAADLDDF

SEQ ID NO: 74 - protein, 14 aa, Homo sapiens
KSAFYILPSIISNE

SEQ ID NO: 75 - protein, 14 aa, Homo sapiens
ESPFVMMSAPPAQY

SEQ ID NO: 76 - protein, 14 aa, Homo sapiens
PNMLRIMASLVLAR

SEQ ID NO: 77 - protein, 13 aa, Homo sapiens
RIKVQMLSDTLKN

SEQ ID NO: 78 - protein, 14 aa, Homo sapiens
LDAYNMMISAGFSL

SEQ ID NO: 79 - protein, 12 aa, Homo sapiens
MFVFLVLLPLVS

SEQ ID NO: 80 - protein, 14 aa, Homo sapiens
FLAFVVFLLVTLAI

SEQ ID NO: 81 - protein, 14 aa, Homo sapiens
FLLVTLAILTALRL

SEQ ID NO: 82 - protein, 14 aa, Homo sapiens
LSYYKLGASQRVAG

SEQ ID NO: 83 - protein, 14 aa, Homo sapiens
AEILLIIMRTFKVS

SEQ ID NO: 84 - protein, 14 aa, Homo sapiens
MKIILFLALITLAT

SEQ ID NO: 85 - protein, 13 aa, Homo sapiens
IIFLALITLATCE

SEQ ID NO: 86 - protein, 14 aa, Homo sapiens
SPIFLIVAAIVFIT

SEQ ID NO: 87 - protein, 14 aa, Homo sapiens
DFYLCFLAFLLFLV

SEQ ID NO: 88 - protein, 14 aa, Homo sapiens
MKFLVFLGIITTVA

SEQ ID NO: 89 - protein, 15 aa, Homo sapiens
KQQTVTLLPAADLDD

SEQ ID NO: 90 - protein, 14 aa, Homo sapiens
SAFYILPSIISNEK

SEQ ID NO: 91 - protein, 14 aa, Homo sapiens
SPFVMMSAPPAQYE

SEQ ID NO: 92 - protein, 14 aa, Homo sapiens
NMLRIMASLVLARK

SEQ ID NO: 93 - protein, 13 aa, Homo sapiens
IKVQMLSDTLKNL

SEQ ID NO: 94 - protein, 14 aa, Homo sapiens
DAYNMMISAGFSLW

SEQ ID NO: 95 - protein, 12 aa, Homo sapiens
FVFLVLLPLVSS

SEQ ID NO: 96 - protein, 14 aa, Homo sapiens
LAFVVFLLVTLAIL

SEQ ID NO: 97 - protein, 14 aa, Homo sapiens
LLVTLAILTALRLC

SEQ ID NO: 98 - protein, 14 aa, Homo sapiens
SYYKLGASQRVAGD

SEQ ID NO: 99 - protein, 14 aa, Homo sapiens
EILLIIMRTFKVSI

SEQ ID NO: 100 - protein, 14 aa, Homo sapiens
KIILFLALITLATC

SEQ ID NO: 101 - protein, 13 aa, Homo sapiens
IFLALITLATCEL

SEQ ID NO: 102 - protein, 14 aa, Homo sapiens
PIFLIVAAIVFITL

SEQ ID NO: 103 - protein, 14 aa, Homo sapiens
FYLCFLAFLLFLVL

SEQ ID NO: 104 - protein, 14 aa, Homo sapiens
KFLVFLGIITTVAA

SEQ ID NO: 105 - protein, 15 aa, Homo sapiens
QQTVTLLPAADLDDF

SEQ ID NO: 106 - protein, 25 aa, Homo sapiens
SQCVNLTTRTQLPPAYTNSFTRGVY

SEQ ID NO: 107 - protein, 23 aa, Homo sapiens
FSNVTWFHAIHVSGTNGTKRFDN

SEQ ID NO: 108 - protein, 31 aa, Homo sapiens
DAVDCALDPLSETKCTLKSFTVEKGIYQTSN

SEQ ID NO: 109 - protein, 40 aa, Homo sapiens
GTNTSNQVAVLYQDVNCTEVPVAIHADQLTPTWRVYSTGS

SEQ ID NO: 110 - protein, 75 aa, Homo sapiens
VCGPKKSTNLVKNKCVNFNFNGLTGTGVLTESNKKFLPFQQFGRDIADTTDAVRDPQTLEILDITPCSFGGVSVI

SEQ ID NO: 111 - protein, 62 aa, Homo sapiens
NLDSKVGGNYNYLYRLFRKSNLKPFERDISTEIYQAGSTPCNGVEGFNCYFPLQSYGFQPTE

SEQ ID NO: 112 - protein, 25 aa, Homo sapiens
YNSASFSTFKCYGVSPTKLNDLCFT

SEQ ID NO: 113 - protein, 35 aa, Homo sapiens
FPNITNLCPFGEVFNATRFASVYAWNRKRISNCVA

SEQ ID NO: 114 - protein, 40 aa, Homo sapiens
VNNTVYDPLQPELDSFKEELDKYFKNHTSPDVDLGDISGI

SEQ ID NO: 115 - protein, 18 aa, Homo sapiens
FSQILPDPSKPSKRSFIE

SEQ ID NO: 116 - protein, 22 aa, Homo sapiens
FGAGAALQIPFAMQMAYRFNGI

SEQ ID NO: 117 - protein, 23 aa, Homo sapiens
SQCVNLTTRTQLPPAYTNSFTRG

SEQ ID NO: 118 - protein, 21 aa, Homo sapiens
FSNVTWFHAIHVSGTNGTKRF

SEQ ID NO: 119 - protein, 29 aa, Homo sapiens
DAVDCALDPLSETKCTLKSFTVEKGIYQT

SEQ ID NO: 120 - protein, 38 aa, Homo sapiens
GTNTSNQVAVLYQDVNCTEVPVAIHADQLTPTWRVYST

SEQ ID NO: 121 - protein, 73 aa, Homo sapiens
VCGPKKSTNLVKNKCVNFNFNGLTGTGVLTESNKKFLPFQQFGRDIADTTDAVRDPQTLEILDITPCSFGGVS

SEQ ID NO: 122 - protein, 60 aa, Homo sapiens
NLDSKVGGNYNYLYRLFRKSNLKPFERDISTEIYQAGSTPCNGVEGFNCYFPLQSYGFQP

SEQ ID NO: 123 - protein, 23 aa, Homo sapiens
YNSASFSTFKCYGVSPTKLNDLC

SEQ ID NO: 124 - protein, 33 aa, Homo sapiens
FPNITNLCPFGEVFNATRFASVYAWNRKRISNC

SEQ ID NO: 125 - protein, 38 aa, Homo sapiens
VNNTVYDPLQPELDSFKEELDKYFKNHTSPDVDLGDIS

SEQ ID NO: 126 - protein, 16 aa, Homo sapiens
FSQILPDPSKPSKRSF

SEQ ID NO: 127 - protein, 20 aa, Homo sapiens
FGAGAALQIPFAMQMAYRFN

SEQ ID NO: 128 - protein, 23 aa, Homo sapiens
CVNLTTRTQLPPAYTNSFTRGVY

SEQ ID NO: 129 - protein, 21 aa, Homo sapiens
NVTWFHAIHVSGTNGTKRFDN

SEQ ID NO: 130 - protein, 29 aa, Homo sapiens
VDCALDPLSETKCTLKSFTVEKGIYQTSN

SEQ ID NO: 131 - protein, 38 aa, Homo sapiens
NTSNQVAVLYQDVNCTEVPVAIHADQLTPTWRVYSTGS

SEQ ID NO: 132 - protein, 73 aa, Homo sapiens
GPKKSTNLVKNKCVNFNFNGLTGTGVLTESNKKFLPFQQFGRDIADTTDAVRDPQTLEILDITPCSFGGVSVI

SEQ ID NO: 133 - protein, 60 aa, Homo sapiens
DSKVGGNYNYLYRLFRKSNLKPFERDISTEIYQAGSTPCNGVEGFNCYFPLQSYGFQPTE

SEQ ID NO: 134 - protein, 23 aa, Homo sapiens
SASFSTFKCYGVSPTKLNDLCFT

SEQ ID NO: 135 - protein, 33 aa, Homo sapiens
NITNLCPFGEVFNATRFASVYAWNRKRISNCVA

SEQ ID NO: 136 - protein, 38 aa, Homo sapiens
NTVYDPLQPELDSFKEELDKYFKNHTSPDVDLGDISGI

SEQ ID NO: 137 - protein, 16 aa, Homo sapiens
QILPDPSKPSKRSFIE

SEQ ID NO: 138 - protein, 20 aa, Homo sapiens
AGAALQIPFAMQMAYRFNGI

SEQ ID NO: 139 - protein, 481 aa, synthetic construct
  REGION 1..481: note = Synthesized Vaccine Candidate 1
EAAAKSLPGVFCGVAAYYLATALLTLAAYYINVFAFPFAAYFLLNKEMYLAAYFLAFVVFLLAAYFIAGLIAIVAAYWLMWLIINLAAYYIDIGNYTVAAYIIFWFSLELAAYNVFAFPFTIAAYFVFLVLLPLAAYALNTLVKQLAAYKLSYGIATVAAYRLQSLQTYVAAYLLLDDFVEIGPGPGPNMLRIMASLVLARKGPGPGAEILLIIMRTFKVSIGPGPGLSYYKLGASQRVAGDGPGPGDFYLCFLAFLLFLVLGPGPGMKFLVFLGIITTVAAGPGPGKSAFYILPSIISNEKKKSQCVNLTTRTQLPPAYTNSFTRGVYKKDAVDCALDPLSETKCTLKSFTVEKGIYQTSNKKGTNTSNQVAVLYQDVNCTEVPVAIHADQLTPTWRVYSTGSKKVCGPKKSTNLVKNKCVNFNFNGLTGTGVLTESNKKFLPFQQFGRDIADTTDAVRDPQTLEILDITPCSFGGVSVI

SEQ ID NO: 140 - protein, 208 aa, synthetic construct
  REGION 1..208: note = Sythesized Vaccine Candidate 2
EAAAKYNSASFSTFKCYGVSPTKLNDLCFTGPGPGCLEASFNYLAAYWLMWLIINLAAYILLLDQALVAAYSLPGVFCGVAAYTLMNVLTLVAAYVLSFCAFAVAAYALNTLVKQAAYFLAFVVFLAAYGLMWLSYFAAYFLWLLWPVGPGPGPNMLRIMASLVLARKGPGPGRIKIVQMLSDTLKNLGPGPGFLLVTLAILTALRLC

SEQ ID NO: 141 - protein, 214 aa, synthetic construct
  REGION 1..214: note = Synthesized Vaccine Candidate 3
EAAAKDAVDCALDPLSETKCTLKSFTVEKGIYQTSNGPGPGCLEASFNYLAAYWLMWLIINLAAYILLLDQALVAAYSLPGVFCGVAAYTLMNVLTLVAAYVLSFCAFAVAAYALNTLVKQAAYFLAFVVFLAAYGLMWLSYFAAYFLWLLWPVGPGPGPNMLRIMASLVLARKGPGPGRIKIVQMLSDTLKNLGPGPGFLLVTLAILTALRLC

SEQ ID NO: 142 - protein, 218 aa, synthetic construct
  REGION 1..218: note = Synthesized Vaccine Candidate 4
EAAAKDAVDCALDPLSETKCTLKSFTVEKGIYQTSNGPGPGYLATALLTLAAYTMADLVYALAAYVLSFCAFAVAAYNLIDSYFVVAAYFVDGVPFVVAAYVLGSLAATVAAYLITGRLQSLAAYFLLVTLAILAAYLMWLSYFIAAAYLALLLLDRLGPGPGPNMLRIMASLVLARKGPGPGRIKIVQMLSDTLKNLGPGPGFLLVTLAILTALRLC

SEQ ID NO: 143 - protein, 207 aa, synthetic construct
  REGION 1..207: note = Synthesized Vaccine Candidate 5
EAAAKFGAGAALQIPFAMQMAYRFNGIGPGPGKLSYGIATVAAYVLWAHGFELAAYLLLDDFVEIAAYYLNTLTLAVAAYLFTMLRKAAYNLNESLIDLAAYFVFLVLLPLAAYSVLLFLAFVAAYFLWLLWPVTAAYLLLDRLNQLGPGPGPNMLRIMASLVLARKGPGPGRIKIVQMLSDTLKNLGPGPGFLLVTLAILTALRLC

SEQ ID NO: 144 - protein, 182 aa, synthetic construct
  REGION 1..182: note = Synthesized Vaccine Candidate 6
EAAAKFAFACPDGVAAYILFLALITLAAYFLALITLATAAYKLFIRQEEVAAYELYSPIFLIAAYFLAFLLFLVAAYMLIIFWFSLAAYYLCFLAFLLAAYFLLFLVLIMAAYIIFWFSLELGPGPGPNMLRIMASLVLARKGPGPGRIKIVQMLSDTLKNLGPGPGFLLVTLAILTALRLC

SEQ ID NO: 145 - protein, 194 aa, synthetic construct
  REGION 1..194: note = Synthesized Vaccine Candidate 7
EAAAKLITGRLQSLAAYRLNEVAKNLAAYNLNESLIDLAAYFIAGLIAIVAAYGLMWLSYFIAAYALNTPKDHIAAYLQLPQGTTLAAYLALLLLDRLAAYLLLDRLNQLAAYRLNQLESKMAAYGMSRIGMEVGPGPGPNMLRIMASLVLARKGPGPGRIKIVQMLSDTLKNLGPGPGFLLVTLAILTALRLC

SEQ ID NO: 146 - protein, 182 aa, synthetic construct
  REGION 1..182: note = Synthesized Vaccine Candidate 8
EAAAKYLATALLTLAAYFLALCADSIAAYVMVELVAELAAYFLKKDAPYIAAYFLGRYMSALAAYALNLGETFVAAYYLQPRTFLLAAYFVFLVLLPLAAYKIADYNYKLAAYRLQSLQTYVGPGPGPNMLRIMASLVLARKGPGPGRIKIVQMLSDTLKNLGPGPGFLLVTLAILTALRLC

SEQ ID NO: 147 - protein, 182 aa, synthetic construct
  REGION 1..182: note = Synthesized Vaccine Candidate 9
EAAAKFLLNKEMYLAAYFLAHIQWMVAAYFLLPSLATVAAYWLMWLIINLAAYILFTRFFYVAAYYLYALVYFLAAYLLYDANYFLAAYALSKGVHFVAAYWLIVGVALLAAYNLLLLFVTVGPGPGPNMLRIMASLVLARKGPGPGRIKIVQMLSDTLKNLGPGPGFLLVTLAILTALRLC

SEQ ID NO: 148 - protein, 182 aa, synthetic construct
  REGION 1..182: note = Synthesized Vaccine Candidate 10
EAAAKTMADLVYALAAYALWEIQQVVAAYVLSFCAFAVAAYNLIDSYFVVAAYFVDGVPFVVAAYKLSYGIATVAAYFTISVTTEIAAYRLDKVEAEVAAYFLAFVVFLLAAYVLLFLAFVVGPGPGPNMLRIMASLVLARKGPGPGRIKIVQMLSDTLKNLGPGPGFLLVTLAILTALRLC

SEQ ID NO: 149 - protein, 182 aa, synthetic construct
  REGION 1..182: note = Synthesized Vaccine Candidate 11
EAAAKVLWAHGFELAAYYLDAYNMMIAAYLLLDDFVEIAAYYLNTLTLAVAAYALLADKFPVAAYYLATALLTLAAYFLALCADSIAAYVMVELVAELAAYFLKKDAPYIAAYFLGRYMSALGPGPGPNMLRIMASLVLARKGPGPGRIKIVQMLSDTLKNLGPGPGFLLVTLAILTALRLC

SEQ ID NO: 150 - protein, 182 aa, synthetic construct
  REGION 1..182: note = Synthesized Vaccine Candidate 12
EAAAKFLLNKEMYLAAYFLAHIQWMVAAYFLLPSLATVAAYWLMWLIINLAAYSMWALIISVAAYYIDIGNYTVAAYYVVDDPCPIAAYFLEYHDVRVAAYFLGIITTVAAAYFAFPFTIYSGPGPGPNMLRIMASLVLARKGPGPGRIKIVQMLSDTLKNLGPGPGFLLVTLAILTALRLC

SEQ ID NO: 151 - protein, 182 aa, synthetic construct
  REGION 1..182: note = Synthesized Vaccine Candidate 13
EAAAKALWEIQQVVAAYVLSFCAFAVAAYYLASGGQPIAAYVLGSLAATVAAYMLFTMLRKLAAYSLVKPSFYVAAYSVLLFLAFVAAYFVLAAVYRIAAYKLLEQWNLVAAYNVFAFPFTIGPGPGPNMLRIMASLVLARKGPGPGRIKIVQMLSDTLKNLGPGPGFLLVTLAILTALRLC

SEQ ID NO: 152 - protein, 182 aa, synthetic construct
  REGION 1..182: note = Synthesized Vaccine Candidate 14
EAAAKFLFLTWICLAAYLMWLSYFIAAAYFLWLLWPVTAAYIFLWLLWPVAAYLIIMRTFKVAAYHLVDFQVTIAAYNLDYIINLIAAYSIWNLDYIIAAYTIAEILLIIAAYRMNSRNYIAGPGPGPNMLRIMASLVLARKGPGPGRIKIVQMLSDTLKNLGPGPGFLLVTLAILTALRLC

SEQ ID NO: 153 - protein, 182 aa, synthetic construct
  REGION 1..182: note = Synthesized Vaccine Candidate 15
EAAAKCLEASFNYLAAYWLMWLIINLAAYILLLDQALVAAYSACVLAAECAAYSLPGVFCGVAAYTLMNVLTLVAAYSMWALIISVAAYSIIAYTMSLAAYALNTLVKQLAAYVLNDILSRLGPGPGPNMLRIMASLVLARKGPGPGRIKIVQMLSDTLKNLGPGPGFLLVTLAILTALRLC

SEQ ID NO: 154 - protein, 217 aa, synthetic construct
  REGION 1..217: note = Synthesized Vaccine Candidate 16
EAAKDAVDCALDPLSETKCTLKSFTVEKGIYQTSNGPGPGSLPITVYYAAAYALLEDPVGTAAYGIFEDRAPVAAYNLLTTPKFTAAYRMLGDVMAVAAYRLNELLAYVAAYALSALLTKLAAYALHTALATVAAYRLLGFADTVAAYALMLRLLRIGPGPGPNMLRIMASLVLARKGPGPGRIKIVQMLSDTLKNLGPGPGFLLVTLAILTALRLC

SEQ ID NO: 155 - protein, 217 aa, synthetic construct; identical to SEQ ID NO: 154
  REGION 1..217: note = Synthesized Vaccine Candidate 17

SEQ ID NO: 156 - DNA, 303 bp, Homo sapiens
  source 1..303: mol_type = genomic DNA
atgaacaggaaggtgaccgccatcgccctggccgccatcatctgggccaccgccgcccagggcttcctgatgttcaagcagggcaggtgcctgtgcatcggccccggcatgaaggccgtgaagatggccgagatcgagaaggccagcgtgatctaccccagcaacggctgcgacaaggtggaggtgatcgtgaccatgaaggcccacaagaggcagaggtgcctggaccccaggagcaagcaggccaggctgatcatgcaggccatcgagaagaagaacttcctgaggaggcagaacatgtga

SEQ ID NO: 157 - DNA, 465 bp, Homo sapiens
  source 1..465: mol_type = genomic DNA
atgaaggtctccgcggcagccctcgctgtcatcctcattgctactgccctctgcgctcctgcatctgcctccccatattcctcggacaccacaccctgctgctttgcctacattgcccgcccactgccccgtgcccacatcaaggagtatttctacaccagtggcaagtgctccaacccagcagtcgtccacaggtcaaggatgccaaagagagagggacagcaagtctggcaggatttcctgtatgactcccggctgaacaagggcaagctttgtcacccgaaagaaccgccaagtgtgtgccaacccagagaagaaatgggttcgggagtacatcaactctttggagatgagctaggatggagagtccttgaacctgaacttacacaaatttgcctgtttctgcttgctcttgtcctagcttgggaggcttcccctcactatcctaccccacccgctccttga

SEQ ID NO: 158 - DNA, 378 bp, Homo sapiens
  source 1..378: mol_type = genomic DNA
atgaagaaaagtggtgttcttttcctcttgggcatcatcttgctggttctgattggagtgcaaggaaccccagtagtgagaaagggtcgctgttcctgcatcagcaccaaccaagggactatccacctacaatccttgaaagaccttaaacaatttgccccaagcccttcctgcgagaaaattgaaatcattgctacactgaagaatggagttcaaacatgtctaaacccagattcagcagatgtgaaggaactgattaaaaagtgggagaaacaggtcagccaaaagaaaaagcaaaagaatgggaaaaaacatcaaaaaaagaaagttctgaaagttcgaaaatctcaacgttctcgtcaaaagaagactacataa

SEQ ID NO: 159 - DNA, 297 bp, Homo sapiens
  source 1..297: mol_type = genomic DNA
atgaatcaaactgccattctgatttgctgccttatctttctgactctaagtggcattcaaggagtacctctctctagaactgtacgctgtacctgcatcagcattagtaatcaacctgttaatccaaggtctttagaaaaacttgaaattattcctgcaagccaattttgtccacgtgttgagatcattgctacaatgaaaaagaagggtgagaagagatgtctgaatccagaatcgaaggccatcaagaatttactgaaagcagttagcaaggaaaggtctaaaagatctccttaa

SEQ ID NO: 160 - DNA, 300 bp, Homo sapiens
  source 1..300: mol_type = genomic DNA
atgaggctcctggcggccgcgctgctcctgctgctgctggcgctgtacaccgcgcgtgtggacgggtccaaatgcaagtgctcccggaagggacccaagatccgctacagcgacgtgaagaagctggaaatgaagccaaagtacccgcactgcgaggagaagatggttatcatcaccaccaagagcgtgtccaggtaccgaggtcaggagcactgcctgcaccccaagctgcagagcaccaagcgcttcatcaagtggtacaacgcctggaacgagaagcgcagggtctacgaagaatag

SEQ ID NO: 161 - protein, 360 aa, Homo sapiens
ATGAAAGTTCTAATCTCTTCCCTCCTCCTGTTGCTGCCACTAATGCTGATGTCCATGGTCTCTAGCAGCCTGAATCCAGGGGTCGCCAGAGGCCACAGGGACCGAGGCCAGGCTTCTAGGAGATGGCTCCAGGAAGGCGGCCAAGAATGTGAGTGCAAAGATTGGTTCCTGAGAGCCCCGAGAAGAAAATTCATGACAGTGTCTGGGCTGCCAAAGAAGCAGTGCCCCTGTGATCATTTCAAGGGCAATGTGAAGAAAACAAGACACCAAAGGCACCACAGAAAGCCAAACAAGCATTCCAGAGCCTGCCAGCAATTTCTCAAACAATGTCAGCTAAGAAGCTTTGCTCTGCCTTTGTAG

SEQ ID NO: 162 - DNA, 453 bp, Homo sapiens
  source 1..453: mol_type = genomic DNA
atgaacctgtggctcctggcctgcctggtggccggcttcctgggagcctgggcccccgctgtccacacccaaggtgtctttgaggactgctgcctggcctaccactaccccattgggtgggctgtgctccggcgcgcctggacttaccggatccaggaggtgagcgggagctgcaatctgcctgctgcgatattctacctccccaagagacacaggaaggtgtgtgggaaccccaaaagcagggaggtgcagagagccatgaagctcctggatgctcgaaataaggtttttgcaaagctccaccacaacacgcagaccttccaagcaggccctcatgctgtaaagaagttgagttctggaaactccaagttatcatcgtccaagtttagcaatcccatcagcagcagtaagaggaatgtctccctcctgatatcagctaattcaggactgtga

SEQ ID NO: 163 - DNA, 384 bp, Homo sapiens
  source 1..384: mol_type = genomic DNA
atgcagcagagaggactcgccatcgtggccttggctgtctgtgcggccctacatgcctcagaagccatacttcccattgcctccagctgttgcacggaggtttcacatcatatttccagaaggctcctggaaagagtgaatatgtgtcgcatccagagagctgatggggattgtgacttggctgctgtcatccttcatgtcaagcgcagaagaatctgtgtcagcccgcacaaccatactgttaagcagtggatgaaagtgcaagctgccaagaaaaatggtaaaggaaatgtttgccacaggaagaaacaccatggcaagaggaacagtaacagggcacatcaggggaaacacgaaacatacggccataaaactccttattag

SEQ ID NO: 164 - DNA, 465 bp, Homo sapiens
  source 1..465: mol_type = genomic DNA
atgttccacgtgagcttcaggtacatcttcggcatcccccccctgatcctggtgctgctgcccgtgaccagcagcgagtgccacatcaaggacaaggagggcaaggcctacgagagcgtgctgatgatcagcatcgacgagctggacaagatgaccggcaccgacagcaactgccccaacaacgagcccaacttcttcaggaagcacgtgtgcgacgacaccaaggaggccgccttcctgaacagggccgccaggaagctgaagcagttcctgaagatgaacatcagcgaggagttcaacgtgcacctgctgaccgtgagccagggcacccagaccctggtgaactgcaccagcaaggaggagaagaacgtgaaggagcagaagaagaacgacgcctgcttcctgaagaggctgctgagggagatcaagacctgctggaacaagatcctgaagggcagcatctga

SEQ ID NO: 165 - DNA, 489 bp, Homo sapiens
  source 1..489: mol_type = genomic DNA
atgagaatttcgaaaccacatttgagaagtatttccatccagtgctacttgtgtttacttctaaacagtcattttctaactgaagctggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttga

SEQ ID NO: 166 - DNA, 462 bp, Homo sapiens
  source 1..462: mol_type = genomic DNA
atgtacaggatgcaactcctgtcttgcattgcactaagtcttgcacttgtcacaaacagtgcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgacttga

SEQ ID NO: 167 - DNA, 1733 bp, synthetic construct
  misc_feature 1..1733: note = CAG promoter
  source 1..1733: mol_type = other DNA
ctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattg

SEQ ID NO: 168 - DNA, 589 bp, synthetic construct
  misc_feature 1..589: note = CMV Promoter
  source 1..589: mol_type = other DNA
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatc

SEQ ID NO: 169 - DNA, 635 bp, Homo sapiens
  source 1..635: mol_type = other DNA; note = SP-B Promoter
gtatagggctgtctgggagccactccagggccacagaaatcttgtctctgactcagggtattttgttttctgttttgtgtaaatgctcttctgactaatgcaaaccatgtgtccatagaaccagaagatttttccaggggaaaaggtaaggaggtggtgagagtgtcctgggtctgcccttccagggcttgccctgggttaagagccaggcaggaagctctcaagagcattgctcaagagtagagggggcctgggaggcccagggaggggatgggaggggaacacccaggctgcccccaaccagatgccctccaccctcctcaacctccctcccacggcctggagaggtgggaccaggtatggaggcttgagagcccctggttggaggaagccacaagtccaggaacatgggagtctgggcagggggcaaaggaggcaggaacaggccatcagccaggacaggtggtaaggcaggcaggagtgttcctgctgggaaaaggtgggatcaagcacctggagggctcttcagagcaaagacaaacactgaggtcgctgccactcctacagagcccccacgccccgcccagctataaggggccatgcaccaagcagggtacccaggctgcagaggtgcc

SEQ ID NO: 170 - DNA, 1559 bp, Homo sapiens
  source 1..1559: mol_type = other DNA; note = CD144 promoter
catccatgcccatggcctcagatgccagccataagctgttgggttccaaacctcgactccaggctggactcacccctgtctcccccaccagcctgacacctccacctgggtatctaacgagcatctcaaactcaacctgcctgagacagaggaatcactatcccctcctcctccaaaaatatccttccatcacactccccatcttgtgctctgatttactaaacggccctgggccctctctttctcagggtctctgcttgcccagctatataataaaacaagtttgggacttcccaaccattcacccatggaaaaacagaagcaactcttcaaaggacagattcccaggatctgccctgggagattccaaatcagttgatctggggtgagcccagtcctctgtagtttttagaagctcctcctatgtctctcctggtcagcagaatcttggcccctcccttccccccagcctcttggttcttctgggctctgatccagcctcagcgtcactgtcttccacgcccctctttgattctcgtttatgtcaaaagccttgtgaggatgaggctgtgattatccccattttacagatgaggaaactgtggctccaggatgacacaactggccagaggtcacatcagaagcagagctgggtcacttgactccacccaatatccctaaatgcaaacatcccctacagaccgaggctggcaccttagagctggagtccatgcccgctctgaccaggagaagccaacctggtcctccagagccaagagcttctgtccctttcccatctcctgaagcctccctgtcacctttaaagtccattcccacaaagacatcatgggatcaccacagaaaatcaagctctggggctaggctgaccccagctagatttttggctcttttataccccagctgggtggacaagcaccttaaacccgctgagcctcagcttcccgggctataaaatgggggtgatgacacctgcctgtagcattccaaggagggttaaatgtgatgctgcagccaagggtccccacagccaggctctttgcaggtgctgggttcagagtcccagagctgaggccgggagtaggggttcaagtggggtgccccaggcagggtccagtgccagccctctgtggagacagccatccggggccgaggcagccgcccaccgcagggcctgcctatctgcagccagcccagccctcacaaaggaacaataacaggaaaccatcccagggggaagtgggccagggccagctggaaaacctgaaggggaggcagccaggcctccctcgccagcggggtgtggctcccctccaaagacggtcggctgacaggctccacagagctccactcacgctcagccctggacggacaggcagtccaacggaacagaaacatccctcagcccacaggcacggtgagtgggggctcccacactcccctccaccccaaacccgccaccctgcgcccaagatgggagggtcctcagcttccccatctgtagaatgggcatcgtcccactcccatgacagagaggctcc

SEQ ID NO: 171 - DNA, 39 bp, synthetic construct
  misc_feature 1..39: note = wild type native leader sequence for the Spike protein
  source 1..39: mol_type = other DNA
atgttcgtgttcctggtgctgctgcccctggtgagcagc

SEQ ID NO: 172 - DNA, 63 bp, synthetic construct
  misc_feature 1..63: note = T2A Linker
  source 1..63: mol_type = other DNA
ggaagcggagagggcaggggaagtcttctaacatgcggggacgtggaggaaaatcccggcccc

SEQ ID NO: 173 - DNA, 69 bp, synthetic construct
  misc_feature 1..69: note = E2A Linker
  source 1..69: mol_type = other DNA
ggaagcggacagtgtactaattatgctctcttgaaattggctggagatgttgagagcaacccaggtccc

SEQ ID NO: 174 - DNA, 66 bp, synthetic construct
  misc_feature 1..66: note = P2A Linker
  source 1..66: mol_type = other DNA
ggaagcggagccacgaacttctctctgttaaagcaagcaggagatgttgaagaaaaccccgggcct

SEQ ID NO: 175 - DNA, 18 bp, synthetic construct
  misc_feature 1..18: note = 6-His Tag
  source 1..18: mol_type = other DNA
catcaccatcaccatcac

SEQ ID NO: 176 - protein, 292 aa, synthetic construct
  REGION 1..292: note = SARS-CoV-like Spike-S1-NTD
SQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLK

SEQ ID NO: 177 - protein, 223 aa, synthetic construct
  REGION 1..223: note = SARS-CoV-2 Spike-S1-RBD
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 178 - protein, 666 aa, synthetic construct
  REGION 1..666: note = CoV Spike S1-S2_S2
FNGLTGTGVLTESNKKFLPFQQFGRDIADTTDAVTRAGCLIGAEHVNNSYECDIPIGAGICASYQTQTNRDPQTLEILDITPCSFGGVSVITPGTNTSNQVAVLYQDVNCTEVPVAIHADQLTPTWRVYSTGSNVFQSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQ

SEQ ID NO: 179 - protein, 1273 aa, synthetic construct
  REGION 1..1273: note = spike glycoprotein with a mutation 682-QQAQ-685 mutation
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVTRAGCLIGAEHVNNSYECDIPIGAGICASYQTQTNRDPQTLEILDITPCSFGGVSVITPGTNTSNQVAVLYQDVNCTEVPVAIHADQLTPTWRVYSTGSNVFQSPQQAQSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 180 - protein, 1273 aa, synthetic construct
  REGION 1..1273: note = spike glycoprotein with two proline substitutions (K986P, V987P)
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 181 - protein, 1273 aa, synthetic construct
  REGION 1..1273: note = spike glycoprotein with four proline substitutions(F817P,A892P, A899P, A942P)
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVTRAGCLIGAEHVNNSYECDIPIGAGICASYQTQTNRDPQTLEILDITPCSFGGVSVITPGTNTSNQVAVLYQDVNCTEVPVAIHADQLTPTWRVYSTGSNVFQSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 182 - protein, 1273 aa, synthetic construct
  REGION 1..1273: note = Synthesized spike glycoprotein with six proline substitutions(F817P, A892P, A899P, A942P, K986P, V987P)
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVTRAGCLIGAEHVNNSYECDIPIGAGICASYQTQTNRDPQTLEILDITPCSFGGVSVITPGTNTSNQVAVLYQDVNCTEVPVAIHADQLTPTWRVYSTGSNVFQSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 183 - protein, 1273 aa, synthetic construct
  REGION 1..1273: note = spike glycoprotein with six proline substitutions(F817P, A892P,A899P, A942P, K986P, V987P) and a 682-QQAQ-685 mutation
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVTRAGCLIGAEHVNNSYECDIPIGAGICASYQTQTNRDPQTLEILDITPCSFGGVSVITPGTNTSNQVAVLYQDVNCTEVPVAIHADQLTPTWRVYSTGSNVFQSPQQAQSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 184 - protein, 9 aa, Homo sapiens
KSYGFQPTY

SEQ ID NO: 185 - protein, 9 aa, Homo sapiens
VVGNHKYRF

SEQ ID NO: 186 - protein, 9 aa, Homo sapiens
YQVGNKPCK

SEQ ID NO: 187 - protein, 9 aa, Homo sapiens
CVIAWNSKK

SEQ ID NO: 188 - protein, 9 aa, Homo sapiens
KGAKGLNCY

SEQ ID NO: 189 - protein, 9 aa, Homo sapiens
SQCVNFTTR

SEQ ID NO: 190 - protein, 9 aa, Homo sapiens
NIADYNYKL

SEQ ID NO: 191 - protein, 9 aa, Homo sapiens
YLPLKSYGF

SEQ ID NO: 192 - protein, 9 aa, Homo sapiens
KCYGVSLNK

SEQ ID NO: 193 - protein, 9 aa, Homo sapiens
IYKTPPIKY

SEQ ID NO: 194 - protein, 9 aa, Homo sapiens
CVADYSFLY

SEQ ID NO: 195 - protein, 9 aa, Homo sapiens
SVYAWDRRK

SEQ ID NO: 196 - protein, 9 aa, Homo sapiens
RFFRKSNLK

SEQ ID NO: 197 - protein, 9 aa, Homo sapiens
DISTEIYQV

SEQ ID NO: 198 - protein, 9 aa, Homo sapiens
YQPHRVVVL

SEQ ID NO: 199 - protein, 9 aa, Homo sapiens
FVIRGDQVK

SEQ ID NO: 200 - protein, 9 aa, Homo sapiens
NATKFSSVY

SEQ ID NO: 201 - protein, 9 aa, Homo sapiens
NLCPFSEIF

SEQ ID NO: 202 - protein, 9 aa, Homo sapiens
ASATVCGPK

SEQ ID NO: 203 - protein, 9 aa, Homo sapiens
KINNCVADY

SEQ ID NO: 204 - protein, 8 aa, Homo sapiens
KSYGFQPT

SEQ ID NO: 205 - protein, 8 aa, Homo sapiens
VVGNHKYR

SEQ ID NO: 206 - protein, 8 aa, Homo sapiens
YQVGNKPC

SEQ ID NO: 207 - protein, 8 aa, Homo sapiens
CVIAWNSK

SEQ ID NO: 208 - protein, 8 aa, synthetic construct
KGAKGLNC

SEQ ID NO: 209 - protein, 8 aa, Homo sapiens
SQCVNFTT

SEQ ID NO: 210 - protein, 8 aa, Homo sapiens
NIADYNYK

SEQ ID NO: 211 - protein, 8 aa, Homo sapiens
YLPLKSYG

SEQ ID NO: 212 - protein, 8 aa, Homo sapiens
KCYGVSLN

SEQ ID NO: 213 - protein, 8 aa, Homo sapiens
IYKTPPIK

SEQ ID NO: 214 - protein, 8 aa, Homo sapiens
CVADYSFL

SEQ ID NO: 215 - protein, 8 aa, Homo sapiens
SVYAWDRR

SEQ ID NO: 216 - protein, 8 aa, Homo sapiens
RFFRKSNL

SEQ ID NO: 217 - protein, 8 aa, Homo sapiens
DISTEIYQ

SEQ ID NO: 218 - protein, 8 aa, Homo sapiens
YQPHRVVV

SEQ ID NO: 219 - protein, 8 aa, Homo sapiens
FVIRGDQV

SEQ ID NO: 220 - protein, 8 aa, Homo sapiens
NATKFSSV

SEQ ID NO: 221 - protein, 8 aa, Homo sapiens
NLCPFSEI

SEQ ID NO: 222 - protein, 8 aa, Homo sapiens
ASATVCGP

SEQ ID NO: 223 - protein, 8 aa, Homo sapiens
KINNCVAD

SEQ ID NO: 224 - protein, 8 aa, Homo sapiens
KSYGFQPT

SEQ ID NO: 225 - protein, 15 aa, Homo sapiens
LCPFSEIFNATKFSS

SEQ ID NO: 226 - protein, 15 aa, Homo sapiens
NCYLPLKSYGFQPTY

SEQ ID NO: 227 - protein, 15 aa, Homo sapiens
GNHKYRFRFFRKSNL

SEQ ID NO: 228 - protein, 15 aa, Homo sapiens
PFERDISTEIYQVGN

SEQ ID NO: 229 - protein, 15 aa, Homo sapiens
KKLDSKVVGNHKYRF

SEQ ID NO: 230 - protein, 15 aa, Homo sapiens
KGLNCYLPLKSYGFQ

SEQ ID NO: 231 - protein, 15 aa, Homo sapiens
LVLLPLVSSQCVNFT

SEQ ID NO: 232 - protein, 15 aa, Homo sapiens
RGDQVKQIAPGQTGN

SEQ ID NO: 233 - protein, 15 aa, Homo sapiens
SASFSTFKCYGVSLN

SEQ ID NO: 234 - protein, 15 aa, Homo sapiens
KLDSKVVGNHKYRFR

SEQ ID NO: 235 - protein, 15 aa, Homo sapiens
FAQVKQIYKTPPIKY

SEQ ID NO: 236 - protein, 15 aa, Homo sapiens
ADYSFLYNSASFSTF

SEQ ID NO: 237 - protein, 15 aa, Homo sapiens
ATKFSSVYAWDRRKI

SEQ ID NO: 238 - protein, 15 aa, Homo sapiens
PHRVVVLSFELLHAS

SEQ ID NO: 239 - protein, 15 aa, Homo sapiens
FERDISTEIYQVGNK

SEQ ID NO: 240 - protein, 15 aa, Homo sapiens
AKGLNCYLPLKSYGF

SEQ ID NO: 241 - protein, 15 aa, Homo sapiens
SIVRFPNITNLCPFS

SEQ ID NO: 242 - protein, 15 aa, Homo sapiens
NNCVADYSFLYNSAS

SEQ ID NO: 243 - protein, 15 aa, Homo sapiens
KGAKGLNCYLPLKSY

SEQ ID NO: 244 - protein, 14 aa, Homo sapiens
LCPFSEIFNATKFS

SEQ ID NO: 245 - protein, 14 aa, Homo sapiens
NCYLPLKSYGFQPT

SEQ ID NO: 246 - protein, 14 aa, Homo sapiens
GNHKYRFRFFRKSN

SEQ ID NO: 247 - protein, 14 aa, Homo sapiens
PFERDISTEIYQVG

SEQ ID NO: 248 - protein, 14 aa, Homo sapiens
KKLDSKVVGNHKYR

SEQ ID NO: 249 - protein, 14 aa, Homo sapiens
KGLNCYLPLKSYGF

SEQ ID NO: 250 - protein, 14 aa, Homo sapiens
LVLLPLVSSQCVNF

SEQ ID NO: 251 - protein, 14 aa, Homo sapiens
RGDQVKQIAPGQTG

SEQ ID NO: 252 - protein, 14 aa, Homo sapiens
SASFSTFKCYGVSL

SEQ ID NO: 253 - protein, 14 aa, Homo sapiens
KLDSKVVGNHKYRF

SEQ ID NO: 254 - protein, 14 aa, Homo sapiens
FAQVKQIYKTPPIK

SEQ ID NO: 255 - protein, 14 aa, Homo sapiens
ADYSFLYNSASFST

SEQ ID NO: 256 - protein, 14 aa, Homo sapiens
ATKFSSVYAWDRRK

SEQ ID NO: 257 - protein, 14 aa, Homo sapiens
PHRVVVLSFELLHA

SEQ ID NO: 258 - protein, 14 aa, Homo sapiens
FERDISTEIYQVGN

SEQ ID NO: 259 - protein, 14 aa, Homo sapiens
AKGLNCYLPLKSYG

SEQ ID NO: 260 - protein, 14 aa, Homo sapiens
SIVRFPNITNLCPF

SEQ ID NO: 261 - protein, 14 aa, Homo sapiens
NNCVADYSFLYNSA

SEQ ID NO: 262 - protein, 14 aa, Homo sapiens
KGAKGLNCYLPLKS

SEQ ID NO: 263 - protein, 24 aa, Homo sapiens
CVNFTTRTQLPPAYTNSFTRGVYY

SEQ ID NO: 264 - protein, 26 aa, Homo sapiens
NITNLCPFSEIFNATKFSSVYAWDRR

SEQ ID NO: 265 - protein, 33 aa, Homo sapiens
INNCVADYSFLYNSASFSTFKCYGVSLNKLNDL

SEQ ID NO: 266 - protein, 18 aa, Homo sapiens
RGDQVKQIAPGQTGNIAD

SEQ ID NO: 267 - protein, 47 aa, Homo sapiens
KKLDSKWGNHKYRFRFFRKSNLKPFERDISTEISTEIYQVGNKPCKG

SEQ ID NO: 268 - protein, 6 aa, Homo sapiens
TYGVGY

SEQ ID NO: 269 - protein, 16 aa, Homo sapiens
LHASATVCGPKKSTNL

SEQ ID NO: 270 - protein, 30 aa, Homo sapiens
VKQIYKTPPIKYFGGFNFSQILPDPSKPSK

SEQ ID NO: 271 - protein, 22 aa, Homo sapiens
CVNFTTRTQLPPAYTNSFTRGV

SEQ ID NO: 272 - protein, 24 aa, Homo sapiens
NITNLCPFSEIFNATKFSSVYAWD

SEQ ID NO: 273 - protein, 31 aa, Homo sapiens
INNCVADYSFLYNSASFSTFKCYGVSLNKLN

SEQ ID NO: 274 - protein, 16 aa, Homo sapiens
RGDQVKQIAPGQTGNI

SEQ ID NO: 275 - protein, 45 aa, Homo sapiens
KKLDSKWGNHKYRFRFFRKSNLKPFERDISTEISTEIYQVGNKPC

SEQ ID NO: 276 - protein, 14 aa, Homo sapiens
LHASATVCGPKKST

SEQ ID NO: 277 - protein, 28 aa, Homo sapiens
VKQIYKTPPIKYFGGFNFSQILPDPSKP

SEQ ID NO: 278 - protein, 22 aa, Homo sapiens
NFTTRTQLPPAYTNSFTRGVYY

SEQ ID NO: 279 - protein, 24 aa, Homo sapiens
TNLCPFSEIFNATKFSSVYAWDRR

SEQ ID NO: 280 - protein, 31 aa, Homo sapiens
NCVADYSFLYNSASFSTFKCYGVSLNKLNDL

SEQ ID NO: 281 - protein, 16 aa, Homo sapiens
DQVKQIAPGQTGNIAD

SEQ ID NO: 282 - protein, 45 aa, Homo sapiens
LDSKWGNHKYRFRFFRKSNLKPFERDISTEISTEIYQVGNKPCKG

SEQ ID NO: 283 - protein, 14 aa, Homo sapiens
ASATVCGPKKSTNL

SEQ ID NO: 284 - protein, 28 aa, Homo sapiens
QIYKTPPIKYFGGFNFSQILPDPSKPSK

SEQ ID NO: 285 - DNA, 119 bp, unidentified
  source 1..119: mol_type = other DNA; note = Influenza
ttcggagctattgctggtttcttggaaggaggatgggaaggaatgattgcaggttggcacggatacacatctcatggagcacatggagtagcagtggcagcagaccttaagagtaccca

SEQ ID NO: 286 - protein, 40 aa, unidentified
  source 1..40: note = Influenza
FGAIAGFLEGGWEGMIAGWHGYTSHGAHGVAVAADLKSTX

SEQ ID NO: 287 - DNA, 1701 bp, unidentified
  source 1..1701: mol_type = other DNA; note = Influenza
atgaaggcaatactagtagttctgctatatacatttgcaaccgcaaatgcagacacattatgtataggttatcatgcgaacaactcaacagacactgtagacacagtactagaaaagaatgtaacagtaacacactctgttaaccttctagaagacaagcataacgggaaactatgcaaactaagaggggtagccccattgcatttgggtaaatgtaacattgctggctggatcctgggaaatccagagtgtgaatcactctccacagcaagctcatggtcctacattgtggaaacatctagttcagacaatggaacgtgttacccaggagatttcatcgattatgaggagctaagagagcaattgagctcagtgtcatcatttgaaaggtttgagatattccccaagacaagttcatggcccaatcatgactcgaacaaaggtgtaacgacagcatgtcctcatgctggagcaaaaagcttctacaaaaatttaatatggctagttaaaaaaggaaattcatacccaaagctcagcaaatcctacattaatgataaagggaaagaagtcctcgtgctatggggcattcaccatccacctactagtgctgaccaacaaagtctctatcagaatgcagatgcatatgtttttgtggggacatcaagatacagcaagaagttcaagccggaaatagcaataaggcccaaagtgagggatcaagaagggagaatgaactattactggacactagtagagccgggagacaaaataacattcgaagcaactggaaatctagtggtaccgagatatgcattcgcaatggaaagaaatgctggatctggtattatcatttcagatacaccagtccacgattgcaatacaacttgtcagacacccaagggtgctataaacaccagcctcccatttcagaacatacatccgatcacaattggaaaatgtccaaaatatgtaaaaagcacaaaattgagactggccacaggattgaggaatgtcccgtccattcaatctagaggcctatttggggccattgccggtttcattgaaggggggtggacagggatggtagatggatggtacggttatcaccatcaaaatgagcaggggtcaggatatgcagccgacctgaagagcacacagaatgccattgacgagattactaacaaagtaaactctgttattgaaaaaatgaatacacagttcacagcagtaggtaaagagttcaaccacctggaaaaaagaatagagaatttaaataaaaaagttgatgatggtttcctggacatttggacttacaatgccgaactgttggttctattggaaaatgaaagaactttggactaccacgattcaaaggtgaagaacttatatgaaaaggtaagaagccagttaaaaaacaatgccaaggaaattggaaacggctgctttgaattttaccacaaatgtgataacacgtgcatggaaagtgtcaaaaatgggacttatgactacccaaaatactcagaggaagcaaaattaaacagagaagaaatagatggggtaaagctggaatcaacaaggatttaccagattttggcgatctattcaaccgtcgccagttcattggtactggtagtctccctgggggcaatcagtttctggatgtgctctaatgggtctctacagtgtagaatatgtatttaa

SEQ ID NO: 288 - DNA, 1762 bp, unidentified
  source 1..1762: mol_type = other DNA; note = Influenza
agcaaaagcaggggataattctattaaccatgaagactatcattgctttgagctacattctatgtctggttttcgctcaaaaacttcctggaaatgacaatagcactgcaacgctgtgccttgggcaccatgcagtaccaaacggaacgatagtgaaaacaatcacgaatgaccaaattgaagttactaatgctactgagctggttcagaattcctcaataggtgaaatatgcgacagtcctcatcagatccttgatggagaaaactgcacactaatagatgctctattgggagaccctcagtgtgatggctttcaaaataagaaatgggacctttttgttgaacgaagcaaagcctacagcaactgttacccttatgatgtgccggattatgcctcccttaggtcactagttgcctcatccggcacactggagtttaacaatgaaagcttcaattgggctggagtcactcaaaacggaacaagttctgcttgcataaggggatctaatagtagtttctttagtagattaaattggttgacccacttaaacttcaagtacccagcattgaacgtgactatgccaaacaatgaacaatttgacaaattgtacatttggggggttcaccacccgggtacggacaaggaccaaatcttcctgtatgctcaatcatcaggaagaatcacagtatctaccaaaagaagccaacaagctgtaatcccgaatatcggatctagacccagaataaggaatatccctagcagaataagcatctattggacaatagtaaaaccgggagacatacttttgattaacagcacagggaatctaattgctcctaggggttacttcaaaatacgaagtgggaaaagctcaataatgagatcagatgcacccattggcaaatgcaagtctgaatgcatcactccaaatggaagcattcccaatgacaaaccattccaaaatgtaaacaggatcacatacggggcctgtcccagatatgttaagcaaagcactctgaaattggcaacaggaatgcgaaatgtaccagagaaacaaactagaggcatatttggcgcaatagcgggtttcatagaaaatggttgggagggaatggtggatggttggtacggtttcaggcatcaaaattctgagggaagaggacaagcagcagatctcaaaagcactcaagcagcaatcgatcaaatcaatgggaagctgaatcgattgatcgggaaaaccaacgagaaattccatcagattgagaaagaattctcagaagtagaagggagaattcaggaccttgagaaatatgttgaggacacaaaaatagatctctggtcatacaacgcagagcttcttgttgccctggaaaaccaacatacaattgatctaactgactcagaaatgaacaaactgtttgaaaaaacaaagaagcaactgagggaaaatgctgaggatatgggcaatggttgtttcaaaatataccacaaatgtgacaatgcctgcataggatcaatcagaaatggaacttatgaccacgatgtatacagggatgaagcattaaacaaccggttccagatcaagggagttgagctgaagtcagggtacaaagattggatcctatggatttcctttgccatatcatgttttttgctttgtgttgctttgttggggttcatcatgtgggcctgccaaaagggcaacattaggtgcaacatttgcatttgagtgcattaattaaaaacacccttgtttctact

SEQ ID NO: 289 - DNA, 1847 bp, unidentified
  source 1..1847: mol_type = other DNA; note = Influenza
attttctaatatccacaaaatgaaggcaataattgtactactcatggtagtaacatccaatgcagatcgaatctgcactgggataacatcgtcaaactcaccacatgtcgtcaaaactgctactcaaggggaggtcaacgtgaccggtgtaataccactgacaacaacacccaccaaatctcattttgcaaatctcaaaggaacagaaaccagggggaaactatgcccaaaatgcctcaactgcacagatctggatgtagccttgggcagaccaaaatgcacagggaaaataccctctgcaagggtttcaatactccatgaagtcagacctgttacatctgggtgctttcctataatgcacgatagaacaaaaattagacagctgcctaaccttctccgaggatacgaacatgtcaggttatcaactcacaacgttatcaatgcaaaagatgcaccaggaagaccctacgaaattggaacctcagggtcttgccctaacattaccaatggaaacggattcttcgcaacaatggcttgggccgtcccaaaaaacaaaacagcaacaaatccattaacaatagaagtaccatacatttgtacagaaggagaagaccaaattaccgtttgggggttccactctgacaacgagacccaaatggcaaagctctatggggactcaaagccccagaagttcacctcatctgccaacggagtgaccacacattacgtttcacagattggtggcttcccaaatcaaacagaagacggaggactaccacaaagtggcagaattgttgttgattacatggtgcagaaatctggaaaaacaggaacaattacctatcaaagaggtattttattgcctcaaaaagtgtggtgcgcaagtggcaggagcaaggtaataaaaggatccttgcccttaattggagaagcagattgcctccatgaaaaatacggtggattaaacaaaagcaagccttactacacaggggaacatgcaaaggccataggaaattgcccaatatgggtgaaaacacccttgaagctggccaatggaaccaaatatagaccccctgcaaaactattaaaggaaagaggtttcttcggagccattgctggtttcttagagggaggatgggaaggaatgattgcaggttggcacggatacacatcccatggggcacatggagtagcggtggcagctgaccttaagagcactcaagaggccataaacaagataacaaaaaatctcaactctttgagtgagctggaagtaaagaatcttcaaagactaagcggtgccatggatgaactccacaacgaaatactagaactagatgagaaagtggatgatctcagagctgacacaataagctcacaaatagaactcgcagtcctgctttccaatgaaggaataataaacagtgaagatgaacatctcttggcgcttgaaagaaagctgaagaaaatgctgggcccctctgctgtagagatagggaatggatgctttgaaaccaaacacaagtgcaaccagacctgcctcgacagaatagctgctggtacctttgatgcaggagaattttctctccccacctttgattcactgaatattactgctgcatctttaaatgacgacggattggacaatcatactatactgctttactactcaactgctgcctccagtttggctgtaacactgatgatagctatctttgttgtttatatggtctccagagacaatgtttcttgctccatttgtctataaggaaagttaagccctgtattttcctttattgtagtgcttgtttgcttgttgtcattacaaagaaacgttattgaaaaat

(19) United States
(12) Patent Application Publication
(10) Pub. No.: US 2023/0146932 A1
(43) Pub. Date: May 11, 2023

(54) MULTI-EPITOPE PAN-CORONAVIRUS VACCINE COMPOSITIONS

(71) Applicant: THE REGENTS OF THE UNIVERSITY OF CALIFORNIA, Oakland, CA (US)

(72) Inventor: Lbachir BenMohamed, Irvine, CA (US)

(21) Appl. No.: 18/046,862

(22) Filed: Oct. 14, 2022

Related U.S. Application Data
(63) Continuation-in-part of application No. PCT/US21/27341, filed on Apr. 14, 2021.
(60) Provisional application No. 63/084,421, filed on Sep. 28, 2020.
(60) Provisional application No. 63/009,907, filed on Apr. 14, 2020.
(60) Provisional application No. 63/349,799, filed on Jun. 7, 2022.
(60) Provisional application No. 63/349,904, filed on Jun. 7, 2022.
(60) Provisional application No. 63/302,454, filed on Jan. 24, 2022.

(51) Int. Cl. A61K 39/12; A61P 31/14; A61P 37/04; C07K 14/165

(52) U.S. Cl. CPC A61K 39/12; A61P 31/14; A61P 37/04; C07K 14/165; A61K 2039/545

(57) ABSTRACT

Multi-epitope, pan-coronavirus recombinant vaccine compositions featuring a combination of highly conserved B cell epitopes, highly conserved CD4+ T cell epitopes, and highly conserved CD8+ T cell epitopes, at least one of which is derived from a non-spike protein. The present invention uses several immuno-informatics and sequence alignment approaches and multiple immunological assays in vitro using human blood and saliva samples from COVID patients and healthy patients to identify several human B cells, CD4+ and CD8+ T cell epitopes that are highly conserved and antigenic in vitro. The Invention also used an in vivo unique mouse model of ACE2/HLA-A0201/HLA-DR triple transgenic mouse model to test the immunogenicity and the protective efficacy against SARS-CoV-2 infection and COVID-Like symptoms, of the identified B and T cell epitopes and of the resulting multi-epitope-pan-Coronavirus vaccine candidates. The vaccine compositions herein have the potential to provide long-lasting B and T cell immunity regardless of Coronaviruses mutations.

### CROSS-REFERENCES TO RELATED APPLICATIONS

This application is a continuation in part and claims benefit of PCT Application No. PCT/US2021/027341 filed Apr. 14, 2021, which claims benefit of U.S. Provisional Application No. 63/084,421 filed Sep. 28, 2020, and U.S. Provisional Application No. 63/009,907 filed Apr. 14, 2020, the specifications of which are incorporated herein in their entirety by reference.

This application is a non-provisional and claims benefit of U.S. Provisional Application No. 63/349,799 filed Jun. 7, 2022, U.S. Provisional Application No. 63/349,904 filed Jun. 7, 2022, and U.S. Provisional Application No. 63/302,454 filed Jan. 24, 2022, the specifications of which are incorporated herein in their entirety by reference.

### REFERENCE TO AN ELECTRONIC SEQUENCE LISTING

The contents of the electronic sequence listing (UCI 20 06A PCT CIP Sequence Listingg.xml: Size: 291,000 bytes: and Date of Creation: Oct. 14, 2022) is herein incorporated by reference in its entirety.

### STATEMENT REGARDING FEDERALLY SPONSORED RESEARCH OR DEVELOPMENT

This invention was made with government support under Grant No. Al158060, Al150091, Al143348, Al147499, Al143326, Al138764, A;124911 and Al110902 awarded by National Institutes of Allergy and Infectious Diseases. The government has certain rights in the invention.

### FIELD OF THE INVENTION

The present invention relates to vaccines, for example viral vaccines, such as those directed to coronaviruses, e.g., universal pan-coronavirus recombinant vaccines.

### BACKGROUND OF THE INVENTION

Over the last two decades, there have been three deadly human outbreaks of Coronaviruses (CoVs) caused by emerging zoonotic CoVs: SARS-CoV, MERS-CoV, and the latest highly transmissible and deadly SARS-CoV-2, which has caused the current COVID-19 global pandemic. All three deadly CoVs originated from bats, the natural hosts, and transmitted to humans via various intermediate animal reservoirs (e.g., pangolins, civet cats and camels). Because there is currently no universal pan-Coronavirus vaccine available, it remains highly possible that other global COVID-like pandemics will emerge in the coming years, caused by yet another spillover of an unknown zoonotic bat-derived SARS-like Coronavirus (SL-CoV) into an unvaccinated human population.

Neutralizing antibodies and antiviral effector CD4⁺ and CD8⁺ T cells appear to be crucial in reducing viral load in the majority of infected asymptomatic and convalescent patients. However, very little information exists on the antigenic landscape and the repertoire of B-cell and CD4⁺ and CD8⁺ T cell epitopes that are conserved among human, bat Coronavirus strains, and other animals Coronavirus strains.

### SUMMARY OF THE INVENTION

Determining the antigen and epitope landscapes that are conserved among human and animal Coronaviruses as well as the repertoire, phenotype and function of B cells and CD4+ and CD8+ T cells that correlate with resistance seen in asymptomatic COVID-19 patients may inform in the development of future pan-Coronavirus vaccines. The present invention describes using several immuno-informatics and sequence alignment approaches to identify several human B cell, CD4+ and CD8+ T cell epitopes that are highly conserved, e.g., highly conserved in: (i) greater than 81,000 SARS-CoV-2 human strains identified in 190 countries on six continents; (ii) six circulating CoVs that caused previous human outbreaks of the “Common Cold”; (iii) nine SL-CoVs isolated from bats; (iv) nine SL-CoV isolated from pangolins; (v) three SL-CoVs isolated from civet cats; and (vi) four MERS strains isolated from camels. Furthermore, the present invention describes the identification of cross-reactive epitopes that: recalled B cell, CD4+ and CD8+ T cells from both COVID-19 patients and healthy individuals who were never exposed to SARS-CoV-2; and induced strong B cell and T cell responses in “humanized” Human Leukocyte Antigen (HLA)-DR/HLA-A*02:01 double transgenic mice.

The present invention is not limited to vaccine compositions for use in humans. The present invention includes vaccine compositions for use in other pet animals such as dogs, cats, etc. Therefore, the present invention can be extended to include T cell epitopes that are restricted to other human HLA class 1 and HLA class II, besides (HLA)-DR/HLA-A*02:01 that covers 73%, so that a full coverage of the 100% human population can be ascertained, regardless of race and ethnicity. It also can be extended to include pets, such as cats and dogs T cell epitopes that are restricted to other human MHC class 1 and MHC class II.

The vaccine compositions herein have the potential to provide long-lasting B and T cell immunity regardless of Coronaviruses mutations. This may be due at least partly because the vaccine compositions target highly conserved structural Coronavirus antigens, such as Coronavirus nucleoprotein (also known as nucleocapsid), and non-structural Coronavirus antigens, such as one of 16 NSPs encoded by the ORF1a/b, in combination with other Coronavirus structural and non-structural antigens with a low mutation rate found in perhaps every human and animal Coronaviruses variants and strains.

The present invention is also related to selecting highly conserved structural (e.g., spike protein) and non-structural Coronavirus antigens inside the virus (e.g., a non-spike protein such as nucleocapsid, envelope and membrane proteins), which may be viral proteins that are normally not necessarily under mutation pressure by the immune system.

The present invention provides multi-epitope, pan-coronavirus recombinant vaccine compositions.

In certain embodiments, the vaccine compositions are for use in humans. In certain embodiments, the vaccine compositions are for use in animals, such as but not limited to mice, cats, dogs, non-human primates, other animals susceptible to coronavirus infection, other animals that may function as preclinical animal models for coronavirus infections, etc.

As used herein, the term “multi-epitope” refers to a composition comprising more than one B and T cell epitope wherein at least: one CD4 and/or CD8 T cell epitope is MHC-restricted and recognized by a TCR, and at least one epitope is a B cell epitope.

As used herein, the term “recombinant vaccine composition” may refer to one or more proteins or peptides encoded by one or more recombinant genes, e.g., genes that have been cloned into one or more systems that support the expression of said gene(s). The term “recombinant vaccine composition” may refer to the recombinant genes or the system that supports the expression of said recombinant genes.

For example, the present invention provides a multi-epitope, pan-coronavirus recombinant vaccine composition comprising at least two of: one or more conserved coronavirus B-cell target epitopes; one or more conserved coronavirus CD4+ T cell target epitopes; and/or one or more conserved coronavirus CD8+ T cell target epitopes. At least one epitope is derived from a non-spike protein, and the composition induces immunity to only the epitopes. In some embodiments, the epitopes are in the form of a single antigen, e.g., the composition comprises an antigen that comprises at least two of: one or more conserved coronavirus B-cell target epitopes; one or more conserved coronavirus CD4+ T cell target epitopes; and/or one or more conserved coronavirus CD8+ T cell target epitopes, wherein at least one epitope is derived from a non-spike protein, and wherein the composition induces immunity to only the epitopes. In certain embodiments, the epitopes are in the form of two or more antigens.

Likewise, the present invention provides a multi-epitope, pan-coronavirus recombinant vaccine composition comprising one or more conserved coronavirus B-cell target epitopes; one or more conserved coronavirus CD4+ T cell target epitopes; and one or more conserved coronavirus CD8+ T cell target epitopes. At least one epitope is derived from a non-spike protein, and the composition induces immunity to only the epitopes. In some embodiments, the epitopes are in the form of a single antigen, e.g., the composition comprises an antigen that comprises: one or more conserved coronavirus B-cell target epitopes; one or more conserved coronavirus CD4⁺ T cell target epitopes; and one or more conserved coronavirus CD8+ T cell target epitopes; wherein at least one epitope is derived from a non-spike protein, and wherein the composition induces immunity to only the epitopes. In certain embodiments, the epitopes are in the form of two or more antigens.

The present invention also provides a multi-epitope, pan-coronavirus recombinant vaccine composition comprising at least two of: whole spike protein; one or more conserved coronavirus CD4+ T cell target epitopes; and/or one or more conserved coronavirus CD8+ T cell target epitopes. At least one epitope is derived from a non-spike protein, and the composition induces immunity to only the epitopes. In some embodiments, the epitopes are in the form of a single antigen, e.g., the composition comprises an antigen that comprises at least two of: whole spike protein; one or more conserved coronavirus CD4+ T cell target epitopes; and/or one or more conserved coronavirus CD8+ T cell target epitopes; wherein at least one epitope is derived from a non-spike protein, and wherein the composition induces immunity to only the epitopes. In certain embodiments, the epitopes are in the form of two or more antigens.

Likewise, the present invention provides a multi-epitope, pan-coronavirus recombinant vaccine composition comprising whole spike protein; one or more conserved coronavirus CD4+ T cell target epitopes; and one or more conserved coronavirus CD8+ T cell target epitopes. At least one epitope is derived from a non-spike protein, and the composition induces immunity to only the epitopes. In some embodiments, the epitopes are in the form of a single antigen, e.g., the composition comprises an antigen that comprises: whole spike protein; one or more conserved coronavirus CD4+ T cell target epitopes; and one or more conserved coronavirus CD8+ T cell target epitopes; wherein at least one epitope is derived from a non-spike protein, and wherein the composition induces immunity to only the epitopes. In certain embodiments, the epitopes are in the form of two or more antigens.

The present invention also provides a multi-epitope, pan-coronavirus recombinant vaccine composition comprising at least two of: at least a portion of spike protein, the portion of spike protein comprising a trimerized SARS-CoV-2 receptor-binding domain (RBD); one or more conserved coronavirus CD4+ T cell target epitopes; and/or one or more conserved coronavirus CD8+ T cell target epitopes. At least one epitope is derived from a non-spike protein, and the composition induces immunity to only the epitopes. In some embodiments, the epitopes are in the form of a single antigen, e.g., the composition comprises an antigen that comprises at least two of: at least a portion of spike protein, the portion of spike protein comprising a trimerized SARS-CoV-2 receptor-binding domain (RBD); one or more conserved coronavirus CD4+ T cell target epitopes; and/or one or more conserved coronavirus CD8+ T cell target epitopes; wherein at least one epitope is derived from a non-spike protein, and wherein the composition induces immunity to only the epitopes. In certain embodiments, the epitopes are in the form of two or more antigens.

Likewise, the present invention provides a multi-epitope, pan-coronavirus recombinant vaccine composition comprising: at least a portion of spike protein, the portion of spike protein comprising a trimerized SARS-CoV-2 receptor-binding domain (RBD); one or more conserved coronavirus CD4+ T cell target epitopes; and one or more conserved coronavirus CD8+ T cell target epitopes. At least one epitope is derived from a non-spike protein, and the composition induces immunity to only the epitopes. In some embodiments, the epitopes are in the form of a single antigen, e.g., the composition comprises an antigen that comprises: at least a portion of spike protein, the portion of spike protein comprising a trimerized SARS-CoV-2 receptor-binding domain (RBD); one or more conserved coronavirus CD4+ T cell target epitopes; and one or more conserved coronavirus CD8+ T cell target epitopes; wherein at least one epitope is derived from a non-spike protein, and wherein the composition induces immunity to only the epitopes. In certain embodiments, the epitopes are in the form of two or more antigens.

Likewise, the present invention includes a multi-epitope, pan-coronavirus recombinant vaccine composition comprising one or more conserved coronavirus B-cell target epitopes; one or more conserved coronavirus CD4+ T cell target epitopes; and one or more conserved coronavirus CD8+ T cell target epitopes, wherein at least one epitope is derived from a non-spike protein, and the composition induces immunity to only the epitopes. In some embodiments, a conserved target epitope is one that is one of the 5 most conserved epitopes (for its epitope type, e.g., B cell, CD4 T cell, CD8 T cell) identified in a sequence alignment and analysis. In some embodiments, a conserved target epitope is one that is one of the 10 most conserved epitopes (for its epitope type, e.g., B cell, CD4 T cell, CD8 T cell) identified in a sequence alignment and analysis. In some embodiments, a conserved target epitope is one that is one of the 15 most conserved epitopes (for its epitope type, e.g., B cell, CD4 T cell, CD8 T cell) identified in a sequence alignment and analysis. In some embodiments, a conserved target epitope is one that is one of the 20 most conserved epitopes (for its epitope type, e.g., B cell, CD4 T cell, CD8 T cell) identified in a sequence alignment and analysis. In some embodiments, a conserved target epitope is one that is one of the 25 most conserved epitopes (for its epitope type, e.g., B cell, CD4 T cell, CD8 T cell) identified in a sequence alignment and analysis. In some embodiments, a conserved target epitope is one that is one of the 30 most conserved epitopes (for its epitope type, e.g., B cell, CD4 T cell, CD8 T cell) identified in a sequence alignment and analysis. In some embodiments, a conserved target epitope is one that is one of the 35 most conserved epitopes (for its epitope type, e.g., B cell, CD4 T cell, CD8 T cell) identified in a sequence alignment and analysis. In some embodiments, a conserved target epitope is one that is one of the 40 most conserved epitopes (for its epitope type, e.g., B cell, CD4 T cell, CD8 T cell) identified in a sequence alignment and analysis. In some embodiments, a conserved target epitope is one that is one of the 50 most conserved epitopes (for its epitope type, e.g., B cell. CD4 T cell, CD8 T cell) identified in a sequence alignment and analysis. Examples of sequence alignments and analyses. Are described herein. For example, steps or methods for selecting or identifying conserved epitopes may first include performing a sequence alignment and analysis of a particular number of coronavirus sequences to determine sequence similarity or identity amongst the group of analyzed sequences. In some embodiments, the sequences used for alignments may include human and animal sequences. In certain embodiments, the sequences used for alignments include one or more SARS-CoV-2 human strains or variants in current circulation; one or more coronaviruses that has caused a previous human outbreak; one or more coronaviruses isolated from animals selected from a group consisting of bats, pangolins, civet cats, minks, camels, and other animal receptive to coronaviruses; and/or one or more coronaviruses that cause the common cold.

Likewise, the present invention includes a multi-epitope, pan-coronavirus recombinant vaccine composition comprising one or more conserved coronavirus B-cell target epitopes; one or more conserved coronavirus CD4+ T cell target epitopes; and one or more conserved coronavirus CD8+ T cell target epitopes, wherein at least one epitope is derived from a non-spike protein, and the composition induces immunity to only the epitopes, and wherein the conserved epitopes are identified by: performing a sequence alignment and analysis of a particular number of coronavirus sequences to determine sequence similarity or identity amongst the group of analyzed sequences. The conserved epitopes are those that are among the most highly conserved epitopes identified in the analysis (for the particular type of epitope, e.g., B cell, CD4 T cell, CD8 T cell). For example, the conserved epitopes may be the 5 most highly conserved epitopes identified (for the particular type of epitope). In some embodiments, the conserved epitopes may be the 10 most highly conserved epitopes identified (for the particular type of epitope). In some embodiments, the conserved epitopes may be the 15 most highly conserved epitopes identified (for the particular type of epitope). In some embodiments, the conserved epitopes may be the 20 most highly conserved epitopes identified (for the particular type of epitope). In some embodiments, the conserved epitopes may be the 25 most highly conserved epitopes identified (for the particular type of epitope). In some embodiments, the conserved epitopes may be the 30 most highly conserved epitopes identified (for the particular type of epitope). In some embodiments, the conserved epitopes may be the 40 most highly conserved epitopes identified (for the particular type of epitope). In some embodiments, the conserved epitopes may be the 50 most highly conserved epitopes identified (for the particular type of epitope). The present invention is not limited to the aforementioned thresholds.

Likewise, the present invention includes a multi-epitope, pan-coronavirus recombinant vaccine composition, the composition comprising: one or more conserved coronavirus B-cell target epitopes and one or more conserved coronavirus CD4⁺ T cell target epitopes, or one or more conserved coronavirus CD8⁺ T cell target epitopes and one or more conserved coronavirus CD4⁺ T cell target epitopes, wherein at least one epitope is derived from a non-spike protein; wherein the composition induces immunity to only the epitopes.

In some embodiments, the alignment and analysis for 50 or more sequences, 100 or more sequences, 200 or more sequences, 300 or more sequences, 400 or more sequences, 500 or more sequences, 1000 or more sequences, 2000 or more sequences, 3000 or more sequences, 4000 or more sequences, 5000 or more sequences, 10,000 or more sequences, 15,000 or more sequences, more than 15,000 sequences, etc., In some embodiments, the sequences used for alignments may include human and animal sequences. In certain embodiments, the sequences used for alignments include one or more SARS-CoV-2 human strains or variants in current circulation; one or more coronaviruses that has caused a previous human outbreak; one or more coronaviruses isolated from animals selected from a group consisting of bats, pangolins, civet cats, minks, camels, and other animal receptive to coronaviruses; and/or one or more coronaviruses that cause the common cold. In some embodiments, the one or more SARS-CoV-2 human strains or variants in current circulation are selected from: variant B.1.177; variant B.1.160, variant B.1.1.7 (UK), variant P.1 (Japan/Brazil), variant B.1.351 (South Africa), variant B.1.427 (California), variant B.1.429 (California), variant B.1.258; variant B.1.221; variant B.1.367; variant B.1.1.277; variant B.1.1.302; variant B.1.525; variant B.1.526, variant S:677H; variant S:677P; B.1.617.2-Delta, variant B.1.1.529-Omicron (BA.1); sub-variant Omicron (BA.1); sub-variant Omicron (BA.2); sub-variant Omicron (BA.3); sub-variant Omicron (BA.4); sub-variant Omicron (BA.5) In some embodiments, the one or more coronaviruses that cause the common cold are selected from: 229E alpha coronavirus, NL63 alpha coronavirus, OC43 beta coronavirus, and HKU1 beta coronavirus

The present invention provides a multi-epitope, pan-coronavirus recombinant vaccine composition comprising an antigen delivery system encoding at least two of: one or more conserved coronavirus B-cell target epitopes; one or more conserved coronavirus CD4+ T cell target epitopes; and/or one or more conserved coronavirus CD8+ T cell target epitopes. At least one epitope is derived from a non-spike protein, and the composition induces immunity to only the epitopes. In some embodiments, the epitopes are in the form of a single antigen, e.g, the composition comprises an antigen that comprises at least two of: one or more conserved coronavirus B-celltarget epitopes; one or more conserved coronavirus CD4⁺ T cell target epitopes; and/or one or more conserved coronavirus CD8+ T cell target epitopes, wherein at least one epitope is derived from a non-spike protein, and wherein the composition induces immunity to only the epitopes. In certain embodiments, the epitopes are in the form of two or more antigens.

Likewise, the present invention provides a multi-epitope, pan-coronavirus recombinant vaccine composition comprising an antigen delivery system encoding one or more conserved coronavirus B-cell target epitopes; one or more conserved coronavirus CD4+ T cell target epitopes; and one or more conserved coronavirus CD8+ T cell target epitopes. At least one epitope is derived from a non-spike protein, and the composition induces immunity to only the epitopes. In some embodiments, the epitopes are in the form of a single antigen, e.g., the composition comprises an antigen that comprises: one or more conserved coronavirus B-cell target epitopes; one or more conserved coronavirus CD4+ T cell target epitopes; and one or more conserved coronavirus CD8+ T cell target epitopes; wherein at least one epitope is derived from a non-spike protein, and wherein the composition induces immunity to only the epitopes. In certain embodiments, the epitopes are in the form of two or more antigens.

The present invention also provides a multi-epitope, pan-coronavirus recombinant vaccine composition comprising an antigen delivery system encoding at least two of: whole spike protein; one or more conserved coronavirus CD4+ T cell target epitopes; and/or one or more conserved coronavirus CD8+ T cell target epitopes. At least one epitope is derived from a non-spike protein, and the composition induces immunity to only the epitopes. In some embodiments, the epitopes are in the form of a single antigen, e.g., the composition comprises an antigen that comprises at least two of: whole spike protein; one or more conserved coronavirus CD4⁺ T cell target epitopes; and/or one or more conserved coronavirus CD8⁺ T cell target epitopes; wherein at least one epitope is derived from a non-spike protein, and wherein the composition induces immunity to only the epitopes. In certain embodiments, the epitopes are in the form of two or more antigens.

Likewise, the present invention provides a multi-epitope, pan-coronavirus recombinant vaccine composition comprising an antigen delivery system encoding whole spike protein; one or more conserved coronavirus CD4+ T cell target epitopes; and one or more conserved coronavirus CD8+ T cell target epitopes. At least one epitope is derived from a non-spike protein, and the composition induces immunity to only the epitopes. In some embodiments, the epitopes are in the form of a single antigen, e.g., the composition comprises an antigen that comprises: whole spike protein; one or more conserved coronavirus CD4⁺ T cell target epitopes; and one or more conserved coronavirus CD8+ T cell target epitopes; wherein at least one epitope is derived from a non-spike protein, and wherein the composition induces immunity to only the epitopes. In certain embodiments, the epitopes are in the form of two or more antigens.

The present invention also provides a multi-epitope, pan-coronavirus recombinant vaccine composition comprising an antigen delivery system encoding at least two of: at least a portion of spike protein, the portion of spike protein comprising a trimerized SARS-CoV-2 receptor-binding domain (RBD); one or more conserved coronavirus CD4+ T cell target epitopes; and/or one or more conserved coronavirus CD8⁺ T cell target epitopes. At least one epitope is derived from a non-spike protein, and the composition induces immunity to only the epitopes. In some embodiments, the epitopes are in the form of a single antigen, e.g., the composition comprises an antigen that comprises at least two of: at least a portion of spike protein, the portion of spike protein comprising a trimerized SARS-CoV-2 receptor-binding domain (RBD); one or more conserved coronavirus CD4+ T cell target epitopes; and/or one or more conserved coronavirus CD8+ T cell target epitopes; wherein at least one epitope is derived from a non-spike protein, and wherein the composition induces immunity to only the epitopes. In certain embodiments, the epitopes are in the form of two or more antigens.

Likewise, the present invention provides a multi-epitope, pan-coronavirus recombinant vaccine composition comprising an antigen delivery system encoding: at least a portion of spike protein, the portion of spike protein comprising a trimerized SARS-CoV-2 receptor-binding domain (RBD); one or more conserved coronavirus CD4+ T cell target epitopes; and one or more conserved coronavirus CD8⁺ T cell target epitopes. At least one epitope is derived from a non-spike protein, and the composition induces immunity to only the epitopes. In some embodiments, the epitopes are in the form of a single antigen, e.g., the composition comprises an antigen that comprises: at least a portion of spike protein, the portion of spike protein comprising a trimerized SARS-CoV-2 receptor-binding domain (RBD); one or more conserved coronavirus CD4+ T cell target epitopes; and one or more conserved coronavirus CD8+ T cell target epitopes; wherein at least one epitope is derived from a non-spike protein, and wherein the composition induces immunity to only the epitopes. In certain embodiments, the epitopes are in the form of two or more antigens.

Likewise, the present invention includes a multi-epitope, pan-coronavirus recombinant vaccine composition comprising an antigen delivery system encoding one or more conserved coronavirus B-cell target epitopes; one or more conserved coronavirus CD4+ T cell target epitopes: and one or more conserved coronavirus CD8+ T cell target epitopes, wherein at least one epitope is derived from a non-spike protein, and the composition induces immunity to only the epitopes, and wherein a conserved target epitope is one that is among the most highly conserved epitopes identified in a sequence alignment and analysis of a particular number of coronavirus sequences (for the particular type of epitope, e.g., B cell. CD4 T cell, CD8 T cell). For example, the conserved epitopes may be the 5 most highly conserved epitopes identified (for the particular type of epitope). In some embodiments, the conserved epitopes may be the 10 most highly conserved epitopes identified (for the particular type of epitope). In some embodiments, the conserved epitopes may be the 15 most highly conserved epitopes identified (for the particular type of epitope). In some embodiments, the conserved epitopes may be the 20 most highly conserved epitopes identified (for the particular type of epitope). In some embodiments, the conserved epitopes may be the 25 most highly conserved epitopes identified (for the particular type of epitope). In some embodiments, the conserved epitopes may be the 30 most highly conserved epitopes identified (for the particular type of epitope). In some embodiments, the conserved epitopes may be the 40 most highly conserved epitopes identified (for the particular type of epitope). In some embodiments, the conserved epitopes may be the 50 most highly conserved epitopes identified (for the particular type of epitope). The present invention is not limited to the aforementioned thresholds.

Likewise, the present invention includes a multi-epitope, pan-coronavirus recombinant vaccine composition comprising an antigen delivery system encoding one or more conserved coronavirus B-cell target epitopes; one or more conserved coronavirus CD4+ T cell target epitopes; and one or more conserved coronavirus CD8+ T cell target epitopes, wherein at least one epitope is derived from a non-spike protein, and the composition induces immunity to only the epitopes, and wherein the conserved epitopes are identified by performing a sequence alignment and analysis of a particular number of coronavirus sequences to determine sequence similarity or identity amongst the group of analyzed sequences. The conserved epitopes are those that are among the most highly conserved epitopes identified in the analysis (for the particular type of epitope, e.g., B cell, CD4 T cell, CD8 T cell).

The present invention provides a multi-epitope, pan-coronavirus recombinant vaccine composition comprising an antigen delivery system encoding (i) at least two of: one or more conserved coronavirus B-cell target epitopes; one or more conserved coronavirus CD4+ T cell target epitopes; and/or one or more conserved coronavirus CD8+ T cell target epitopes; (ii) a T cell attracting chemokine; and (iii) a composition that promotes T cell proliferation. At least one epitope is derived from a non-spike protein, and the composition induces immunity to only the epitopes. In some embodiments, the epitopes are in the form of a single antigen, e.g., the composition comprises an antigen that comprises at least two of: one or more conserved coronavirus B-cell target epitopes; one or more conserved coronavirus CD4+ T cell target epitopes; and/or one or more conserved coronavirus CD8+ T cell target epitopes, wherein at least one epitope is derived from a non-spike protein, and wherein the composition induces immunity to only the epitopes. In certain embodiments, the epitopes are in the form of two or more antigens.

Likewise, the present invention provides a multi-epitope, pan-coronavirus recombinant vaccine composition comprising an antigen delivery system encoding (i) one or more conserved coronavirus B-cell target epitopes; one or more conserved coronavirus CD4+ T cell target epitopes; and one or more conserved coronavirus CD8+ T cell target epitopes (ii) a T cell attracting chemokine; and (iii) a composition that promotes T cell proliferation. At least one epitope is derived from a non-spike protein, and the composition induces immunity to only the epitopes. In some embodiments, the epitopes are in the form of a single antigen, e.g., the composition comprises an antigen that comprises: one or more conserved coronavirus B-cell target epitopes; one or more conserved coronavirus CD4+ T cell target epitopes; and one or more conserved coronavirus CD8+ T cell target epitopes; wherein at least one epitope is derived from a non-spike protein, and wherein the composition induces immunity to only the epitopes. In certain embodiments, the epitopes are in the form of two or more antigens.

The present invention also provides a multi-epitope, pan-coronavirus recombinant vaccine composition comprising an antigen delivery system encoding (i) at least two of: whole spike protein; one or more conserved coronavirus CD4+ T cell target epitopes; and/or one or more conserved coronavirus CD8+ T cell target epitopes (ii) a T cell attracting chemokine; and (iii) a composition that promotes T cell proliferation. At least one epitope is derived from a non-spike protein, and the composition induces immunity to only the epitopes. In some embodiments, the epitopes are in the form of a single antigen, e.g., the composition comprises an antigen that comprises at least two of: whole spike protein; one or more conserved coronavirus CD4⁺ T cell target epitopes; and/or one or more conserved coronavirus CD8⁺ T cell target epitopes; wherein at least one epitope is derived from a non-spike protein, and wherein the composition induces immunity to only the epitopes. In certain embodiments, the epitopes are in the form of two or more antigens.

Likewise, the present invention provides a multi-epitope, pan-coronavirus recombinant vaccine composition comprising an antigen delivery system encoding (i) whole spike protein; one or more conserved coronavirus CD4+ T cell target epitopes; and one or more conserved coronavirus CD8+ T cell target epitopes (ii) a T cell attracting chemokine; and (iii) a composition that promotes T cell proliferation. At least one epitope is derived from a non-spike protein, and the composition induces immunity to only the epitopes. In some embodiments, the epitopes are in the form of a single antigen, e.g., the composition comprises an antigen that comprises: whole spike protein; one or more conserved coronavirus CD4+ T cell target epitopes; and one or more conserved coronavirus CD8+ T cell target epitopes; wherein at least one epitope is derived from a non-spike protein, and wherein the composition induces immunity to only the epitopes. In certain embodiments, the epitopes are in the form of two or more antigens.

The present invention also provides a multi-epitope, pan-coronavirus recombinant vaccine composition comprising an antigen delivery system encoding (i) at least two of: at least a portion of spike protein, the portion of spike protein comprising a trimerized SARS-CoV-2 receptor-binding domain (RBD); one or more conserved coronavirus CD4+ T cell target epitopes; and/or one or more conserved coronavirus CD8+ T cell target epitopes (ii) a T cell attracting chemokine; and (iii) a composition that promotes T cell proliferation. At least one epitope is derived from a non-spike protein, and the composition induces immunity to only the epitopes. In some embodiments, the epitopes are in the form of a single antigen, e.g., the composition comprises an antigen that comprises at least two of: at least a portion of spike protein, the portion of spike protein comprising a trimerized SARS-CoV-2 receptor-binding domain (RBD); one or more conserved coronavirus CD4+ T cell target epitopes; and/or one or more conserved coronavirus CD8+ T cell target epitopes; wherein at least one epitope is derived from a non-spike protein, and wherein the composition induces immunity to only the epitopes. In certain embodiments, the epitopes are in the form of two or more antigens.

Likewise, the present invention provides a multi-epitope, pan-coronavirus recombinant vaccine composition comprising an antigen delivery system encoding: (i) at least a portion of spike protein, the portion of spike protein comprising a trimerized SARS-CoV-2 receptor-binding domain (RBD); one or more conserved coronavirus CD4+ T cell target epitopes; and one or more conserved coronavirus CD8⁺ T cell target epitopes (ii) a T cell attracting chemokine; and (iii) a composition that promotes T cell proliferation. At least one epitope is derived from a non-spike protein, and the composition induces immunity to only the epitopes. In some embodiments, the epitopes are in the form of a single antigen, e.g., the composition comprises an antigen that comprises: at least a portion of spike protein, the portion of spike protein comprising a trimerized SARS-CoV-2 receptor-binding domain (RBD); one or more conserved coronavirus CD4+ T cell target epitopes; and one or more conserved coronavirus CD8+ T cell target epitopes; wherein at least one epitope is derived from a non-spike protein, and wherein the composition induces immunity to only the epitopes. In certain embodiments, the epitopes are in the form of two or more antigens.

Likewise, the present invention includes a multi-epitope, pan-coronavirus recombinant vaccine composition comprising an antigen delivery system encoding (i) one or more conserved coronavirus B-cell target epitopes; one or more conserved coronavirus CD4+ T cell target epitopes; and one or more conserved coronavirus CD8+ T cell target epitopes(ii) a T cell attracting chemokine; and (iii) a composition that promotes T cell proliferation, wherein at least one epitope is derived from a non-spike protein, and the composition induces immunity to only the epitopes, and wherein a conserved target epitope is one that is among the most highly conserved epitopes identified in a sequence alignment and analysis of a particular number of coronavirus sequences (for the particular type of epitope, e.g., B cell, CD4 T cell, CD8 T cell). For example, the conserved epitopes may be the 5 most highly conserved epitopes identified (for the particular type of epitope). In some embodiments, the conserved epitopes may be the 10 most highly conserved epitopes identified (for the particular type of epitope). In some embodiments, the conserved epitopes may be the 15 most highly conserved epitopes identified (for the particular type of epitope). In some embodiments, the conserved epitopes may be the 20 most highly conserved epitopes identified (for the particular type of epitope). In some embodiments, the conserved epitopes may be the 25 most highly conserved epitopes identified (for the particular type of epitope). In some embodiments, the conserved epitopes may be the 30 most highly conserved epitopes identified (for the particular type of epitope). In some embodiments, the conserved epitopes may be the 40 most highly conserved epitopes identified (for the particular type of epitope). In some embodiments, the conserved epitopes may be the 50 most highly conserved epitopes identified (for the particular type of epitope). The present invention is not limited to the aforementioned thresholds.

Likewise, the present invention includes a multi-epitope, pan-coronavirus recombinant vaccine composition comprising an antigen delivery system encoding (i) one or more conserved coronavirus B-cell target epitopes; one or more conserved coronavirus CD4+ T cell target epitopes; and one or more conserved coronavirus CD8+ T cell target epitopes (ii) a T cell attracting chemokine; and (iii) a composition that promotes T cell proliferation, wherein at least one epitope is derived from a non-spike protein, and the composition induces immunity to only the epitopes, and wherein the conserved epitopes are identified by performing a sequence alignment and analysis of a particular number of coronavirus sequences to determine sequence similarity or identity amongst the group of analyzed sequences. The conserved epitopes are those that are among the most highly conserved epitopes identified in the analysis (for the particular type of epitope, eg., B cell, CD4 T cell, CD8 T cell). For example, the conserved epitopes may be the 5 most highly conserved epitopes identified (for the particular type of epitope). In some embodiments, the conserved epitopes may be the 10 most highly conserved epitopes identified (for the particular type of epitope). In some embodiments, the conserved epitopes may be the 15 most highly conserved epitopes identified (for the particular type of epitope). In some embodiments, the conserved epitopes may be the 20 most highly conserved epitopes identified (for the particular type of epitope). In some embodiments, the conserved epitopes may be the 25 most highly conserved epitopes identified (for the particular type of epitope). In some embodiments, the conserved epitopes may be the 30 most highly conserved epitopes identified (for the particular type of epitope). In some embodiments, the conserved epitopes may be the 40 most highly conserved epitopes identified (for the particular type of epitope). In some embodiments, the conserved epitopes may be the 50 most highly conserved epitopes identified (for the particular type of epitope). The present invention is not limited to the aforementioned thresholds. In some embodiments, the alignment and analysis for 50 or more sequences, 100 or more sequences, 200 or more sequences, 300 or more sequences, 400 or more sequences, 500 or more sequences, 1000 or more sequences, 2000 or more sequences, 3000 or more sequences, 4000 or more sequences, 5000 or more sequences, 10,000 or more sequences, 15,000 or more sequences, more than 15,000 sequences, etc., In some embodiments, the sequences used for alignments may include human and animal sequences. In certain embodiments, the sequences used for alignments include one or more SARS-CoV-2 human strains or variants in current circulation; one or more coronaviruses that has caused a previous human outbreak; one or more coronaviruses isolated from animals selected from a group consisting of bats, pangolins, civet cats, minks, camels, and other animal receptive to coronaviruses; and/or one or more coronaviruses that cause the common cold.

Non-spike proteins include any of the coronavirus proteins otherthan spike, such as but not limited to Envelope protein, Membrane protein, Nucleocapsid protein, ORF1a protein, ORF1ab protein, ORF6 protein, ORF7a protein, ORF7b protein, ORF8 protein, etc.

For the vaccine compositions herein, in certain embodiments, the epitopes are each asymptomatic epitopes. In certain embodiments, the composition lacks symptomatic epitopes.

As discussed herein, the one or more conserved epitopes, e.g., one or more conserved B cell target epitopes, one or more conserved CD4+ T cell target epitopes, one or more CD8⁺ T cell target epitopes, are highly conserved among human and animal coronaviruses.

For any of the embodiments herein, the epitopes that are selected may be those that achieve a particular score in a binding assay (for binding to an HLA molecule, for example.)

In certain embodiments, one or more conserved epitopes, e.g., one or more conserved B cell target epitopes, one or more conserved CD4+ T cell target epitopes, one or more CD8⁺ T cell target epitopes, are derived from at least one of SARS-CoV-2 protein.

In certain embodiments, one or more conserved epitopes, e.g., one or more conserved B cell target epitopes, one or more conserved CD4+ T cell target epitopes, one or more CD8⁺ T cell target epitopes, are derived from one or more of: one or more SARS-CoV-2 human strains or variants in current circulation; one or more coronaviruses that has caused a previous human outbreak; one or more coronaviruses isolated from animals selected from a group consisting of bats, pangolins, civet cats, minks, camels, and other animal receptive to coronaviruses; and/or one or more coronaviruses that cause the common cold.

Examples of SARS-CoV-2 human strains or variants in current circulation include but are not limited to variant B.1.177; variant B.1.160, variant B.1.1.7 (UK), variant P.1 (Japan/Brazil), variant B.1.351 (South Africa), variant B.1.427 (California), variant B.1.429 (California), variant B.1.258; variant B.1.221; variant B.1.367; variant B.1.1.277; variant B.1.1.302; variant B.1.525; variant B.1.526, variant S:677H; variant S:677P; B.1.617.2-Delta, variant B.1.1.529-Omicron (BA.1); sub-variant Omicron (BA.1); sub-variant Omicron (BA.2); sub-variant Omicron (BA.3); sub-variant Omicron (BA.4); sub-variant Omicron (BA.5). Examples of coronaviruses that cause the common cold include 229E alpha coronavirus, NL63 alpha coronavirus, OC43 beta coronavirus, and HKU1 beta coronavirus.

In certain embodiments, one or more conserved epitopes, e.g., one or more conserved B cell target epitopes, one or more conserved CD4+ T cell target epitopes, one or more CD8+ T cell target epitopes, are derived from Variants Of Concern or Variants Of Interest.

The target epitopes, e.g., the one or more conserved B cell target epitopes, one or more conserved CD4+ T cell target epitopes, one or more CD8+ T cell target epitopes, may be derived from structural proteins, non-structural proteins, or a combination thereof. For example, in some embodiments, the one or more conserved B cell target epitopes, one or more conserved CD4+ T cell target epitopes, one or more CD8+ T cell target epitopes, may be derived from a SARS-CoV-2 protein selected from: ORF1ab protein. Spike glycoprotein, ORF3a protein, Envelope protein, Membrane glycoprotein, ORF6 protein, ORF7a protein, ORF7b protein, ORF8 protein. Nucleocapsid protein and ORF10 protein. The ORF1ab protein comprises nonstructural protein (Nsp) 1, Nsp2, Nsp3, Nsp4, Nsp5, Nsp6, Nsp7, Nsp8, Nsp9, Nsp10, Nsp11, Nsp12, Nsp13, Nsp14, Nsp15 and Nsp16.

In some embodiments, the target epitopes, e.g., the one or more conserved B cell target epitopes, one or more conserved CD4+ T cell target epitopes, one or more CD8+ T cell target epitopes, are restricted to human HLA class 1 and 2 haplotypes In some embodiments, the target epitopes, eg, the one or more conserved B cell target epitopes, one or more conserved CD4+ T cell target epitopes, one or more CD8+ T cell target epitopes, are derived from SARS-CoV-2 and restricted to human HLA class 1 and 2 haplotypes. In some embodiments, the target epitopes, e.g., the one or more conserved B cell target epitopes, one or more conserved CD4+ T cell target epitopes, one or more CD8+ T cell target epitopes, are restricted to cat or dog MHC class 1 and 2 haplotypes. In some embodiments, the target epitopes, e.g., the one or more conserved B cell target epitopes, one or more conserved CD4+ T cell target epitopes, one or more CD8+ T cell target epitopes, are derived from SARS-CoV-2 and restricted to cat or dog MHC class 1 and 2 haplotypes.

In some embodiments, a portion of the target epitopes, e.g., the one or more conserved B cell target epitopes, one or more conserved CD4+ T cell target epitopes, one or more CD8+ T cell target epitopes, are restricted to human HLA class 1 and 2 haplotypes. In some embodiments, a portion of the target epitopes, e.g., the one or more conserved B cell target epitopes, one or more conserved CD4+ T cell target epitopes, one or more CD8+ T cell target epitopes, are derived from SARS-CoV-2 and restricted to human HLA class 1 and 2 haplotypes.

In certain embodiments, the composition comprises 2-20 CD8+ T cell target epitopes. In certain embodiments, the composition comprises 2-20 CD4+ T cell target epitopes. In certain embodiments, the composition comprises 2-20 B cell target epitopes.

In certain embodiments, the one or more conserved coronavirus CD8+ T cell target epitopes are selected from: spike glycoprotein, Envelope protein, ORF1ab protein, ORF7a protein, ORF8a protein, ORF10 protein, or a combination thereof. In certain embodiments, the one or more conserved coronavirus CD8+ T cell target epitopes are selected from: S2-10, S1220-1228, S1000-1008, S958-966, E20-28, ORF1ab1675-1683, ORF1ab2363-2371, ORF1ab3013-3021, ORF1ab3183-3191, ORF1ab5470-5478, ORF1ab6749-6757, ORF7b26-34, ORF8a73-81, ORF103-11, and ORF105-13. In certain embodiments, the one or more conserved coronavirus CD8+ T cell target epitopes are selected from SEQ ID NO: 2-29 or SEQ ID NO: 184-203. In certain embodiments, the one or more conserved coronavirus CD8+ T cell target epitopes are selected from SEQ ID NO: 30-57 or SEQ ID NO: 204-224.

In certain embodiments, the one or more conserved coronavirus CD4+ T cell target epitopes are selected from: spike glycoprotein, Envelope protein, Membrane protein, Nucleocapsid protein, ORF1a protein, ORF1ab protein, ORF6 protein, ORF7a protein, ORF7b protein, ORF8 protein, or a combination thereof. In certain embodiments, the one or more conserved coronavirus CD4+ T cell target epitopes are selected from: ORF1a1350-1365, ORF1ab5019-5033, ORF612-26, ORF1ab6088-6102. ORF1ab6420-6434, ORF1a1801-1815, S1-13, E26-40, E20-34. M176-190, N388-403, ORF7a3-17, ORF7a1-15, ORF7b8-22, ORF7a98-112, and ORF81-15. In certain embodiments, the one or more conserved coronavirus CD4+ T cell target epitopes are selected from SEQ ID NO: 58-73 or SEQ ID NO: 225-243. In certain embodiments, the one or more conserved coronavirus CD4+ T cell target epitopes are selected from SEQ ID NO: 74-105 or SEQ ID NO: 244-262.

In certain embodiments, the one or more conserved coronavirus B cell target epitopes are selected from Spike glycoprotein. In certain embodiments, the one or more conserved coronavirus B cell target epitopes are selected from: S287-317, S524-598, S601-640, S802-819, S888-909, S369-393, S440-501, S1133-1172, S329-363, and S13-37. In certain embodiments, the one or more coronavirus B cell target epitopes are selected from SEQ ID NO: 106-116 or SEQ ID NO: 263-270. In certain embodiments, the one or more coronavirus B cell target epitopes are selected from SEQ ID NO: 117-138 or SEQ ID NO: 271-284.

As previously discussed, in certain embodiments, the one or more conserved coronavirus B cell target epitopes are in the form of a large sequence, e.g., whole spike protein or partial spike protein (eg., a portion of whole spike protein). In some embodiments, the whole spike protein or portion thereof is in its stabilized conformation In certain embodiments, the transmembrane anchor of the spike protein (or portion thereof) has an intact S1-S2 cleavage site. In certain embodiments, the spike glycoprotein has two consecutive proline substitutions at amino acid positions 986 and 987, e.g., for stabilization. In certain embodiments, the spike protein or portion thereof has an amino acid substitution at amino acid position Tyr-83. In certain embodiments, the spike protein or portion thereof has an amino acid substitution at amino acid position Tyr-489. In certain embodiments, the spike protein or portion thereof has an amino acid substitution at amino acid position Gln-24. In certain embodiments, the spike protein or portion thereof has an amino acid substitution at amino acid position Asn-487. In certain embodiments, the spike protein or portion thereof has an amino acid substitution at one or more of: Tyr-83, Tyr-489, Gln-24, Gln-493, and Asn-487, e.g., the spike protein or portion thereof may comprise Tyr-489 and Asn-487, the spike protein or portion thereof may comprise Gln-493, the spike protein or portion thereof may comprise Tyr-505, etc. Tyr-489 and Asn-487may help with interaction with Tyr 83 and Gln-24 on ACE-2. Gln-493 may help with interaction with Glu-35 and Lys-31 on ACE-2. Tyr-505 may help with interaction with Glu-37 and Arg-393 on ACE-2.

In certain embodiments, the composition comprises a mutation 682-RRAR-685 → 682-QQAQ-685 in the S1-S2 cleavage site. In certain embodiments, the composition comprises at least one proline substitution. In certain embodiments, the composition comprises at least two proline substitutions, e.g., at position K986 and V987.

In certain embodiments, a target epitope derived from the spike glycoprotein is RBD. In certain embodiments, a target epitope derived from the spike glycoprotein is NTD. In certain embodiments, a target epitope derived from the spike glycoprotein is one or more epitopes, e.g., comprising both the RBD and NTD regions. In certain embodiments, a target epitope derived from the spike glycoprotein is recognized by neutralizing and blocking antibodies. In certain embodiments, a target epitope derived from the spike glycoprotein induces neutralizing and blocking antibodies. In certain embodiments, a target epitope derived from the spike glycoprotein induces neutralizing and blocking antibodies that recognize and neutralize the virus. In certain embodiments, a target epitope derived from the spike glycoprotein induces neutralizing and blocking antibodies that recognize the spike protein.

In certain embodiments, each of the target epitopes are separated by a linker. In certain embodiments, a portion of the target epitopes are separated by a linker. In certain embodiments, the linker is from 2-10 amino acids in length In certain embodiments, the linker is from 3-12 amino acids in length. In certain embodiments, the linker is from 5-15 amino acids in length. In certain embodiments, the linker is 10 or more amino acids in length. Non-limiting examples of linkers include AAY, KK, and GPGPG.

In some embodiments, the composition comprises the addition of a T4 fibritin-derived foldon trimerization domain. In some embodiments, the addition of a T4 fibritin-derived foldon trimerization domain increases immunogenicity by multivalent display.

In certain embodiments, the composition further comprises a T cell attracting chemokine. For example, the composition may further comprise one or a combination of CCL5, CXCL9, CXCL10, CXCL11, or a combination thereof.

In certain embodiments, the composition further comprises a composition that promotes T cell proliferation. For example, the composition may further comprise IL-7, IL-15, IL-2, or a combination thereof.

In certain embodiments, the composition further comprises a molecular adjuvant. For example, the composition may further comprise one or a combination of CpG (e.g., CpG polymer) or flagellin.

In certain embodiments, the composition comprises a tag. For example, the epitopes may be in the form of a single antigen, wherein the composition comprises a tag. In certain embodiments, the epitopes are in the form of two or more antigens, wherein one or more of the antigens comprise a tag. Non-limiting examples of tags include a His tag.

In certain embodiments, the “antigen delivery system” may refer to two delivery systems, e.g., a portion of the epitopes (or other components such as chemokines, etc.) may be encoded by one delivery system and a portion of the epitopes (or other components) may be encoded by a second delivery system (or a third delivery system, etc.).

Referring to the antigen delivery system, in certain embodiments the antigen delivery system is an adeno-associated viral vector-based antigen delivery system. Non-limiting examples include an adeno-associated virus vector type 8 (AAV8 serotype) or an adeno-associated virus vector type 9 (AAV9 serotype). In certain embodiments, the antigen delivery system is a vesicular stomatitis virus (VSV) vector. In certain embodiments, the antigen delivery system is an adenovirus (e.g., Ad26, Ad5, Ad35, etc.)

The target epitopes are operatively linked to a promoter. In certain embodiments, the promoter is a generic promoter (e.g., CMV, CAG, etc.). In certain embodiments, the promoter is a lung-specific promoter (e.g., SpB, CD144). In certain embodiments, all of the target epitopes are operatively linked to the same promoter. In certain embodiments, a portion of the target epitopes are operatively linked to a first promoter and a portion of the target epitopes are operatively linked to a second promoter. In certain embodiments, the target epitopes are operatively linked to two or more promoters, e.g., a portion are operatively linked to a first promoter, a portion are operatively linked to a second promoter, etc. In certain embodiments, the target epitopes are operatively linked to three or more promoters, e.g., a portion is operatively linked to a first promoter, a portion is operatively linked to a second promoter, a portion is operatively linked to a third promoter, etc. In certain embodiments, the first promoter is the same as the second promoter. In certain embodiments the second promoter is different from the first promoter. In certain embodiments, the promoter is a generic promoter (eg., CMV, CAG, etc) In certain embodiments, the promoter is a lung-specific promoter (e.g., SpB, CD144) promoter.

In certain embodiments, the antigen delivery system encodes a T cell attracting chemokine. In certain embodiments, the antigen delivery system encodes a composition that promotes T cell proliferation. In certain embodiments, the antigen delivery system encodes both a T cell attracting chemokine and a composition that promotes T cell proliferation. In certain embodiments, the antigen delivery system encodes a molecular adjuvant. In certain embodiments, the antigen delivery system encodes a T cell attracting chemokine, a composition that promotes T cell proliferation and a molecular adjuvant. In certain embodiments, the antigen delivery system encodes a T cell attracting chemokine and a molecular adjuvant. In some embodiments, the antigen delivery system encodes a composition that promotes T cell proliferation and a molecular adjuvant.

In certain embodiments, the T cell attracting chemokine is CCL5, CXCL9, CXCL10. CXCL11, or a combination thereof. In certain embodiments, the composition that promotes T cell proliferation is IL-7 or IL-15 or IL-2. In some embodiments, the molecular adjuvant is CpG (e.g., CpG polymer), flagellin, etc.).

In certain embodiments, the T cell attracting chemokine is operatively linked to a lung-specific promoter (eg, SpB, CD144). In certain embodiments, the T cell attracting chemokine is operatively linked to a generic promoter (e.g, CMV, CAG, etc.). In certain embodiments, the composition that promotes T cell proliferation is operatively linked to a lung-specific promoter (e.g., SpB, CD144). In certain embodiments, the composition that promotes T cell proliferation is operatively linked to a generic promoter (e.g., CMV, CAG, etc.). In certain embodiments, the molecular adjuvant is operatively linked to a lung-specific promoter (e.g., SpB, CD144). In certain embodiments, the molecular adjuvant is operatively linked to a generic promoter (e.g., CMV, CAG, etc.). In certain embodiments, the T cell attracting chemokine and the composition that promotes T cell proliferation are driven by the same promoter. In certain embodiments, the T cell attracting chemokine and the composition that promotes T cell proliferation are driven by different promoters. In certain embodiments, the molecular adjuvant, the T cell attracting chemokine, and the composition that promotes T cell proliferation are driven by the same promoter. In certain embodiments, the molecular adjuvant, the T cell attracting chemokine, and the composition that promotes T cell proliferation are driven by different promoters. In certain embodiments, the molecular adjuvant and the composition that promotes T cell proliferation are driven by different promoters. In certain embodiments, the molecular adjuvant and the T cell attracting chemokine are driven by different promoters.

In certain embodiments, the T cell attracting chemokine and the composition promoting T cell proliferation are separated by a linker. In certain embodiments, the linker comprises T2A. In certain embodiments, the linker comprises E2A. In certain embodiments, the linker comprises P2A. In certain embodiments, the linker is selected from T2A, E2A, and P2A.

Referring to the antigen delivery system, in certain embodiments, a linker is disposed between each open reading frame. In certain embodiments, a different linker is disposed between each open reading from. In certain embodiments, the same linker may be used between particular open reading frames and a different linker may be used between other open reading frames.

In some embodiments, the vaccine composition is administered using modified RNA, adeno-associated virus, or an adenovirus.

The composition herein may be used to prevent a coronavirus disease in a subject. The composition herein may be used to prevent a coronavirus infection prophylactically in a subject. The composition herein may be used to elicit an immune response in a subject The term “subject” herein may refer to a human, a non-human primate, an animal such as a mouse, rat, cat, dog, other animal that is susceptible to coronavirus infection, or other animal used for preclinical modeling. The composition herein may prolong an immune response induced by the multi-epitope pan-coronavirus recombinant vaccine composition and increase T-cell migration to the lungs. In certain embodiments, the composition induces resident memory T cells (Trm). In some embodiments, the vaccine composition induces efficient and powerful protection against the coronavirus disease or infection. In some embodiments, the vaccine composition induces production of antibodies (Abs), CD4+ T helper (Th1) cells, and CD8+ cytotoxic T-cells (CTL). In some embodiments, the composition that promotes T cell proliferation helps to promote long term immunity. In some embodiments, the T-cell attracting chemokine helps pull T-cells from circulation into the lungs.

In certain embodiments, the composition further comprises a pharmaceutical carrier.

The present invention includes any of the vaccine compositions described herein, e.g., the aforementioned vaccine compositions for delivery with nanoparticles, e.g., lipid nanoparticles. For example, the present invention includes the vaccine compositions herein encapsulated in a lipid nanoparticle.

In some embodiments, the vaccine composition comprises a nucleoside-modified mRNA vaccine composition comprising a vaccine composition as described herein.

The present invention includes the compositions described herein comprising and/or encoding a trimerized SARS-CoV-2 receptor-binding domain (RBD) and one or more highly conserved SARS-CoV-2 sequences selected from structural proteins (e.g., nucleoprotein, etc.) and non-structural protein (e.g., Nsp4, etc.). In some embodiments, the trimerized SARS-CoV-2 receptor-binding domain (RBD) sequence is modified by the addition of a T4 fibritin-derived foldon trimerization domain. In some embodiments, the addition of a T4 fibritin-derived foldon trimerization domain increases immunogenicity by multivalent display.

In certain embodiments, the composition incorporates a good manufacturing practice-grade mRNA drug substance that encodes the trimerized SARS-CoV-2 spike glycoprotein RBD antigen together with the one or more highly conserved structural and non-structural SARS-CoV-2 antigens. In certain embodiments, the sequence for an antigen is GenBank accession number, MN908947.3.

The present invention also features methods of producing multi-epitope, pan-coronavirus recombinant vaccine compositions of the present invention.

For example, in some embodiments, the method comprises selecting at least two of: one or more conserved coronavirus B-cell epitopes; one or more conserved coronavirus CD4+ T cell epitopes; one or more conserved coronavirus CD8+ T cell epitopes. At least one epitope is derived from a non-spike protein, and the composition induces immunity to only the epitopes. The method further comprises synthesizing an antigen or antigens comprising the selected epitopes (or a combination of antigens that collectively comprise the selected epitopes). In some embodiments, the method comprises selecting: one or more conserved coronavirus B-cell epitopes; one or more conserved coronavirus CD4⁺ T cell epitopes; and one or more conserved coronavirus CD8+ T cell epitopes. At least one epitope is derived from a non-spike protein, and the composition induces immunity to only the epitopes. The method further comprises synthesizing an antigen comprising the selected epitopes (or a combination of antigens that collectively comprise the selected epitopes). In some embodiments, the method further comprises introducing the vaccine composition to a pharmaceutical carrier. The steps for selecting the one or more conserved epitopes are disclosed herein. Methods for synthesizing recombinant proteins are well known to one of ordinary skill in the art. The vaccine compositions are disclosed herein. In some embodiments, the vaccine composition is in the form of DNA, RNA, modified RNA, protein (or peptide), or a combination thereof.

In some embodiments, the method comprises selecting: one or more conserved coronavirus B-cell epitopes; one or more conserved coronavirus CD4⁺ T cell epitopes; and one or more conserved coronavirus CD8+ T cell epitopes. At least one epitope is derived from a non-spike protein, and the composition induces immunity to only the epitopes. The method further comprises synthesizing an antigen delivery system encoding the selected epitopes. In some embodiments, the method further comprises introducing the vaccine composition to a pharmaceutical carrier. The steps for selecting the one or more conserved epitopes are disclosed herein. Methods for synthesizing antigen delivery systems are well known to one of ordinary skill in the art. The vaccine compositions are disclosed herein. In some embodiments, the vaccine composition is in the form of DNA, RNA, modified RNA, protein (or peptide), or a combination thereof.

As an example, steps or methods for selecting or identifying conserved epitopes may first include performing a sequence alignment and analysis of a particular number of coronavirus sequences, e.g.. 50 or more sequences, 100 or more sequences, 200 or more sequences, 300 or more sequences, 400 or more sequences, 500 or more sequences, 1000 or more sequences, 2000 or more sequences, 3000 or more sequences, 4000 or more sequences, 5000 or more sequences, 10,000 or more sequences, 15,000 or more sequences, more than 15,000 sequences, etc., to determine sequence similarity or identity amongst the group of analyzed sequences. In some embodiments, the sequences used for alignments may include human and animal sequences. In certain embodiments, the sequences used for alignments include one or more SARS-CoV-2 human strains or variants in current circulation; one or more coronaviruses that has caused a previous human outbreak; one or more coronaviruses isolated from animals selected from a group consisting of bats, pangolins, civet cats, minks, camels, and other animal receptive to coronaviruses; and/or one or more coronaviruses that cause the common cold. In some embodiments, the one or more SARS-CoV-2 human strains or variants in current circulation are selected from: variant B.1.177; variant B.1.160, variant B.1.1.7 (UK), variant P.1 (Japan/Brazil), variant B.1.351 (South Africa), variant B.1.427 (California), variant B.1.429 (California), variant B.1.258; variant B.1.221; variant B.1.367; variant B.1.1.277; variant B.1.1.302; variant B.1.525; variant B.1.526, variant S:677H; variant S:677P; B.1.6172-Delta, variant B.1.1.529-Omicron (BA.1); sub-variant Omicron (BA.1); sub-variant Omicron (BA.2); sub-variant Omicron (BA.3); sub-variant Omicron (BA.4); sub-variant Omicron (BA.5). In some embodiments, the one or more coronaviruses that cause the common cold are selected from: 229E alpha coronavirus, NL63 alpha coronavirus, OC43 beta coronavirus, and HKU1 beta coronavirus. In some embodiments, the conserved CD4+ T cell epitopes may be considered the 5 most highly conserved CD4+ T cell epitopes of the identified epitopes in the alignment. In some embodiments, the conserved CD4+ T cell epitopes may be considered the 10 most highly conserved CD4+ T cell epitopes of the identified epitopes in the alignment. In some embodiments, the conserved CD4+ T cell epitopes may be considered the 15 most highly conserved CD4+ T cell epitopes of the identified epitopes in the alignment. In some embodiments, the conserved CD4+ T cell epitopes may be considered the 20 most highly conserved CD4+ T cell epitopes of the identified epitopes in the alignment. In some embodiments, the conserved CD4+ T cell epitopes may be considered the 25 most highly conserved CD4+ T cell epitopes of the identified epitopes in the alignment. In some embodiments, the conserved CD4⁺ T cell epitopes may be considered the 30 most highly conserved CD4+ T cell epitopes of the identified epitopes in the alignment. In some embodiments, the conserved CD8+ T cell epitopes may be considered the 5 most highly conserved CD8+ T cell epitopes of the identified epitopes in the alignment. In some embodiments, the conserved CD8+ T cell epitopes may be considered the 10 most highly conserved CD8+ T cell epitopes of the identified epitopes in the alignment. In some embodiments, the conserved CD8⁺ T cell epitopes may be considered the 15 most highly conserved CD8+ T cell epitopes of the identified epitopes in the alignment. In some embodiments, the conserved CD8⁺ T cell epitopes may be considered the 20 most highly conserved CD8⁺ T cell epitopes of the identified epitopes in the alignment. In some embodiments, the conserved CD8+ T cell epitopes may be considered the 25 most highly conserved CD8+ T cell epitopes of the identified epitopes in the alignment. In some embodiments, the conserved CD8+ T cell epitopes may be considered the 30 most highly conserved CD8+ T cell epitopes of the identified epitopes in the alignment. In some embodiments, the conserved B cell epitopes may be considered the 5 most highly conserved B cell epitopes of the identified epitopes in the alignment. In some embodiments, the conserved B cell epitopes may be considered the 10 most highly conserved B cell epitopes of the identified epitopes in the alignment. In some embodiments, the conserved B cell epitopes may be considered the 15 most highly conserved B cell epitopes of the identified epitopes in the alignment. In some embodiments, the conserved B cell epitopes may be considered the 20 most highly conserved B cell epitopes of the identified epitopes in the alignment In some embodiments, the conserved B cell epitopes may be considered the 25 most highly conserved B cell epitopes of the identified epitopes in the alignment. In some embodiments, the conserved B cell epitopes may be considered the 30 most highly conserved B cell epitopes of the identified epitopes in the alignment.

The present invention also features methods for preventing coronavirus disease. The method comprises administering to a subject a therapeutically effective amount of a multi-epitope, pan-coronavirus recombinant vaccine composition according to the present invention, wherein the composition elicits an immune response in the subject and helps prevent coronavirus disease.

The present invention also features methods for preventing a coronavirus infection prophylactically in a subject. In some embodiments, the method comprises administering to the subject a prophylactically effective amount of a multi-epitope, pan-coronavirus recombinant vaccine composition according to the present invention, wherein the vaccine composition prevents coronavirus infection.

The present invention also features methods for eliciting an immune response in a subject, including administering to the subject a composition according to the present invention, wherein the vaccine composition elicits an immune response in the subject. The present invention also features methods comprising: administering to a subject a multi-epitope, pan-coronavirus recombinant vaccine composition according to the present invention, wherein the composition prevents virus replication in the lungs, the brain, and other compartments where the virus replicates. The present invention also features methods comprising: administering to the subject a multi-epitope, pan-coronavirus recombinant vaccine composition according to the present invention, wherein the composition prevents cytokine storm in the lungs, the brain, and other compartments where the virus replicates. The present invention also features methods comprising: administering to the subject a multi-epitope, pan-coronavirus recombinant vaccine composition according to the present invention, wherein the composition prevents inflammation or inflammatory response in the lungs, the brain, and other compartments where the virus replicates. The present invention also features methods comprising: administering to the subject a multi-epitope, pan-coronavirus recombinant vaccine composition according to the present invention, wherein the composition improves homing and retention of T cells in the lungs, the brain, and other compartments where the virus replicates. The present invention also features methods for preventing coronavirus disease in a subject; the method comprising: administering to the subject a multi-epitope, pan-coronavirus recombinant vaccine composition according to the present invention, wherein the composition induces memory B and T cells. The present invention also features methods for prolonging an immune response induced by a pan-coronavirus recombinant vaccine and increasing T-cell migration to the lungs, the method comprising: co-expressing a T-cell attracting chemokine, a composition that promotes T cell proliferation, and a pan-coronavirus recombinant vaccine according to the present invention. The present invention also features methods for prolonging the retention of memory T-cell into the lungs induced by a pan coronavirus vaccine and increasing virus-specific tissue resident memory T-cells (TRM cells), the method comprising: co-expressing a T-cell attracting chemokine, a composition that promotes T cell proliferation, and a pan-coronavirus recombinant vaccine according to the present invention. The present invention also features methods comprising: administering to the subject a pan-coronavirus recombinant vaccine composition according to the present invention, wherein the composition prevents the development of mutation and variants of a coronavirus.

For the sake of brevity, it is noted that the vaccine compositions referred to in the aforementioned methods include the vaccine compositions previously discussed, the embodiments described below, and the embodiments in the figures.

In some embodiments, the vaccine composition is administered through an intravenous route (i.v.), an intranasal route (i.n.), or a sublingual route (s.l.) route. In some embodiments, the vaccine composition is administered using modified RNA, adeno-associated virus, or an adenovirus.

As previously discussed, the composition herein may be used to prevent a coronavirus disease in a subject. The composition herein may be used to prevent a coronavirus infection prophylactically in a subject. The composition herein may be used to elicit an immune response in a subject. The term “subject” herein may refer to a human, a non-human primate, an animal such as a mouse, rat, cat, dog, other animal that is susceptible to coronavirus infection, or other animal used for preclinical modeling. The composition herein may prolong an immune response induced by the multi-epitope pan-coronavirus recombinant vaccine composition and increase T-cell migration to the lungs. In certain embodiments, the composition induces resident memory T cells (Trm). In some embodiments, the vaccine composition induces efficient and powerful protection against the coronavirus disease or infection. In some embodiments, the vaccine composition induces production of antibodies (Abs), CD4+ T helper (Th1) cells, and CD8+ cytotoxic T-cells (CTL). In some embodiments, the composition that promotes T cell proliferation helps to promote long term immunity. In some embodiments, the T-cell attracting chemokine helps pull T-cells from circulation into the lungs.

The present invention also features oligonucleotide compositions. For example, the present invention includes oligonucleotides disclosed in the sequence listings The present invention also includes oligonucleotides in the form of antigen delivery systems. The present invention also includes oligonucleotides encoding the conserved epitopes disclosed herein. The present invention also includes oligonucleotide compositions comprising one or more oligonucleotides encoding any of the vaccine compositions according to the present invention. In some embodiments, the oligonucleotide comprises DNA. In some embodiments, the oligonucleotide comprises modified DNA. In some embodiments, the oligonucleotide comprises RNA. In some embodiments, the oligonucleotide comprises modified RNA. In some embodiments, the oligonucleotide comprises mRNA. In some embodiments, the oligonucleotide comprises modified mRNA.

The present invention also features peptide compositions. For example, the present invention includes peptides disclosed in the sequence listings. The present invention also includes peptide compositions comprising any of the vaccine compositions according to the present invention. The present invention also includes peptide compositions comprising any of the conserved epitopes according to the present invention.

For the sake of brevity, it is noted that the vaccine compositions referred to in the aforementioned oligonucleotide and peptide compositions include the vaccine compositions previously discussed, the embodiments described below, and the embodiments in the figures.

The present invention features a multi-epitope, pan-coronavirus recombinant vaccine composition comprising SEQ ID NO: 139. The present invention also features a multi-epitope, pan-coronavirus recombinant vaccine composition comprising SEQ ID NO: 140. The present invention also features a multi-epitope, pan-coronavirus recombinant vaccine composition comprising SEQ ID NO: 141. The present invention also features a multi-epitope, pan-coronavirus recombinant vaccine composition comprising SEQ ID NO: 142. The present invention also features a multi-epitope, pan-coronavirus recombinant vaccine composition comprising SEQ ID NO: 143. The present invention also features a multi-epitope, pan-coronavirus recombinant vaccine composition comprising SEQ ID NO: 144. The present invention also features a multi-epitope, pan-coronavirus recombinant vaccine composition comprising SEQ ID NO: 145. The present invention also features a multi-epitope, pan-coronavirus recombinant vaccine composition comprising SEQ ID NO: 146. The present invention also features a multi-epitope, pan-coronavirus recombinant vaccine composition comprising SEQ ID NO: 147. The present invention also features a multi-epitope, pan-coronavirus recombinant vaccine composition comprising SEQ ID NO: 148. The present invention also features a multi-epitope, pan-coronavirus recombinant vaccine composition comprising SEQ ID NO: 149 The present invention also features a multi-epitope, pan-coronavirus recombinant vaccine composition comprising SEQ ID NO: 150. The present invention also features a multi-epitope, pan-coronavirus recombinant vaccine composition comprising SEQ ID NO: 151. The present invention also features a multi-epitope, pan-coronavirus recombinant vaccine composition comprising SEQ ID NO: 152. The present invention also features a multi-epitope, pan-coronavirus recombinant vaccine composition comprising SEQ ID NO: 153. The present invention also features a multi-epitope, pan-coronavirus recombinant vaccine composition comprising SEQ ID NO: 154. The present invention also features a multi-epitope, pan-coronavirus recombinant vaccine composition comprising SEQ ID NO: 155.

The present invention also features a multi-epitope, pan-coronavirus recombinant vaccine composition comprising a sequence at least 99% identical to SEQ ID NO: 139. The present invention also features a multi-epitope, pan-coronavirus recombinant vaccine composition comprising a sequence at least 99% identical to SEQ ID NO: 140. The present invention also features a multi-epitope, pan-coronavirus recombinant vaccine composition comprising a sequence at least 99% identical to SEQ ID NO: 141. The present invention also features a multi-epitope, pan-coronavirus recombinant vaccine composition comprising a sequence at least 99% identical to SEQ ID NO: 142. The present invention also features a multi-epitope, pan-coronavirus recombinant vaccine composition comprising a sequence at least 99% identical to SEQ ID NO: 143. The present invention also features a multi-epitope, pan-coronavirus recombinant vaccine composition comprising a sequence at least 99% identical to SEQ ID NO: 144. The present invention also features a multi-epitope, pan-coronavirus recombinant vaccine composition comprising a sequence at least 99% identical to SEQ ID NO: 145. The present invention also features a multi-epitope, pan-coronavirus recombinant vaccine composition comprising a sequence at least 99% identical to SEQ ID NO: 146. The present invention also features a multi-epitope, pan-coronavirus recombinant vaccine composition comprising a sequence at least 99% identical to SEQ ID NO: 147. The present invention also features a multi-epitope, pan-coronavirus recombinant vaccine composition comprising a sequence at least 99% identical to SEQ ID NO: 148. The present invention also features a multi-epitope, pan-coronavirus recombinant vaccine composition comprising a sequence at least 99% identical to SEQ ID NO: 149. The present invention also features a multi-epitope, pan-coronavirus recombinant vaccine composition comprising a sequence at least 99% identical to SEQ ID NO: 150. The present invention also features a multi-epitope, pan-coronavirus recombinant vaccine composition comprising a sequence at least 99% identical to SEQ ID NO: 151. The present invention also features a multi-epitope, pan-coronavirus recombinant vaccine composition comprising a sequence at least 99% identical to SEQ ID NO: 152. The present invention also features a multi-epitope, pan-coronavirus recombinant vaccine composition comprising a sequence at least 99% identical to SEQ ID NO: 153 The present invention also features a multi-epitope, pan-coronavirus recombinant vaccine composition comprising a sequence at least 99% identical to SEQ ID NO: 154. The present invention also features a multi-epitope, pan-coronavirus recombinant vaccine composition comprising a sequence at least 99% identical to SEQ ID NO: 155.

The present invention also features a method comprising: administering a first pan-coronavirus recombinant vaccine dose using a first delivery system, and administering a second vaccine dose using a second delivery system, wherein the first and second delivery system are different. In some embodiments, the first delivery system may comprise a RNA, a modified mRNA, or a peptide delivery system. In some embodiments, the second delivery system may comprise a RNA, a modified mRNA, or a peptide delivery system. In some embodiments, the peptide delivery system is an adenovirus or an adeno-associated virus. In some embodiments, the adenovirus delivery system is Ad26, Ad5. Ad35, or a combination thereof. In some embodiments, the adeno-associated delivery system is AAV8 or AAV9. In some embodiments, the peptide delivery system is a vesicular stomatitis virus (VSV) vector. In some embodiments, the second vaccine dose is administered 14 days after the first vaccine dose.

The present invention also features a method comprising: administering a pan-coronavirus recombinant vaccine composition according to the present invention; and administering at least one T-cell attracting chemokine after administering the pan-coronavirus recombinant vaccine composition In some embodiments, the vaccine composition is administered via a RNA, a modified mRNA, or a peptide delivery system. In some embodiments, the T-cell attracting chemokine is administered via a RNA, a modified mRNA, or a peptide delivery system. In some embodiments, the peptide delivery system is an adenovirus or an adeno-associated virus. In some embodiments, the adenovirus delivery system is Ad26, Ad5. Ad35, or a combination thereof.

In some embodiments, the adeno-associated delivery system is AAV8 or AAV9. In some embodiments, the peptide delivery system is a vesicular stomatitis virus (VSV) vector. In some embodiments, the T-cell attracting chemokine is administered 8 days after administering days after the vaccine composition. In some embodiments, the T-cell attracting chemokine is administered 14 days after administering days after the vaccine composition. In some embodiments, the T-cell attracting chemokine is administered 30 days after administering days after the vaccine composition. In some embodiments, the T-cell attracting chemokine is CCL5, CXCL9, CXCL10, CXCL11, or a combination thereof. The present invention also features a method comprising: administering a pan-coronavirus recombinant vaccine composition according to the present invention; administering at least one T-cell attracting chemokine after administering the pan-coronavirus recombinant vaccine composition; and administering at least one cytokine after administering the T-cell attracting chemokine. In some embodiments, the vaccine composition is administered via a RNA, a modified mRNA, or a peptide delivery system. In some embodiments, the T-cell attracting chemokine is administered via a RNA, a modified mRNA, or a peptide delivery system. In some embodiments, the cytokine is administered via a RNA, a modified mRNA, or a peptide delivery system. In some embodiments, the peptide delivery system is an adenovirus or an adeno-associated virus. In some embodiments, the adenovirus delivery system is Ad26, Ad5, Ad35, or a combination thereof. In some embodiments, the adeno-associated delivery system is AAV8 or AAV9. In some embodiments, the peptide delivery system is a vesicular stomatitis virus (VSV) vector. In some embodiments, the T-cell attracting chemokine is administered 14 days after administering the vaccine composition. In some embodiments, the T-cell attracting chemokine is CCL5, CXCL9, CXCL10, CXCL11, or a combination thereof. In some embodiments, the cytokine is administered 10 days after administering the T-cell attracting chemokine. In some embodiments, the cytokine is IL-7, IL-15, IL2 or a combination thereof.

The present invention also features a method comprising: administering a pan-coronavirus recombinant vaccine composition according to the present invention; administering one or more T-cell attracting chemokine after administering the pan-coronavirus recombinant vaccine composition; and administering one or more mucosal chemokine(s). In some embodiments, the vaccine composition is administered using modified RNA, adeno-associated virus, or an adenovirus. In some embodiments, the T-cell attracting chemokine is administered via a RNA, a modified mRNA, or a peptide delivery system. In some embodiments, the mucosal chemokine is administered via a RNA, a modified mRNA, or a peptide delivery system. In some embodiments, the adeno-associated virus is AAV8 or AAV9. In some embodiments, the adenovirus is Ad26, Ad5. Ad35, or a combination thereof. In some embodiments, the T-cell attracting chemokine is administered 14 days after administering the vaccine composition. In some embodiments, the T-cell attracting chemokine is CCL5, CXCL9, CXCL10, CXCL11, or a combination thereof. In some embodiments, the mucosal chemokine is administered 10 days after administering the T-cell attracting chemokine In some embodiments, the mucosal chemokine is CCL25, CCL28, CXCL14, or CXCL17, or a combination thereof.

For the sake of brevity, it is noted that the vaccine compositions referred to in the aforementioned methods include the vaccine compositions previously discussed, the embodiments described below, and the embodiments in the figures.

As previously discussed, in some embodiments, the vaccine compositions are for use in humans. In some embodiments, the vaccine compositions are for use in animals, e.g., cats, dogs, etc. In some embodiments, the vaccine comprises human CXCL-11 and/or human IL-7 (or IL-15, IL-2). In some embodiments, the vaccine composition comprises animal CLCL-11 and/or animal IL-7 (or IL-15, IL-2).

The present invention includes vaccine compositions in the form of a rVSV-panCoV vaccine composition. The present invention includes vaccine compositions in the form of a rAdV-panCoV vaccine composition.

The present invention also includes nucleic acids for use in the vaccine compositions herein. The present invention also includes vectors for use in the vaccine compositions herein. The present invention also includes fusion proteins for use in the vaccine compositions herein. The present invention also includes immunogenic compositions for use in the vaccine compositions herein.

The vaccine compositions herein may be designed to elicit both high levels of virus-blocking and virus-neutralizing antibodies as well as CD4+ T cells and CD8+ T cells in adults 18 to 55 years. The vaccine compositions herein may be designed to elicit both high levels of virus-blocking and virus-neutralizing antibodies as well as CD4+ T cells and CD8+ T cells in adults 55 to 65 years of age. The vaccine compositions herein may be designed to elicit both high levels of virus-blocking and virus-neutralizing antibodies as well as CD4+ T cells and CD8+ T cells in adults 65 to 85 years of age. The vaccine compositions herein may be designed to elicit both high levels of virus-blocking and virus-neutralizing antibodies as well as CD4+ T cells and CD8⁺ T cells in adults 85 to 100 years of age. The vaccine compositions herein may be designed to elicit both high levels of virus-blocking and virus-neutralizing antibodies as well as CD4+ T cells and CD8+ T cells in children 12 to 18 years of age. The vaccine compositions herein may be designed to elicit both high levels of virus-blocking and virus-neutralizing antibodies as well as CD4+ T cells and CD8+ T cells in children under 12 years of age.

The present invention is not limited to vaccine compositions. For example, in certain embodiments, one or more of the conserved epitopes are used for detecting coronavirus and/or diagnosing coronavirus infection.

The present invention also features a multi-epitope, pan-coronavirus recombinant vaccine composition, the composition comprising at least two of: one or more conserved coronavirus B-cell target epitopes selected from SEQ ID NO: 2-57 (S2-10, S1220-1228, S1000-1008, S958-966, E20-28, ORF1ab1675-1683, ORF1ab2363-2371, ORF1ab3013-3021, ORF1ab3183-3191, ORF1ab5470-5478, ORF1ab6749-6757, ORF7b26-34. ORF8a73-81, ORF103-11, and ORF105-13) or SEQ ID NO: 184-224; one or more conserved coronavirus CD4+ T cell target epitopes selected from SEQ ID NO: 58-105 (ORF1a1350-1365, ORF1ab5019-5033, ORF612-26, ORF1ab6088-6102, ORF1ab8420-8434, ORF1a1801-1815, S1-13, E26-40, E20-34, M176-190, N388-403, ORF7a3-17, ORF7a1-15, ORF7b8-22, ORF7a98-112, and ORF81-15), or SEQ ID NO: 225-262; and one or more conserved coronavirus CD8+ T cell target epitopes selected from SEQ ID NO: 106-138 (S287-317, S524-598, S601-640, S802-819. S888-909, S369-393, S440-501, S1133-1172, S329-363, and S13-37), or SEQ ID NO: 263-284; wherein at least one epitope is derived from a non-spike protein; wherein the composition induces immunity to only the epitopes. The present invention also features a multi-epitope, pan-coronavirus recombinant vaccine composition, the composition comprising: one or more conserved coronavirus B-cell target epitopes selected from SEQ ID NO: 2-57 (S2-10, S1220-1228, S1000-1008, S958-966, E20-28, ORF1ab1675-1683, ORF1ab2363-2371, ORF1ab3013-3021, ORF1ab3183-3191, ORF1ab5470-5478, ORF1ab6749-6757, ORF7b26-34, ORF8a73-81, ORF103-11, and ORF105-13), or SEQ ID NO: 184-224; one or more conserved coronavirus CD4+ T cell target epitopes selected from SEQ ID NO: 58-105 (ORF1a1350-1365, ORF1ab5019-5033, ORF612-26, ORF1ab6088-6102, ORF1ab6420-6434, ORF1a1801-1815, S1-13, E26-40, E20-34, M176-190, N388-403, ORF7a3-17, ORF7a1-15, ORF7b8-22, ORF7a98-112, and ORF81-15), or SEQ ID NO: 225-262; and one or more conserved coronavirus CD8+ T cell target epitopes selected from SEQ ID NO: 106-138 (S287-317, S524-598, S601-640, S802-819, S888-909, S369-393, S440-501, S1133-1172, S329-363, and S13-37), or SEQ ID NO: 263-284; wherein at least one epitope is derived from a non-spike protein; wherein the composition induces immunity to only the epitopes.

The present invention also features a multi-epitope, pan-coronavirus recombinant vaccine composition, the composition comprising: one or more conserved coronavirus B-cell target epitopes and one or more conserved coronavirus CD4+ T cell target epitopes, or one or more conserved coronavirus CD8+ T cell target epitopes and one or more conserved coronavirus CD4+ T cell target epitopes, wherein: the one or more conserved coronavirus B-cell target epitopes selected from SEQ ID NO: 2-57 (S2-10, S1220-1228, S1000-1008, S958-966, E20-28, ORF1ab1675-1683, ORF1ab2363-2371, ORF1ab3013-3021. ORF1ab3183-3191, ORF1ab5470-5478, ORF1ab6749-6757, ORF7b26-34, ORF8a73-81, ORF103-11, and ORF105-13), or SEQ ID NO: 184-224; the one or more conserved coronavirus CD4+ T cell target epitopes selected from SEQ ID NO: 58-105 (ORF1a1350-1365, ORF1ab5019-5033, ORF612-26, ORF1ab6088-6102, ORF1ab6420-6434, ORF1a1801-1815, S1-13, E26-40, E20-34, M176-190, N388-403, ORF7a3-17, ORF7a1-15, ORF7b8-22, ORF7a98-112, and ORF81-15.), or SEQ ID NO: 225-262; and the one or more conserved coronavirus CD8+ T cell target epitopes selected from SEQ ID NO: 106-138 (S287-317, S524-598, S601-640, S802-819, S888-909, S369-393, S440-501, S1133-1172, S329-363, and S13-37) or SEQ ID NO: 263-284; wherein at least one epitope is derived from a non-spike protein: wherein the composition induces immunity to only the epitopes.

In some embodiments, the composition comprises 2-20 CD8+ T cell target epitopes. In some embodiments, the composition comprises 2-20 CD4+ T cell target epitopes. In some embodiments, the composition comprises 2-20 B cell target epitopes. In some embodiments, one or more of the epitopes is in the form of a large sequence. In some embodiments, the one or more coronavirus B cell target epitopes is in the form of whole spike protein or partial spike protein In some embodiments, the partial spike protein comprises a trimerized SARS-CoV-2 receptor-binding domain (RBD) In some embodiments, the whole spike protein or partial spike protein has an intact S1-S2 cleavage site. In some embodiments, the spike protein or portion thereof is stabilized with proline substitutions at amino acid positions 986 and 987. In some embodiments, the vaccine composition is for humans. In some embodiments, the vaccine composition is for animals.

The present invention also features a multi-epitope, pan-coronavirus recombinant vaccine composition, the composition comprising at least two of: one or more conserved coronavirus B-cell target epitopes; one or more conserved coronavirus CD4+ T cell target epitopes; and/or one or more conserved coronavirus CD8+ T cell target epitopes; wherein at least one epitope is derived from a non-spike protein; wherein the composition induces immunity to only the epitopes. The present invention also features a multi-epitope, pan-coronavirus recombinant vaccine composition, the composition comprising: one or more conserved coronavirus B-cell target epitopes; one or more conserved coronavirus CD4+ T cell target epitopes; and/or one or more conserved coronavirus CD8+ T cell target epitopes; wherein at least one epitope is derived from a non-spike protein; wherein the composition induces immunity to only the epitopes.

In some embodiments, the one or more conserved epitopes are highly conserved among human and animal coronaviruses. In some embodiments, the conserved epitope is one that is among the most highly conserved epitopes identified in a sequence alignment and analysis of a particular number of coronavirus sequences (for the particular type of epitope, e.g., B cell, CD4 T cell, CD8 T cell). For example, the conserved epitopes may be the 5 most highly conserved epitopes identified (for the particular type of epitope). In some embodiments, the conserved epitopes may be the 10 most highly conserved epitopes identified (for the particular type of epitope). In some embodiments, the conserved epitopes may be the 15 most highly conserved epitopes identified (for the particular type of epitope). In some embodiments, the conserved epitopes may be the 20 most highly conserved epitopes identified (for the particular type of epitope). In some embodiments, the conserved epitopes may be the 25 most highly conserved epitopes identified (for the particular type of epitope). In some embodiments, the conserved epitopes may be the 30 most highly conserved epitopes identified (for the particular type of epitope). In some embodiments, the conserved epitopes may be the 40 most highly conserved epitopes identified (for the particular type of epitope). In some embodiments, the conserved epitopes may be the 50 most highly conserved epitopes identified (for the particular type of epitope). The present invention is not limited to the aforementioned thresholds. In some embodiments, the one or more conserved epitopes are derived from at least one of SARS-CoV-2 protein. In some embodiments, the one or more conserved epitopes are derived from one or more of: one or more SARS-CoV-2 human strains or variants in current circulation; one or more coronaviruses that has caused a previous human outbreak; one or more coronaviruses isolated from animals selected from a group consisting of bats, pangolins, civet cats, minks, camels, and other animal receptive to coronaviruses; or one or more coronaviruses that cause the common cold. In some embodiments, the one or more SARS-CoV-2 human strains or variants in current circulation are selected from: variant B.1.177; variant B.1.160, variant B.1.1.7 (UK), variant P.1 (Japan/Brazil), variant B.1.351 (South Africa), variant B.1.427 (California), variant B.1.429 (California), variant B.1.258; variant B.1.221; variant B.1.367; variant B.1.1.277; variant B.1.1.302; variant B.1.525; variant B 1.526, variant S:677H; variant S:677P; B.1.617.2-Delta, variant B1.1.529-Omicron (BA1); sub-variant Omicron (BA.1); sub-variant Omicron (BA.2); sub-variant Omicron (BA.3): sub-variant Omicron (BA.4); sub-variant Omicron (BA.5). In some embodiments, the one or more coronaviruses that cause the common cold are selected from: 229E alpha coronavirus, NL63 alpha coronavirus, OC43 beta coronavirus, and HKU1 beta coronavirus. In some embodiments, the vaccine composition is for humans. In some embodiments, the vaccine composition is for animals.

The present invention also features a multi-epitope, pan-coronavirus recombinant vaccine composition, the composition comprising an antigen delivery system encoding at least two of: one or more conserved coronavirus B-cell target epitopes selected from SEQ ID NO: 2-57 (S2-10, S1220-1228, S1000-1008, S958-966, E20-28, ORF1ab1675-1683, ORF1ab2363-2371, ORF1ab3013-3021, ORF1ab3183-3191, ORF1ab5470-5478, ORF1ab6749-6757, ORF7b26-34, ORF8a73-81, ORF103-11, and ORF105-13), or SEQ ID NO: 184-224: one or more conserved coronavirus CD4+ T cell target epitopes selected from SEQ ID NO: 58-105 (ORF1a1350-1365, ORF1ab5019-5033, ORF612-26, ORF1ab6088-6102, ORF1ab6420-6434, ORF1a1801-1815, S1-13, E26-40, E20-34, M176-190, N388-403, ORF7a3-17, ORF7a1-15, ORF7b8-22, ORF7a98-112, and ORF81-15), or SEQ ID NO: 225-262; and/or one or more conserved coronavirus CD8+ T cell target epitopes selected from SEQ ID NO: 106-138 (S287-317, S524-598, S601-640, S802-819, S888-909, S369-393, S440-501, S1133-1172, S329-363, and S13-37), or SEQ ID NO: 263-284; wherein at least one epitope is derived from a non-spike protein; wherein the composition induces immunity to only the epitopes. The present invention also features a multi-epitope, pan-coronavirus recombinant vaccine composition, the composition comprising an antigen delivery system encoding: one or more conserved coronavirus B-cell target epitopes selected from SEQ ID NO: 2-57 (S2-10, S1220-1228, S1000-1008, S958-966, E20-28, ORF1ab1675-1683, ORF1ab2363-2371, ORF1ab3013-3021, ORF1ab3183-3191, ORF1ab5470-5478, ORF1ab6749-6757, ORF7b26-34, ORF8a73-81, ORF103-11, and ORF105-13), or SEQ ID NO: 184-224; one or more conserved coronavirus CD4+ T cell target epitopes selected from SEQ ID NO: 58-105 (ORF1a1350-1365, ORF1ab5019-5033. ORF612-26, ORF1ab6088-6102. ORF1ab6420-6434, ORF1a1801-1815, S1-13, E26-40, E20-34, M176-190, N388-403, ORF7a3-17, ORF7a1-15, ORF7b8-22, ORF7a98-112, and ORF81-15), SEQ ID NO: 225-262; and/or one or more conserved coronavirus CD8+ T cell target epitopes selected from SEQ ID NO: 106-138 (S287-317, S524-598, S601-640, S802-819, S888-909, S369-393, S440-501, S1133-1172, S329-363, and S13-37), or SEQ ID NO: 263-284; wherein at least one epitope is derived from a non-spike protein; wherein the composition induces immunity to only the epitopes.

In some embodiments, the antigen delivery system is an adeno-associated viral vector-based antigen delivery system. In some embodiments, the adeno-associated viral vector is an adeno-associated virus vector type 8 (AAV8 serotype) or an adeno-associated virus vector type 9 (AAV9 serotype). In some embodiments, the antigen delivery system is an mRNA delivery system. In some embodiments, the antigen delivery system further encodes a T cell attracting chemokine. In some embodiments, the antigen delivery system further encodes a composition that promotes T cell proliferation. In some embodiments, the antigen delivery system further encodes a molecular adjuvant. In some embodiments, the epitopes are operatively linked to a lung-specific promoter

The present invention also features a multi-epitope, pan-coronavirus recombinant vaccine composition comprising one of SEQ ID NO: 139-155.

The present invention also includes the corresponding nucleic acid sequences for any of the protein sequences herein. The present invention also includes the corresponding protein sequences for any of the nucleic acid sequences herein.

Embodiments herein may comprise whole spike protein or a portion of spike protein. Whole spike protein and a portion thereof is not limited to a wild type or original sequence and may include spike protein or a portion thereof with one or more modifications and/or mutations, such as point mutations, deletions, etc., including the mutations described herein such as those for improving stability.

Embodiments of the present invention can be freely combined with each other if they are not mutually exclusive.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### DESCRIPTION OF THE SEVERAL VIEWS OF THE DRAWING(S)

The features and advantages of the present invention will become apparent from a consideration of the following detailed description presented in connection with the accompanying drawings in which:

FIG. 1 shows a schematic view of an example of a multi-epitope pan-coronavirus recombinant vaccine composition. CD8+ T cell epitopes are shown with a square, CD4+ T cell epitopes are shown with a circle and B-cell epitopes are shown with a diamond. Each shape (square, circle, or diamond) may represent a variety of different epitopes and is not limited to a singular epitope. The multi-epitope pan-coronavirus vaccines are not limited to a specific combination of epitopes as shown. The multi-epitope pan-coronavirus vaccines may comprise a various number of individual CD8+, CD4+, or B cell epitopes.

FIG. 2A shows an evolutionary comparison of genome sequences among beta-Coronavirus strains isolated from humans and animals. A phylogenetic analysis performed between SARS-CoV-2 strain sp (obtained from humans (Homo Sapiens (black)), along with the animal’s SARS-like Coronaviruses genome sequence (SL-CoVs) sequences obtained from bats (Rhinolophus affinis, Rhinolophus malayanus (red)), pangolins (Manis javanica (blue)), civet cats (Paguma larvata (green)), and camels (Camelus dromedarius (Brown)). The included SARS-CoV/MERS-CoV strains are from previous outbreaks (obtained from humans (Urbani, MERS-CoV, OC43, NL63, 229E, HKU1-genotype-B), bats (WIV16, WIV1, YNLF-31C, Rs672, recombinant strains), camel (Camelus dromedarius, (KT368891.1, MN514967.1, KF917527.1, NC_028752.1), and civet (Civet007, A022, B039)). The human SARS-CoV-2 genome sequences are represented from six continents.

FIG. 2B shows an evolutionary analysis performed among the human-SARS-CoV-2 genome sequences reported from six continents and SARS-CoV-2 genome sequences obtained from bats (Rhinolophus affinis, Rhinolophus malayanus), and pangolins (Manis javanica)).

FIG. 3A shows lungs, heart, kidneys, intestines, brain, and testicles express ACE2 receptors and are targeted by SARS-CoV-2 virus. SARS-CoV-2 virus docks on the Angiotensin converting enzyme 2 (ACE2) receptor via spike surface protein.

FIG. 3B shows a System Biology Analysis approach utilized in the present invention.

FIG. 4A shows examples of binding capacities of virus-derived CD4+ T cell epitope peptides to soluble HLA-DR molecules. CD4+ T cell peptides were submitted to ELISA binding assays specific for HLA-DR molecules. Reference non-viral peptides were used to validate each assay. Data are expressed as relative activity (ratio of the IC₅₀ of the peptides to the IC₅₀ of the reference peptide) and are the means of two experiments. Peptide epitopes with high affinity binding to HLA-DR molecules have IC₅₀ below 250 and are indicated in bold. IC₅₀ above 250 indicates peptide epitopes that failed to bind to tested HLA-DR molecules.

FIG. 4B shows an example of potential epitopes binding with high affinity to HLA-A*0201 and stabilizing expression on the surface of target cells: Predicted and measured binding affinity of genome-derived peptide epitopes to soluble HLA-A*0201 molecule (IC₅₀ nM). The binding capacities of a virus CD8 T cell epitope peptide to soluble HLA-A*0201 molecules. CD8 T cell peptides were submitted to ELISA binding assays specific for HLA-A*0201 molecules. Reference non-viral peptides were used to validate each assay. Data are expressed as relative activity (ratio of the IC₅₀ to the peptide to the IC₅₀ of the reference peptide) and are the means of two experiments. Peptide epitopes with high affinity binding to HLA-A*0201 molecules have IC₅₀ below 100 and are indicated in bold. IC₅₀ above 100 indicates peptide epitopes that failed to bind to tested HLA-A*0201 molecules.

FIG. 5 shows a sequence homology analysis to screen conservancy of potential SARS-CoV-2-derived human CD8+ T cell epitopes. Shown are the comparison of sequence homology for the potential CD8+ T cell epitopes among 81,963 SARS-CoV-2 strains (that currently circulate in 190 countries on 6 continents), the 4 major “common cold” Coronaviruses that cased previous outbreaks (i.e. hCoV-OC43, hCoV-229E, hCoV-HKU1-Genotype B, and hCoV-NL63), and the SL-CoVs that were isolated from bats, civet cats, pangolins and camels. Epitope sequences highlighted in yellow present a high degree of homology among the currently circulating 81,963 SARS-CoV-2 strains and at least a 50% conservancy among two or more humans SARS-CoV strains from previous outbreaks, and the SL-CoV strains isolated from bats, civet cats, pangolins and camels, as described herein. Homo Sapiens- black, bats (Rhinolophus affinis, Rhinolophus malayanus-red), pangolins (Manis javanica-blue), civet cats (Paguma larvata-green), and camels (Camelus dromedarius-brown).

FIG. 6A shows docking of highly conserved SARS-CoV-2-derived human CD8+ T cell epitopes to HLA-A*02:01 molecules, e.g., docking of the 27 high-affinity CD8+ T cell binder peptides to the groove of HLA-A*02:01 molecules.

FIG. 6B shows a summary of the interaction similarity scores of the 27 high-affinity CD8+ T cell epitope peptides to HLA-A*02:01 molecules determined by protein-peptide molecular docking analysis. Black columns depict CD8+ T cell epitope peptides with high interaction similarity scores.

FIG. 7A shows an experimental design show CD8+ T cells are specific to highly conserved SARS-CoV-2 epitopes detected in COVID-19 patients and unexposed healthy individuals: PBMCs from HLA-A*02:01 positive COVID-19 patients (n = 30) and controls unexposed healthy individuals (n = 10) were isolated and stimulated overnight with 10 µM of each of the 27 SARS-CoV-2-derived CD8+ T cell epitopes. The number of IFN-γ-producing cells were quantified using ELISpot assay.

FIG. 7B shows the results from FIG. 7A Dotted lines represent a threshold to evaluate the relative magnitude of the response: a mean SFCs between 25 and 50 correspond to a medium/intermediate response whereas a strong response is defined for a mean SFCs > 50.

FIG. 7C shows the results from experiments where PBMCs from HLA-A*02:01 positive COVID-19 patients were further stimulated for an additional 5 hours in the presence of mAbs specific to CD107a and CD107b, and Golgi-plug and Golgi-stop. Tetramers specific to Spike epitopes, CD107a/b and CD69 and TNF-α expression were then measured by FACS. Representative FACS plot showing the frequencies of Tetramer+CD8+ T cells, CD107a/b+CD8+ T cells, CD69+CD8+ T cells and TNF-α +CD8+ T cells following priming with a group of 4 Spike CD8+ T cell epitope peptides. Average frequencies of tetramer+CD8+ T cells, CD107a/b+CD8+ T cells, CD69+CD8+ T cells and TNF-α +CD8+ T cells.

FIG. 8A shows a timeline of immunization and immunological analyses for experiments testing the immunogenicity of genome-wide identified human SARS-CoV-2 CD8+ T epitopes in HLA-A*02:01/HLA-DRB1 double transgenic mice. Eight groups of age-matched HLA-A*02:01 transgenic mice (n = 3) were immunized subcutaneously, on days 0 and 14, with a mixture of four SARS-CoV-2-derived human CD8+ T cell peptide epitopes mixed with PADRE CD4+ T helper epitope, delivered in alum and CpG1826 adjuvants. As a negative control, mice received adjuvants alone (mock-immunized).

FIG. 8B shows the gating strategy used to characterize spleen-derived CD8+ T cells. Lymphocytes were identified by a low forward scatter (FSC) and low side scatter (SSC) gate. Singlets were selected by plotting forward scatter area (FSC-A) vs. forward scatter height (FSC-H). CD8 positive cells were then gated by the expression of CD8 and CD3 markers.

FIG. 8C shows a representative ELISpot image (left panel) and average frequencies (right panel) of IFN-γ-producing cell spots from splenocytes (106 cells/well) stimulated for 48 hours with 10 µM of 10 immunodominant CD8+ T cell peptides and 1 subdominant CD8+ T cell peptide out of the total pool of 27 CD8+ T cell peptides derived from SARS-CoV-2 structural and non-structural proteins. The number on the top of each ELISpot image represents the number of IFN-γ-producing spot forming T cells (SFC) per one million splenocytes.

FIG. 8D shows a representative FACS plot (left panel) and average frequencies (right panel) of IFN-y and TNF-a production by, and CD107a/b and CD69 expression on 10 immunodominant CD8+ T cell peptides and 1 subdominant CD8+ T cell peptide out of the total pool of 27 CD8+ T cell peptides derived from SARS-CoV-2 structural and non-structural proteins determined by FACS. Numbers indicate frequencies of IFN-γ+CD8+ T cells, CD107+CD8+ T cells, CD69+CD8+ T cells and TNF-α +CD8+ T cells, detected in 3 immunized mice.

FIG. 9 shows the SARS-CoV/SARS-CoV-2 genome encodes two large non-structural genes ORF1a (green) and ORF1b (gray), encoding 16 non-structural proteins (NSP1- NSP16). The genome encodes at least six accessory proteins (shades of light grey) that are unique to SARS-CoV/SARS-CoV-2 in terms of number, genomic organization, sequence, and function. The common SARS-CoV, SARS-CoV-2 and SL-CoVs-derived human B (blue), CD4+ (green) and CD8+ (black) T cell epitopes are shown. Structural and non-structural open reading frames utilized in this study were from SARS-CoV-2-Wuhan-Hu-1 strain (NCBI accession number MN908947.3, SEQ ID NO: 1). The amino acid sequence of the SARS-CoV-2-Wuhan-Hu-1 structural and non-structural proteins was screened for human B, CD4+ and CD8+ T cell epitopes using different computational algorithms as described herein. Shown are genome-wide identified SARS-CoV-2 human B cell epitopes (in blue). CD4+ T cell epitopes (in green), CD8+ T cell epitopes (in black) that are highly conserved between human and animal Coronaviruses.

FIG. 10 shows the Identification of highly conserved potential SARS-CoV-2-derived human CD4+ T cell epitopes that bind with high affinity to HLA-DR molecules: Out of a total of 9,594 potential HLA-DR-restricted CD4+ T cell epitopes from the whole genome sequence of SARS-CoV-2-Wuhan-Hu-1 strain (MN908947.3), 16 epitopes that bind with high affinity to HLA-DRB1 molecules were selected. The conservancy of the 16 CD4+ T cell epitopes was analyzed among human and animal Coronaviruses. Shown are the comparison of sequence homology for the 16 CD4+ T cell epitopes among 81,963 SARS-CoV-2 strains (that currently circulate in 6 continents), the 4 major “common cold” Coronaviruses that cased previous outbreaks (i.e. hCoV-OC43, hCoV-229E, hCoV-HKU1, and hCoV-NL63), and the SL-CoVs that were isolated from bats, civet cats, pangolins and camels. Epitope sequences highlighted in green present high degree of homology among the currently circulating 81,963 SARS-CoV-2 strains and at least a 50% conservancy among two or more humans SARS-CoV strains from previous outbreaks, and the SL-CoV strains isolated from bats, civet cats, pangolins and camels, as described in Materials and Methods. Homo Sapiens- black, bats (Rhinolophus affinis, Rhinolophus malayanus -red), pangolins (Manis javanica-blue), civet cats (Paguma larvata-green), and camels (Camelus dromedarius-brown).

FIG. 11A the molecular docking of highly conserved SARS-CoV-2 CD4+ T cell epitopes to HLA-DRB1 molecules. Molecular docking of 16 CD4+ T cell epitopes, conserved among human SARS-CoV-2 strains, previous humans SARS/MERS-CoV and bat SL-CoVs into the groove of the HLA-DRB1 protein crystal structure (PDB accession no: 4UQ3) was determined using the GalaxyPepDock server. The 16 CD4+ T cell epitopes are promiscuous restricted to HLA-DRB1*01:01, HLA-DRB1*11:01, HLA-DRB1*15:01, HLA-DRB1*03:01 and HLA-DRB1*04:01 alleles. The CD4+ T cell peptides are shown in ball and stick structures, and the HLA-DRB1 protein crystal structure is shown as a template. The prediction accuracy is estimated from a linear model as the relationship between the fraction of correctly predicted binding site residues and the template-target similarity measured by the protein structure similarity score (TM score) and interaction similarity score (Sinter) obtained by linear regression. Sinter shows the similarity of the amino acids of the CD8+ T cell peptides aligned to the contacting residues in the amino acids of the HLA-DRB1 template structure.

FIG. 11B shows histograms representing interaction similarity score of CD4+ T cells specific epitopes observed from the protein-peptide molecular docking analysis.

FIG. 12A shows an experimental design to show CD4+ T cells are specific to highly conserved SARS-CoV-2 epitopes detected in COVID-19 patients and unexposed healthy individuals: PBMCs from HLA-DRB1 positive COVID-19 patients (n = 30) and controls unexposed healthy individuals (n = 10) were isolated and stimulated for 48 hrs. with 10 µM of each of the 16 SARS-CoV-2-derived CD4+ T cell epitopes. The number of IFN--producing cells were quantified using ELISpot assay.

FIG. 12B shows the results from FIG. 12A. Dotted lines represent a threshold to evaluate the relative magnitude of the response: a mean SFCs between 25 and 50 correspond to a medium/intermediate response, whereas a strong response is defined for a mean SFCs > 50. PBMCs from HLA-DRB1-positive COVID-19 patients

FIG. 12C shows the results from further stimulating for an additional 5 hours in the presence of mAbs specific to CD107a and CD107b, and Golgi-plug and Golgi-stop. Tetramers specific to two Spike epitopes, CD107a/b and CD69 and TNF-alpha expressions were then measured by FACS. Representative FACS plot showing the frequencies of Tetramer+CD4+ T cells, CD107a/b+CD4+ T cells, CD69+CD4+ T cells and TNF-α +CD4+ T cells following priming with a group of 2 Spike CD4+ T cell epitope peptides Average frequencies are shown for tetramer+CD4+ T cells, CD107a/b+CD4+ T cells, CD69+CD4+ T cells and TNF-α +CD4+ T cells.

FIG. 13A shows a timeline of immunization and immunological analyses for testing immunogenicity of genome-wide identified human SARS-CoV-2 CD4+ T epitopes in HLA-A*02:01/HLA-DRB1 double transgenic mice. Four groups of age-matched HLA-DRB1 transgenic mice (n = 3) were immunized subcutaneously, on days 0 and 14, with a mixture of four SARS-CoV-2-derived human CD4+ T cell peptide epitopes delivered in alum and CpG1826 adjuvants. As a negative control, mice received adjuvants alone (mock-immunized).

FIG. 13B shows the gating strategy used to characterize spleen-derived CD4+ T cells. CD4 positive cells were gated by the CD4 and CD3 expression markers.

FIG. 13C shows the representative ELISpot images (left panel) and average frequencies (right panel) of IFN-γ-producing cell spots from splenocytes (106 cells/well) stimulated for 48 hours with 10 µM of 7 immunodominant CD4+ T cell peptides and 1 subdominant CD4+ T cell peptide out of the total pool of 16 CD4+ T cell peptides derived from SARS-CoV-2 structural and non-structural proteins. The number of IFN-γ-producing spot forming T cells (SFC) per one million of total cells is presented on the top of each ELISpot image.

FIG. 13D shows the representative FACS plot (left panel) and average frequencies (right panel) show IFN-γ and TNF-α-production by, and CD107a/b and CD69 expression on 7 immunodominant CD4+ T cell peptides and 1 subdominant CD4+ T cell peptide out of the total pool of 16 CD4+ T cell peptides derived from SARS-CoV-2 determined by FACS. The numbers indicate percentages of IFN-γ+CD4+ T cells, CD107+CD4+ T cells, CD69+CD4+ T cells and TNF- a+CD4+ T cells detected in 3 immunized mice.

FIG. 14 shows the conservation of Spike-derived B cell epitopes among human, bat, civet cat, pangolin, and camel coronavirus strains: Multiple sequence alignment performed using ClustalW among 29 strains of SARS coronavirus (SARS-CoV) obtained from human, bat, civet, pangolin, and camel. This includes 7 human SARS/MERS-CoV strains (SARS-CoV-2-Wuhan (MN908947.3), SARS-HCoV-Urbani (AY278741.1), CoV-HKU1-Genotype-B (AY884001). CoV-OC43 (KF923903), CoV-NL63 (NC005831), CoV-229E (KY983587), MERS (NC019843)); 8 bat SARS-CoV strains (BAT-SL-CoV-WIV16 (KT444582), BAT-SL-CoV-WlV1 (KF367457.1), BAT-SL-CoV-YNLF31C (KP886808.1), BAT-SARS-CoV-RS672 (FJ588686.1), BAT-CoV-RATG13 (MN996532.1), BAT-CoV-YN01 (EPIISL412976), BAT-CoV-YN02 (EPIISL412977), BAT-CoV-19-ZXC21 (MG772934.1); 3 Civet SARS-CoV strains (SARS-CoV-Civet007 (AY572034.1), SARS-CoV-A022 (AY686863.1), SARS-CoV-B039 (AY686864.1)); 9 pangolin SARS-CoV strains (PCoV-GX-P2V(MT072864.1), PCoV-GX-P5E(MT040336.1), PCoV-GX-P5L (MT040335.1), PCoV-GX-P1E (MT040334.1), PCoV-GX-P4L (MT040333.1), PCoV-MP789 (MT084071.1), PCoV-GX-P3B (MT072865.1), PCoV-Guangdong-P2S (EPIISL410544), PCoV-Guangdong (EPIISL410721)); 4 camel SARS-CoV strains (CamelCoV-HKU23 (KT368891.1), DcCoV-HKU23 (MN514967.1), MERS-CoV-Jeddah (KF917527.1). Riyadh/RY141 (NC028752.1)) and 1 recombinant strain (FJ211859.1)). Regions highlighted with blue color represent the sequence homology. The B cell epitopes, which showed at least 50% conservancy among two or more strains of the SARS Coronavirus or possess receptor-binding domain (RBD) specific amino acids were selected as candidate epitopes.

FIG. 15A shows the docking of SARS-CoV-2 Spike glycoprotein-derived B cell epitopes to human ACE2 receptor, e.g., molecular docking of 22 B-cell epitopes, identified from the SARS-CoV-2 Spike glycoprotein, with ACE2 receptors. B cell epitope peptides are shown in ball and stick structures whereas the ACE2 receptor protein is shown as a template. S471-501 and S369-393 peptide epitopes possess receptor binding domain region specific amino acid residues. The prediction accuracy is estimated from a linear model as the relationship between the fraction of correctly predicted binding site residues and the template-target similarity measured by the protein structure similarity score and interaction similarity score (Sinter) obtained by linear regression. Sinter shows the similarity of amino acids of the B-cell peptides aligned to the contacting residues in the amino acids of the ACE2 template structure. Higher Sinter score represents a more significant binding affinity among the ACE2 molecule and B-cell peptides.

FIG. 15B shows the summary of the interaction similarity score of 22 B cells specific epitopes observed from the protein-peptide molecular docking analysis. B cell epitopes with high interaction similarity scores are indicated in black.

FIG. 16A shows the timeline of immunization and immunological analyses for testing to show IgG antibodies are specific to SARS-CoV-2 Spike protein-derived B-cell epitopes in immunized B6 mice and in convalescent COVID-19 patients. A total of 22 SARS-CoV-2 derived B-cell epitope peptides selected from SARS-CoV-2 Spike protein and tested in B6 mice were able to induce antibody responses. Four groups of age-matched B6 mice (n = 3) were immunized subcutaneously, on days 0 and 14, with a mixture of 4 or 5 SARS-CoV-2 derived B-cell peptide epitopes emulsified in alum and CpG1826 adjuvants. Alum/CpG1826 adjuvants alone were used as negative controls (mock-immunized).

FIG. 16B shows the frequencies of IgG-producing CD3(-)CD138(+)B220(+) plasma B cells were determined in the spleen of immunized mice by flow cytometry. For example, FIG. 16B shows the gating strategy was as follows: Lymphocytes were identified by a low forward scatter (FSC) and low side scatter (SSC) gate. Singlets were selected by plotting forward scatter area (FSC-A) versus forward scatter height (FSC-H). B cells were then gated by the expression of CD3(-) and B220(+) cells and CD138 expression on plasma B cells determined.

FIG. 16C shows the frequencies of IgG-producing CD3(-)CD138(+)B220(+) plasma B cells were determined in the spleen of immunized mice by flow cytometry. For example, FG 15C shows a representative FACS plot (left panels) and average frequencies (right panel) of plasma B cells detected in the spleen of immunized mice. The percentages of plasma CD138(-)B220(+)B cells are indicated on the top left of each dot plot.

FIG. 16D shows SARS-CoV-2 derived B-cell epitopes-specific IgG responses were quantified in immune serum, 14 days post-second immunization (i.e. day 28), by ELISpot (Number of lgG(+)Spots). Representative ELISpot images (left panels) and average frequencies (right panel) of anti-peptide specific IgG-producing B cell spots (1×106 splenocytes/well) following 4 days in vitro B cell polyclonal stimulation with mouse Poly-S (Immunospot). The top/left of each ELISpot image shows the number of IgG-producing B cells per half a million cells. ELISA plates were coated with each individual immunizing peptide

FIG. 16E shows the B-cell epitopes-specific IgG concentrations (µg/mL) measured by ELISA in levels of IgG detected in peptide-immunized B6 mice, after subtraction of the background measured from mock-vaccinated mice. The dashed horizontal line indicates the limit of detection.

FIG. 16F and FIG. 16G show the B-cell epitopes-specific IgG concentrations (µg/mL) measured by ELISA in Level of IgG specific to each of the 22 Spike peptides detected SARS-CoV-2 infected patients (n=40), after subtraction of the background measured from healthy non-exposed individuals (n=10). Black bars and gray bars show high and medium immunogenic B cell peptides, respectively. The dashed horizontal line indicates the limit of detection.

FIG. 17 shows an example of a whole spike protein comprising mutations including 6 proline mutations. The 6 proline mutations comprise single point mutations F817P, A892P, A899P, A942P, K986P and V987P. Additionally, wherein the spike protein or portion thereof comprises a 682-QQAQ-685 mutation of the furin cleavage site for protease resistance. In some embodiments, the K986P and V987P Mutations allow for perfusion stabilization. Note MFVFLVLLPLVSS (SEQ ID NO: 63), ATGTTCGTGTTCCTGGTGCTGCTGCCCCTGGTGAGCAGC (SEQ ID NO: 290), and CAGCAGGCCCAG (SEQ ID NO: 291) are shown in FIG. 17.

FIG. 18 shows a schematic representation of a prototype Coronavirus vaccine of the present invention (SEQ ID NO: 139) This candidate was delivered in ACE2/HLA1/2 triple transgenic mice using 3 different antigen delivery systems: (1) peptides injected subcutaneously; (2) modified mRNA injected subcutaneously; and (3) AAV9 administered intranasally the Virological, Clinical and Immunological results obtained point to an excellent protection against both virus replication in the lungs and COVID-like symptoms (Such as loss of weight), deaths. This protection correlated with an excellent B and T cell immunogenicity of this first multi-epitope pan-Coronavirus vaccine candidate #B1, with antibodies, CD4 T cell and CD8 T cells specific to multiple epitopes encoded by this vaccine were induced and correlated with protection. This candidate was used to immunize mice.

FIG. 19 shows a schematic representation of a prototype Coronavirus vaccine of the present invention; a construct showing a “string-of-pearls” set of CD4+ and CD8+ T cell epitopes expressed as multi-epitopes. The present invention is not limited to the prototype coronavirus vaccines as shown.

FIG. 20 shows schematic views of non-limiting examples of vaccine compositions showing an optional molecular adjuvant, T cell attracting chemokine, and/or composition for promoting T cell proliferation, as well as non-limiting examples of orientations of said optional molecular adjuvant. T cell attracting chemokine, and/or composition for promoting T cell proliferation.

FIG. 21 shows a non-limiting example of an adeno-associated virus vector comprising a multi-epitope pan-coronavirus vaccine composition operably linked to a lung specific promoter (e.g. SP-B promoter or a CD144 promoter). Additionally, the multi-epitope pan-coronavirus vaccine composition comprises a His tag. The adeno-associated virus vector also comprises an adjuvant (e.g. CpG) operable linked to a lung specific promoter (eg. SP-B promoter or a CD144 promoter).

FIG. 22 shows a non-limiting example of an adeno-associated virus vector comprising a multi-epitope pan-coronavirus vaccine composition operably linked to a lung specific promoter (e.g., s SP-B promoter or a CD144 promoter). Additionally, the multi-epitope pan-coronavirus vaccine composition comprises a His tag. The adeno-associated virus vector also comprises an adjuvant (e.g., flagellin) operable linked to a second lung specific promoter (e.g. SP-B promoter or a CD144 promoter).

FIG. 23 shows a non-limiting example of an adeno-associated virus vector comprising a multi-epitope pan-coronavirus vaccine composition operably linked to a generic promoter (e.g. a CMV promoter or a CAG promoter). Additionally, the multi-epitope pan-coronavirus vaccine composition comprises a His tag. The adeno-associated virus vector also comprises at least one T cell enhancement composition (e.g IL-7, or CXCL11) operably linked to a second generic promoter (e.g. a CMV promoter or a CAG promoter). The additional T-cell enhancement composition improves the immunogenicity and long-term memory of the multi-epitope pan-coronavirus vaccine composition by co-expressing IL-7 cytokine and T-cell attracting chemokine CXCL11, both driven with another CMV promoter and linked with a T2A spacer in AAV9 vector.

FIG. 24 shows a non-limiting example of an adeno-associated virus vector comprising a multi-epitope pan-coronavirus vaccine composition operably linked to a generic promoter (eg a CMV promoter or a CAG promoter). Additionally, the multi-epitope pan-coronavirus vaccine composition comprises a His tag and at least one T cell enhancement composition (e.g. IL-7, or CXCL11). to improve the immunogenicity and long-term memory the multi-epitope pan-coronavirus vaccine composition is driven with a single CMV promoter and co-expressed in AAV9 vector with IL-7 cytokine and T-cell attracting chemokine CXCL11 driven with same CMV promoter and linked with a T2A spacer.

FIG. 25 shows non-limiting examples of how the target epitopes of the compositions described herein may be arranged. In addition to a string of epitopes (i.e. “string-of-peals”), the composition of the present invention may also feature a spike protein or portion thereof in combination with target epitopes

FIG. 26A shows an non-limiting example of a method of vaccinating mice to test safety, immunogenicity, and protective efficacy of the vaccine compositions described herein. The vaccine may be delivered using mRNA, peptides, or an adenovirus delivery system. A vaccine candidate composition is given to HLA-ACE-2 mice one day 0, 14 days later a second dose of the vaccine is given to the mice. In some embodiments, the second dose of the vaccine may be given using the same delivery system (e.g. mRNA, peptide, or adenovirus). In other embodiments, the second dose of the vaccine is given using a different delivery system. Ten days after the second dose the mice are exposed to SARS-CoV-2. Post-infection virus-load, weight loss, and death are measured and recorded for vaccinated and unvaccinated mice.

FIG. 26B shows virus load detected in the lungs of vaccinated (SEQ ID NO: 139) and unvaccinated mice using two different vaccine delivery systems adenovirus (left) and peptide (right). ACE-2 mice that were vaccinated showed significantly lower SARS-CoV-2 particles (WA-USA strain) detected in the lungs compared to mock-vaccinated ACE-2 mice between days 6-8 post-infections.

FIG. 26C shows virus load detected in the brains of vaccinated (SEQ ID NO: 139) and unvaccinated mice using two different vaccine delivery systems adenovirus (left) and peptide (right). ACE-2 mice that were vaccinated showed significantly lower SARS-CoV-2 particles (WA-USA strain) detected in the brains compared to mock-vaccinated ACE-2 mice between days 6-8 post-infections

FIG. 27A shows the average weight loss in SAR-CoV-2 infected ACE2 mice following immunization with a multi-epitope pan-coronavirus vaccine (SEQ ID NO: 139) delivered as a adenovirus (AAV9), a peptide, or an mRNA. ACE-2 mice that were vaccinated, regardless of the delivery system, maintained their average body weight after exposure to SARS-CoV-2 compared to the mock-vaccinated ACE-2 mice.

FIG. 27B shows the average survival of SAR-CoV-2 infected ACE2 mice. ACE-2 mice that were vaccinated, with either an adenovirus or peptide vaccine, had a higher survival rate compared to mock-vaccinated ACE-2 mice

FIG. 28A shows that the multi-epitope pan-coronavirus (SEQ ID NO: 139) induces SARS-CoV-2 specific antibody response that correlates with protection in ACE-2 transgenic mice.

FIG. 28B shows the multi-epitope pan-coronavirus vaccine (SEQ ID NO: 139) is able to induce SARS-CoV-2 specific CD 8 T cell response that correlates with protection in ACE-2 transgenic mice.

FIG. 28C shows the multi-epitope pan-coronavirus vaccine (SEQ ID NO: 139) is able to induce SARS-CoV-2 specific CD 4 T cell response that correlates with protection in ACE-2 transgenic mice.

FIG. 29A shows microscopic pathological observation from H&E staining of mouse lungs (n=3) infected with SARS CoV2 immunized with AAV8-SpB and peptide vaccines (SEQ ID NO: 139) prior to infection. AAV8-SpB and peptide immunized mice show protection from pulmonary pathological changes when infected with SARS-CoV2. Hollow arrows indicate proteinaceous exudates in alveolar space, black arrows indicate cellular debris (lymphocytes and red blood cells) in air spaces. The top rows of images show less pathological changes (clear alveolar airspace, less inflammation) in AAV8 vaccinated SARS-CoV2 challenged mice. The middle row shows reduced pathological changes (clear alveolar airspace, less inflammation) in peptide-vaccinated SARS-CoV2 challenged mice. The bottom row shows Increased pathological changes (inflamed alveolar airspace) in non-vaccinated SARS-CoV2 challenged mice.

FIG. 29B shows microscopic pathological observation from CD3 staining of mouse lungs (n=3) infected with SARS CoV2 immunized with AAV8-SpB and peptide vaccines (SEQ ID NO: 139) The top row shows CD3 T cells lining alveoli epithelial cells in AAV8 vaccinated mice. The middle row shows CD3 T cells lining alveoli epithelial cells in AAV8 vaccinated mice. The bottom row shows CD3 T cells found in the inflamed alveolar airspace of non-vaccinated mice.

FIG. 30A shows a non-limiting example of a method for delivering the vaccine composition described herein using a “prime/pull” regimen in humans. The method comprises administering a pan-coronavirus recombinant vaccine composition and further administering at least one T-cell attracting chemokine (e.g. CXCL11) after administering the pan-coronavirus recombinant vaccine composition.

FIG. 30B shows a non-limiting example of a method for delivering the vaccine composition described herein using a “prime/boost” regimen in humans. The method comprises administering a first composition, e.g., a first pan-coronavirus recombinant vaccine composition dose using a first delivery system and further administering a second composition, e.g., a second vaccine composition dose using a second delivery system. In some embodiments, the first delivery system and the second delivery system are different.

FIG. 30C shows a non-limiting example of a method for delivering the vaccine composition described herein using a “prime/pull/keep” regimen in humans to increase the size and maintenance of lung-resident B-cells, CD4+ T cells and CD8+ T cells to protect against SARS-CoV-2. The method comprises administering a pan-coronavirus recombinant vaccine composition and administering at least one T-cell attracting chemokine (e.g. CXCL11 or CXCL17) after administering the pan-coronavirus recombinant vaccine composition.

FIG. 30D shows a non-limiting example of a method for delivering the vaccine composition described herein using a “prime/pull/boost” regimen in humans to increase the size and maintenance of lung-resident B-cells, CD4+ T cells and CD8+ T cells to protect against SARS-CoV-2. The method comprises administering a pan-coronavirus recombinant vaccine composition and administering at least one T-cell attracting chemokine (e.g. CXCL11 or CXCL17) after administering the pan-coronavirus recombinant vaccine composition. The method further comprises administering at least one cytokine after administering the T-cell attracting chemokine (e.g. IL-7, IL-5, or IL-2).

FIG. 31A shows a non-limiting example of a method for delivering the vaccine composition described herein using a “prime/pull” regimen in domestic animals (e.g. cats or dogs). The method comprises administering a pan-coronavirus recombinant vaccine composition and further administering at least one T-cell attracting chemokine (e.g. CXCL11) after administering the pan-coronavirus recombinant vaccine composition.

FIG. 31B shows a non-limiting example of a method for delivering the vaccine composition described herein using a “prime/boost” regimen in domestic animals (e.g. cats or dogs). The method comprises administering a first composition, e.g., a first pan-coronavirus recombinant vaccine composition dose using a first delivery system and further administering a second composition, e.g., a second vaccine composition dose using a second delivery system. In some embodiments, the first delivery system and the second delivery system are different.

FIG. 31C shows a non-limiting example of a method for delivering the vaccine composition described herein using a “prime/pull/keep” regimen in domestic animals (e.g. cats or dogs) to increase the size and maintenance of lung-resident B-cells, CD4+ T cells and CD8+ T cells to protect against SARS-CoV-2. The method comprises administering a pan-coronavirus recombinant vaccine composition and administering at least one T-cell attracting chemokine (e.g. CXCL11 or CXCL17) after administering the pan-coronavirus recombinant vaccine composition.

FIG. 31D shows a non-limiting example of a method for delivering the vaccine composition described herein using a “prime/pull/boost” regimen in domestic animals (e.g. cats or dogs) to increase the size and maintenance of lung-resident B-cells, CD4+ T cells and CD8+ T cells to protect against SARS-CoV-2. The method comprises administering a pan-coronavirus recombinant vaccine composition and administering at least one T-cell attracting chemokine (e.g. CXCL11 or CXCL17) after administering the pan-coronavirus recombinant vaccine composition. The method further comprises administering at least one cytokine after administering the T-cell attracting chemokine (e.g. IL-7, IL-5, or IL-2).

FIG. 32A shows predicted population coverage (PPC) value of human CD8+ T cell epitopes

FIG. 32B shows predicted population coverage (PPC) value of human CD4+ T cell epitopes

FIG. 32C shows predicted population coverage (PPC) value of human CD8+ T cell epitopes in Pan-Coronavirus Vaccine candidate (SEQ ID NO: 139).

FIG. 32D shows predicted population coverage (PPC) value of human CD4+ T cell epitopes in Pan-Coronavirus Vaccine candidate (SEQ ID NO: 139).

FIG. 33A shows identification of highly conserved potential SARS-CoV-2-derived human CD8+ T cell epitopes that bind with high affinity to HLA-A*02:01 molecules: ninety-one, genome-wide In-silico predicted, and highly conserved SARS-CoV-2-derived CD8+ T cell epitope peptides were synthetized and were tested for their binding affinity in vitro to HLA-A*02:01 molecules expressed on the surface of T2 cells.

FIG. 33B shows identification of highly conserved potential SARS-CoV-2-derived human CD8+ T cell epitopes that bind with high affinity to HLA-A*02:01 molecules. Out of the 91 CD8+ T cell epitopes, 4 epitopes were selected as high binders s to HLA-A*02:01 molecules, even at the lowest molarity of 3 uM. Further, 20 epitopes with high and 3 epitopes with moderate binding affinity found to stabilize the expression of HLA- A*02:01 molecules on the surface of the T2 cells. The levels of HLA-A*02:01 surface expression was determined by mean fluorescence intensity (MFI), measured by flow cytometry on T2 cells following an overnight incubation of T2 cells at 26° C. with decreasing peptide epitopes molarity (30, 15 and 5 µM) as shown in graphs. Percent MFI increase was calculated as follows: Percent MFI increase = (MFI with the given peptide - MFI without peptide) / (MFI without peptide) X 100.

FIGS. 34A-34C show screening for the CD8+ T cell, CD4+ T cell, and B-cell epitopes against highly transmissible variants of SARS-CoV-2: Keeping in mind the high degree of transmissibility of SARS-CoV-2 variants namely, Lineage B.1.1.7 from United Kingdom(variant 20I/501Y.V1), Lineage B.1.351 from South Africa(variant 20H/501Y.V2), Lineage B.1.1.28 from Brazil(P.1 variant 20J/501Y.V3), CAL.20C variant from California, and Spike protein mutation D614G; it is of importance to evaluate whether our screened epitopes are conserved for these variants or not, which in turn will ascertain the immunogenicity/antigenicity of our candidate epitopes. Results show (FIG. 34A) 26 out of 27 CD8+ T cell epitopes, and (FIG. 34B) 15 out of 16 CD4+ T cell epitopes are 100% conserved against all the higher transmissible variants. (FIG. 34C) Similarly, 8 B-cell epitopes showed 100% conservancy against all the highly pathogenic SARS-CoV-2 variants.

FIG. 35 shows the time course for therapeutic COVID-19 vaccine in SARS-CoV-2 infected HLA-DR/HLA-A*0201/hACE2 triple transgenic mice.

FIG. 36 shows the results of a sequence alignment of various influenza viruses and variants and the resulting conserved region.

FIG. 37 shows non-limiting examples of recombinant hybrid vaccine compositions described herein. The proteins may be covalently or non-covalently linked together for administration of the vaccine composition.

### TERMS

Unless otherwise explained, all technical and scientific terms used herein have the same meaning as commonly understood by one of ordinary skill in the art to which a disclosed invention belongs. The singular terms “a,” “an,” and “the” include plural referents unless context clearly indicates otherwise. Similarly, the word “or” is intended to include “and” unless the context clearly indicates otherwise. The term “comprising” means that other elements can also be present in addition to the defined elements presented. The use of “comprising” indicates inclusion rather than limitation. Stated another way, the term “comprising” means “including principally, but not necessary solely”. Furthermore, variation of the word “comprising”, such as “comprise” and “comprises”, have correspondingly the same meanings. In one respect, the technology described herein related to the herein described compositions, methods, and respective component(s) thereof, as essential to the invention, yet open to the inclusion of unspecified elements, essential or not (“comprising”).

Suitable methods and materials for the practice and/or testing of embodiments of the disclosure are described below. Such methods and materials are illustrative only and are not intended to be limiting. Other methods and materials similar or equivalent to those described herein can be used. For example, conventional methods well known in the art to which the disclosure pertains are described in various general and more specific references, including, for example. Sambrook et al., Molecular Cloning: A Laboratory Manual, 2d ed., Cold Spring Harbor Laboratory Press, 1989; Sambrook et al., Molecular Cloning: A Laboratory Manual, 3d ed., Cold Spring Harbor Press, 2001; Ausubel et al., Current Protocols in Molecular Biology, Greene Publishing Associates, 1992 (and Supplements to 2000); Ausubel et al., Short Protocols in Molecular Biology: A Compendium of Methods from Current Protocols in Molecular Biology, 4th ed., Wiley & Sons, 1999; Harlow and Lane, Antibodies: A Laboratory Manual, Cold Spring Harbor Laboratory Press, 1990; and Harlow and Lane, Using Antibodies: A Laboratory Manual, Cold Spring Harbor Laboratory Press, 1999, Gene Expression Technology (Methods in Enzymology, Vol. 185, edited by D Goeddel, 1991. Academic Press, San Diego, Calif.), “Guide to Protein Purification” in Methods in Enzymology (M. P. Deutshcer, ed., (1990) Academic Press, Inc.); PCR Protocols: A Guide to Methods and Applications (Innis, et al. 1990. Academic Press, San Diego, Calif.), Culture of Animal Cells: A Manual of Basic Technique, 2^{nd} Ed. (R. I. Freshney. 1987. Liss, Inc. New York, N.Y.), Gene Transfer and Expression Protocols, pp. 109-128, ed. E. J. Murray, The Humana Press Inc., Clifton, N.J.), and the Ambion 1998 Catalog (Ambion, Austin, Tex.), the disclosures of which are incorporated in their entirety herein by reference.

Although methods and materials similar or equivalent to those described herein can be used to practice or test the disclosed technology, suitable methods and materials are described below. The materials, methods, and examples are illustrative only and not intended to be limiting.

As used herein, the terms “immunogenic protein, polypeptide, or peptide” or “antigen” refer to polypeptides or other molecules (or combinations of polypeptides and other molecules) that are immunologically active in the sense that once administered to the host, it is able to evoke an immune response of the humoral and/or cellular type directed against the protein. In embodiments, the protein fragment has substantially the same immunological activity as the total protein. Thus, a protein fragment according to the disclosure can comprise or consist essentially of or consist of at least one epitope or antigenic determinant. An “immunogenic” protein or polypeptide, as used herein, may include the full-length sequence of the protein, analogs thereof, or immunogenic fragments thereof. “Immunogenic fragment” refers to a fragment of a protein which includes one or more epitopes and thus elicits the immunological response described above.

Synthetic antigens are also included within the definition, for example, poly-epitopes, flanking epitopes, and other recombinant or synthetically derived antigens. Immunogenic fragments for purposes of the disclosure may feature at least about 1 amino acid, at least about 3 amino acids, at least about 5 amino acids, at least about 10-15 amino acids, or about 15-25 amino acids or more amino acids, of the molecule. There is no critical upper limit to the length of the fragment, which could comprise nearly the full-length of the protein sequence, or the full-length of the protein sequence, or even a fusion protein comprising at least one epitope of the protein.

As used herein, the term “epitope” refers to the site on an antigen or hapten to which specific B cells and/or T cells respond. The term is also used interchangeably with “antigenic determinant” or “antigenic determinant site”. Antibodies that recognize the same epitope can be identified in a simple immunoassay showing the ability of one antibody to block the binding of another antibody to a target antigen.

As used herein, the term “immunological response” to a composition or vaccine refers to the development in the host of a cellular and/or antibody-mediated immune response to a composition or vaccine of interest. Usually, an “immunological response” includes but is not limited to one or more of the following effects: the production of antibodies, B cells, helper T cells, and/or cytotoxic T cells, directed specifically to an antigen or antigens included in the composition or vaccine of interest. The host may display either a therapeutic or protective immunological response so resistance to new infection will be enhanced and/or the clinical severity of the disease reduced. Such protection will be demonstrated by either a reduction or lack of symptoms normally displayed by an infected host, a quicker recovery time and/or a lowered viral titer in the infected host.

As used herein, the term “variant” refers to a substantially similar sequence. For polynucleotides, a variant comprises a deletion and/or addition and/or change of one or more nucleotides at one or more sites within the native polynucleotide and/or a substitution of one or more nucleotides at one or more sites in the native polynucleotide. As used herein, a “native” polynucleotide or polypeptide comprises a naturally occurring nucleotide sequence or an amino acid sequence, respectively. Variants of a particular polynucleotide of the disclosure (e.g., the reference polynucleotide) can also be evaluated by comparison of the percent sequence identity between the polypeptide encoded by a variant polynucleotide and the polypeptide encoded by the reference polynucleotide “Variant” protein is intended to mean a protein derived from the native protein by deletion or addition of one or more amino acids at one or more sites in the native protein and/or substitution of one or more amino acids at one or more sites in the native protein. Variant proteins encompassed by the present disclosure are biologically active, that is they have the ability to elicit an immune response.

The HLA-DR/HLA-A*0201/hACE2 triple transgenic mouse model referred to herein is a novel susceptible animal model for pre-clinical testing of human COVID-19 vaccine candidates derived from crossing ACE2 transgenic mice with the unique HLA-DR/HLA-A*0201 double transgenic mice. ACE2 transgenic mice are a hACE2 transgenic mouse model expressing human ACE2 receptors in the lung, heart, kidney and intestine (Jackson Laboratory, Bar Harbor, ME). The HLA-DR/HLA-A*0201 double transgenic mice are “humanized” HLA double transgenic mice expressing Human Leukocyte Antigen HLA-A*0201 class I and HLA DR*0101 class II in place of the corresponding mouse MHC molecules (which are knocked out). The HLA-A*0201 haplotype was chosen because it is highly represented (> 50%) in the human population, regardless of race or ethnicity. The HLA-DR/HLA-A*0201/hACE2 triple transgenic mouse model is a “humanized” transgenic mouse model and has three advantages: (1) it is susceptible to human SARS-CoV2 infection; (2) it develops symptoms similar to those seen in COVID-19 in humans; and (3) it develops CD4⁺ T cells and CD8⁺ T cells response to human epitopes. The novel HLA-DR/HLA-A*0201/hACE2 triple transgenic mouse model of the present invention may be used in the pre-clinical testing of safety, immunogenicity and protective efficacy of the human multi-epitope COVID-19 vaccine candidates of the present invention.

As used herein, the terms “treat” or “treatment” or “treating” refers to both therapeutic treatment and prophylactic or preventative measures, wherein the object is to prevent or slow the development of the disease, such as slow down the development of a disorder, or reducing at least one adverse effect or symptom of a condition, disease or disorder, e.g., any disorder characterized by insufficient or undesired organ or tissue function. Treatment is generally “effective” if one or more symptoms or clinical markers are reduced as that term is defined herein. Alternatively, a treatment is “effective” if the progression of a disease is reduced or halted. That is, “treatment” includes not just the improvement of symptoms or decrease of markers of the disease, but also a cessation or slowing of progress or worsening of a symptom that would be expected in absence of treatment. Beneficial or desired clinical results include, but are not limited to, alleviation of one or more symptom(s), diminishment of extent of disease, stabilized (e.g., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. “Treatment” also includes ameliorating a disease, lessening the severity of its complications, preventing it from manifesting, preventing it from recurring, merely preventing it from worsening, mitigating an inflammatory response included therein, or a therapeutic effort to affect any of the aforementioned, even if such therapeutic effort is ultimately unsuccessful.

As used herein, the term “carrier” or “pharmaceutically acceptable carrier” or “pharmaceutically acceptable vehicle” refers to any appropriate or useful carrier or vehicle for introducing a composition to a subject. Pharmaceutically acceptable carriers or vehicles may be conventional but are not limited to conventional vehicles For example, E. W. Martin, Remington’s Pharmaceutical Sciences, Mack Publishing Co., Easton, PA, 15th Edition (1975) and D. B. Troy, ed. Remington: The Science and Practice of Pharmacy, Lippincott Williams & Wilkins, Baltimore MD and Philadelphia, PA, 21^{st} Edition (2006) describe compositions and formulations suitable for pharmaceutical delivery of one or more therapeutic compounds or molecules. Carriers (e.g., pharmaceutical carriers, pharmaceutical vehicles, pharmaceutical compositions, pharmaceutical molecules, etc.) are materials generally known to deliver molecules, proteins, cells and/or drugs and/or other appropriate material into the body. In general, the nature of the carrier will depend on the nature of the composition being delivered as well as the particular mode of administration being employed. In addition to biologically-neutral carriers, pharmaceutical compositions administered may contain minor amounts of non- toxic auxiliary substances, such as wetting or emulsifying agents, preservatives, and pH buffering agents and the like. Patents that describe pharmaceutical carriers include, but are not limited to: U.S. Pat. No. 6,667,371; U.S. Patent No. 6,613,355; U.S. Pat. No. 6,596,296; U.S. Pat. No. 6,413,536; U.S. Pat. No. 5,968,543; U.S. Pat. No. 4,079,038; U.S. Pat. No. 4,093,709; U.S. Pat. No. 4,131,648; U.S. Pat. No. 4,138,344; U.S. Pat. No. 4,180,646; U.S. Pat. No. 4,304,767; U.S. Pat. No. 4,946,931, the disclosures of which are incorporated in their entirety by reference herein. The carrier may, for example, be solid, liquid (e.g., a solution), foam, a gel, the like, or a combination thereof. In some embodiments, the carrier comprises a biological matrix (e.g., biological fibers, etc.). In some embodiments, the carrier comprises a synthetic matrix (e.g., synthetic fibers, etc.). In certain embodiments, a portion of the carrier may comprise a biological matrix and a portion may comprise synthetic matrix.

As used herein “coronavirus” may refer to a group of related viruses such as but not limited to severe acute respiratory syndrome (SARS), middle east respiratory syndrome (MERS), and severe acute respiratory syndrome coronavirus 2 (SARS-CoV-2). All the coronaviruses cause respiratory tract infection that range from mild to lethal in mammals. Several non-limiting examples of Coronavirus strains are described herein. In some embodiments, the compositions may protect against any Sarbecoviruses including but not limited to SARS-CoV1 or SARS-CoV2.

As used herein, “severe acute respiratory syndrome coronavirus 2 (SARS-CoV2)” is a betacoronavirus that causes Coronavirus Disease 19 (COVID-19).

A “subject” is an individual and includes, but is not limited to, a mammal (e.g., a human, horse, pig, rabbit, dog, sheep, goat, non-human primate, cow, cat, guinea pig, or rodent), a fish, a bird, a reptile or an amphibian. The term does not denote a particular age or sex. Thus, adult and newborn subjects, as well as fetuses, whether male or female, are intended to be included. A “patient” is a subject afflicted with a disease or disorder. The term “patient” includes human and veterinary subjects.

The terms “administering*, and “administration” refer to methods of providing a pharmaceutical preparation to a subject. Such methods are well known to those skilled in the art and include, but are not limited to, administering the compositions orally, parenterally (e.g., intravenously and subcutaneously), by intramuscular injection, by intraperitoneal injection, intrathecally, transdermally, extracorporeally, topically or the like.

A composition can also be administered by topical intranasal administration (intranasally) or administration by inhalant. As used herein, “topical intranasal administration” means delivery of the compositions into the nose and nasal passages through one or both of the nares and can comprise delivery by a spraying mechanism (device) or droplet mechanism (device), or through aerosolization of the composition. Administration of the compositions by inhalant can be through the nose or mouth via delivery by a spraying or droplet mechanism. As used herein. “an inhaler” can be a spraying device or a droplet device for delivering a composition comprising the vaccine composition, in a pharmaceutically acceptable carrier, to the nasal passages and the upper and/or lower respiratory tracts of a subject. Delivery can also be directly to any area of the respiratory system (e.g., lungs) via intratracheal intubation. The exact amount of the compositions required will vary from subject to subject, depending on the species, age, weight and general condition of the subject, the severity of the disorder being treated, the particular composition used, its mode of administration and the like. Thus, it is not possible to specify an exact amount for every composition. However, an appropriate amount can be determined by one of ordinary skill in the art using only routine experimentation given the teachings herein.

A composition can also be administered by buccal delivery or by sublingual delivery. As used herein “buccal delivery” may refer to a method of administration in which the compound is delivered through the mucosal membranes lining the cheeks. In some embodiment, for a buccal delivery the vaccine composition is placed between the gum and the cheek of a patient. As used herein “sublingual delivery” may refer to a method of administration in which the compound is delivered through the mucosal membrane under the tongue. In some embodiments, for a sublingual delivery the vaccine composition is administered under the tongue of a patient.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, for example, U.S. Pat. No. 3,610,795, which is incorporated by reference herein.

### DETAILED DESCRIPTION OF THE INVENTION

Before the present compounds, compositions, and/or methods are disclosed and described, it is to be understood that this invention is not limited to specific synthetic methods or to specific compositions, as such may, of course, vary. It is also to be understood that the terminology used herein is for the purpose of describing particular embodiments only and is not intended to be limiting Embodiments of the present invention can be freely combined with each other if they are not mutually exclusive.

### Multi-Epitope Pan-Coronavirus Vaccines

The present invention features preemptive multi-epitope pan-Coronavirus vaccines, methods of use, and methods of producing said vaccines, methods of preventing coronavirus infections, etc. The present invention also provides methods of testing said vaccines, e.g., using particular animal models and clinical trials. The vaccine compositions herein can induce efficient and powerful protection against the coronavirus disease or infection, e.g., by inducing the production of antibodies (Abs), CD4⁺ T helper (Th1) cells, and CD⁺8 cytotoxic T-cells (CTL).

The vaccine compositions, e.g., the antigens, herein feature multiple epitopes, which helps provide multiple opportunities for the body to develop an immune response for preventing an infection.

In certain embodiments, the epitopes are conserved epitopes, e.g., epitopes that are highly conserved among human coronaviruses and/or animal coronaviruses (e.g., coronaviruses isolated from animals susceptible to coronavirus infections). The vaccines herein may be designed to be effective against past, current, and future coronavirus outbreaks.

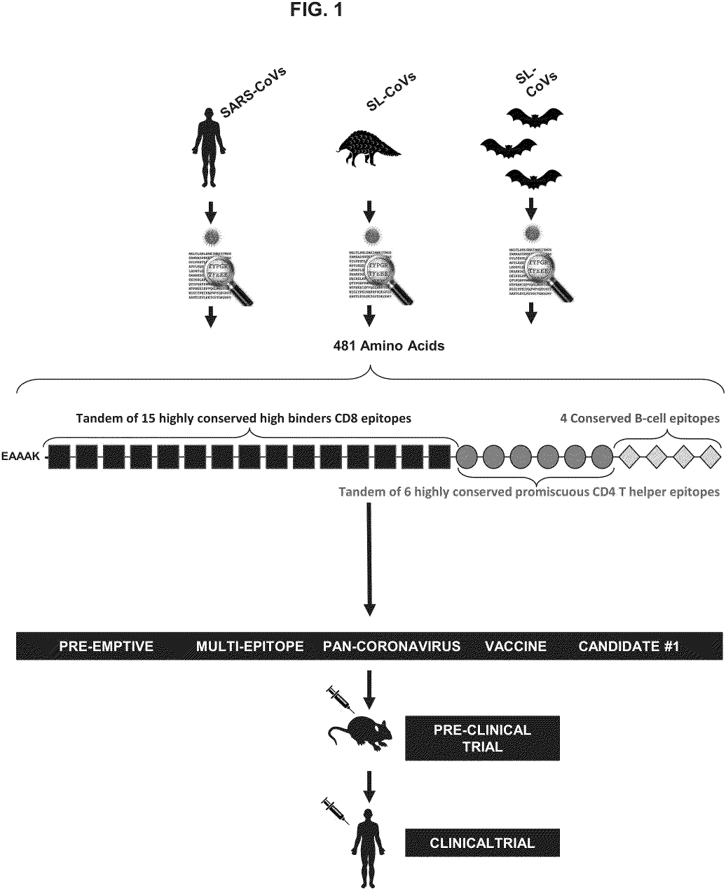

The present invention describes the identification of conserved B cell, CD4⁺ T cell, and CD8⁺ T cell epitopes. For example, FIG. 1 shows a schematic of the development of a pre-emptive multi-epitope pan coronavirus vaccine featuring multiple conserved B cell epitopes, multiple conserved CD8+ T cell epitopes, and multiple CD4⁺ T cell epitopes. The epitopes are derived from sequence analysis of many coronaviruses.

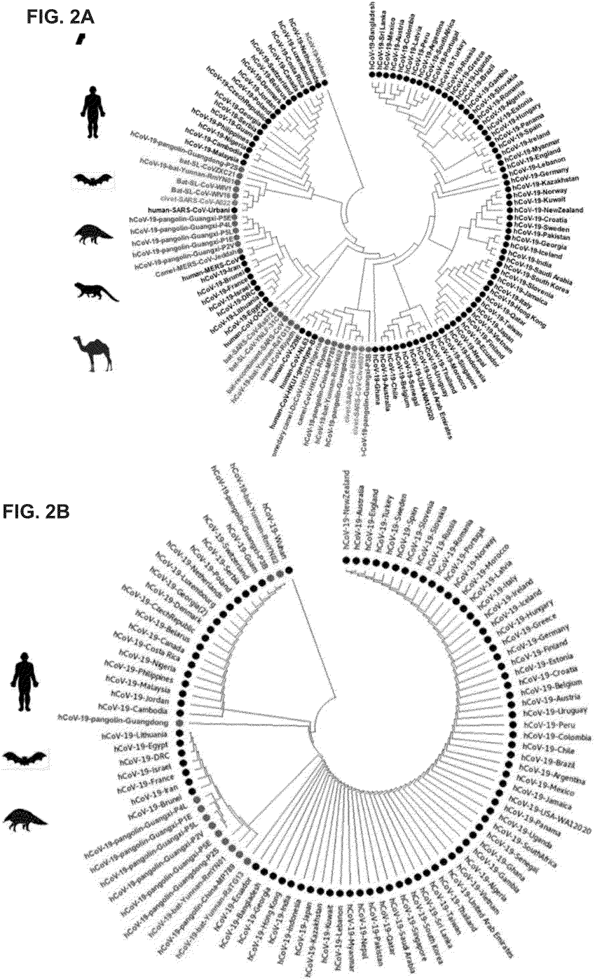

Coronaviruses used for determining conserved epitopes may include human SARS-CoVs as well as animal CoVs (e.g., bats, pangolins, civet cats, minks, camels, etc.) as described herein. As an example, FIG. 2A and FIG. 2B show an evolutionary comparison of genome sequences among beta-coronavirus strains isolated from humans and animals. FIG. 2A shows a phylogenetic analysis performed between SARS-CoV-2 strains (obtained from humans (Homo Sapiens (black)), along with the animal’s SARS-like Coronaviruses genome sequence (SL-CoVs) sequences obtained from bats (Rhinolophus affinis, Rhinolophus malayanus (red)), pangolins (Manis javanica (blue)), civet cats (Paguma larvata (green)), and camels (Camelus dromedarius (Brown)). The included SARS-CoV/MERS-CoV strains are from previous outbreaks (obtained from humans (Urbani, MERS-CoV, OC43, NL63, 229E, HKU1-genotype-B), bats (WIV16, WIV1, YNLF-31C, Rs672, recombinant strains), camel (Camelus dromedarius, (KT368891.1, MN514967.1, KF917527.1, NC_028752.1), and civet (Civet007, A022, B039)). The human SARS-CoV-2 genome sequences are represented from six continents. A phylogenetic analysis was performed among SARS-CoV-2 strains from human and other species with previous strains of SARS/MERS-CoV showing minimum genetic distance between the first SARS-CoV-2 isolate Wuhan-Hu-1 reported from the Wuhan Seafood market with bat strains hCoV-19-bat-Yunnan-RmYN02, bat-CoV-19-ZXC21, and hCoV-19-bat-Yunnan-RaTG13. This makes the bat strains the nearest precursor to the human-SARS-CoV-2 strain. Genetic distances based on Maximum Composite Likelihood model among the human, bat, pangolin, civet cat and camel genome sequences were evaluated. The results indicate least genetic distance among SARS-CoV-2 isolate Wuhan-Hu-1 and bat strains bat-CoV-19-ZXC21 (0.1), hCoV-19-bat-Yunnan-RaTG13 (0.1), and hCoV-19-bat-Yunnan-RmYN02 (0.2). FIG. 2B shows an evolutionary analysis performed among the human-SARS-CoV-2 genome sequences reported from six continents and SARS-CoV-2 genome sequences obtained from bats (Rhinolophus affinis, Rhinolophus malayanus), and pangolins (Manis javanica)).

Additionally, other coronaviruses may be used for determining conserved epitopes (including human SARS-CoVs as well as animal CoVs (e.g., bats, pangolins, civet cats, minks, camels, etc.)) that meet the criteria to be classified as “variants of concern” or “variants of interest.” Coronavirus variants that appear to meet one or more of the undermentioned criteria may be labeled “variants of interest” or “variants under investigation” pending verification and validation of these properties. In some embodiments, the criteria may include increased transmissibility, increased morbidity, increased mortality, increased risk of “long COVID”, ability to evade detection by diagnostic tests, decreased susceptibility to antiviral drugs (if and when such drugs are available), decreased susceptibility to neutralizing antibodies, either therapeutic (e.g., convalescent plasma or monoclonal antibodies) or in laboratory experiments, ability to evade natural immunity (e.g., causing reinfections), ability to infect vaccinated individuals, Increased risk of particular conditions such as multisystem inflammatory syndrome or long-haul COVID or Increased affinity for particular demographic or clinical groups, such as children or immunocompromised individuals. Once validated, variants of interest are renamed “variant of concern” by monitoring organizations, such as the CDC.

The conserved epitopes may be derived from structural (e.g., spike glycoprotein, envelope protein, membrane protein, nucleoprotein) or non-structural proteins of the coronaviruses (e.g., any of the 16 NSPs encoded by ORF1a/b).

In some embodiments, the target epitopes are each highly conserved among one or a combination of: SARS-CoV-2 human strains, SL-CoVs isolated from bats, SL-CoVs isolated from pangolin, SL-CoVs isolated from civet cats, and MERS strains isolated from camels. For example, in certain embodiments, the target epitopes are each highly conserved among one or a combination of: at least 50,000 SARS-CoV-2 human strains, five SL-CoVs isolated from bats, five SL-CoVs isolated from pangolin, three SL-CoVs isolated from civet cats, and four MERS strains isolated from camels. In certain embodiments, the target epitopes are each highly conserved among one or a combination of: at least 80,000 SARS-CoV-2 human strains, five SL-CoVs isolated from bats, five SL-CoVs isolated from pangolin, three SL-CoVs isolated from civet cats, and four MERS strains isolated from camels. In certain embodiments, the target epitopes are each highly conserved among one or a combination of: at least 50,000 SARS-CoV-2 human strains in circulation during the COVI-19 pandemic, at least one CoV that caused a previous human outbreak, five SL-CoVs isolated from bats, five SL-CoVs isolated from pangolin, three SL-CoVs isolated from civet cats, and four MERS strains isolated from camels. In certain embodiments, the target epitopes are each highly conserved among at least 1 SARS-CoV-2 human strain in current circulation, at least one CoV that has caused a previous human outbreak, at least one SL-CoV isolated from bats, at least one SL-CoV isolated from pangolin, at least one SL-CoV isolated from civet cats, and at least one MERS strain isolated from camels. In certain embodiments, the target epitopes are each highly conserved among at least 1,000 SARS-CoV-2 human strains in current circulation, at least two CoVs that has caused a previous human outbreak, at least two SL-CoVs isolated from bats, at least two SL-CoVs isolated from pangolin, at least two SL-CoVs isolated from civet cats, and at least two MERS strains isolated from camels. In certain embodiments, the target epitopes are each highly conserved among one or a combination of: at least one SARS-CoV-2 human strain in current circulation, at least one CoV that has caused a previous human outbreak, at least one SL-CoV isolated from bats, at least one SL-CoV isolated from pangolin, at least one SL-CoV isolated from civet cats, and at least one MERS strain isolated from camels. The present invention is not limited to the aforementioned coronavirus strains that may be used to identify conserved epitopes.

In certain embodiments, one or more of the conserved epitopes are derived from one or more SARS-CoV-2 human strains or variants in current circulation; one or more coronaviruses that has caused a previous human outbreak; one or more coronaviruses isolated from animals selected from a group consisting of bats, pangolins, civet cats, minks, camels, and other animal receptive to coronaviruses; and/or one or more coronaviruses that cause the common cold. SARS-CoV-2 human strains and variants in current circulation may include the original SARS-CoV-2 strain (SARS-CoV-2 isolate Wuhan-Hu-1), and several variants of SARS-CoV-2 including but not limited to Spain variant B.1.177; Australia variant B.1.160, England variant B.1.1.7; South Africa variant B.1.351; Brazil variant P.1; California variant B.1.427/B 1.429; Scotland variant B.1.258; Belgium/Netherlands variant B.1.221: Norway/France variant B.1.367; Norway/Denmark.UK variant B.1.1.277: Sweden variant B.1.1.302; North America, Europe, Asia, Africa, and Australia variant B.1.525; a New York variant B.1.526; variant B.1.525; variant B.1.526, variant S:677H; variant S:677P; B.1.617.2-Delta, variant B.1.1.529-Omicron (BA.1); sub-variant Omicron (BA.1); sub-variant Omicron (BA.2); sub-variant Omicron (BA.3); sub-variant Omicron (BA.4); sub-variant Omicron (BA.5). The present invention is not limited to the aforementioned variants of SARS-CoV-2 and encompasses variants identified in the future. The one or more coronaviruses that cause the common cold may include but are not limited to strains 229E (alpha coronavirus), NL63 (alpha coronavirus), OC43 (beta coronavirus), HKU1 (beta coronavirus).

As used herein, the term “conserved” refers to an epitope that is among the most highly conserved epitopes identified in a sequence alignment and analysis for its particular epitopes type (e.g., B cell, CD4 T cell, CD8 T cell). For example, the conserved epitopes may be the 5 most highly conserved epitopes identified (for the particular type of epitope). In some embodiments, the conserved epitopes may be the 10 most highly conserved epitopes identified (for the particular type of epitope). In some embodiments, the conserved epitopes may be the 15 most highly conserved epitopes identified (for the particular type of epitope). In some embodiments, the conserved epitopes may be the 20 most highly conserved epitopes identified (for the particular type of epitope). In some embodiments, the conserved epitopes may be the 25 most highly conserved epitopes identified (for the particular type of epitope). In some embodiments, the conserved epitopes may be the 30 most highly conserved epitopes identified (for the particular type of epitope). In some embodiments, the conserved epitopes may be the 40 most highly conserved epitopes identified (for the particular type of epitope). In some embodiments, the conserved epitopes may be the 50 most highly conserved epitopes identified (for the particular type of epitope). In some embodiments, the conserved epitopes may be the 50% most highly conserved epitopes identified (for the particular type of epitope). In some embodiments, the conserved epitopes may be the 60% most highly conserved epitopes identified (for the particular type of epitope). In some embodiments, the conserved epitopes may be the 70% most highly conserved epitopes identified (for the particular type of epitope). In some embodiments, the conserved epitopes may be the 80% most highly conserved epitopes identified (for the particular type of epitope). In some embodiments, the conserved epitopes may be the 90% most highly conserved epitopes identified (for the particular type of epitope). In some embodiments, the conserved epitopes may be the 95% most highly conserved epitopes identified (for the particular type of epitope). In some embodiments, the conserved epitopes may be the 99% most highly conserved epitopes identified (for the particular type of epitope). The present invention is not limited to the aforementioned thresholds.

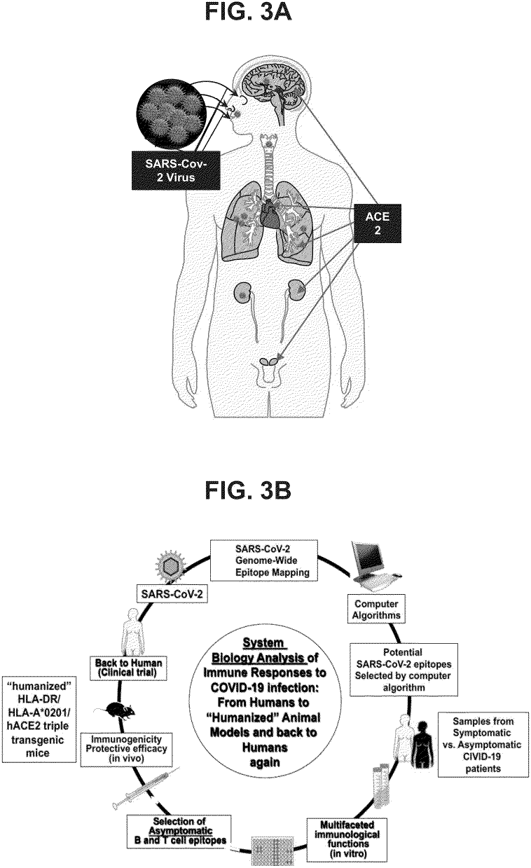

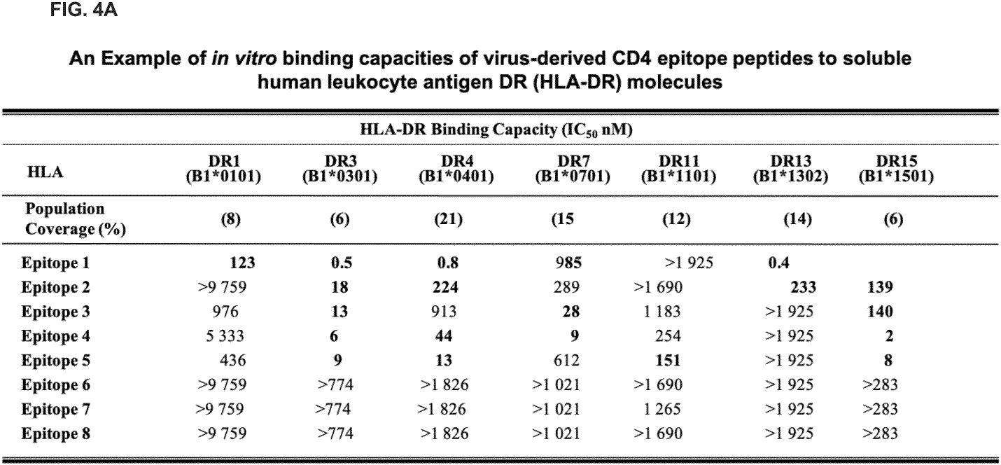

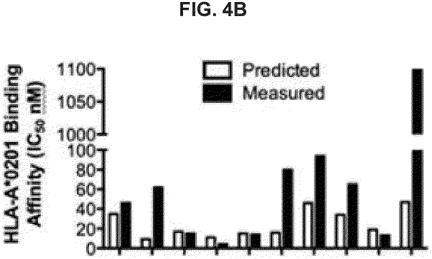

FIG. 3B shows an example of a systems biology approach utilized in the present invention.

In some embodiments, the composition comprises one or more conserved coronavirus B-cell target epitopes; one or more conserved coronavirus CD4⁺ T cell target epitopes; and one or more conserved coronavirus CD8⁺ T cell target epitopes. In some embodiments, the composition comprises one or more conserved coronavirus B-cell target epitopes and one or more conserved coronavirus CD4⁺ T cell target epitopes. In some embodiments, the composition comprises one or more conserved coronavirus B-cell target epitopes and one or more conserved coronavirus CD8⁺ T cell target epitopes. In some embodiments, the composition comprises one or more conserved coronavirus CD8⁺ target epitopes and one or more conserved coronavirus CD4⁺ T cell target epitopes. In some embodiments, the composition comprises one or more conserved coronavirus CD8⁺ target epitopes. In some embodiments, the composition comprises one or more conserved coronavirus CD4⁺ target epitopes. In some embodiments, the composition comprises one or more conserved coronavirus B cell target epitopes

As will be discussed herein, in certain embodiments, the composition comprises whole spike protein, one or more coronavirus CD4⁺ T cell target epitopes; and one or more coronavirus CD8⁺ T cell target epitopes. In certain embodiments, the composition comprises at least a portion of the spike protein (e.g., wherein the portion comprises a trimerized SARS-CoV-2 receptor-binding domain (RBD)), one or more coronavirus CD4⁺ T cell target epitopes; and one or more coronavirus CD8⁺ T cell target epitopes.

In certain embodiments, the composition comprises one or more coronavirus B cell target epitopes, one or more coronavirus CD4⁺ T cell target epitopes; and one or more coronavirus CD8⁺ T cell target epitopes. For example, in certain embodiments, the composition comprises 4 B cell target epitopes, 15 CD8⁺ T cell target epitopes, and 6 CD4⁺ T cell target epitopes. The present invention is not limited to said combination of epitopes.

In certain embodiments, the composition comprises 1-10 B cell target epitopes. In certain embodiments, the composition comprises 2-10 B cell target epitopes. In certain embodiments, the composition comprises 2-15 B cell target epitopes. In certain embodiments, the composition comprises 2-20 B cell target epitopes. In certain embodiments, the composition comprises 2-30 B cell target epitopes. In certain embodiments, the composition comprises 2-15 B cell target epitopes. In certain embodiments, the composition comprises 2-5 B cell target epitopes. In certain embodiments, the composition comprises 5-10 B cell target epitopes. In certain embodiments, the composition comprises 5-15 B cell target epitopes. In certain embodiments, the composition comprises 5-20 B cell target epitopes. In certain embodiments, the composition comprises 5-25 B cell target epitopes. In certain embodiments, the composition comprises 5-30 B cell target epitopes. In certain embodiments, the composition comprises 10-20 B cell target epitopes. In certain embodiments, the composition comprises 10-30 B cell target epitopes.

In certain embodiments, the composition comprises 1-10 CD8⁺ T cell target epitopes. In certain embodiments, the composition comprises 2-10 CD8⁺ T cell target epitopes. In certain embodiments, the composition comprises 2-15 CD8⁺ T cell target epitopes. In certain embodiments, the composition comprises 2-20 CD8⁺ T cell target epitopes. In certain embodiments, the composition comprises 2-30 CD8⁺ T cell target epitopes. In certain embodiments, the composition comprises 2-15 CD8⁺ T cell target epitopes. In certain embodiments, the composition comprises 2-5 CD8⁺ T cell target epitopes. In certain embodiments, the composition comprises 5-10 CD8⁺ T cell target epitopes. In certain embodiments, the composition comprises 5-15 CD8⁺ T cell target epitopes. In certain embodiments, the composition comprises 5-20 CD8⁺ T cell target epitopes. In certain embodiments, the composition comprises 5-25 CD8⁺ T cell target epitopes. In certain embodiments, the composition comprises 5-30 CD8⁺ T cell target epitopes. In certain embodiments, the composition comprises 10-20 CD8⁺ T cell target epitopes. In certain embodiments, the composition comprises 10-30 CD8⁺ T cell target epitopes.

In certain embodiments, the composition comprises 1-10 CD4⁺ T cell target epitopes. In certain embodiments, the composition comprises 2-10 CD4⁺ T cell target epitopes. In certain embodiments, the composition comprises 2-15 CD4⁺ T cell target epitopes. In certain embodiments, the composition comprises 2-20 CD4⁺ T cell target epitopes. In certain embodiments, the composition comprises 2-30 CD4⁺ T cell target epitopes. In certain embodiments, the composition comprises 2-15 CD4⁺ T cell target epitopes. In certain embodiments, the composition comprises 2-5 CD4⁺ T cell target epitopes. In certain embodiments, the composition comprises 5-10 CD4⁺ T cell target epitopes. In certain embodiments, the composition comprises 5-15 CD4⁺ T cell target epitopes. In certain embodiments, the composition comprises 5-20 CD4⁺ T cell target epitopes. In certain embodiments, the composition comprises 5-25 CD4⁺ T cell target epitopes. In certain embodiments, the composition comprises 5-30 CD4⁺ T cell target epitopes. In certain embodiments, the composition comprises 10-20 CD4⁺ T cell target epitopes. In certain embodiments, the composition comprises 10-30 CD4⁺ T cell target epitopes.

Table 1 below further describes various non-limiting combinations of numbers of CD4⁺ T cell target epitopes, CD8⁺ T cell target epitopes, and B cell target epitopes. The present invention is not limited to the examples described herein.

TABLE 1

| Example | # B Cell Epitopes | # CD8⁺ T Cell Epitopes | # CD4⁺ T Cell Epitopes |
|---|---|---|---|
| 1 | 4 | 15 | 6 |
| 2 | 5 | 10 | 7 |
| 3 | 4 | 12 | 8 |
| 4 | 1 | 16 | 9 |
| 5 | 2 | 2 | 2 |
| 6 | 1 | 5 | 5 |
| 7 | 4 | 6 | 6 |
| 8 | 3 | 12 | 4 |
| 9 | 3 | 3 | 3 |
| 10 | 1 | 14 | 8 |
| 11 | 2 | 10 | 5 |
| 12 | 4 | 9 | 3 |
| 13 | 3 | 3 | 7 |
| 14 | 5 | 11 | 4 |
| 15 | 2 | 8 | 6 |
| 16 | 3 | 9 | 8 |
| 17 | 2 | 10 | 4 |
| 18 | 4 | 6 | 7 |
| 19 | 3 | 14 | 3 |
| 20 | 2 | 4 | 5 |

The epitopes may be each separated by a linker. In certain embodiments, the linker allows for an enzyme to cleave between the target epitopes. The present invention is not limited to particular linkers or particular lengths of linkers. As an example, in certain embodiments, one or more epitopes may be separated by a linker 2 amino acids in length. In certain embodiments, one or more epitopes may be separated by a linker 3 amino acids in length. In certain embodiments, one or more epitopes may be separated by a linker 4 amino acids in length. In certain embodiments, one or more epitopes may be separated by a linker 5 amino acids in length. In certain embodiments, one or more epitopes may be separated by a linker 6 amino acids in length. In certain embodiments, one or more epitopes may be separated by a linker 7 amino acids in length. In certain embodiments, one or more epitopes may be separated by a linker 8 amino acids in length. In certain embodiments, one or more epitopes may be separated by a linker 9 amino acids in length. In certain embodiments, one or more epitopes may be separated by a linker 10 amino acids in length In certain embodiments, one or more epitopes may be separated by a linker from 2 to 10 amino acids in length.

Linkers are well known to one of ordinary skill in the art. Non-limiting examples of linkers include AAY, KK, and GPGPG. For example, in certain embodiments, one or more CD8⁺ T cell epitopes are separated by AAY. In some embodiments, one or more CD4⁺ T cell epitopes are separated by GPGPG. In certain embodiments, one or more B cell epitopes are separated by KK. In certain embodiments, KK is a linker between a CD4⁺ T cell epitope and a B cell epitope. In certain embodiments, KK is a linker between a CD8⁺ T cell epitope and a B cell epitope. In certain embodiments, KK is a linker between a CD8⁺ T cell epitope and a CD4⁺ T cell epitope. In certain embodiments, AAY is a linker between a CD4 T cell epitope and a B cell epitope. In certain embodiments, AAY is a linker between a CD8⁺ T cell epitope and a B cell epitope. In certain embodiments, AAY is a linker between a CD8⁺ T cell epitope and a CD4⁺ T cell epitope. In certain embodiments, GPGPG is a linker between a CD4⁺ T cell epitope and a B cell epitope. In certain embodiments, GPGPG is a linker between a CD8⁺ T cell epitope and a B cell epitope. In certain embodiments, GPGPG is a linker between a CD8⁺ T cell epitope and a CD4 T cell epitope.

The target epitopes may be derived from structural proteins, non-structural proteins, or a combination thereof. For example, structural proteins may include spike proteins (S), envelope proteins (E), membrane proteins (M), or nucleoproteins (N).

In some embodiments, the target epitopes are derived from at least one SARS-CoV-2 protein. The SARS-CoV-2 proteins may include ORF1ab protein. Spike glycoprotein, ORF3a protein, Envelope protein, Membrane glycoprotein, ORF6 protein, ORF7a protein, ORF7b protein, ORF8 protein, Nucleocapsid protein, and ORF10 protein. The ORF1ab protein provides nonstructural proteins (Nsp) such as Nsp1, Nsp2, Nsp3 (Papain-like protease), Nsp4, Nsp5 (3C-like protease), Nsp6, Nsp7, Nsp8, Nsp9, Nsp10, Nsp11, Nsp12 (RNA polymerase), Nsp13 (5′ RNA triphosphatase enzyme), Nsp14 (guanosineN7-methyltransferase), Nsp15 (endoribonuclease), and Nsp16 (2′-O-ribose-methyltransferase).

The SARS-CoV-2 has a genome length of 29,903 or more base pairs (bps) ssRNA (SEQ ID NO: 1). Generally, the region between 266-21555 bps codes for ORF1ab polypeptide; the region between 21563-25384 bps codes for one of the structural proteins (spike protein or surface glycoprotein); the region between 25393-26220 bps codes for the ORF3a gene; the region between 26245-26472 bps codes for the envelope protein; the region between 26523-27191 codes for the membrane glycoprotein (or membrane protein); the region between 27202-27387 bps codes for the ORF6 gene; the region between 27394-27759 bps codes for the ORF7a gene; the region between 27894-28259 bps codes for the ORF8 gene; the region between 28274-29533 bps codes for the nucleocapsid phosphoprotein (or the nucleocapsid protein); and the region between 29558-29674 bps codes for the ORF10 gene.

The one or more CD8⁺ T cell target epitopes may be derived from a protein selected from: spike glycoprotein. Envelope protein, ORF1ab protein, ORF7a protein, ORF8a protein, ORF10 protein, or a combination thereof. The one or more CD4⁺ T cell target epitopes may be derived from a protein selected from: spike glycoprotein, Envelope protein, Membrane protein, Nucleocapsid protein, ORF1a protein. ORF1ab protein, ORF6 protein, ORF7a protein, ORF7b protein, ORF8 protein, or a combination thereof. The one or more B cell target epitopes may be derived from wherein the spike protein or portion thereof.

The conserved epitopes may be restricted to human HLA class 1 and 2 haplotypes. In some embodiments, the conserved epitopes are restricted to cat and dog MHC class 1 and 2 haplotypes.

For any of the embodiments herein, the epitopes that are selected may be those that achieve a particular score in a binding assay (for binding to an HLA molecule, for example.) For example, in some embodiments, the epitopes selected have an IC₅₀ score of 250 or less in an ELISA binding assay (e.g., an ELISA binding assay specific for HLA-DR/peptide combination, HLA-A*0201/peptide combination, etc), or the equivalent of the IC₅₀ score of 250 or less in a different binding assay. Binding assays are well known to one of ordinary skill in the art.

FIG. 4A shows examples of binding capacities of virus-derived CD4+ T cell epitope peptides to soluble HLA-DR molecules. CD4+ T cell peptides were submitted to ELISA binding assays specific for HLA-DR molecules. Reference non-viral peptides were used to validate each assay. Data are expressed as relative activity (ratio of the IC₅₀ of the peptides to the IC₅₀ of the reference peptide) and are the means of two experiments. Peptide epitopes with high affinity binding to HLA-DR molecules have IC₅₀ below 250 and are indicated in bold. IC₅₀ above 250 indicates peptide epitopes that failed to bind to tested HLA-DR molecules.

FIG. 4B shows an example of potential epitopes binding with high affinity to HLA-A*0201 and stabilizing expression on the surface of target cells: Predicted and measured binding affinity of genome-derived peptide epitopes to soluble HLA-A*0201 molecule (IC₅₀ nM). The binding capacities of a virus CD8 T cell epitope peptide to soluble HLA-A*0201 molecules. CD8 T cell peptides were submitted to ELISA binding assays specific for HLA-A*0201 molecules. Reference non-viral peptides were used to validate each assay. Data are expressed as relative activity (ratio of the IC₅₀ to the peptide to the IC₅₀ of the reference peptide) and are the means of two experiments. Peptide epitopes with high affinity binding to HLA-A*0201 molecules have ICso below 100 and are indicated in bold. ICso above 100 indicates peptide epitopes that failed to bind to tested HLA-A*0201 molecules.

### CD8+ Epitopes

Examples of methods for identifying potential CD8+ T cell epitopes and screening conservancy of potential CD8+ T cell epitopes are described herein. The present invention is not limited to the particular software systems disclosed, and other software systems are accessible to one of ordinary skill in the art for such methods. The present invention is not limited to the specific haplotypes used herein. For example, one of ordinary skill in the art may select alternative molecules (e.g., HLA molecules) for molecular docking studies.

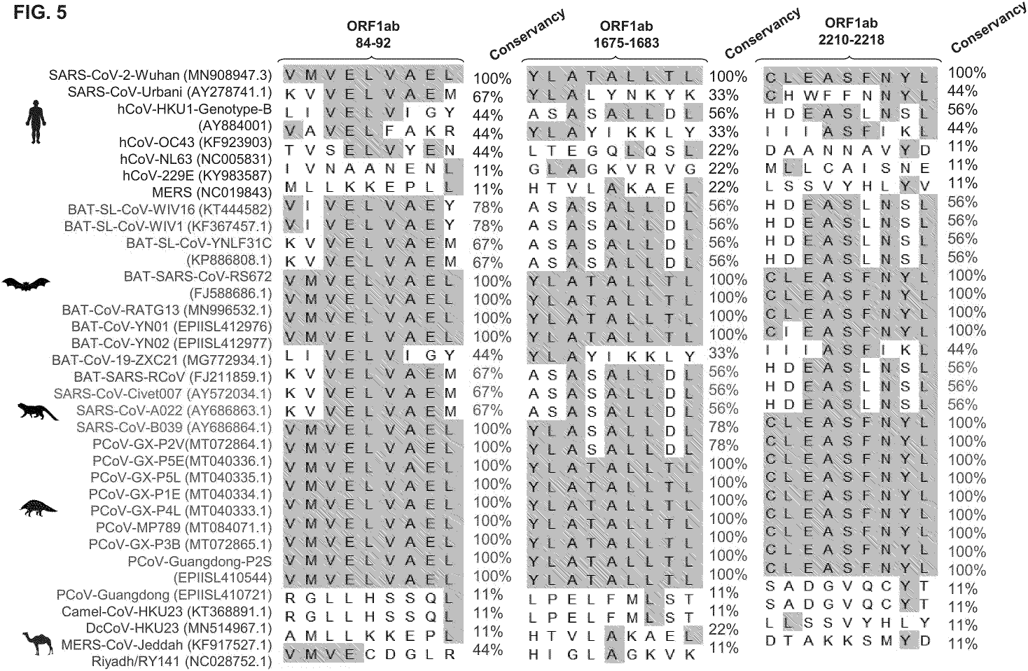

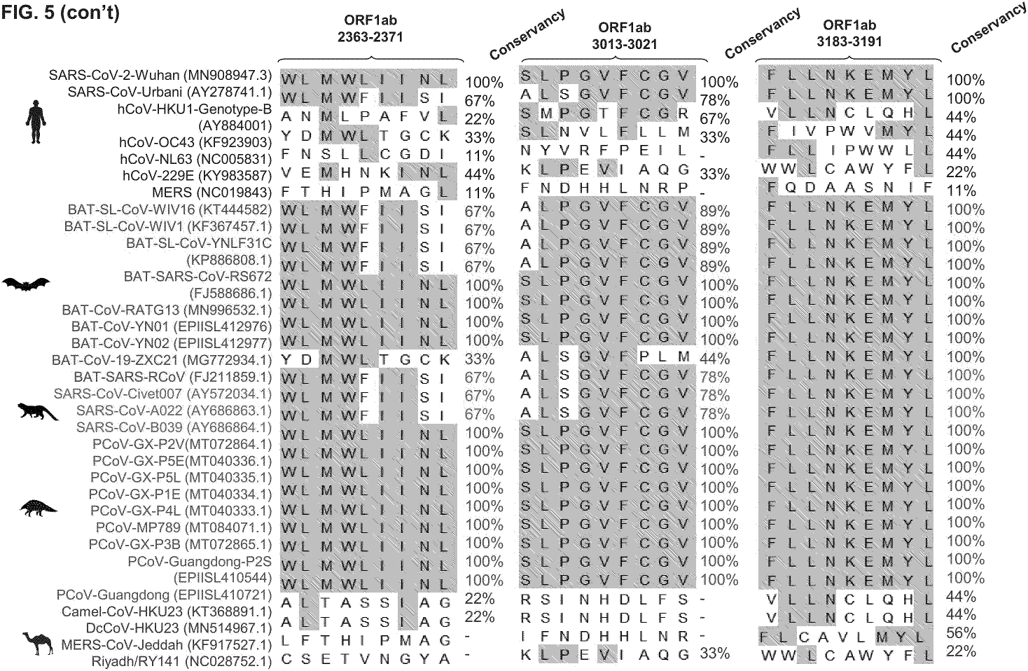

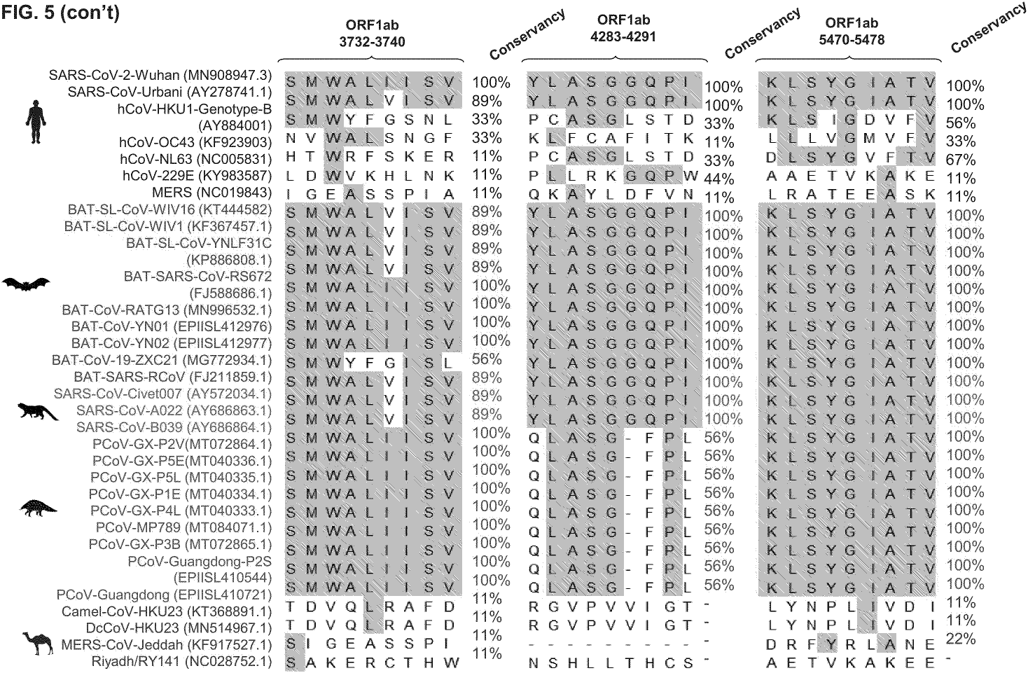

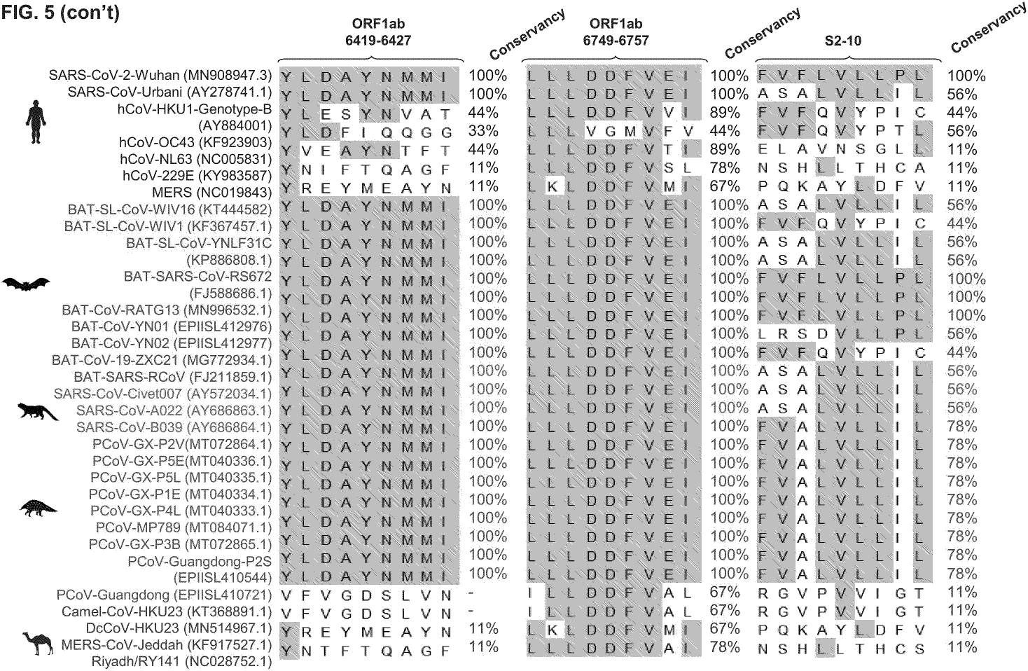

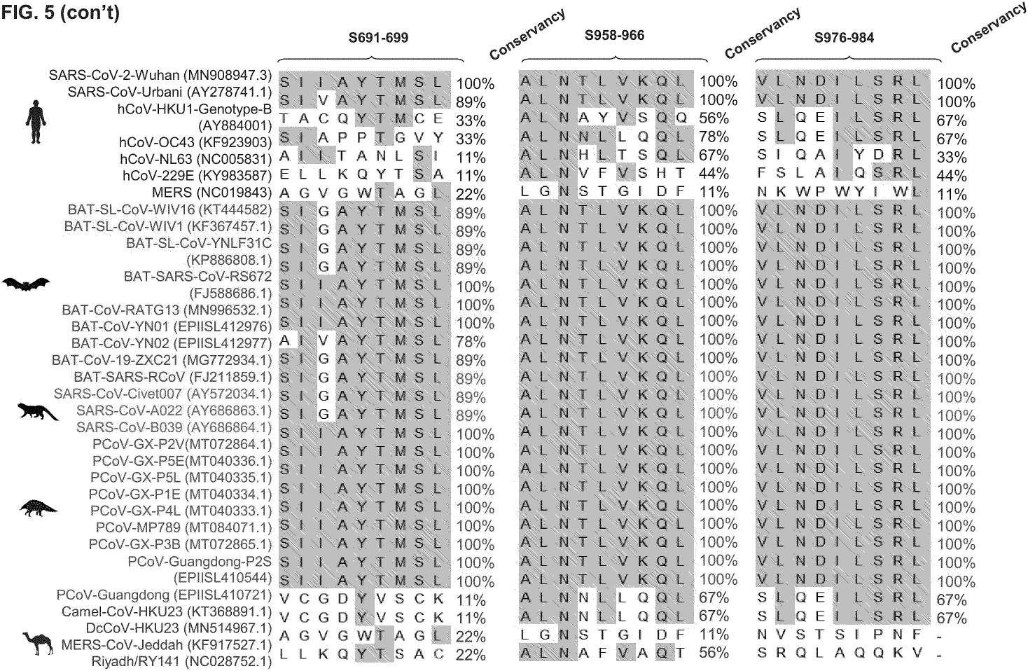

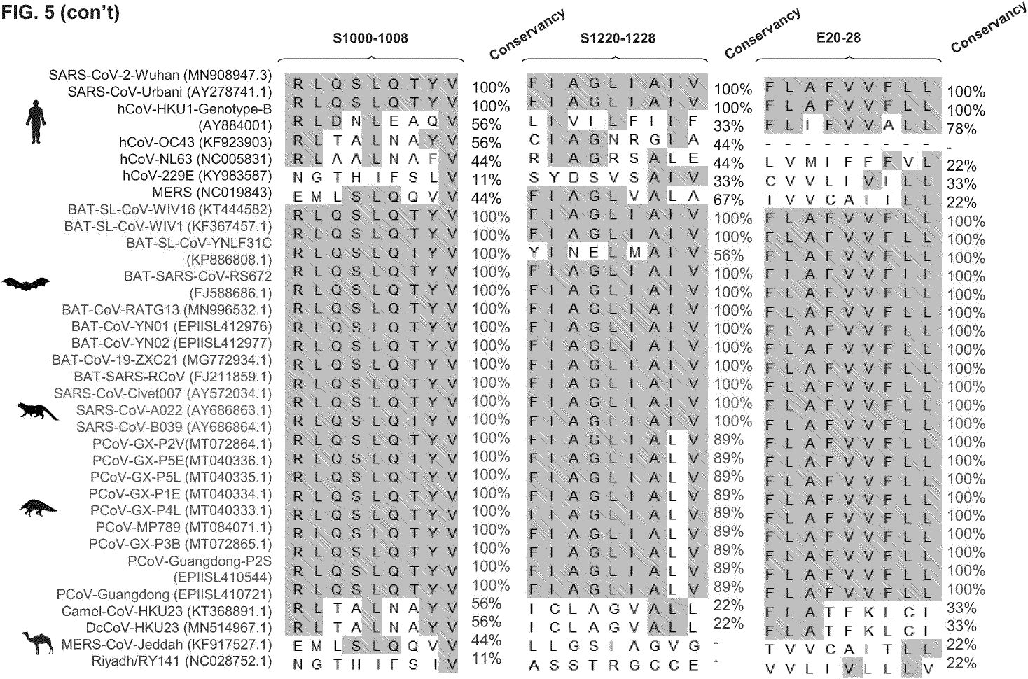

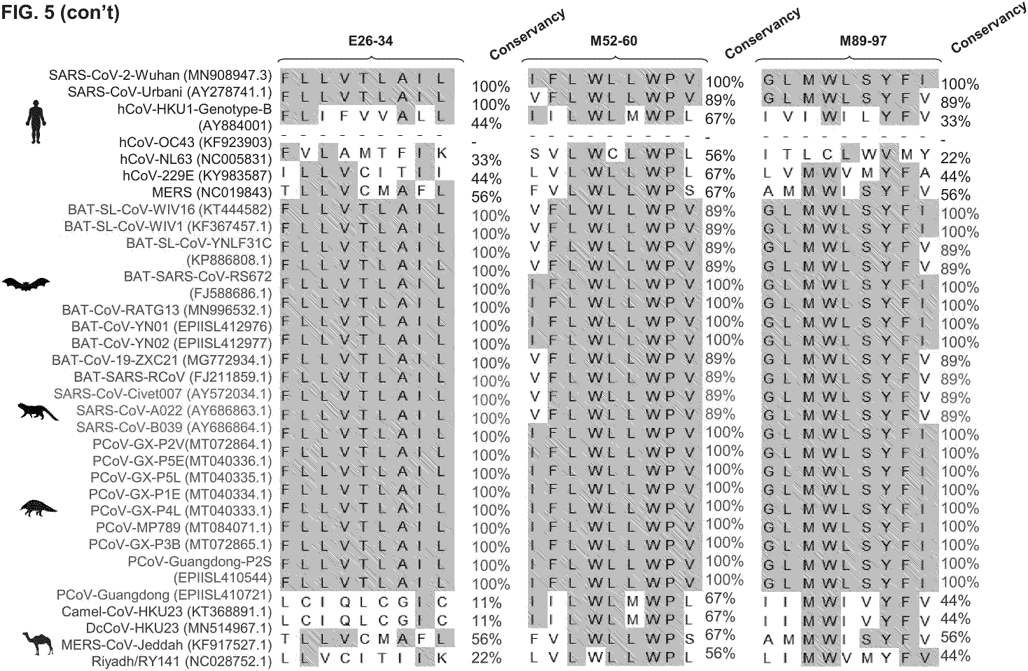

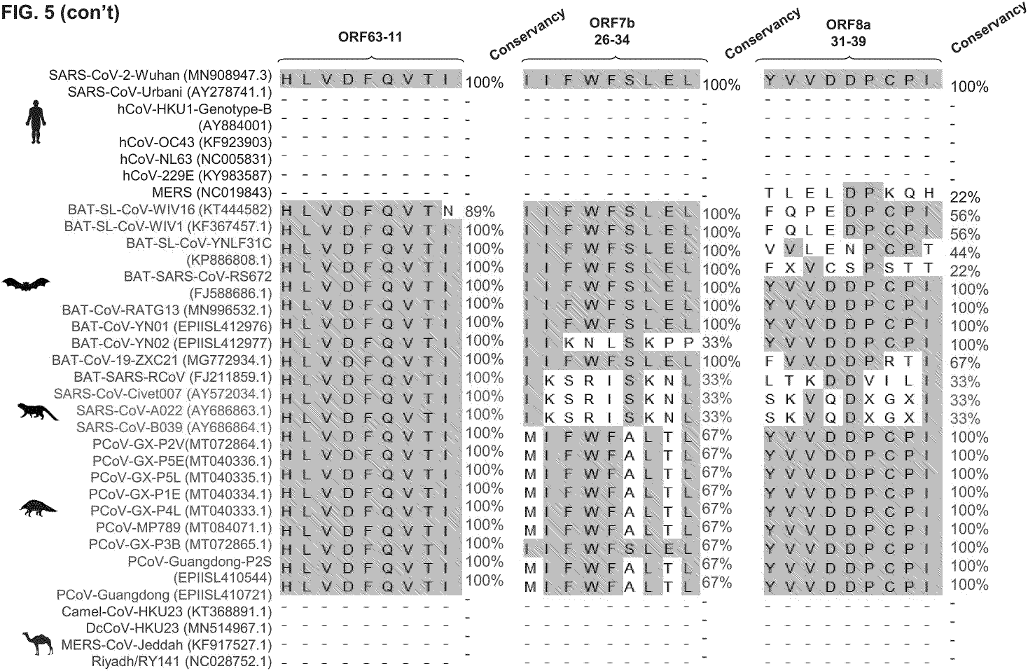

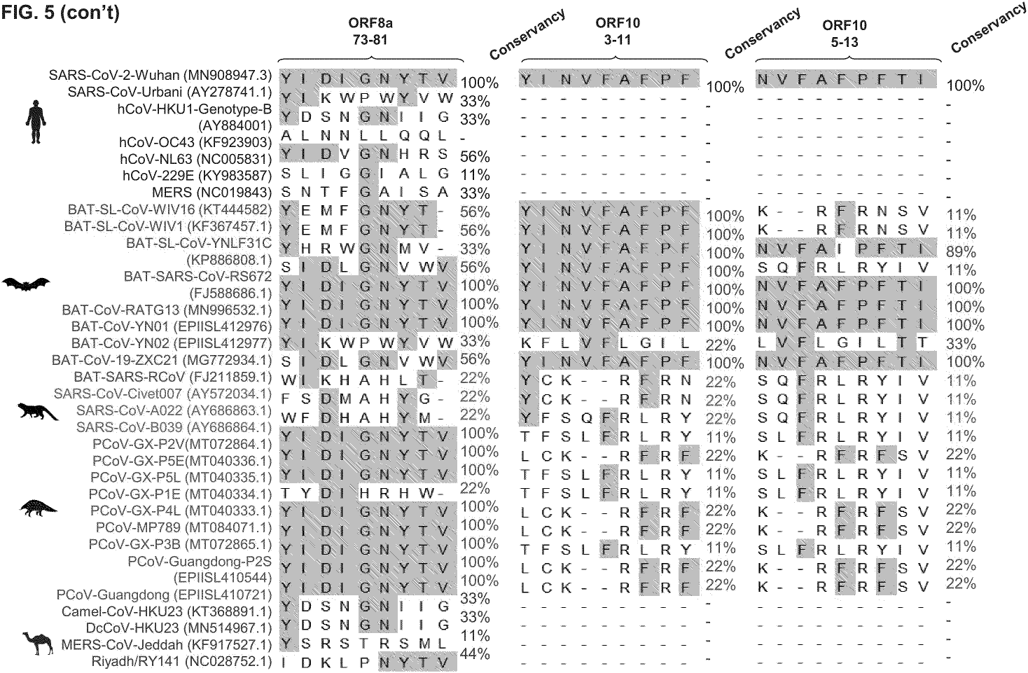

FIG. 5 shows sequence homology analysis for screening conservancy of potential CD8+ T cell epitopes, e.g., the comparison of sequence homology for the potential CD8+ T cell epitopes among 81,963 SARS-CoV-2 strains (that currently circulate in 190 countries on 6 continents), the 4 major “common cold” Coronaviruses that cased previous outbreaks (e.g., hCoV-OC43, hCoV-229E, hCoV-HKU1-Genotype B, and hCoV-NL63), and the SL-CoVs that were isolated from bats, civet cats, pangolins and camels. Epitope sequences highlighted present a high degree of homology among the currently circulating 81,963 SARS-CoV-2 strains and at least a 50% conservancy among two or more humans SARS-CoV strains from previous outbreaks, and the SL-CoV strains isolated from bats, civet cats, pangolins and camels.

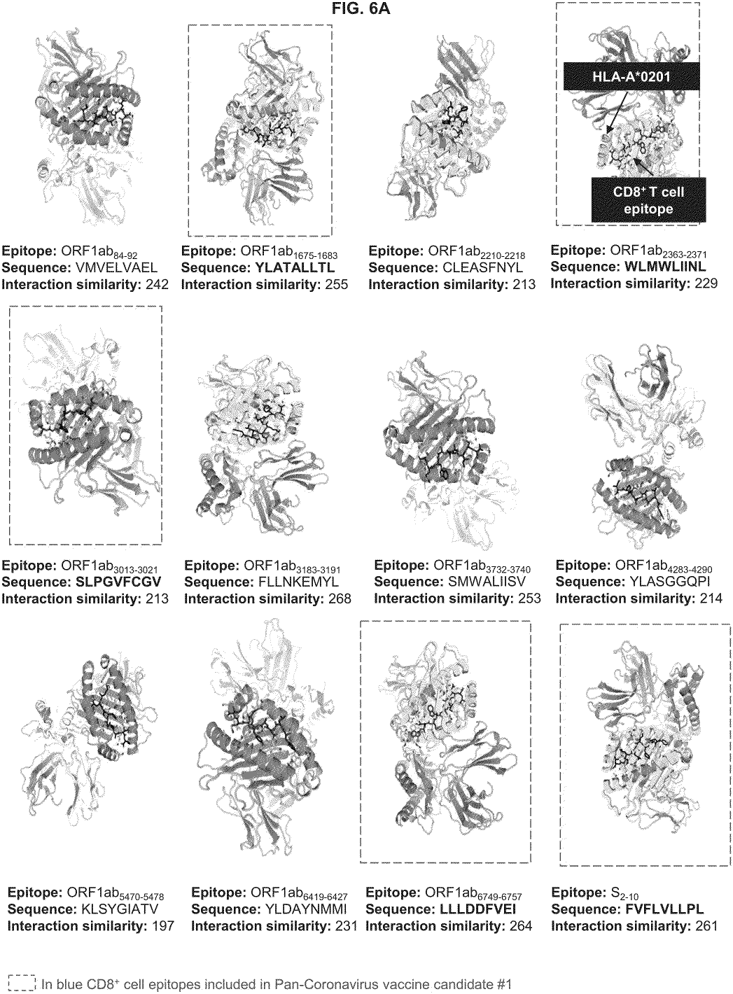

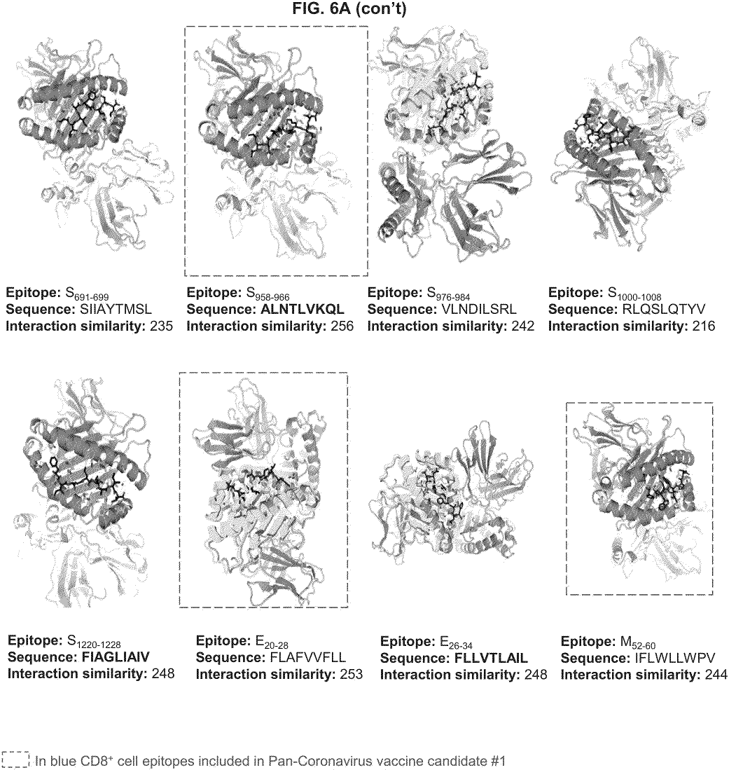

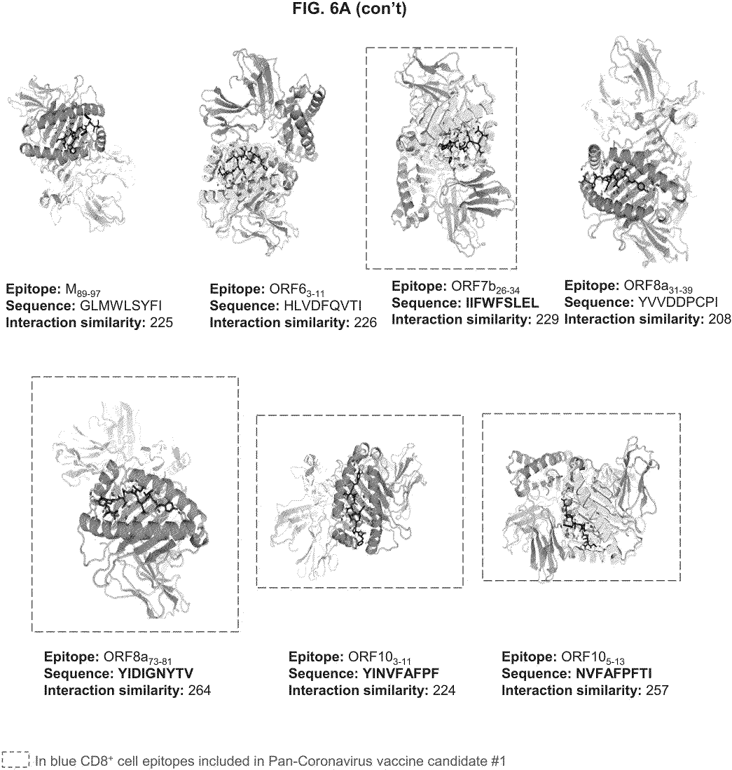

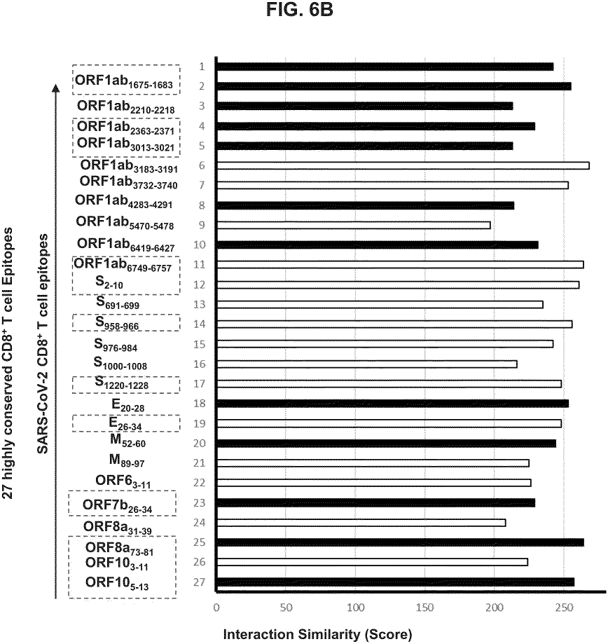

From the analysis, 27 CD8+ T cell epitopes were selected as being highly conserved. FIG. 6A and FIG. 6B show the docking of the conserved epitopes to the groove of HLA-A*02:01 molecules as well as the interaction scores determined by protein-peptide molecular docking analysis.

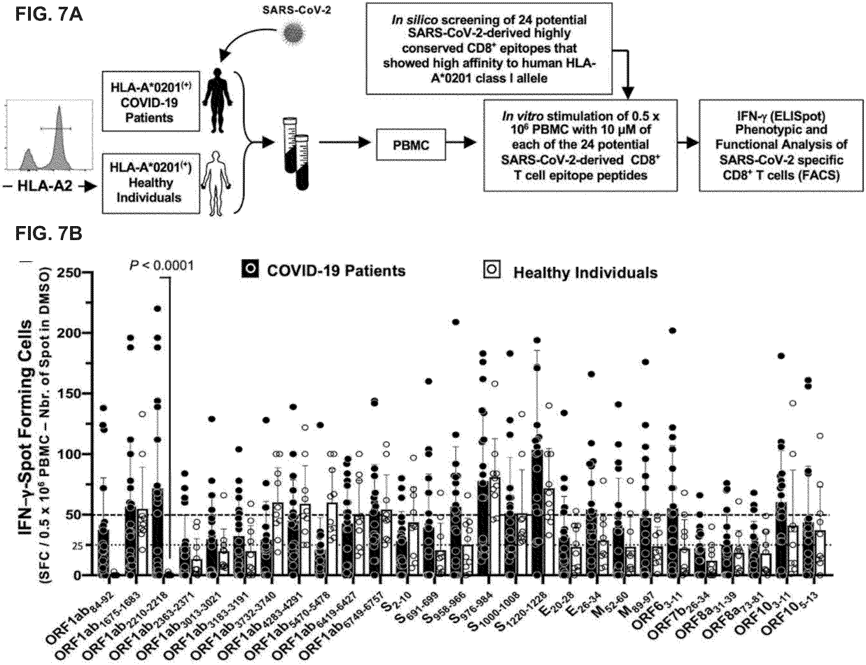

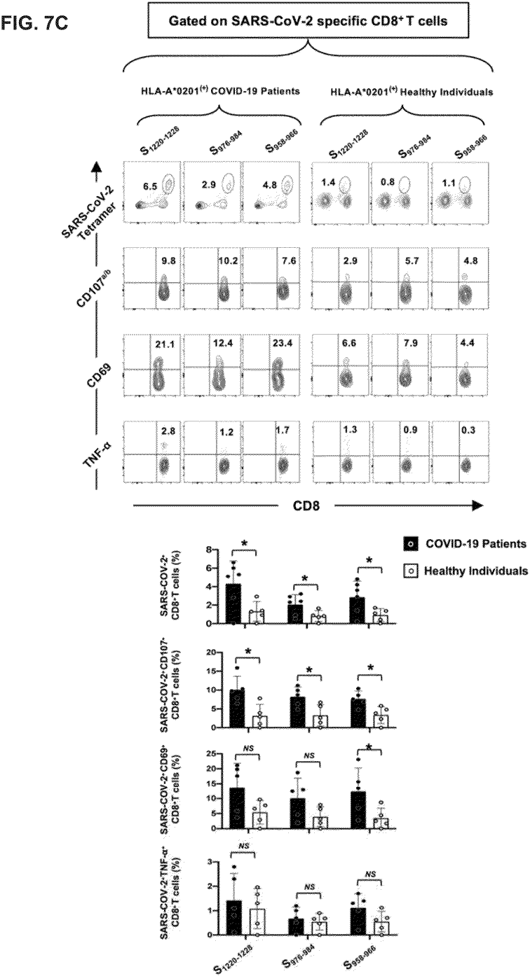

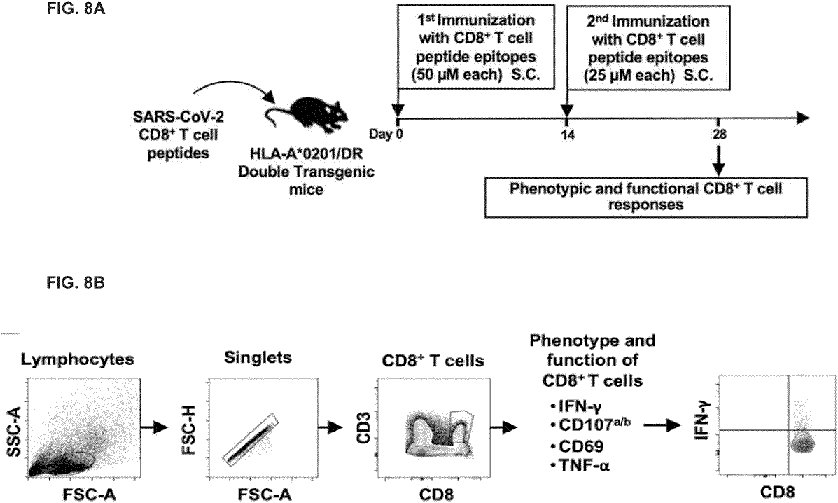

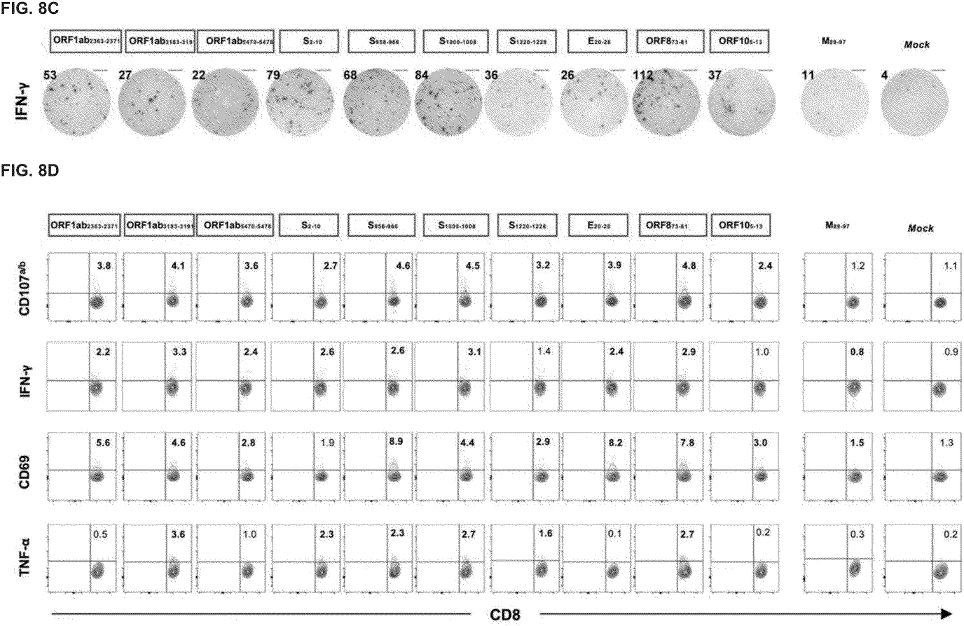

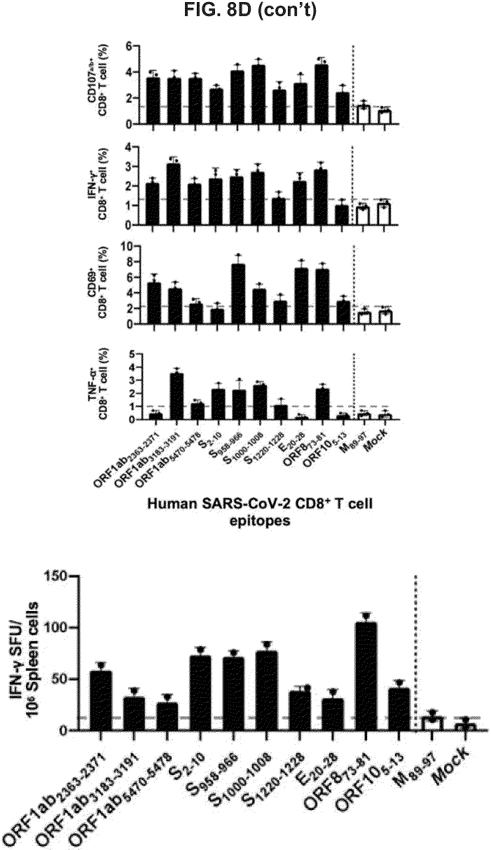

FIG. 7A, FIG. 7B, and FIG. 7C show that CD8+ T cells specific to several highly conserved SARS-CoV-2 epitopes disclosed herein were detected in COVID-19 patients and unexposed healthy individuals. FIG. 8A, FIG. 8B, FIG. 8C, and FIG. 8D show immunogenicity of the identified SARS-CoV-2 CD8+ T cell epitopes.

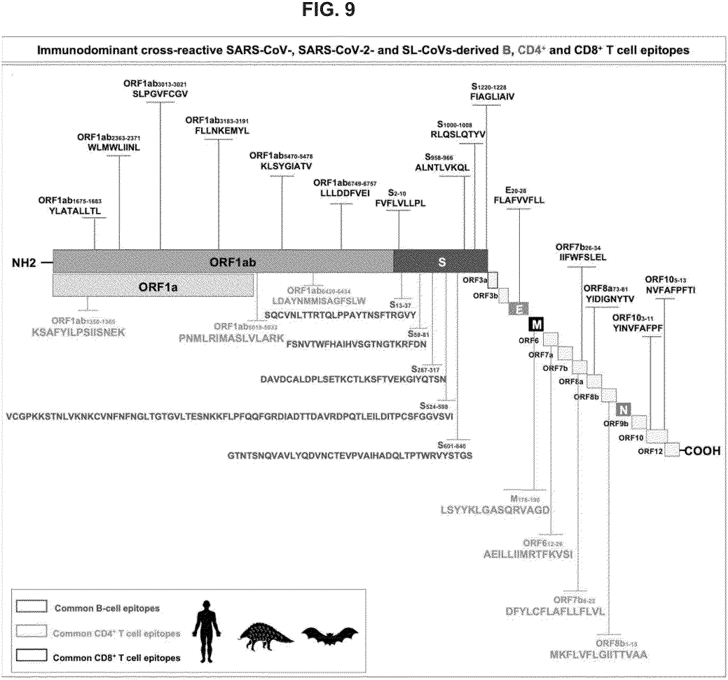

The CD8⁺ T cell target epitopes discussed above include S₂₋₁₀, S₁₂₂₀₋₁₂₂₈, S₁₀₀₀₋₁₀₀₈, S₉₅₈₋₉₆₆, E₂₀₋₂₈, ORF1ab₁₆₇₅₋₁₆₈₃, ORF1ab₂₃₆₃₋₂₃₇₁, ORF1ab₃₀₁₃₋₃₀₂₁, ORF1ab₃₁₈₃₋₃₁₉₁, ORF1ab₅₄₇₀₋₅₄₇₈, ORF1ab₆₇₄₉₋₆₇₅₇, ORF7b₂₆₋₃₄, ORF8a₇₃₋₈₁, ORF10₃₋₁₁, and ORF10₅₋₁₃ FIG. 9 shows the genome-wide location of the epitopes. Thus, in certain embodiments, the vaccine composition may comprise one or more CD8⁺ T cell epitopes selected from: S₂₋₁₀, S₁₂₂₀₋₁₂₂₈, S₁₀₀₀₋₁₀₀₈, S₉₅₈₋₉₆₆, E₂₀₋₂₈, ORF1ab₁₆₇₅₋₁₆₈₃, ORF1ab₂₃₆₃₋₂₃₇₁, ORF1ab₃₀₁₃₋₃₀₂₁, ORF1ab₃₁₈₃₋₃₁₉₁, ORF1ab₅₄₇₀₋₅₄₇₈, ORF1ab₆₇₄₉₋₆₇₅₇, ORF7b₂₆₋₃₄, ORF8a₇₃₋₈₁, ORF10₃₋₁₁, ORF10₅₋₁₃, or a combination thereof. Table 2 below describes the sequences for the aforementioned epitope regions.

TABLE 2

| CD8⁺ T Cell Epitope | Epitope Sequence | SEQ ID NO: | CD8⁺ T Cell Epitope | Epitope Sequence | SEQ ID NO: |
|---|---|---|---|---|---|
| ORF1ab₈₄₋₉₂ | VMVELVAEL | 2 | ORF8a₇₃₋₈₁ | YIDIGNYTV | 27 |
| ORF1ab₁₆₇₅₋₁₆₈₃ | YLATALLTL | 3 | ORF10₃₋₁₁ | YINVFAFPF | 28 |
| ORF1ab₂₂₁₀₋₂₂₁₈ | CLEASFNYL | 4 | ORF10₅₋₁₃ | NVFAFPFTI | 29 |
| ORF1ab₂₃₆₃₋₂₃₇₁ | WLMWLIINL | 5 | | | |
| ORF1 ab₃₀₁₃₋₃₀₂₁ | SLPGVFCGV | 6 | S | KSYGFQPTY | 184 |
| ORF1ab₃₁₈₃₋₃₁₉₁ | FLLNKEMYL | 7 | S | VVGNHKYRF | 185 |
| ORF1ab₃₇₃₂₋₃₇₄₀ | SMWALIISV | 8 | S | YQVGNKPCK | 186 |
| ORF1ab₄₂₈₃₋₄₂₉₁, | YLASGGQPI | 9 | S | CVIAWNSKK | 187 |
| ORF1ab₅₄₇₀₋₅₄₇₈ | KLSYGIATV | 10 | S | KGAKGLNCY | 188 |
| ORF1ab₆₄₁₉₋₆₄₂₇ | YLDAYNMMI | 11 | S | SQCVNFTTR | 189 |
| ORF1ab₆₇₄₉₋₆₇₅₇ | LLLDDFVEI | 12 | S | NIADYNYKL | 190 |
| S₂₋₁₀ | FVFLVLLPL | 13 | S | YLPLKSYGF | 191 |
| S₆₉₁₋₆₉₉ | SIIAYTMSL | 14 | S | KCYGVSLNK | 192 |
| S₉₅₈₋₉₆₆ | ALNTLVKQL | 15 | S | IYKTPPIKY | 193 |
| S₉₇₆₋₉₈₄ | VLNDILSRL | 16 | S | CVADYSFLY | 194 |
| S₁₀₀₀₋₁₀₀₈ | RLQSLQTYV | 17 | S | SVYAWDRRK | 195 |
| S₁₂₂₀₋₁₂₂₈ | FIAGLIAIV | 18 | S | RFFRKSNLK | 196 |
| E₂₀₋₂₈ | FLAFWFLL | 19 | S | DISTEIYQV | 197 |
| E₂₆₋₃₄ | FLLVTLAIL | 20 | S | YQPHRVVVL | 198 |
| E₂₆₋₃₄ | FLLNKEMYL | 21 | S | FVIRGDQVK | 199 |
| M₅₂₋₆₀ | IFLWLLWPV | 22 | S | NATKFSSVY | 200 |
| M₈₉₋₉₇ | GLMWLSYFI | 23 | S | NLCPFSEIF | 201 |
| ORF6₃₋₁₁ | HLVDFQVTI | 24 | S | ASATVCGPK | 202 |
| ORF7b₂₆₋₃₄ | IIFWFSLEL | 25 | S | KINNCVADY | 203 |
| ORF8a₃₁₋₃₉ | YVVDDPCPI | 26 | | | |

The present invention is not limited to the aforementioned CD8* T cell epitopes. For example, the present invention also includes variants of the aforementioned CD8⁺ T cell epitopes, for example sequences wherein the aforementioned CD8⁺ T cell epitopes are truncated by one amino acid (examples shown below in Table 3).

TABLE 3

| CD8⁺ T Cell Epitope Origin: | Sequence with Single AA Truncation | SEQ ID NO: | CD8⁺ T Cell Epitope Origin: | Sequence with Single AA Truncation | SEQ ID NO: |
|---|---|---|---|---|---|
| ORF1 ab₈₄₋₉₂ | VMVELVAE | 30 | ORF8a₇₃₋₈₁ | IDIGNYTV | 55 |
| ORF1ab₁₆₇₅₋₁₆₈₃ | LATALLTL | 31 | ORF10₃₋₁₁ | YINVFAFP | 56 |
| ORF1ab₂₂₁₀₋₂₂₁₈ | CLEASFNY | 32 | ORF10₅₋₁₃ | VFAFPFTI | 57 |
| ORF1ab₂₃₆₃₋₂₃₇₁ | LMWLIINL | 33 | | | |
| ORF1ab₃₀₁₃₋₃₀₂₁ | SLPGVFCG | 34 | S | KSYGFQPT | 204 |
| ORF1ab₃₁₈₃₋₃₁₉₁ | LLNKEMYL | 35 | S | VVGNHKYR | 205 |
| ORF1 ab₃₇₃₂₋₃₇₄₀ | SMWALIIS | 36 | S | YQVGNKPC | 206 |
| ORF1 ab₄₂₈₃₋₄₂₉₁ | LASGGQPI | 37 | S | CVIAWNSK | 207 |
| ORF1ab₅₄₇₀₋₅₄₇₈ | KLSYGIAT | 38 | S | KGAKGLNC | 208 |
| ORF1ab₆₄₁₉₋₆₄₂₇ | LDAYNMMI | 39 | S | SQCVNFTT | 209 |
| ORF1ab₆₇₄₉₋₆₇₅₇ | LLLDDFVE | 40 | S | NIADYNYK | 210 |
| S₂₋₁₀ | VFLVLLPL | 41 | S | YLPLKSYG | 211 |
| S₆₉₁₋₆₉₉ | SIIAYTMS | 42 | S | KCYGVSLN | 212 |
| S₉₅₈₋₉₆₆ | LNTLVKQL | 43 | S | IYKTPPIK | 213 |
| S₉₇₆₋₉₈₄ | VLNDILSR | 44 | S | CVADYSFL | 214 |
| S₁₀₀₀₋₁₀₀₈ | LQSLQTYV | 45 | S | SVYAWDRR | 215 |
| S₁₂₂₀₋₁₂₂₈ | FIAGLIAI | 46 | S | RFFRKSNL | 216 |
| E₂₀₋₂₈ | LAFVVFLL | 47 | S | DISTEIYQ | 217 |
| E₂₆₋₃₄ | FLLVTLAI | 48 | S | YQPHRVVV | 218 |
| E₂₆₋₃₄ | LLNKEMYL | 49 | S | FVIRGDQV | 219 |
| M₅₂₋₆₀ | IFLWLLWP | 50 | S | NATKFSSV | 220 |
| M₈₉₋₉₇ | LMWLSYFI | 51 | S | NLCPFSEI | 221 |
| ORF6₃₋₁₁ | HLVDFQVT | 52 | S | ASATVCGP | 222 |
| ORF7b₂₆₋₃₄ | IFWFSLEL | 53 | S | KINNCVAD | 223 |
| ORF8a₃₁₋₃₉ | YVVDDPCP | 54 | S | KSYGFQPT | 224 |

The present invention is not limited to the aforementioned CD8⁺ T cell epitopes.

### CD4+ Epitopes

Examples of methods for identifying potential CD4+ T cell epitopes and screening conservancy of potential CD4+ T cell epitopes are described herein. The present invention is not limited to the particular software systems disclosed, and other software systems are accessible to one of ordinary skill in the art for such methods. The present invention is not limited to the specific haplotypes used herein. For example, one of ordinary skill in the art may select alternative molecules (e.g., HLA molecules) for molecular docking studies.

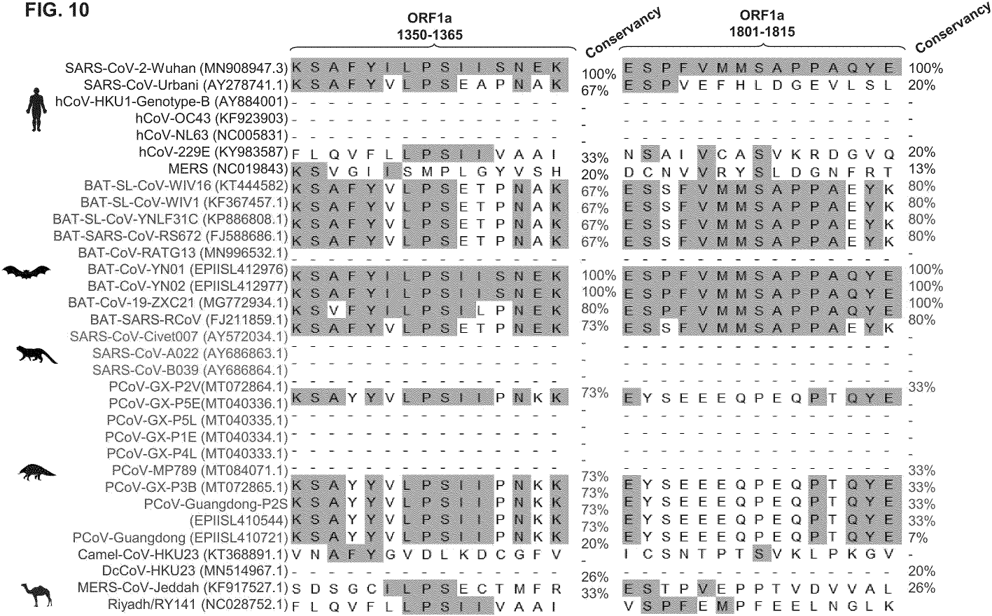

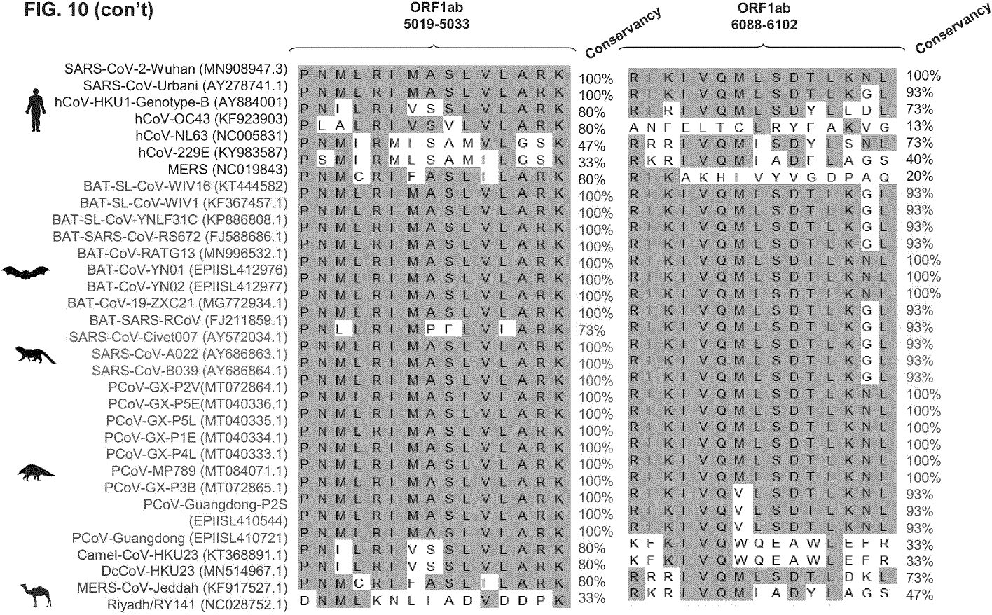

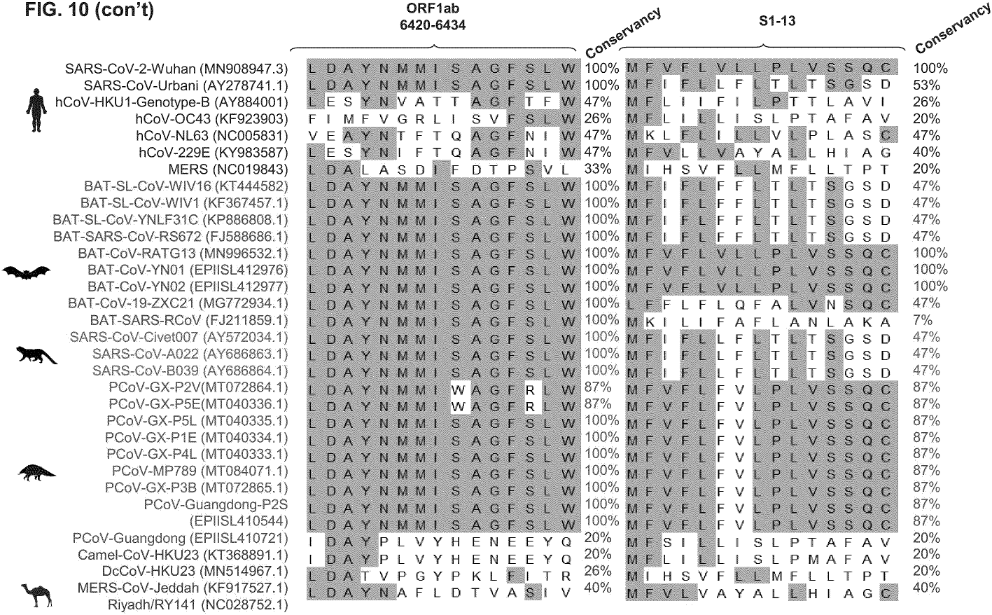

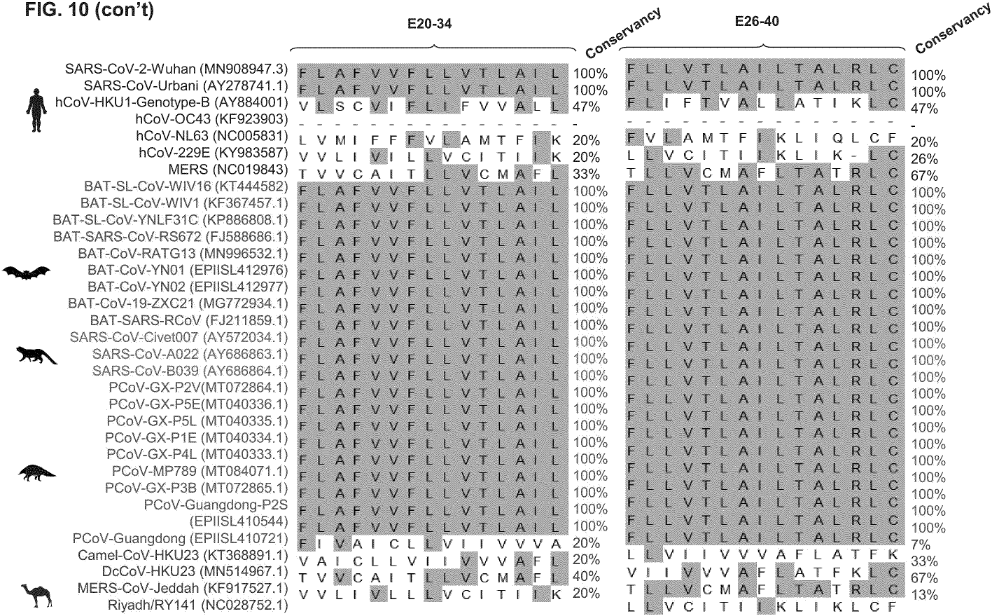

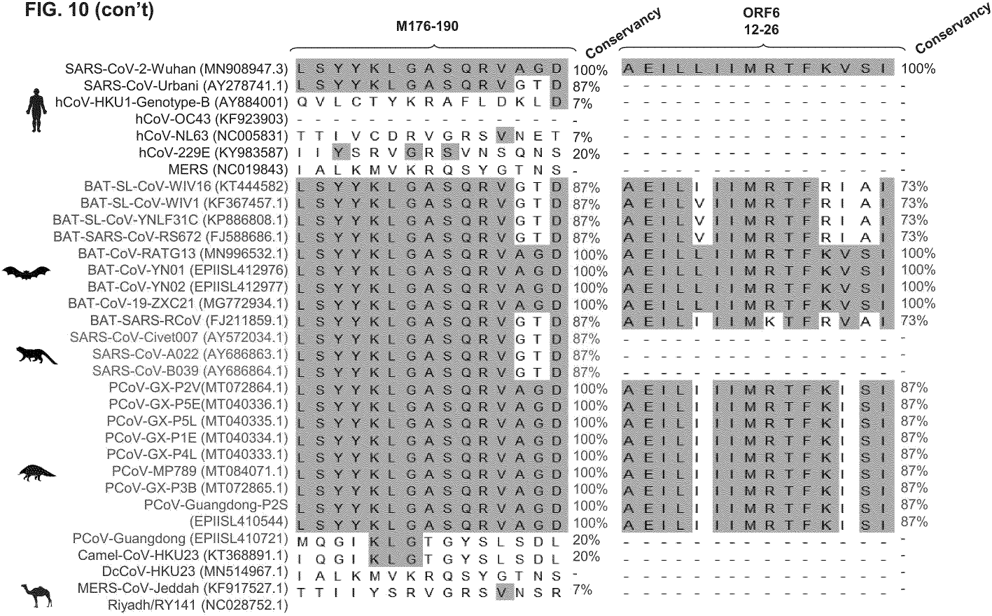

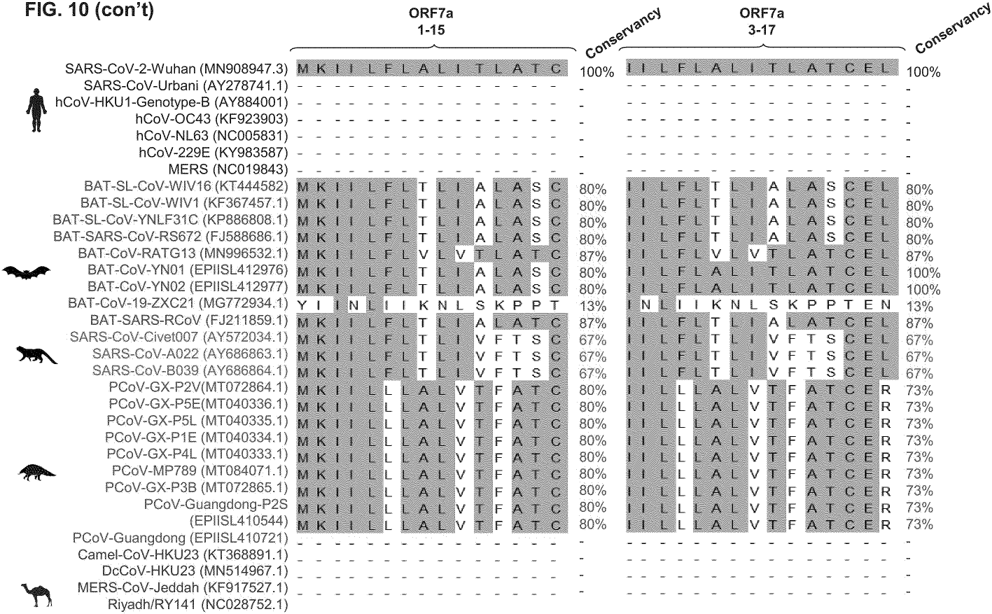

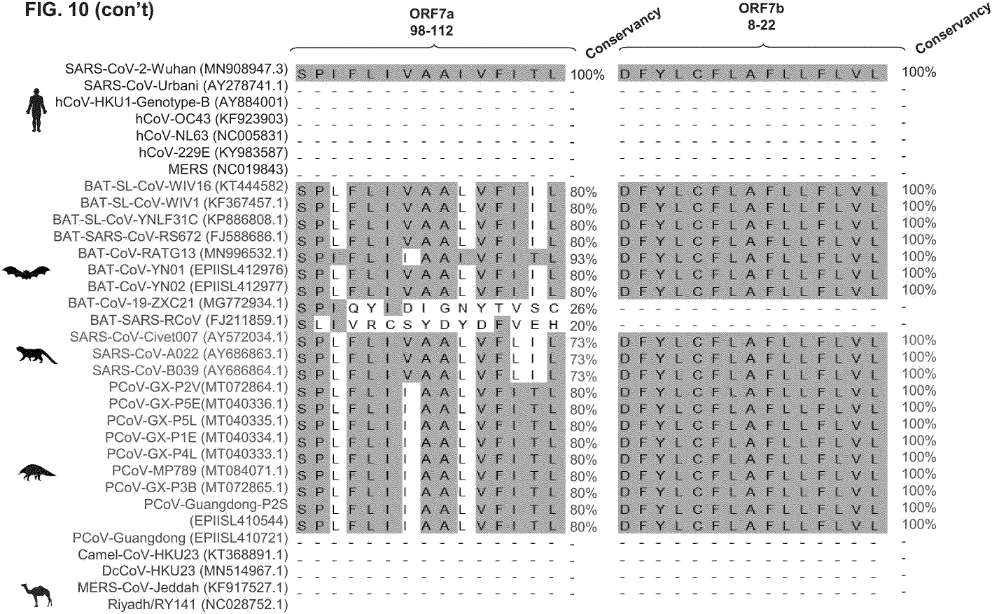

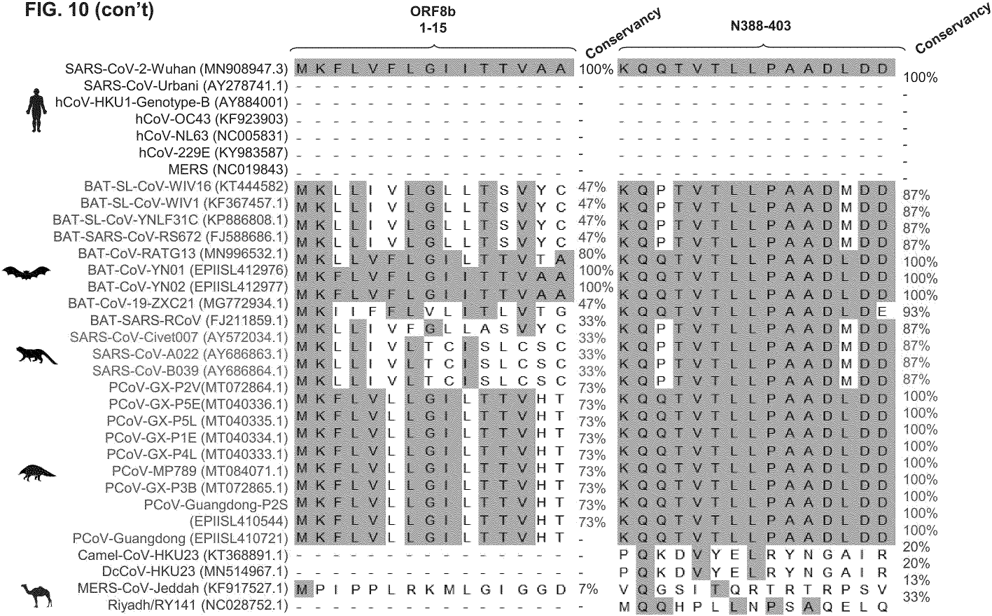

FIG. 10 shows the identification of highly conserved potential SARS-CoV-2-derived human CD4+ T cell epitopes that bind with high affinity to HLA-DR molecules. Out of a total of 9,594 potential HLA-DR-restricted CD4+ T cell epitopes from the whole genome sequence of SARS-CoV-2-Wuhan-Hu-1 strain (MN908947.3), 16 epitopes that bind with high affinity to HLA-DRB1 molecules were selected. The conservancy of the 16 CD4+ T cell epitopes was analyzed among human and animal Coronaviruses. Shown are the comparison of sequence homology for the 16 CD4+ T cell epitopes among 81,963 SARS-CoV-2 strains (that currently circulate in 6 continents), the 4 major “common cold” Coronaviruses that cased previous outbreaks (i.e. hCoV-OC43, hCoV-229E, hCoV-HKU1, and hCoV-NL63). and the SL-CoVs that were isolated from bats, civet cats, pangolins and camels. Epitope sequences highlighted in green present high degree of homology among the currently circulating 81,963 SARS-CoV-2 strains and at least a 50% conservancy among two or more humans SARS-CoV strains from previous outbreaks, and the SL-CoV strains isolated from bats, civet cats, pangolins and camels.

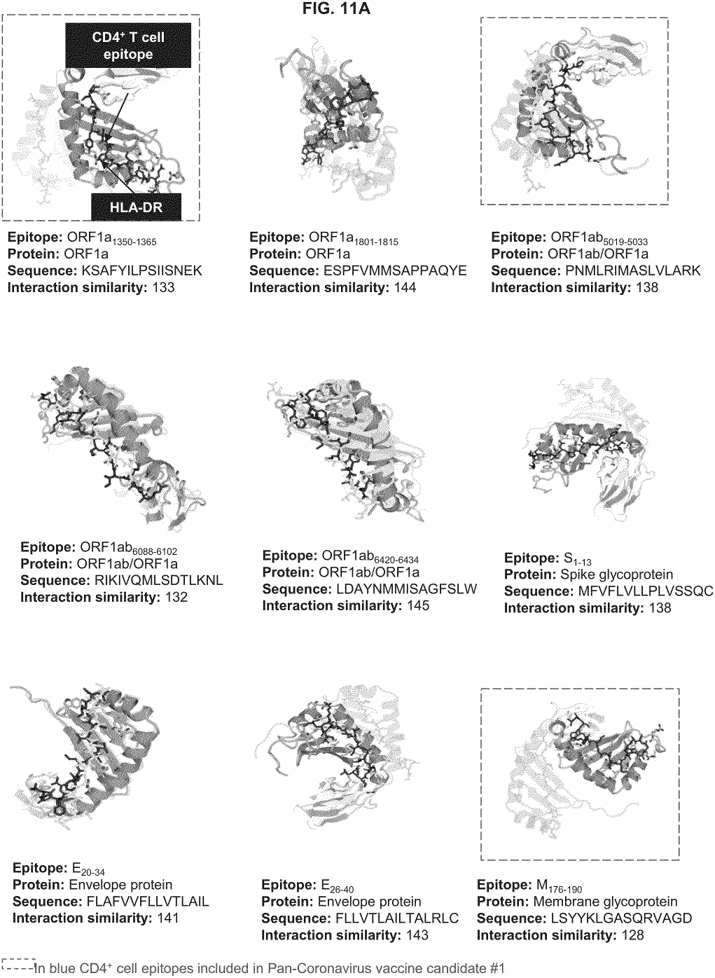

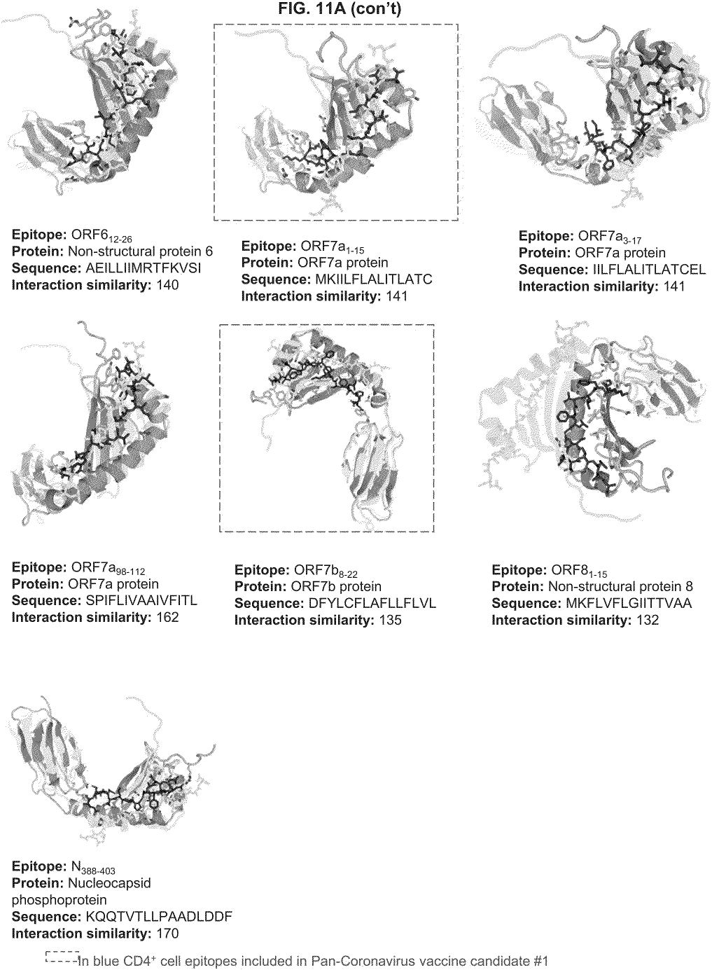

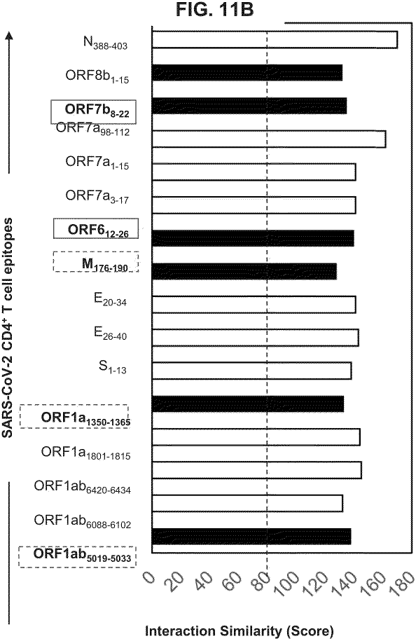

From the analysis, 16 CD4+ T cell epitopes were selected as being highly conserved. FIG. 11A and FIG. 11B show the docking of the conserved epitopes to the groove of HLA-DR molecules as well as the interaction scores determined by protein-peptide molecular docking analysis.

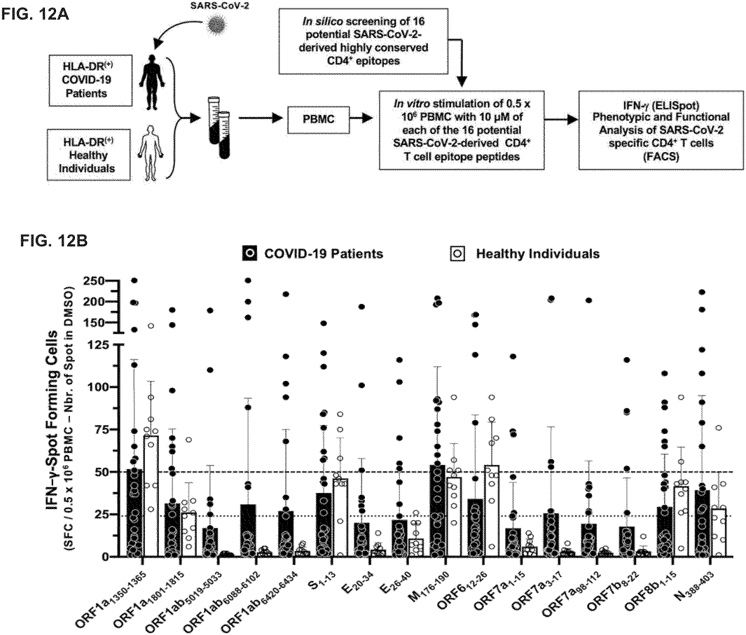

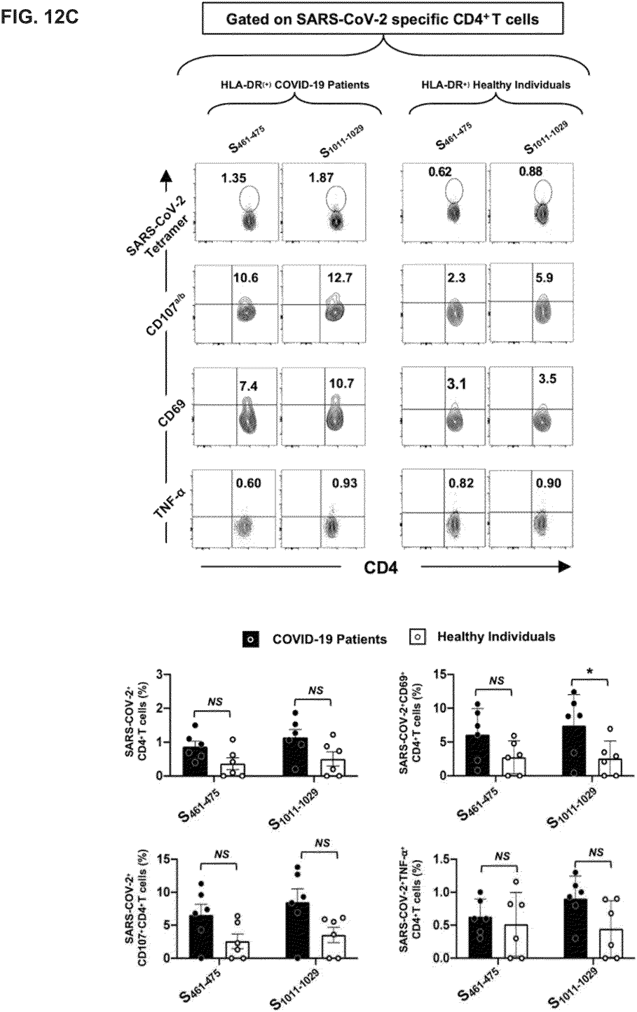

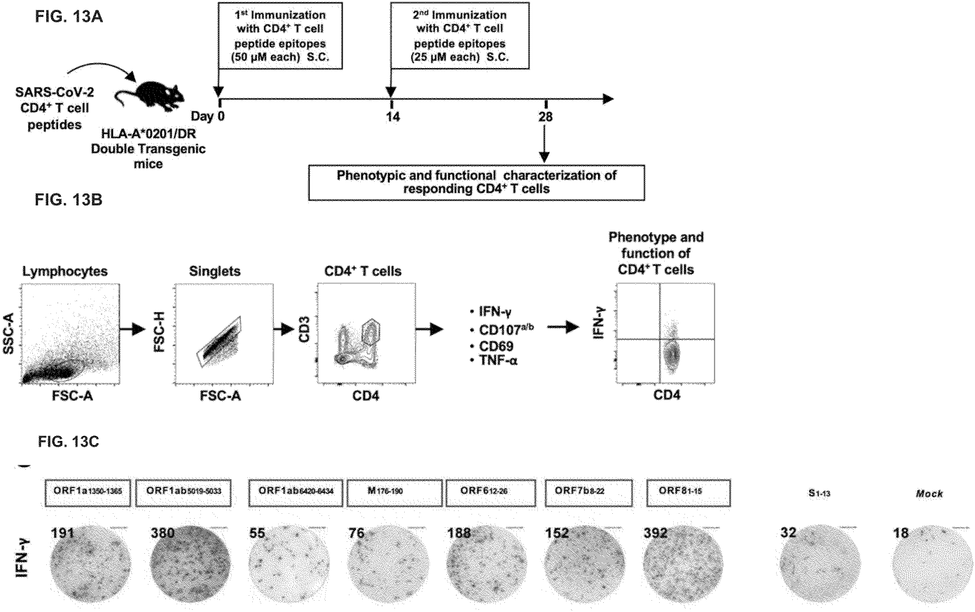

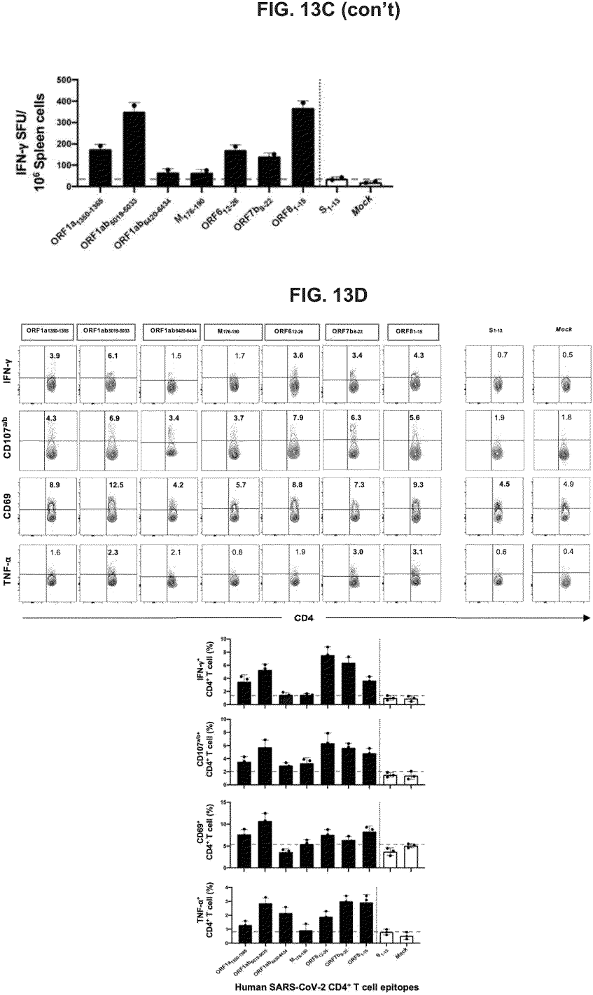

FIG. 12A, FIG. 12B, and FIG. 12C show that CD4+ T cells specific to several highly conserved SARS-CoV-2 epitopes disclosed herein were detected in COVID-19 patients and unexposed healthy individuals. FIG. 13A, FIG. 13B, FIG. 13C, and FIG. 13D show immunogenicity of the identified SARS-CoV-2 CD4+ T cell epitopes.

The CD4⁺ T cell target epitopes discussed above include ORF1a₁₃₅₀₋₁₃₆₅, ORF1ab₅₀₁₉₋₅₀₃₃. ORF6₁₂₋ ₂₆, ORF1ab₆₀₈₈₋₆₁₀₂, ORF1ab₆₄₂₀₋₆₄₃₄, ORF1a₁₈₀₁₋₁₈₁₅, S₁₋₁₃, E₂₆₋₄₀, E₂₀₋₃₄, M₁₇₆₋₁₉₀, N₃₈₈₋₄₀₃, ORF7a₃₋₁₇, ORF7a₁₋₁₅, ORF7b₈₋₂₂, ORF7a₉₈₋₁₁₂, and ORF8₁₋₁₅. FIG. 9 shows the genome-wide location of the epitopes. Thus, in certain embodiments, the vaccine composition may comprise one or more CD4⁺ T cell target epitopes selected from ORF1a₁₃₅₀₋₁₃₈₅, ORF1ab₅₀₁₉₋₅₀₃₃. ORF6₁₂₋₂₈, ORF1ab₆₀₈₈₋₆₁₀₂, ORF1ab₆₄₂₀₋₆₄₃₄, ORF1a_{1801-1815,} S₁₋₁₃, E₂₆₋₄₀, E₂₀₋₃₄, M₁₇₆₋₁₉₀, N₃₈₈₋₄₀₃, ORF7a₃₋₁₇, ORF7a₁₋₁₅, ORF7b₈₋₂₂, ORF7a₉₈₋₁₁₂, ORF8₁₋ ₁₅, or a combination thereof. Table 4 below describes the sequences for the aforementioned epitope regions.

TABLE 4

| CD4⁺ T Cell Epitope | Epitope Sequence | SEQ ID NO: | CD4⁺ T Cell Epitope | Epitope Sequence | SEQ ID NO: |
|---|---|---|---|---|---|
| ORF1a₁₃₅₀₋₁₃₆₅ | KSAFYILPSIISNEK | 58 | S | NCYLPLKSYGFQPTY | 226 |
| ORF1a₁₈₀₁₋₁₈₁₅ | ESPFVMMSAPPAQYE | 59 | S | GNHKYRFRFFRKSNL | 227 |
| ORF1ab₅₀₁₉₋₅₀₃₃ | PNMLRIMASLVLARK | 60 | S | PFERDISTEIYQVGN | 228 |
| ORF1ab₆₀₈₈₋₆₁₀₂ | RIKVQMLSDTLKNL | 61 | S | KKLDSKVVGNHKYRF | 229 |
| ORF1aba₆₄₂₀₋₆₄₃₄ | LDAYNMMISAGFSLW | 62 | S | KGLNCYLPLKSYGFQ | 230 |
| S₁₋₁₃ | MFVFLVLLPLVSS | 63 | S | LVLLPLVSSQCVNFT | 231 |
| E₂₀₋₃₄ | FLAFVVFLLVTLAIL | 64 | S | RGDQVKQIAPGQTGN | 232 |
| E₂₆₋₄₀ | FLLVTLAILTALRLC | 65 | S | SASFSTFKCYGVSLN | 233 |
| M₁₇₆₋₁₉₀ | LSYYKLGASQRVAGD | 66 | S | KLDSKVVGNHKYRFR | 234 |
| ORF6₁₂₋₂₆ | AEILLIIMRTFKVSI | 67 | S | FAQVKQIYKTPPIKY | 235 |
| ORF7a₁₋₁₅ | MKIILFLALITLATC | 68 | S | ADYSFLYNSASFSTF | 236 |
| ORF7a₃₋₁₇ | IIFLALITLATCEL | 69 | S | ATKFSSVYAWDRRKI | 237 |
| ORF7a₉₈₋₁₁₂ | SPIFLIVAAIVFITL | 70 | S | PHRVWLSFELLHAS | 238 |
| ORF7b₈₋₂₂ | DFYLCFLAFLLFLVL | 71 | S | FERDISTEIYQVGNK | 239 |
| ORF8b₁₋₁₅ | MKFLVFLGIITTVAA | 72 | S | AKGLNCYLPLKSYGF | 240 |
| N₃₈₈₋₄₀₃₁ | KQQTVTLLPAADLDDF | 73 | S | SIVRFPNITNLCPFS | 241 |
| | | | S | NNCVADYSFLYNSAS | 242 |
| S | LCPFSEIFNATKFSS | 225 | S | KGAKGLNCYLPLKSY | 243 |

The present invention is not limited to the aforementioned CD4* T cell epitopes. For example, the present invention also includes variants of the aforementioned CD4⁺ T cell epitopes, for example sequences wherein the aforementioned CD4⁺ T cell epitopes are truncated by one or more amino acids or extended by one or more amino acids (examples shown below in Table 5).

TABLE 5

| CD4⁺ T Cell Epitope Origin | Sequence with Single AA Truncation | SEQ ID NO: | CD4⁺ T Cell Epitope Origin | Sequence with Single AA Truncation | SEQ ID NO: |
|---|---|---|---|---|---|
| ORF1a₁₃₅₀₋₁₃₆₅ | KSAFYILPSIISNE | 74 | ORF1a₁₃₅₀₋₁₃₆₅ | SAFYILPSIISNEK | 90 |
| ORF1a₁₈₀₋₁₈₁₅ | ESPFVMMSAPPAQY | 75 | ORF1a₁₈₀₁₋₁₈₁₅ | SPFVMMSAPPAQYE | 91 |
| ORF1ab₅₀₁₉₋₅₀₃₃ | PNMLRIMASLVLAR | 76 | ORF1ab₅₀₁₈₋₅₀₃₃ | NMLRIMASLVLARK | 92 |
| ORF1ab₆₀₈₈₋₆₁₀₂ | RIKVQMLSDTLKN | 77 | ORF1ab₆₀₈₈₋₆₁₀₂ | IKVQMLSDTLKNL | 93 |
| ORF1ab₆₄₂₀₋₆₄₃₄ | LDAYNMMISAGFSL | 78 | ORF1ab₆₄₂₀₋₆₄₃₄ | DAYNMMISAGFSLW | 94 |
| S₁₋₁₃ | MFVFLVLLPLVS | 79 | S₁₋₁₃ | FVFLVLLPLVSS | 95 |
| E₂₀₋₃₄ | FLAFVVFLLVTLAI | 80 | E₂₀₋₃₄ | LAFWFLLVTLAIL | 96 |
| E₂₆₋₄₀ | FLLVTLAILTALRL | 81 | E_{26–40} | LLVTLAILTALRLC | 97 |
| M₁₇₆₋₁₉₀ | LSYYKLGASQRVAG | 82 | M_{178–190} | SYYKLGASQRVAGD | 98 |
| ORF6₁₂₋₂₆ | AEILLIIMRTFKVS | 83 | ORF6_{12–25} | EILLIIMRTFKVSI | 99 |
| ORF7a₁₋₁₅ | MKIILFLALITLAT | 84 | ORF7a₁₋₁₅ | KIILFLALITLATC | 100 |
| ORF7a₃₋₁₇ | IIFLALITLATCE | 85 | ORF7a₃₋₁₇ | IFLALITLATCEL | 101 |
| ORF7a₉₈₋₁₁₂ | SPIFLIVAAIVFIT | 86 | ORF7a₉₈₋₁₁₂ | PIFLIVAAIVFITL | 102 |
| ORF7b₈₋₂₂ | DFYLCFLAFLLFLV | 87 | ORF7b₈₋₂₂ | FYLCFLAFLLFLVL | 103 |
| ORF8b₁₋₁₅ | MKFLVFLGIITTVA | 88 | ORF8b₁₋₁₅ | KFLVFLGIITTVAA | 104 |
| N₃₈₈₋₄₀₃₁ | KQQTVTLLPAADLDD | 89 | N₃₈₈₋₄₀₃₁ | QQTVTLLPAADLDDF | 105 |
| S | LCPFSEIFNATKFS | 244 | S | FAQVKQIYKTPPIK | 254 |
| S | NCYLPLKSYGFQPT | 245 | S | ADYSFLYNSASFST | 255 |
| S | GNHKYRFRFFRKSN | 246 | S | ATKFSSVYAWDRRK | 256 |
| S | PFERDISTEIYQVG | 247 | S | PHRVVVLSFELLHA | 257 |
| S | KKLDSKVVGNHKYR | 248 | S | FERDISTEIYQVGN | 258 |
| S | KGLNCYLPLKSYGF | 249 | S | AKGLNCYLPLKSYG | 259 |
| S | LVLLPLVSSQCVNF | 250 | S | SIVRFPNITNLCPF | 260 |
| S | RGDQVKQIAPGQTG | 251 | S | NNCVADYSFLYNSA | 261 |
| S | SASFSTFKCYGVSL | 252 | S | KGAKGLNCYLPLKS | 262 |
| S | KLDSKVVGNHKYRF | 253 | | | |

The present invention is not limited to the aforementioned CD4⁺ T cell epitopes.

### B Cell Epitopes

Examples of methods for identifying potential B cell epitopes and screening conservancy of potential B cell epitopes are described herein. The present invention is not limited to the particular software systems disclosed, and other software systems are accessible to one of ordinary skill in the art for such methods.

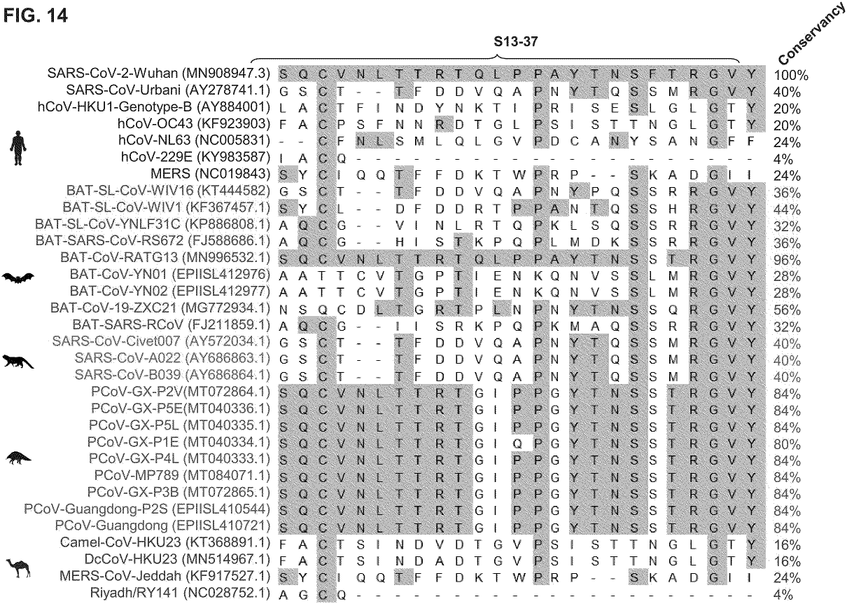

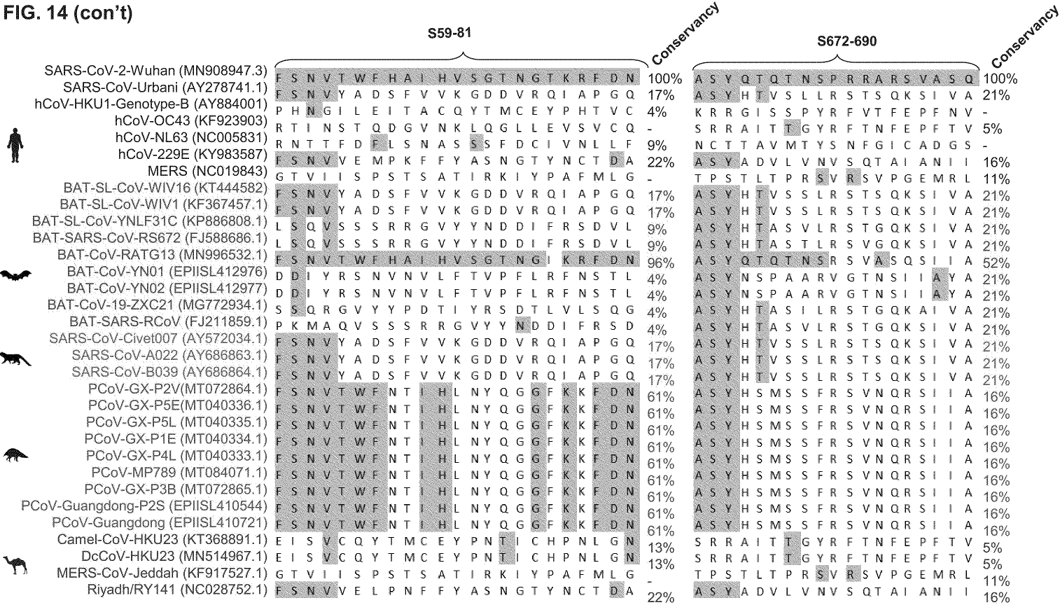

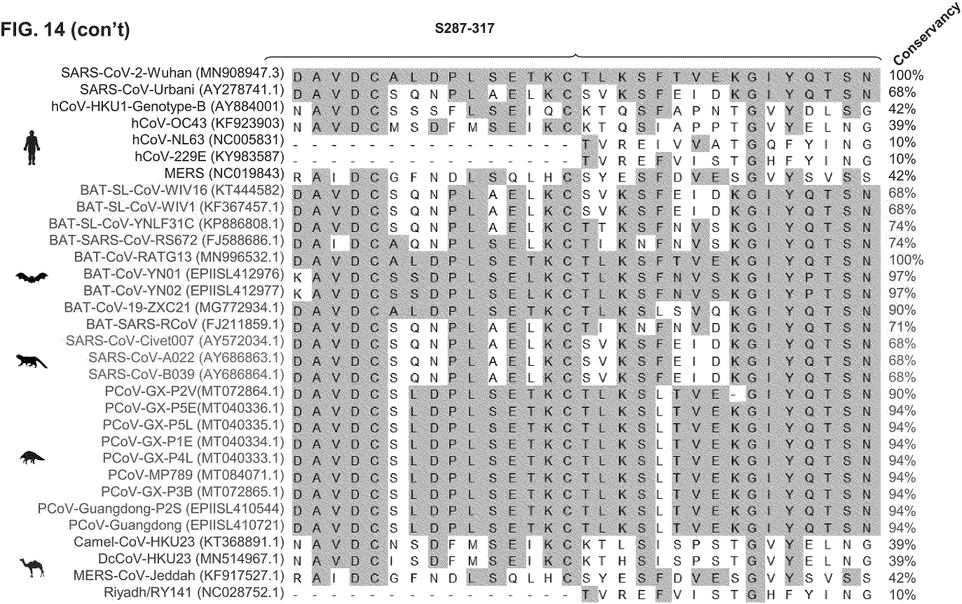

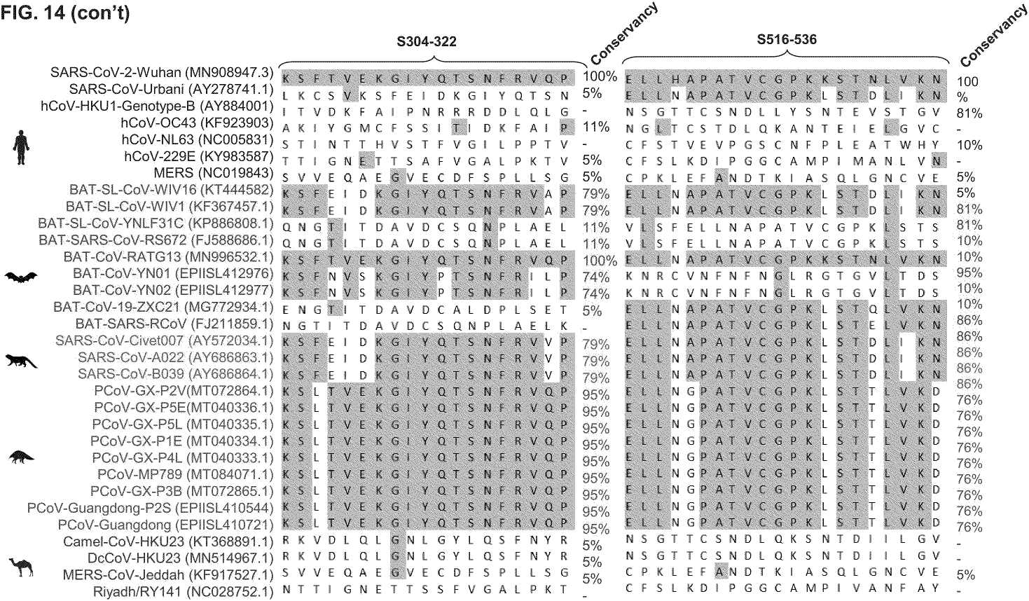

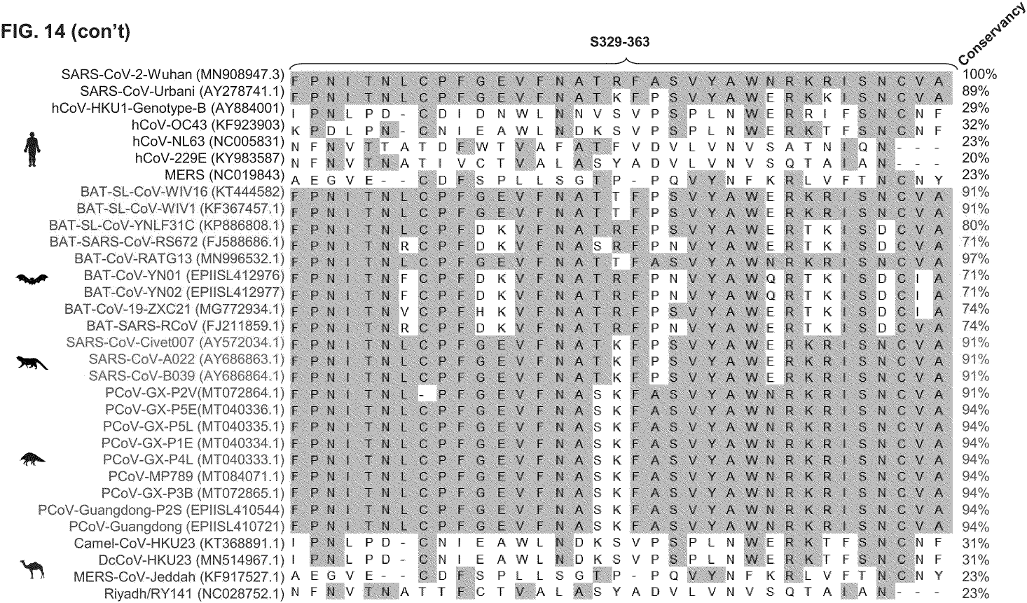

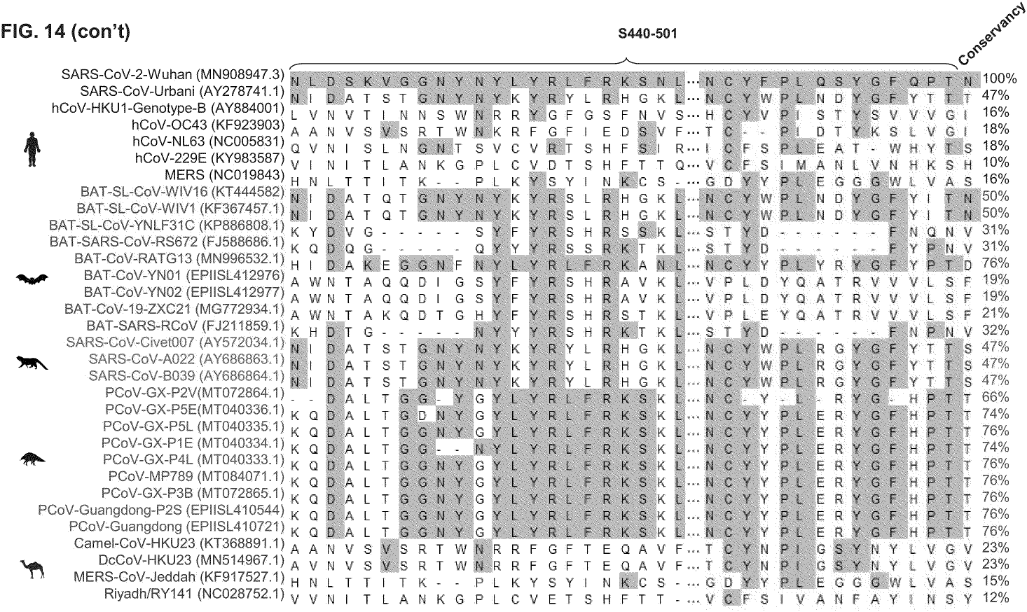

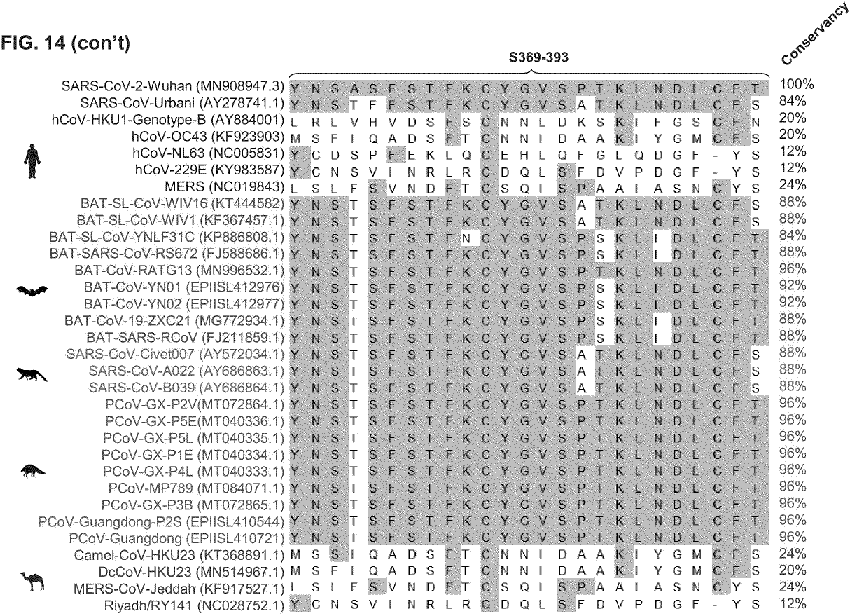

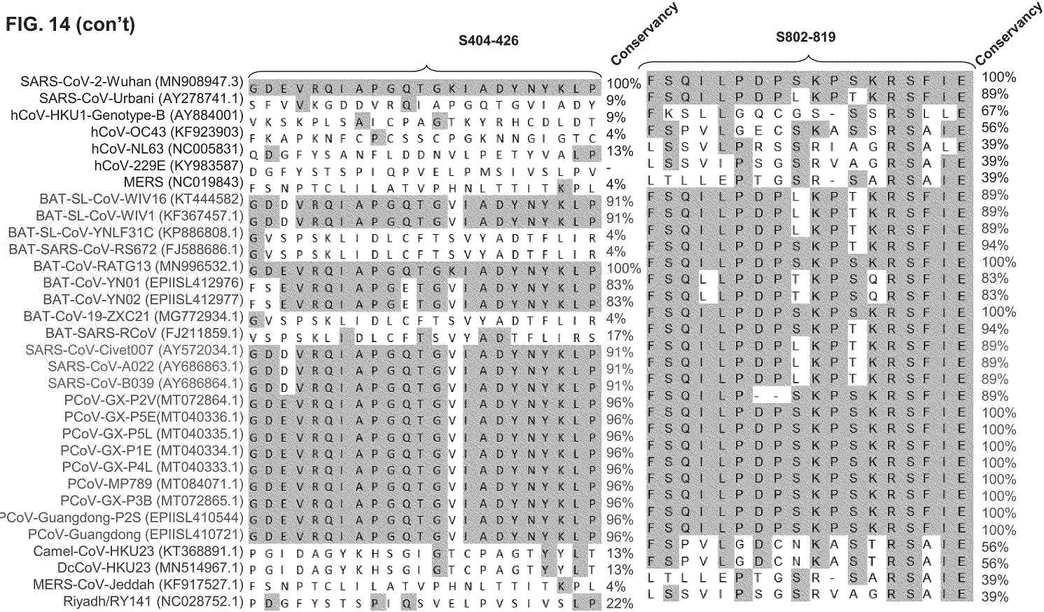

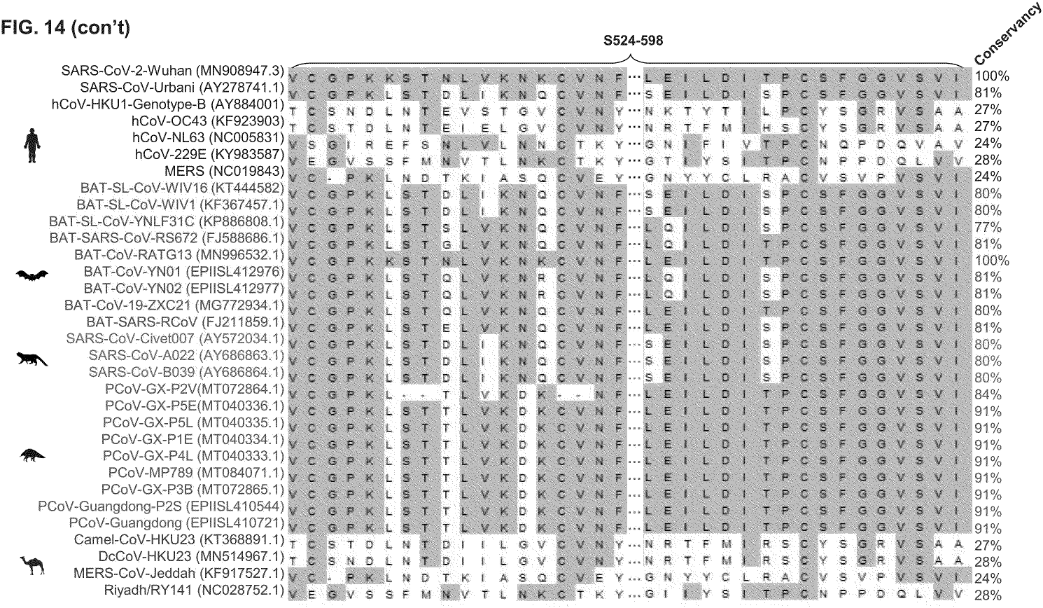

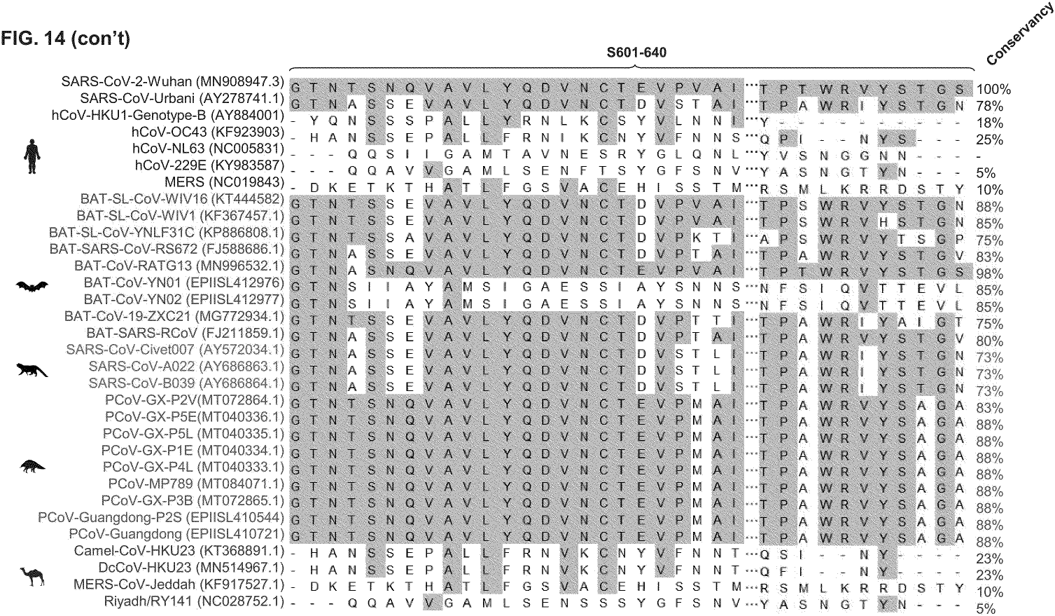

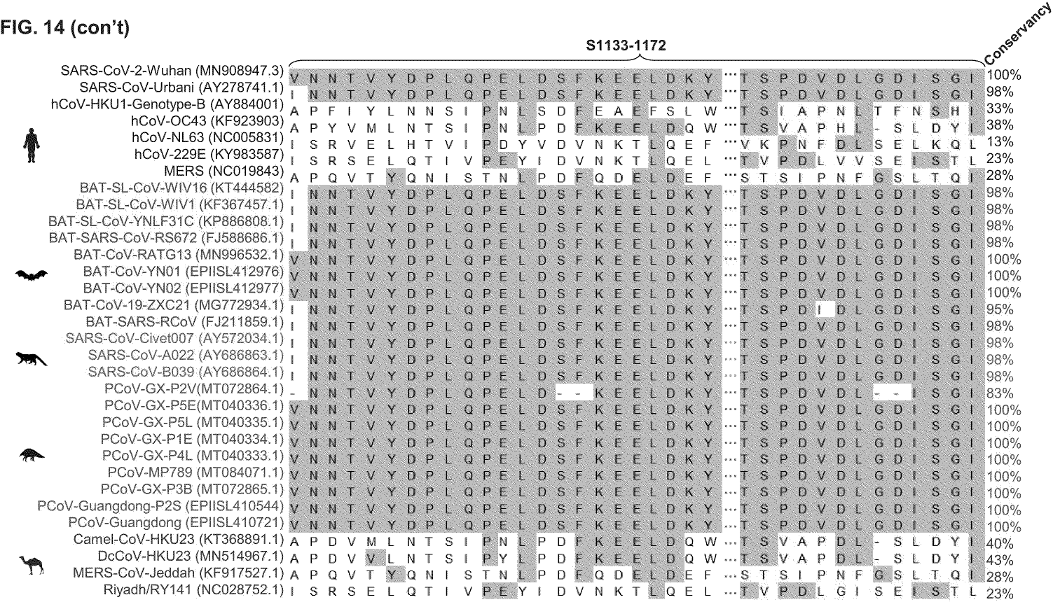

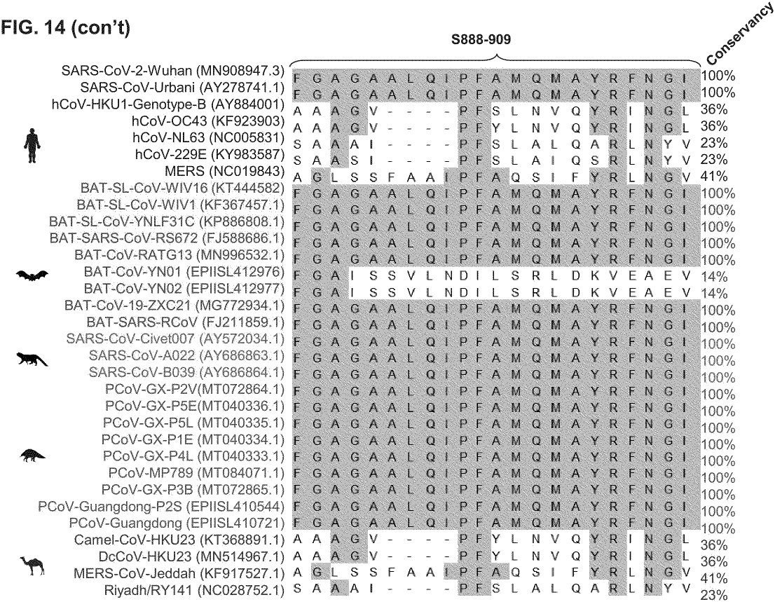

FIG. 14 shows the conservation of Spike-derived B cell epitopes among human, bat, civet cat, pangolin, and camel coronavirus strains. Multiple sequence alignment performed using ClustalW among 29 strains of SARS coronavirus (SARS-CoV) obtained from human, bat, civet, pangolin, and camel. This includes 7 human SARS/MERS-CoV strains (SARS-CoV-2-Wuhan (MN908947.3), SARS-HCoV-Urbani (AY278741.1), CoV-HKU1-Genotype-B (AY884001), CoV-OC43 (KF923903), CoV-NL63 (NC005831), CoV-229E (KY983587), MERS (NC019843)); 8 bat SARS-CoV strains (BAT-SL-CoV-WIV16 (KT444582), BAT-SL-CoV-WIV1 (KF367457.1), BAT-SL-CoV-YNLF31C (KP886808.1), BAT-SARS-CoV-RS672 (FJ588686.1), BAT-CoV-RATG13 (MN996532.1), BAT-CoV-YN01 (EPIISL412976), BAT-CoV-YN02 (EPIISL412977), BAT-CoV-19-ZXC21 (MG772934.1); 3 Civet SARS-CoV strains (SARS-CoV-Civet007 (AY572034.1), SARS-CoV-A022 (AY686863.1), SARS-CoV-B039 (AY686864.1)); 9 pangolin SARS-CoV strains (PCoV-GX-P2V(MT072864.1), PCoV-GX-P5E(MT040336.1), PCoV-GX-P5L (MT040335.1), PCoV-GX-P1E (MT040334.1), PCoV-GX-P4L (MT040333.1), PCoV-MP789 (MT084071.1), PCoV-GX-P3B (MT072865.1), PCoV-Guangdong-P2S (EPIISL410544), PCoV-Guangdong (EPIISL410721)); 4 camel SARS-CoV strains (Camel-CoV-HKU23 (KT368891.1), DcCoV-HKU23 (MN514967.1), MERS-CoV-Jeddah (KF917527.1), Riyadh/RY141 (NC028752.1)) and 1 recombinant strain (FJ211859.1)). Regions highlighted with blue color represent the sequence homology. The B cell epitopes, which showed at least 50% conservancy among two or more strains of the SARS Coronavirus or possess receptor-binding domain (RBD) specific amino acids were selected as candidate epitopes.

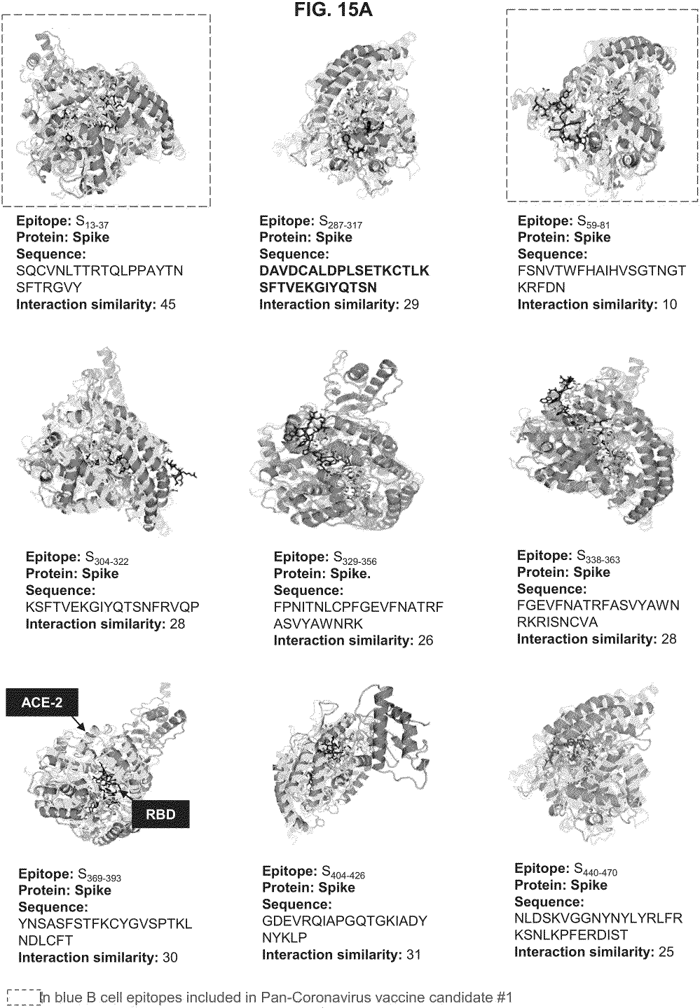

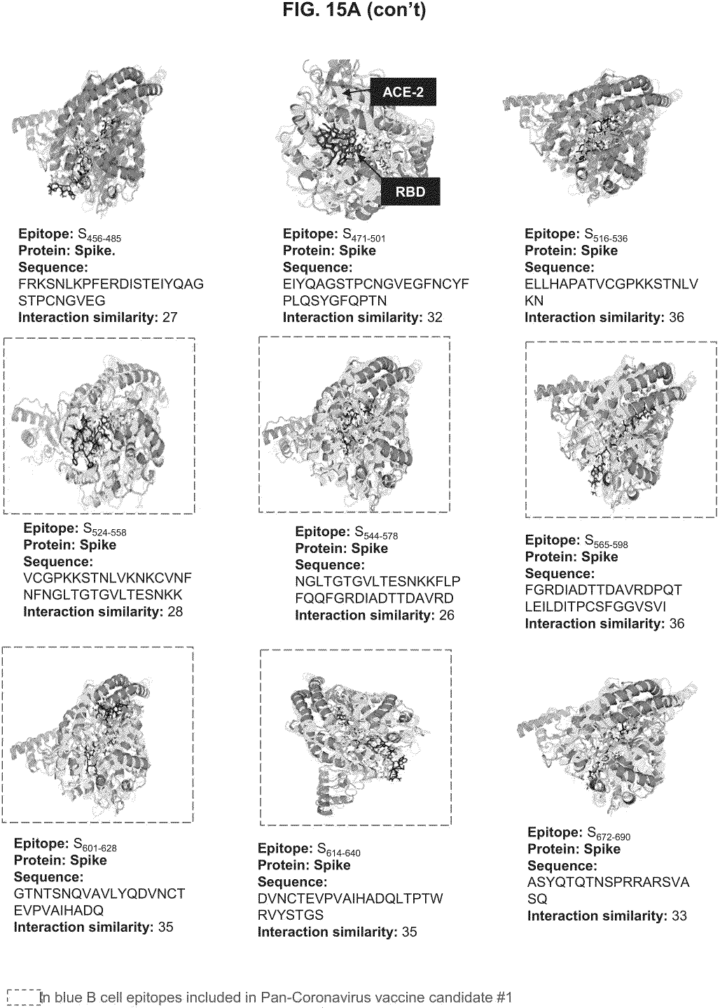

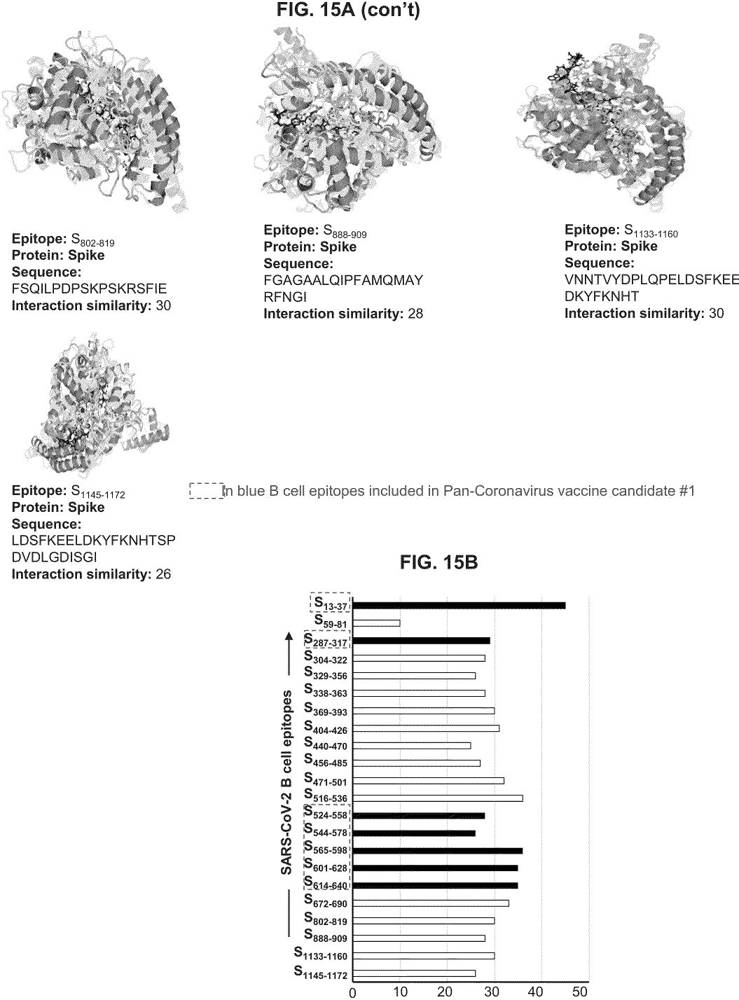

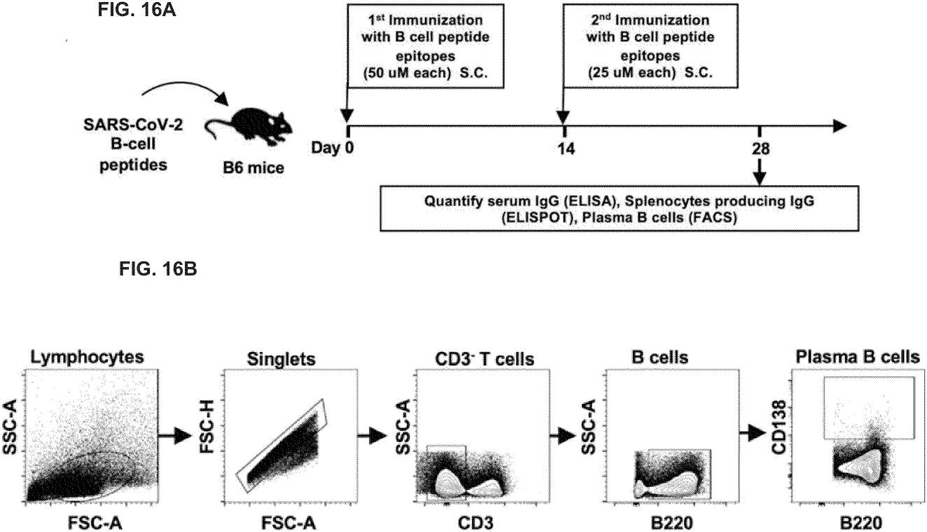

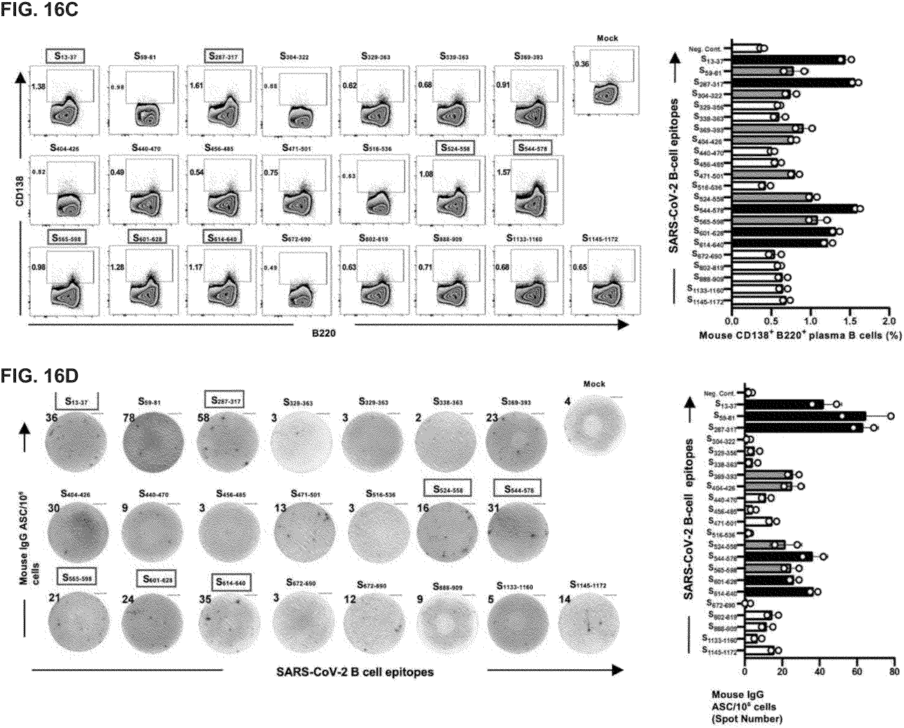

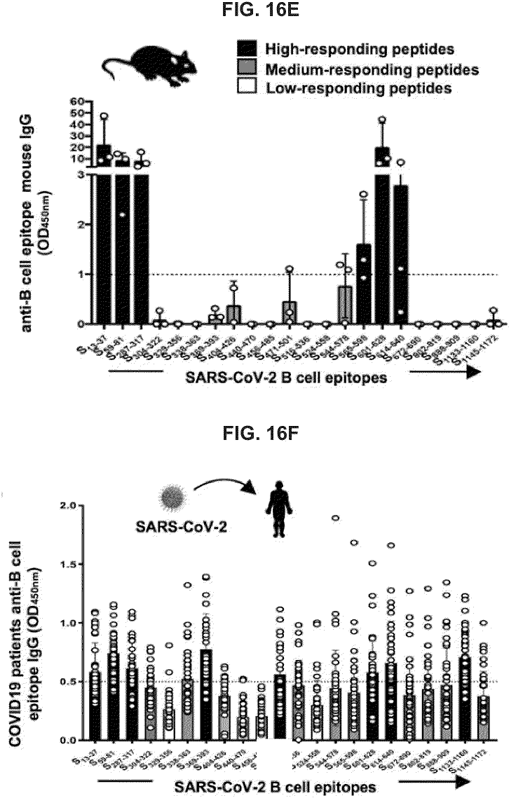

From the analysis, 22 B cell epitopes were selected as being highly conserved. FIG. 15A and FIG. 15B show the docking of the conserved epitopes to the ACE2 receptor as well as the interaction scores determined by protein-peptide molecular docking analysis. FIG. 16A, FIG. 16B, FIG. 16C, FIG. 16D, FIG. 16E, FIG. 16F, and FIG. 16G show immunogenicity of the identified SARS-CoV-2 B cell epitopes.

The B cell target epitopes discussed above include S₂₈₇₋₃₁₇, S₅₂₄₋₅₉₈, S₆₀₁₋₆₄₀, S₈₀₂₋₈₁₉, S₈₈₈₋₉₀₉, S₃₆₉₋₃₉₃, S₄₄₀₋₅₀₁, S₁₁₃₃₋₁₁₇₂, S₃₂₉₋₃₆₃, S₅₉₋₈₁, and S₁₃₋₃₇. FIG. 9 shows the genome-wide location of the epitopes. Thus, in certain embodiments, the vaccine composition may comprise one or more B cell target epitopes selected from: S₂₈₇₋₃₁₇, S₅₂₄₋₅₉₈, S₆₀₁₋₆₄₀, S₈₀₂₋₈₁₉, S₈₈₈₋₉₀₉, S₃₆₉₋₃₉₃, S₄₄₀₋₅₀₁, S_{1133-1172,} S₃₂₉₋₃₆₃, S₅₉₋₈₁, and S₁₃₋₃₇. In some embodiments, the B cell epitope is whole spike protein. In some embodiments, the B cell epitope is a portion of the spike protein. Table 6 below describes the sequences for the aforementioned epitope regions.

TABLE 6

| B Cell Epitope | Epitope Sequence | SEQ ID NO: | B Cell Epitope | Epitope Sequence | SEQ ID NO: |
|---|---|---|---|---|---|
| S₁₃₋₃₇ | SQCVNLTTRTQLPPAYTNSFT RGVY | 106 | S | CVNFTTRTQLPPAYTNSFT RGVYY | 263 |
| S₅₉₋₈₁ | FSNVTWFHAIHVSGTNGTKRF DN | 107 | S | NITNLCPFSEIFNATKFSSV YAWDRR | 264 |
| S₂₈₇₋₃₁₇ | DAVDCALDPLSETKCTLKSFT VEKGIYQTSN | 108 | S | INNCVADYSFLYNSASFST FKCYGVSLNKLNDL | 265 |
| S₆₀₁₋₆₄₀ | GTNTSNQVAVLYQDVNCTEV PVAIHADQLTPTWRVYSTGS | 109 | S | RGDQVKQIAPGQTGNIAD | 266 |
| S₅₂₄₋₅₉₈ | VCGPKKSTNLVKNKCVNFNFN GLTGTGVLTESNKKFLPFQQF GRDIADTTDAVRDPQTLEILDI TPCSFGGVSVI | 110 | S | KKLDSKWGNHKYRFRFFR KSNLKPFERDISTEISTEIY QVGNKPCKG | 267 |
| S₄₄₀₋₅₀₁ | NLDSKVGGNYNYLYRLFRKSN LKPFERDISTEIYQAGSTPCNG VEGFNCYFPLQSYGFQPTE | 111 | S | TYGVGY | 268 |
| S₃₆₉₋₃₉₃ | YNSASFSTFKCYGVSPTKLND LCFT | 112 | S | LHASATVCGPKKSTNL | 269 |
| S₃₂₉₋₃₆₃ | FPNITNLCPFGEVFNATRFASV YAWNRKRISNCVA | 113 | S | VKQIYKTPPIKYFGGFNFS QILPDPSKPSK | 270 |
| S₁₁₃₃₋₁₁₇₂ | VNNTVYDPLQPELDSFKEELD KYFKNHTSPDVDLGDISGI | 114 | | | |
| S₈₀₂₋₈₁₉ | FSQILPDPSKPSKRSFIE | 115 | | | |
| S₈₈₈₋₉₀₉ | FGAGAALQIPFAMQMAYRFN GI | 116 | | | |

The present invention is not limited to the aforementioned B cell epitopes. For example, the present invention also includes variants of the aforementioned B cell epitopes, for example sequences wherein the aforementioned B cell epitopes are truncated by one or more amino acids or extended by one or more amino acids (examples shown below in Table 7).

TABLE 7

| Origin of Epitope | Sequence with AA Truncation | SEQ ID NO: | Origin of Epitope | Sequence with AA Truncation | SEQ ID NO: |
|---|---|---|---|---|---|
| S₁₃₋₃₇ | SQCVNLTTRTQLPPAYTNSFT RG | 117 | S₁₁₋₃₇ | CVNLTTRTQLPPAYTNSFT RGVY | 128 |
| S₅₉₋₇₉ | FSNVTWFHAIHVSGTNGTKRF | 118 | S₈₁₋₉₁ | NVTWFHAIHVSGTNGTKR FDN | 129 |
| S₂₈₇₋₃₁₅ | DAVDCALDPLSETKCTLKSFT VEKGIYQT | 119 | S₂₈₉₋₃₁₇ | VDCALDPLSETKCTLKSFT VEKGIYQTSN | 130 |
| S₆₀₁₋₆₃₈ | GTNTSNQVAVLYQDVNCTEV PVAIHADQLTPTWRVYST | 120 | S₆₀₃₋₆₄₀ | NTSNQVAVLYQQVNCTEV PVAIHADQLTPTWRVYSTG S | 131 |
| S₅₂₄₋₅₉₈ | VCGPKKSTNLVKNKCVNFNFN GLTGTGVLTESNKKFLPFQQF GRDIADTTDAVRDPQTLEILDI TPCSFGGVS | 121 | S₅₂₆₋₅₉₈ | GPKKSTNLVKNKCVNFNF NGLTGTGVLTESNKKFLPF QQFGRDIADTTDAVRDPQ TLEILDITPCSFGGVSVI | 132 |
| S₄₄₀₋₄₉₉ | NLDSKVGGNYNYLYRLFRKSN LKPFERDISTEIYQAGSTPCNG VEGFNCYFPLQSYGFQP | 122 | S₄₄₂₋₅₀₁ | DSKVGGNYNYLYRLFRKS NLKPFERDISTEIYQAGSTP CNGVEGFNCYFPLOSYGF QPTE | 133 |
| S₃₆₉₋₃₉₁ | YNSASFSTFKCYGVSPTKLND LC | 123 | S₃₇₁₋₃₉₃ | SASFSTFKCYGVSPTKLND LCFT | 134 |
| S₃₂₉₋₃₆₁ | FPNITNLCPFGEVFNATRFASV YAWNRKRISNC | 124 | S₃₃₁₋₃₆₃ | NITNLCPFGEVFNATRFAS VYAWNRKRISNCVA | 135 |
| S₁₁₃₃₋₁₁₇₀ | VNNTVYDPLQPELDSFKEELD KYFKNHTSPDVDLGDIS | 125 | S₁₁₃₅₋ ₁₁₇₂ | NTVYDPLQPELDSFKEELD KYFKNHTSPDVDLGDISGI | 136 |
| S₈₀₂₋₈₁₇ | FSQILPDPSKPSKRSF | 126 | S₈₆₄₋₈₁₉ | QILPDPSKPSKRSFIE | 137 |
| S₈₈₈₋₉₀₇ | FGAGAALQIPFAMQMAYRFN | 127 | S₈₉₀₋₉₀₉ | AGAALQIPFAMQMAYRFN GI | 138 |
| S | CVNFTTRTQLPPAYTNSFTRG V | 271 | S | NFTTRTQLPPAYTNSFTRG VYY | 278 |
| S | NITNLCPFSEIFNATKFSSVYA WD | 272 | S | TNLCPFSEIFNATKFSSVYA WDRR | 279 |
| S | INNCVADYSFLYNSASFSTFK | 273 | S | NCVADYSFLYNSASFSTFK | 280 |
| | CYGVSLNKLN | | | CYGVSLNKLNDL | |
| S | RGDQVKQIAPGQTGNI | 274 | S | DQVKQIAPGQTGNIAD | 281 |
| S | KKLDSKWGNHKYRFRFFRKS NLKPFERDISTEISTEIYQVGN KPC | 275 | S | LDSKWGNHKYRFRFFRKS NLKPFERDISTEISTEIYQV GNKPCKG | 282 |
| S | LHASATVCGPKKST | 276 | S | ASATVCGPKKSTNL | 283 |
| S | VKQIYKTPPIKYFGGFNFSQIL PDPSKP | 277 | S | QIYKTPPIKYFGGFNFSQIL PDPSKPSK | 284 |

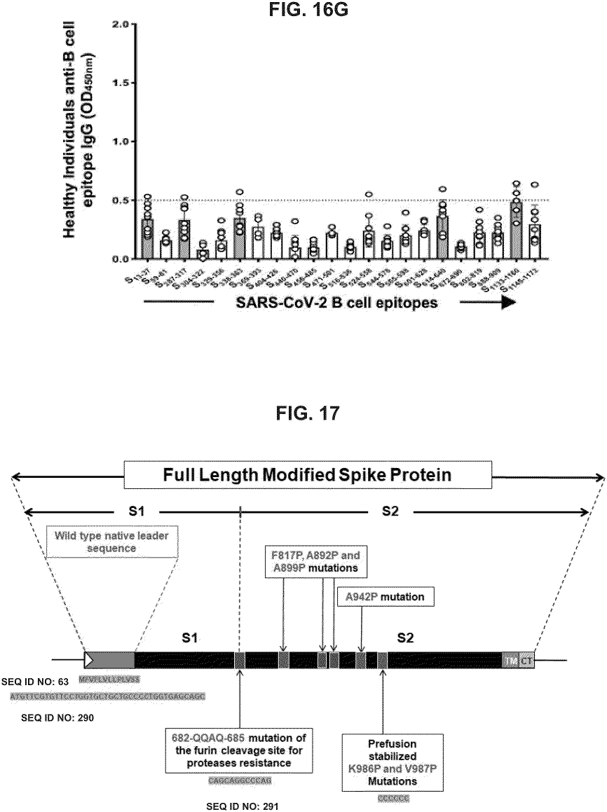

As previously discussed, in some embodiments, the B cell epitope is in the form of whole spike protein. In some embodiments, the B cell epitope is in the form of a portion of spike protein. In some embodiments, the transmembrane anchor of wherein the spike protein or portion thereof has an intact S1-S2 cleavage site. In some embodiments, wherein the spike protein or portion thereof is in its stabilized conformation. In some embodiments, the spike protein or portion thereof is stabilized with proline substitutions at amino acid positions 986 and 987 at the top of the central helix in the S2 subunit. In some embodiments, the composition comprises a trimerized SARS-CoV-2 receptor-binding domain (RBD). In some embodiments, the trimerized SARS-CoV-2 receptor-binding domain (RBD) sequence is modified by the addition of a T4 fibritin-derived foldon trimerization domain. In some embodiments, the addition of a T4 fibritin-derived foldon trimerization domain increases immunogenicity by multivalent display. FIG. 17 shows a non-limiting example of a spike protein comprising one or more mutations.

In some embodiments, wherein the spike protein or portion thereof comprises Tyr-489 and Asn-487 (e.g., Tyr-489 and Asn-487 help with interaction with Tyr 83 and Gln-24 on ACE-2). In some embodiments, wherein the spike protein or portion thereof comprises Gin-493 (e.g., Gln-493 helps with interaction with Glu-35 and Lys-31 on ACE-2). In some embodiments, wherein the spike protein or portion thereof comprises Tyr-505 (e.g., Tyr-505 helps with interaction with Glu-37 and Arg-393 on ACE-2). In some embodiments, the composition comprises a mutation 682-RRAR-685 → 682-QQAQ-685 in the S1-S2 cleavage site.

In some embodiments, the composition comprises at least one proline substitution. In some embodiments, the composition comprises at least two proline substitutions. For example, the proline substitution may be at position K986 and V987.

As previously discussed, in some embodiments, the composition comprises spike protein or portion thereof. Non-limiting examples of spike proteins are listed in Table 8.

TABLE 8

| | Sequence: | SEQ ID NO: |
|---|---|---|
| SARS-CoV-like Spike-S1-NTD 13 bp-304 bp | SQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNWIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLK | 176 |
| SARS-CoV-2 Spike-S1-RBD 319 bp-541 bp | RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF | 177 |
| CoV Spike S1-S2_S2 543 bp-1,208 bp | FNGLTGTGVLTESNKKFLPFQQFGRDIADTTDAVTRAGCLIGAEHVNNSYECDIPIGAGICASYQTQTNRDPQTLEILDITPCSFGGVSVITPGTNTSNQVAVLYQDVNCTEVPVAIHADQLTPTWRVYSTGSNVFQSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDWNQNAQALNTLVKQLSSNSNFGAISSVLNDILSRLDKVEAEVQIDRLITGRLQSLQTYVTQQLIRAAEIRASANLAATKMSECVLGQSKRVDFCGKGYHLMSFPQSAPHGWFLHVTYVPAQEKNFTTAPAICHDGKAHFPREGVFVSNGTHWFVTQRNFYEPQIITTDNTFVSGNCDWIGIVNNTVYDPLQPELDSFKEELDKYFKNHTSPDVDLGDISGINASWNIQKEIDRLNEVAKNLNESLIDLQELGKYEQ | 178 |
| spike glycoprotein with a mutation 682-RRAR-685 → 682-QQAQ-685 in the S1-S2 cleavage site | VFLVLLPLVSSQCVNLTTRTQLPPAYTNSFTRGVYYPDKVFRSS VLHSTQDLFLPFFSNVTWFHAIHVSGTNGTKRFDNPVLPFNDGVYFASTEKSNIIRGWIFGTTLDSKTQSLLIVNNATNWIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRWVLSFELLHAPATVCGPKKSTNLVKNKCVNFNFNGLTGTGVLTESNKKFLPFQQFGRDIADTTDAVTRAGCLIGAEHVNNSYECDIPIGAGICASYTQTNRDPQTLEILDITPCSFGGVSVITPGTNTSNQVAVLYQDVNCTEVPVAIHADQLTPTWRVYSTGSNVFQSPQQAQSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDWIGIVNNTVYDPLQPELDSFKEELDKYFKNHTSPDVDLGDISGINASWNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT | 179 |
| spike glycoprotein with two proline substitutions (K986P, V987P) | MFLVLLPLVSSQCVNLTTRTQLPPAYTNSFTRGVYYPDKVFRSS VLHSTQDLFLPFFSNVTWFHAIHVSGTNGTKRFDNPVLPFNDGVYFASTEKSNIIRGWIFGTTLDSKTQSLLIVNNATNWIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRWVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDWIGIVNNTVYDPLQPELDSFKEELDKYFKNHTSPDVDLGDISGINASWNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT | 180 |
| | MFVFLVLLPLVSSQCVNLTTRTQLPPAYTNSFTRGVYYPDKVFRSS VLHSTQDLFLPFFSNVTWFHAIHVSGTNGTKRFDNPVLPFNDGVYFASTEKSNIIRGWIFGTTLDSKTQSLLIVNNATNWIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRWVLSFELLHAPATVCGPKKSTNLVKNKCVNFNFNGLTGTGVLTESNKKFLPFQQFGRDIADTTDAVTRAGCLIGAEHVNNSYECDIPIGAGICASYQTQTNRDPQTLEILDITPCSFGGVSVITPGTNTSNQVAVLYQDVNCTEVPVAIHADQLTPTWRVYSTGSNVFQSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDWNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDWIGIVNNTVYDPLQPELDSFKEELDKYFKNHTSPDVDLGDISGINASWNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT | 181 |
| spike glycoprotein with six proline substitutions (F817P, A892P, A899P, A942P, K986P, V987P) | MFVFLVLLPLVSSQCVNLTTRTQLPPAYTNSFTRGVYYPDKVFRSS VLHSTQDLFLPFFSNVTWFHAIHVSGTNGTKRFDNPVLPFNDGVYFASTEKSNIIRGWIFGTTLDSKTQSLLIVNNATNWIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRWVLSFELLHAPATVCGPKKSTNLVKNKCVNFNFNGLTGTGVLTESNKKFLPFQQFGRDIADTTDAVTRAGCLIGAEHVNNSYECDIPIGAGICASYQTQTNRDPQTLEILDITPCSFGGVSVITPGTNTSNQVAVLYQDVNCTEVPVAIHADQLTPTWRVYSTGSNVFQSPRRARSVASQSIIAYTMSLGAENSVAYSNNSIAIPTNFTISVTTEILPVSMTKTSVDCTMYICGDSTECSNLLLQYGSFCTOLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDWNQNAQALNTLVKQLSSNFGAISSVLNDILSRLDPPEAEVQIDRLITGRLQSLQTYVTQQLIRAAEIRASANLAATKMSECVLGOSKRVDFCGKGYHLMSFPQSAPHGVVFLHVTYVPAQEKNFTTAPAICHDGKAHFPREGVFVSNGTHWFVTQRNFYEPQIITTDNTFVSGNCDWIGIVNNTVYDPLQPELDSFKEELDKYFKNHTSPDVDLGDISGINASWNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT | 182 |
| spike glycoprotein with six proline substitutions (F817P, A892P, A899P, A942P, K986P, V987P) and a 682-RRAR-685 → 682-QQAQ-685 mutation | MFVFLVLLPLVSSQCVNLTTRTQLPPAYTNSFTRGVYYPDKVFRSS VLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRWVLSFELLHAPATVCGPKKSTNLVKNKCVNFNFNGLTGTGVLTESNKKFLPFQQFGRDIADTTDAVTRAGCLIGAEHVNNSYECDIPIGAGICASYQTQTNRDPQTLEILDITPCSFGGVSVITPGTNTSNQVAVLYQDVNCTEVPVAIHADQLTPTWRVYSTGSNVFQSPQQAQSVASQSIIAYTMSLGAENSVAYSNNSIAIPTNFTISVTTEILPVSMTKTSVDCTMYICGDSTECSNLLLQYGSFCTOLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGOSKRVDFCGKGYHLMSFPQSAPHGVVFLHVTYVPAQEKNFTTAPAICHDGKAHFPREGVFVSNGTHWFVTQRNFYEPQIITTDNTFVSGNCDWIGIVNNTVYDPLQPELDSFKEELDKYFKNHTSPDVDLGDISGINASWNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT | 183 |
| Wild type native leader sequence | MFVFLVLLPLVSS | 63 |

### Vaccine Candidates

As previously discussed, the present invention provides vaccine compositions comprising: at least one B cell epitope and at least one CD4+ T cell epitope, at least one B cell epitope and at least one CD8+ T cell epitope, at least one CD4+ T cell epitope and at least one CD8+ T cell epitope, or at least one B cell epitope, at least one CD4+ T cell epitope, and at least one CD8+ T cell epitope.

In certain embodiments, at least one epitope is derived from a non-spike protein. In certain embodiments, the composition induces immunity to only the epitopes.

FIG. 18 shows a schematic representation of a prototype Coronavirus vaccine of the present invention. This first candidate was delivered in ACE2/HLA1/2 triple transgenic mice using 3 different antigen delivery systems (1) peptides injected subcutaneously; (2) modified mRNA injected subcutaneously; and (3) AAV9 administered intra nasally the Virological. Clinical and Immunological results obtained point to an excellent protection against both virus replication in the lungs and COVID-like symptoms (Such as loss of weight), deaths. This protection correlated with an excellent Band T cell immunogenicity of this first multi-epitope pan-Coronavirus vaccine candidate #B1, with antibodies, CD4 T cell and CD8 T cells specific to multiple epitopes encoded by this vaccine were induced and correlated with protection.

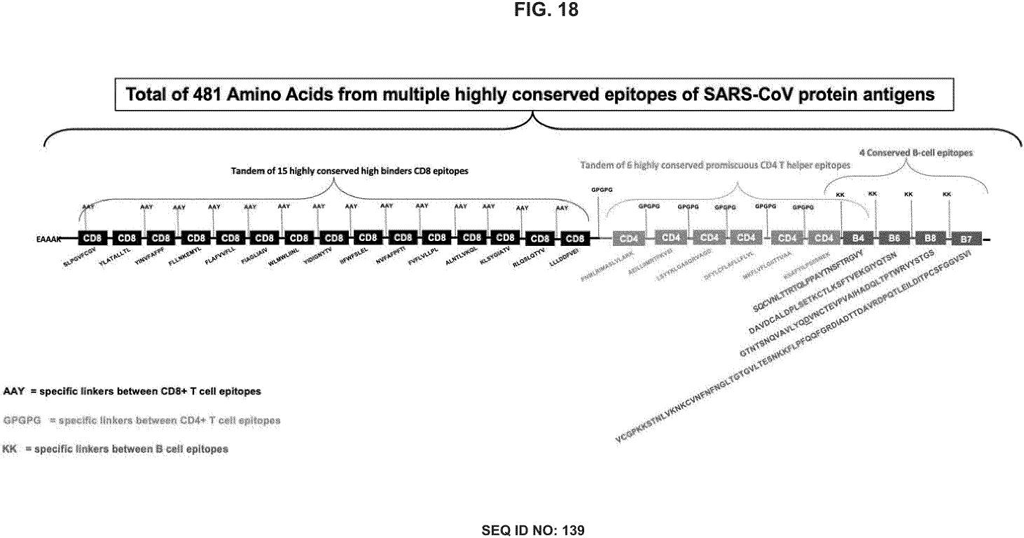

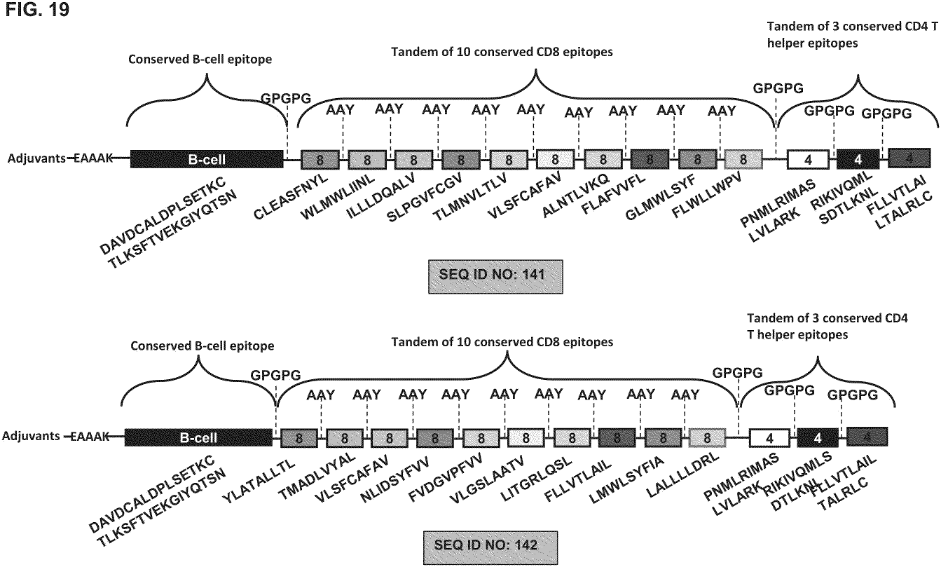

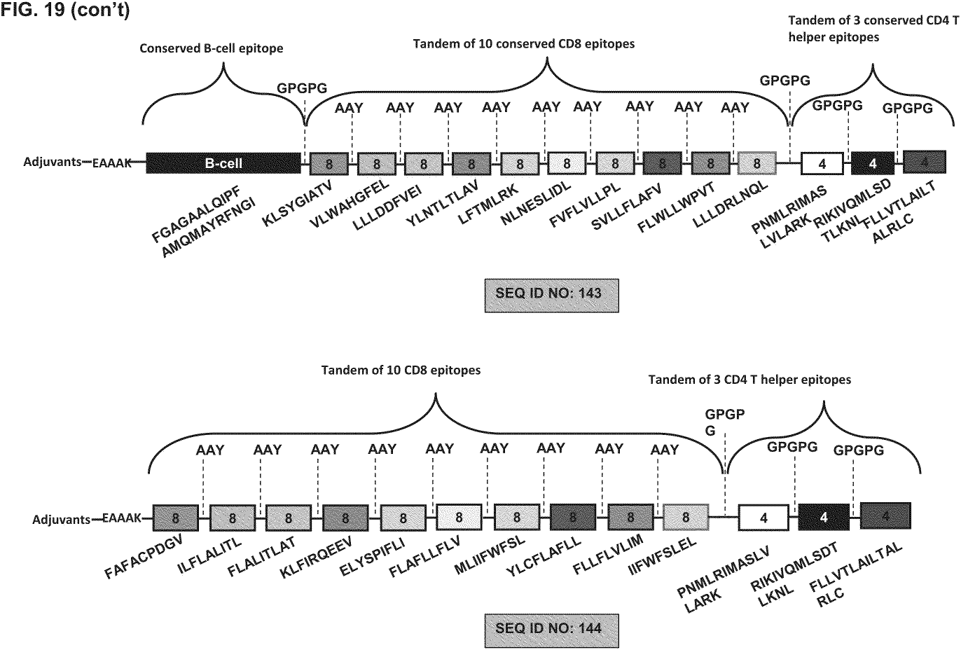

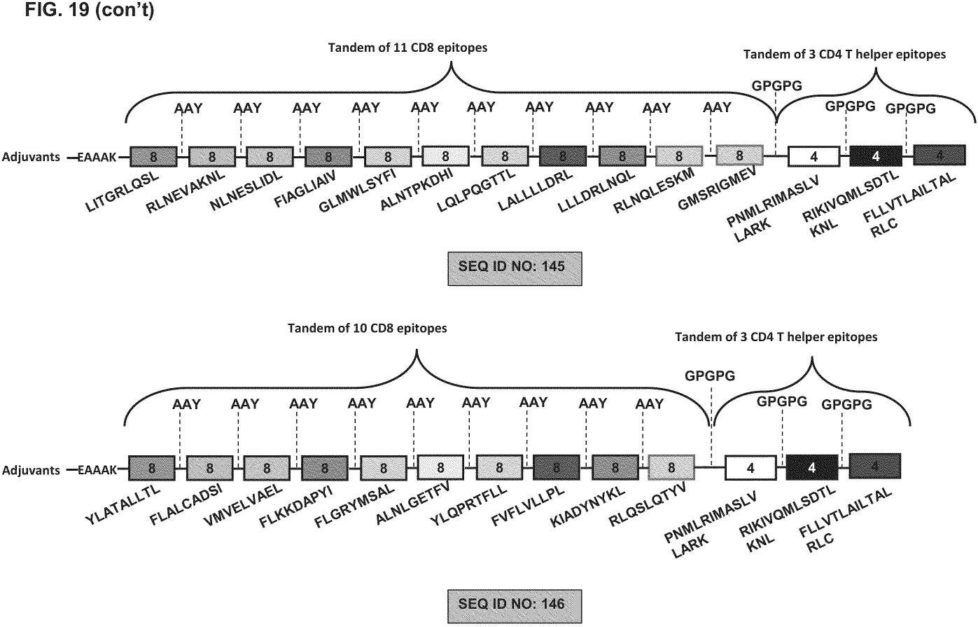

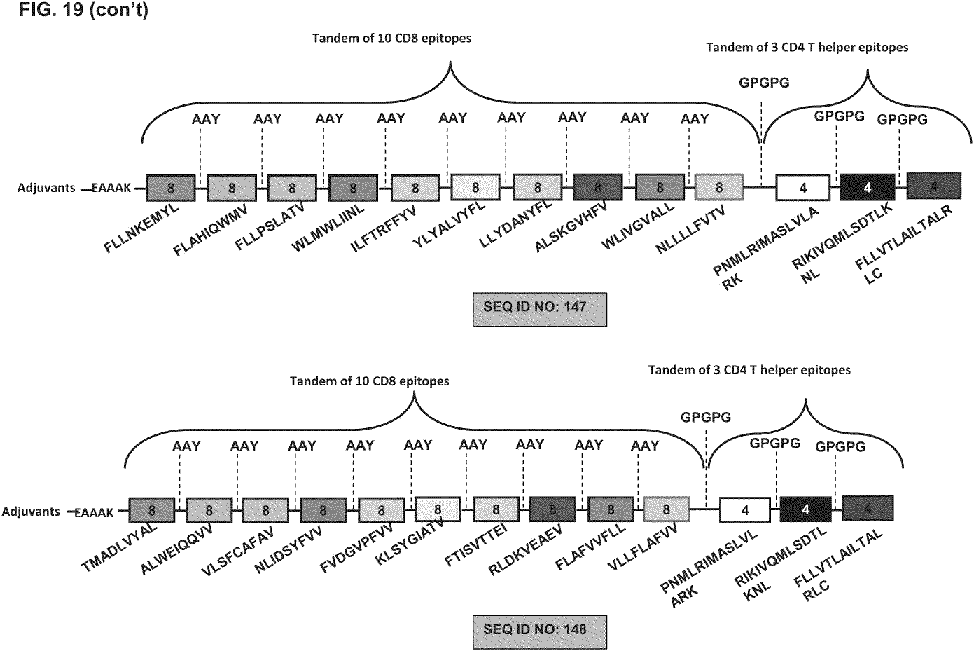

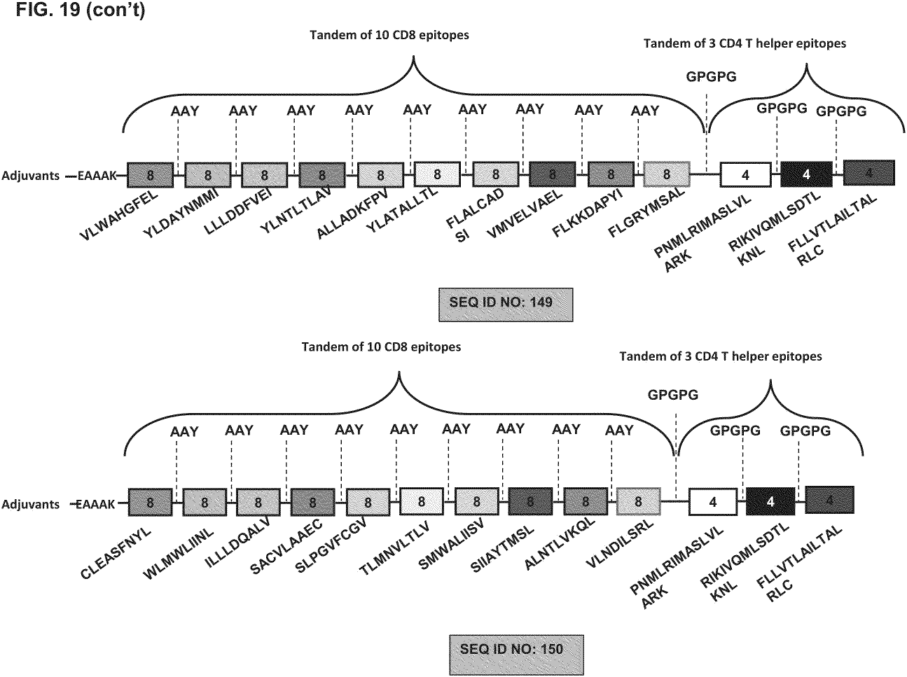

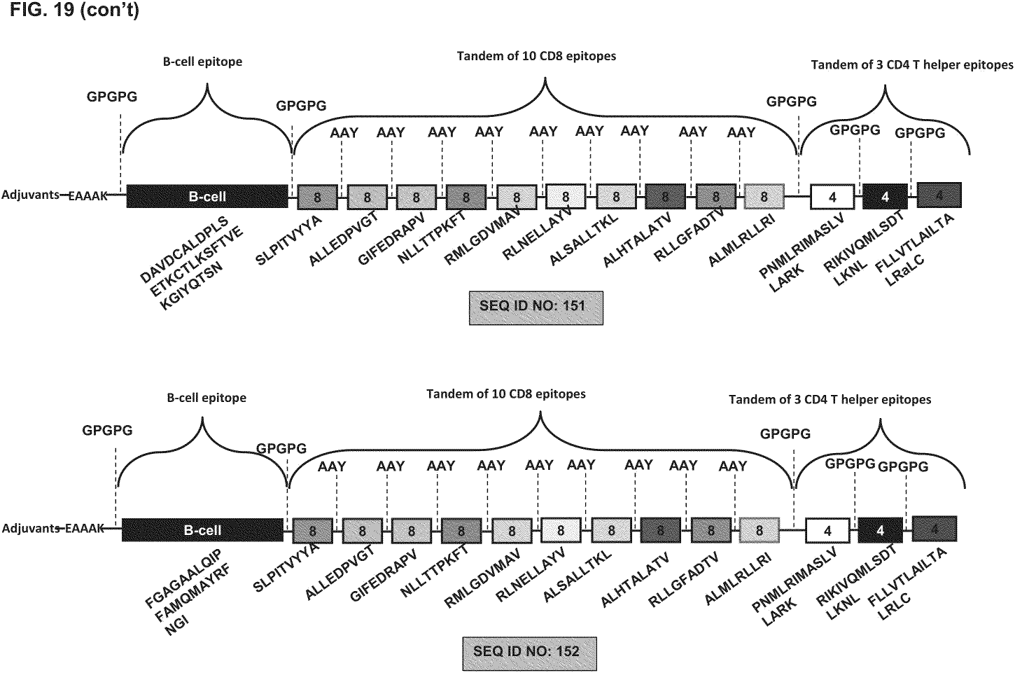

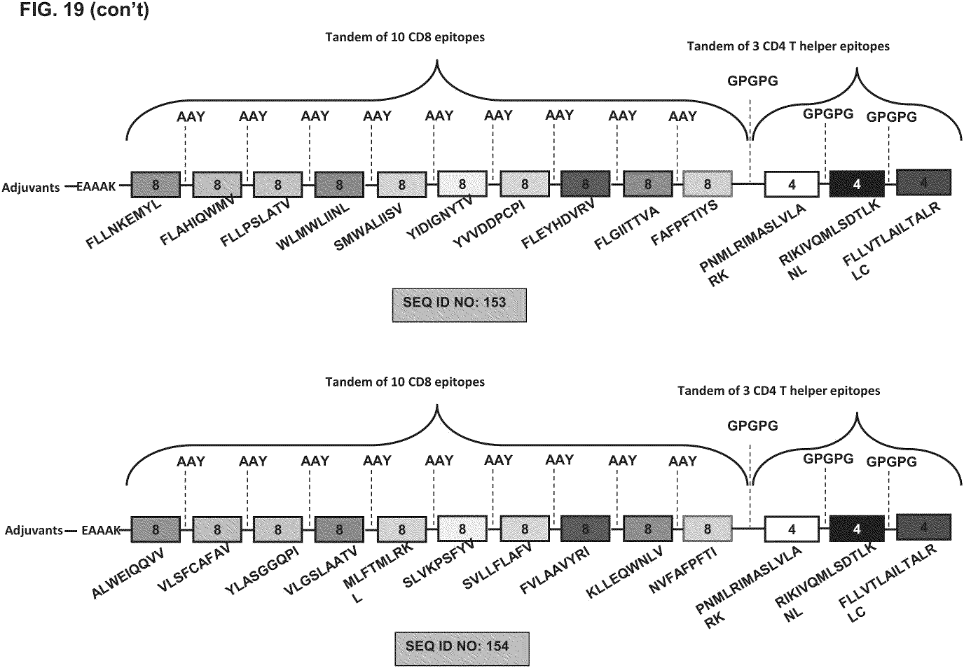

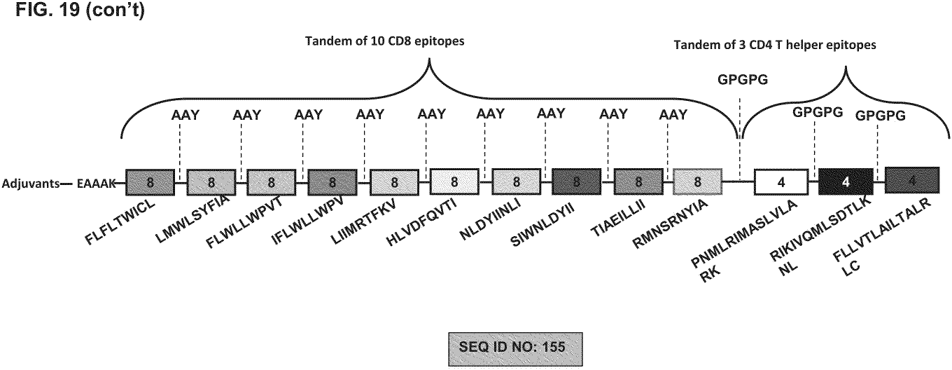

Table 9 and FIG. 19 show examples of vaccine compositions described herein. The present invention is not limited to the examples in Table 9. Residues in bold are linkers, residues that are underlined refer to the CD8+ T cell epitope region, residues in plain text refer to CD4+ T cell epitopes, and residues that are italicized refer to B cell epitopes. As an example, an AAY linker is added between CD8+ T cell epitopes, and a GPGPG linker is added between the B-cell epitope and CD8+ T cell epitopes as well as between all CD4+ T cell helper epitopes. The linkers may enhance epitope presentation and remove junctional epitopes.

TABLE 9

| Vaccine Candidate | Sequence: | SEQ ID NO: |
|---|---|---|
| 1 | EAAAKSLPGVFCGVAAYYLATALLTLAAYYINVFAFPFAAYFLLNKEMYLAAYFLAFVVLAAYFIAGLIAIVAAYWLMWLIINIAAYYIDIGNYTVAAYIIFWFSLELAAYNVFAFPFTIAAYFVFLVLLPLAAYALNTLVKQLAAYKLSYGIATVAAYRLQSLQTYVAAYLLLDDFVEIGPGPGPNMLRIMASLVLARKGPGPGAEILLIIMRTFKVSIGPGPGLSYYKLGASQRVAGDGPGPGDFYLCFLAFLLFLVLGPGPGMKFLVFLGIITTVAAGPGPGKSAFYILPSIISNEKKKSQCVNLTTRTQLPPAYTNSFTRGVYKKDAVDCALDPLSETKCTLKSFTVEKGIYQTSNKKGTNTSNQVAVLYQDVNCTEVPVAIHADQLTPTWRVYSTGSKKVCGPKKSTNLVKNKCVNFNFNGLTGTGVLTESNKKFLPFQQFGRDIADTTDAVRDPQTLEILDITPCSFGGVSVI | 139 |
| 2 | EAAAKYNSASFSTFKCYGVSPTKLNDLCFTGPGPGCLEASFNYLAAYWLMWLIINLAAYILLLDQALVAAYSLPGVFCGVAAYTLMNVLTLVAAYVLSFCAFAVAAYALNTLVKQAAYFLAFVVFLAAYGLMWLSYFAAYFLWLLWPVGPGPGPNMLRIMASLVLARKGPGPGRIKIVQMLSDTLKNLGPGPGFLLVTLAILTALRLC | 140 |
| 3 | EAAAKDAVDCALDPLSETKCTLKSFTVEKGIYQTSNGPGPGCLEASFNYLAAYWLMWLIINLAAYILLLDQALVAAYSLPGVFCGVAAYTLMNVLTLVAAYVLSFCAFAVAAYALNTLVKQAAYFLAFVVFLAAYGLMWLSYFAAYFLWLLWPVGPGPGPNMLRIMASLVLARKGPGPGRIKIVQMLSDTLKNLGPGPGFLLVTLAILTALRLC | 141 |
| 4 | EAAAKDAVDCALDPLSETKCTLKSFTVEKGIYQTSNGPGPGYLATALLTLAAYTMADLVYALAAYVLSFCAFAVAAYNLIDSYFVVAAYFVDGVPFVVAAYVLGSLAATVAAYLITGRLQSLAAYFLLVTLAILAAYLMWLSYFIAAAYLALLLLDRLGPGPGPNMLRIMASLVLARKGPGPGRIKIVQMLSDTLKNLGPGPGFLLVTLAILTALRLC | 142 |
| 5 | EAAAKFGAGAALQIPFAMQMAYRFNGIGPGPGKLSYGIATVAAYVLWAHGFELAAYLLLDDFVEIAAYYLNTLTLAVAAYLFTMLRKAAYNLNESLIDLAAYFVFLVLLPLAAYSVLLFLAFVAAYFLWLLWPVTAAYLLLDRLNQLGPGPGPNMLRIMASLVLARKGPGPGRIKIVQMLSDTLKNLGPGPGFLLVTLAILTALRLC | 143 |
| 6 | EAAAKFAFACPDGVAAYILFLALITLAAYFLALITLATAAYKLFIRQEEVAAYELYSPIFLIAAYFLAFLLFLVAAYMLIIFWFSLAAYYLCFLAFLLAAYFLLFLVLIMAAYIIFWFSLELGPGPGPNMLRIMASLVLARKGPGPGRIKIVQMLSDTLKNLGPGPGFLLVTLAILTALRLC | 144 |
| 7 | EAAAKLITGRLQSLAAYRLNEVAKNLAAYNLNESLIDLAAYFIAGLIAIVAAYGLMWLSYFIAAYALNTPKDHIAAYLQLPQGTTLAAYLALLLLDRLAAYLLLDRLNQLAAYRLNQLESKMAAYGMSRIGMEVGPGPGPNMLRIMASLVLARKGPGPGRIKIVQMLSDTLKNLGPGPGFLLVTLAILTALRLC | 145 |
| 8 | EAAAKYLATALLTLAAYFLALCADSIAAYVMVELVAELAAYFLKKDAPYIAAYFLGRYMSALAAYALNLGETFVAAYYLQPRTFLLAAYFVFLVLLPLAAYKIADYNYKLAAYRLQSLQTYVGPGPGPNMLRIMASLVLARKGPGPGRIKIVQMLSDTLKNLGPGPGFLLVTLAILTALRLC | 146 |
| 9 | EAAAKFLLNKEMYLAAYFLAHIQWMVAAYFLLPSLATVAAYWLMWLIINLAAYILFTRFFYVAAYYLYALVYFLAAYLLYDANYFLAAYALSKGVHFVAAYWLIVGVALLAAYNLLLLFVTVGPGPGPNMLRIMASLVLARKGPGPGRIKIVQMLSDTLKNLGPGPGFLLVTLAILTALRLC | 147 |
| 10 | EAAAKTMADLVYALAAYALWEIQQVVAAYVLSFCAFAVAAYNLIDSYFVVAAYFVDGVPFVVAAYKLSYGIATVAAYFTISVTTEIAAYRLDKVEAEVAAYFLAFVVFLLAAYVLLFLAFVVGPGPGPNMLRIMASLVLARKGPGPGRIKIVQMLSDTLKNLGPGPGFLLVTLAILTALRLC | 148 |
| 11 | EAAAKVLWAHGFELAAYYLDAYNMMIAAYLLLDDFVEIAAYYLNTLTLAVAAYALLADKFPVAAYYLATALLTLAAYFLALCADSIAAYVMVELVAELAAYFLKKDAPYIAAYFLGRYMSALGPGPGPNMLRIMASLVLARKGPGPGRIKIVQMLSDTLKNLGPGPGFLLVTLAILTALRLC | 149 |
| 12 | EAAAKFLLNKEMYLAAYFLAHIQWMVAAYFLLPSLATVAAYWLMWLIINLAAYSMWALIISVAAYYIDIGNYTVAAYYVVDDPCPIAAYFLEYHDVRVAAYFLGIITTVAAAYFAFPFTIYSGPGPGPNMLRIMASLVLARKGPGPGRIKIVQMLSDTLKNLGPGPGFLLVTLAILTALRLC | 150 |
| 13 | EAAAKALWEIQQVVAAYVLSFCAFAVAAYYLASGGQPIAAYVLGSLAATVAAYMLFTMLRKLAAYSLVKPSFYVAAYSVLLFLAFVAAYFVLAAVYRIAAYKLLEQWNLVAAYNVFAFPFTIGPGPGPNMLRIMASLVLARKGPGPGRIKIVQMLSDTLKNLGPGPGFLLVTLAILTALRLC | 151 |
| 14 | EAAAKFLFLTWICLAAYLMWLSYFIAAAYFLWLLWPVTAAYIFLWLLWPVAAYLIIMRTFKVAAYHLVDFQVTIAAYNLDYIINLIAAYSIWNLDYIIAAYTIAEILLIIAAYRMNSRNYIAGPGPGPNMLRIMASLVLARKGPGPGRIKIVQMLSDTLKNLGPGPGFLLVTLAILTALRLC | 152 |
| 15 | EAAAKCLEASFNYLAAYWLMWLIINLAAYILLLDQALVAAYSACVLAAECAAYSLPGVFCGVAAYTLMNVLTLVAAYSMWALIISVAAYSIIAYTMSLAAYALNTLVKQLAAYVLNDILSRLGPGPGPNMLRIMASLVLARKGPGPGRIKIVQMLSDTLKNLGPGPGFLLVTLAILTALRLC | 153 |
| 16 | EAAKDAVDCALDPLSETKCTLKSFTVEKGIYQTSNGPGPGSLPITVYYAAAYALLEDPVGTAAYGIFEDRAPVAAYNLLTTPKFTAAYRMLGDVMAVAAYRLNELLAYVAAYALSALLTKLAAYALHTALATVAAYRLLGFADTVAAYALMLRLLRIGPGPGPNMLRIMASLVLARKGPGPGRIKIVQMLSDTLKNLGPGPGFLLVTLAILTALRLC | 154 |
| 17 | EAAKFGAGAALQIPFAMQMAYRFNGIGPGPGSLPITVYYAAAYALLEDPVGTAAYGIFEDRAPVAAYNLLTTPKFTAAYRMLGDVMAVAAYRLNELLAYVAAYALSALLTKLAAYALHTALATVAAYRLLGFADTVAAYALMLRLLRIGPGPGPNMLRIMASLVLARKGPGPGRIKIVQMLSDTLKNLGPGPGFLLVTLAILTALRLC | 155 |

The vaccine compositions described herein protects against disease caused by one or more coronavirus variants or coronavirus subvariants. In some embodiments, the coronavirus variants or coronavirus subvariants comprise past or currently circulating coronavirus variants or coronavirus subvariants including but not limited to alpha, beta, gamma, delta, and omicron. In other embodiments, the coronavirus variants or coronavirus subvariants comprise future variants or future subvariants of human and animal coronavirus.

The vaccine compositions described herein may also protect against infection and reinfection of coronavirus variants or coronavirus subvariants. In some embodiments, the coronavirus variants or coronavirus subvariants comprise past or currently circulating coronavirus variants or coronavirus subvariants including but not limited to alpha, beta, gamma, delta, and omicron. In other embodiments, the coronavirus variants or coronavirus subvariants comprise future variants or future subvariants of human and animal coronavirus.

The vaccine compositions described herein protects against infection or reinfection of one or more coronavirus variant or coronavirus subvariant. In some embodiments, the vaccine composition described herein against infection or reinfection of multiple coronavirus variants or coronavirus subvariants. In other embodiments, the vaccine composition described herein composition protects against infection or re-infection caused by one coronavirus variants or coronavirus subvariants.

In some embodiments, the vaccine composition induces strong and long-lasting protection mediated by antibodies (Abs), CD4+ T helper (Th1) cells, and/or CD8+ cytotoxic T-cells (CTL).

### Molecular Adjuvants and T Cell Enhancements

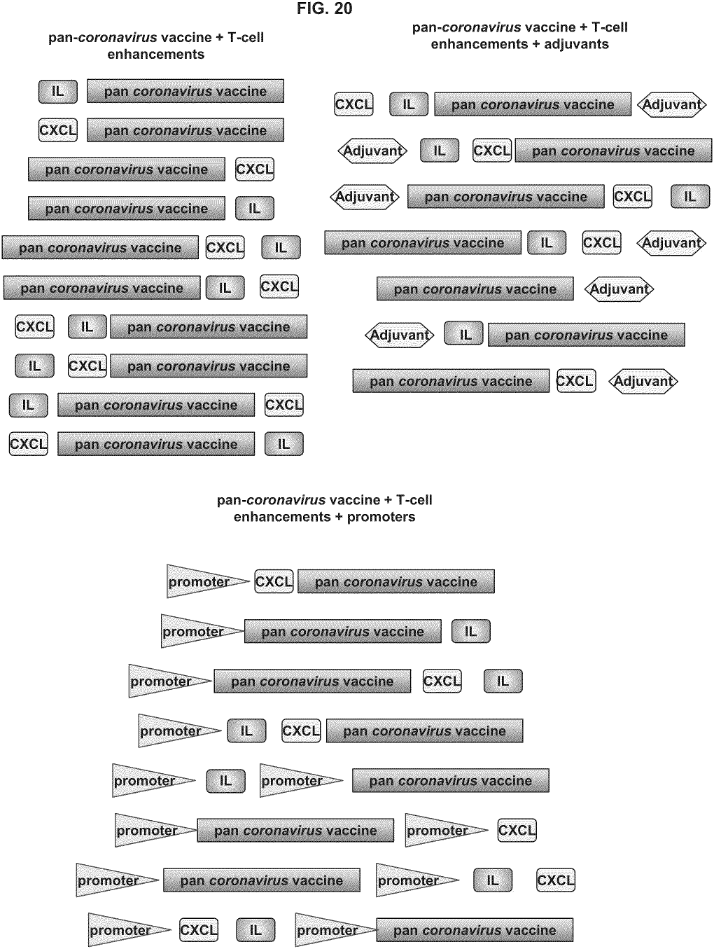

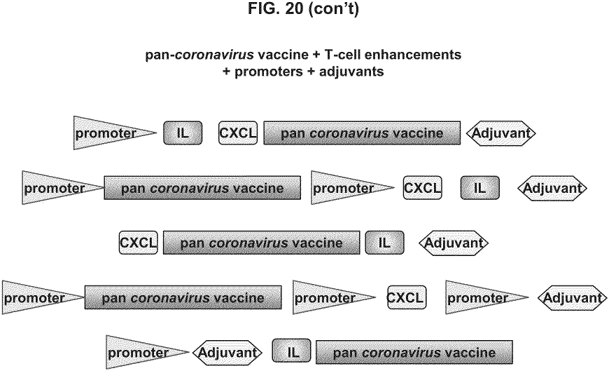

In certain embodiments, the vaccine composition comprises a molecular adjuvant and/or one or more T Cell enhancement compositions (see FIG. 20). The adjuvant and/or enhancement compositions may help improve the immunogenicity and/or long-term memory of the vaccine composition. Non-limiting examples of molecular adjuvants include CpG, such as a CpG polymer, and flagellin.

In some embodiments, the vaccine composition comprises a T cell attracting chemokine The T cell attracting chemokine helps pull the T cells from the circulation to the appropriate tissues, e.g., the lungs, heart, kidney, and brain. Non-limiting examples of T cell attracting chemokines include CCL5, CXCL9, CXCL10, CXCL11, CCL25, CCL28, CXCL14, CXCL17, or a combination thereof.

In some embodiments, the vaccine composition comprises a composition that promotes T cell proliferation. Non-limiting examples of compositions that promote T cell proliferation include IL-7, IL-15, IL-2, or a combination thereof.

In some embodiments, the vaccine composition comprises a composition that promotes T cell homing in the lungs. Non-limiting examples of compositions that promote T cell homing include CCL25, CCL28, CXCL14, CXCL17 or a combination thereof.

Table 10 shows non-limiting examples of T-cell enhancements that may be used to create a vaccine composition described herein.

TABLE 10

| T-cell enhancement | Sequence | SEQ ID NO: |
|---|---|---|
| CXCL11 | ATGAACAGGAAGGTGACCGCCATCGCCCTGGCCGCCATCATCTGGGCCA CCGCCGCCCAGGGCTTCCTGATGTTCAAGCAGGGCAGGTGCCTGTGCAT CGGCCCCGGCATGAAGGCCGTGAAGATGGCCGAGATCGAGAAGGCCAG CGTGATCTACCCCAGCAACGGCTGCGACAAGGTGGAGGTGATCGTGACC ATGAAGGCCCACAAGAGGCAGAGGTGCCTGGACCCCAGGAGCAAGCAGGCCAGGCTGATCATGCAGGCCATCGAGAAGAAGAACTTCCTGAGGAGGCAGAACATGTGA | 156 |
| CCL5 | ATGAAGGTCTCCGCGGCAGCCCTCGCTGTCATCCTCATTGCTACTGCCCTCTGCGCTCCTGCATCTGCCTCCCCATATTCCTCGGACACCACACCCTGCTGCTTTGCCTACATTGCCCGCCCACTGCCCCGTGCCCACATCAAGGAGTATTTCTACACCAGTGGCAAGTGCTCCAACCCAGCAGTCGTCCACAGGTCAAGGATGCCAAAGAGAGAGGGACAGCAAGTCTGGCAGGATTTCCTGTATGACTCCCGGCTGAACAAGGGCAAGCTTTGTCACCCGAAAGAACCGCCAAGTGTGTGCCAACCCAGAGAAGAAATGGGTTCGGGAGTACATCAACTCTTTGGAGATGAGCTAGGATGGAGAGTCCTTGAACCTGAACTTACACAAATTTGCCTGTTTCTGCTTGCTCTTGTCCTAGCTTGGGAGGCTTCCCCTCACTATCCTACCCCACCCGCTCCTTGA | 157 |
| CXCL9 | ATGAAGAAAAGTGGTGTTCTTTTCCTCTTGGGCATCATCTTGCTGGTTCTG ATTGGAGTGCAAGGAACCCCAGTAGTGAGAAAGGGTCGCTGTTCCTGCATCAGCACCAACCAAGGGACTATCCACCTACAATCCTTGAAAGACCTTAAACA ATTTGCCCCAAGCCCTTCCTGCGAGAAAATTGAAATCATTGCTACACTGAA GAATGGAGTTCAAACATGTCTAAACCCAGATTCAGCAGATGTGAAGGAACT GATTAAAAAGTGGGAGAAACAGGTCAGCCAAAAGAAAAAGCAAAAGAATG GGAAAAAACATCAAAAAAAGAAAGTTCTGAAAGTTCGAAAATCTCAACGTT CTCGTCAAAAGAAGACTACATAA | 158 |
| CXCL10 | ATGAATCAAACTGCCATTCTGATTTGCTGCCTTATCTTTCTGACTCTAAGTGGCATTCAAGGAGTACCTCTCTCTAGAACTGTACGCTGTACCTGCATCAGCATTAGTAATCAACCTGTTAATCCAAGGTCTTTAGAAAAACTTGAAATTATTCCTGCAAGCCAATTTTGTCCACGTGTTGAGATCATTGCTACAATGAAAAAGAAGGGTGAGAAGAGATGTCTGAATCCAGAATCGAAGGCCATCAAGAATTTACTGAAAGCAGTTAGCAAGGAAAGGTCTAAAAGATCTCCTTAA | 159 |
| CXCL14 | ATGAGGCTCCTGGCGGCCGCGCTGCTCCTGCTGCTGCTGGCGCTGTACA CCGCGCGTGTGGACGGGTCCAAATGCAAGTGCTCCCGGAAGGGACCCAA GATCCGCTACAGCGACGTGAAGAAGCTGGAAATGAAGCCAAAGTACCCGCACTGCGAGGAGAAGATGGTTATCATCACCACCAAGAGCGTGTCCAGGTACCGAGGTCAGGAGCACTGCCTGCACCCCAAGCTGCAGAGCACCAAGCGCTTCATCAAGTGGTACAACGCCTGGAACGAGAAGCGCAGGGTCTACGAAGAATAG | 160 |
| CXCL17 | ATGAAAGTTCTAATCTCTTCCCTCCTCCTGTTGCTGCCACTAATGCTGATG TCCATGGTCTCTAGCAGCCTGAATCCAGGGGTCGCCAGAGGCCACAGGG ACCGAGGCCAGGCTTCTAGGAGATGGCTCCAGGAAGGCGGCCAAGAATG TGAGTGCAAAGATTGGTTCCTGAGAGCCCCGAGAAGAAAATTCATGACAG TGTCTGGGCTGCCAAAGAAGCAGTGCCCCTGTGATCATTTCAAGGGCAAT GTGAAGAAAACAAGACACCAAAGGCACCACAGAAAGCCAAACAAGCATTCCAGAGCCTGCCAGCAATTTCTCAAACAATGTCAGCTAAGAAGCTTTGCTCTGCCTTTGTAG | 161 |
| CCL25 | ATGAACCTGTGGCTCCTGGCCTGCCTGGTGGCCGGCTTCCTGGGAGCCT GGGCCCCCGCTGTCCACACCCAAGGTGTCTTTGAGGACTGCTGCCTGGC CTACCACTACCCCATTGGGTGGGCTGTGCTCCGGCGCGCCTGGACTTAC CGGATCCAGGAGGTGAGCGGGAGCTGCAATCTGCCTGCTGCGATATTCTACCTCCCCAAGAGACACAGGAAGGTGTGTGGGAACCCCAAAAGCAGGGAGGTGCAGAGAGCCATGAAGCTCCTGGATGCTCGAAATAAGGTTTTTGCAAAGCTCCACCACAACACGCAGACCTTCCAAGCAGGCCCTCATGCTGTAAAGAAGTTGAGTTCTGGAAACTCCAAGTTATCATCGTCCAAGTTTAGCAATCCCATCAGCAGCAGTAAGAGGAATGTCTCCCTCCTGATATCAGCTAATTCAGGACTGTGA | 162 |
| CCL28 | ATGCAGCAGAGAGGACTCGCCATCGTGGCCTTGGCTGTCTGTGCGGCCC TACATGCCTCAGAAGCCATACTTCCCATTGCCTCCAGCTGTTGCACGGAG GTTTCACATCATATTTCCAGAAGGCTCCTGGAAAGAGTGAATATGTGTCGCATCCAGAGAGCTGATGGGGATTGTGACTTGGCTGCTGTCATCCTTCATGTCAAGCGCAGAAGAATCTGTGTCAGCCCGCACAACCATACTGTTAAGCAGTGGATGAAAGTGCAAGCTGCCAAGAAAAATGGTAAAGGAAATGTTTGCCACAGGAAGAAACACCATGGCAAGAGGAACAGTAACAGGGCACATCAGGGGAAACACGAAACATACGGCCATAAAACTCCTTATTAG | 163 |
| IL-7 | ATGTTCCACGTGAGCTTCAGGTACATCTTCGGCATCCCCCCCCTGATCCT GGTGCTGCTGCCCGTGACCAGCAGCGAGTGCCACATCAAGGACAAGGAG GGCAAGGCCTACGAGAGCGTGCTGATGATCAGCATCGACGAGCTGGACA AGATGACCGGCACCGACAGCAACTGCCCCAACAACGAGCCCAACTTCTTCAGGAAGCACGTGTGCGACGACACCAAGGAGGCCGCCTTCCTGAACAGGGCCGCCAGGAAGCTGAAGCAGTTCCTGAAGATGAACATCAGCGAGGAGTTCAACGTGCACCTGCTGACCGTGAGCCAGGGCACCCAGACCCTGGTGAACTGCACCAGCAAGGAGGAGAAGAACGTGAAGGAGCAGAAGAAGAACGACGCCTGCTTCCTGAAGAGGCTGCTGAGGGAGATCAAGACCTGCTGGAACAAGATCCTGAAGGGCAGCATCTGA | 164 |
| IL-15 | ATGAGAATTTCGAAACCACATTTGAGAAGTATTTCCATCCAGTGCTACTTGTGTTTACTTCTAAACAGTCATTTTCTAACTGAAGCTGGCATTCATGTCTTCATTTTGGGCTGTTTCAGTGCAGGGCTTCCTAAAACAGAAGCCAACTGGGTGAATGTAATAAGTGATTTGAAAAAAATTGAAGATCTTATTCAATCTATGCATATTGATGCTACTTTATATACGGAAAGTGATGTTCACCCCAGTTGCAAAGTAACAGCAATGAAGTGCTTTCTCTTGGAGTTACAAGTTATTTCACTTGAGTCCGGAGATGCAAGTATTCATGATACAGTAGAAAATCTGATCATCCTAGCAAACAACAGTTTGTCTTCTAATGGGAATGTAACAGAATCTGGATGCAAAGAATGTGAGGAACTGGAGGAAAAAAATATTAAAGAATTTTTGCAGAGTTTTGTACATATTGTCCAAATGTTCATCAACACTTCTTGA | 165 |
| IL-2 | ATGTACAGGATGCAACTCCTGTCTTGCATTGCACTAAGTCTTGCACTTGTCACAAACAGTGCACCTACTTCAAGTTCTACAAAGAAAACACAGCTACAACTGGAGCATTTACTGCTGGATTTACAGATGATTTTGAATGGAATTAATAATTACAAGAATCCCAAACTCACCAGGATGCTCACATTTAAGTTTTACATGCCCAAGAAGGCCACAGAACTGAAACATCTTCAGTGTCTAGAAGAAGAACTCAAACCTCTGGAGGAAGTGCTAAATTTAGCTCAAAGCAAAAACTTTCACTTAAGACCCAGGGACTTAATCAGCAATATCAACGTAATAGTTCTGGAACTAAAGGGATCTGAAACAACATTCATGTGTGAATATGCTGATGAGACAGCAACCATTGTAGAATTTCTGAACAGATGGATTACCTTTTGTCAAAGCATCATCTCAACACTGACTTGA | 166 |

In some embodiments, the T-cell enhancement compositions described herein (e.g. CXCL9, CXCL10, IL-7, IL-2) may be integrated into a separate delivery system from the vaccine compositions. In other embodiments, the T-cell enhancement compositions described herein (e.g. CXCL9, CXCL10, IL-7, IL-2) may be integrated into the same delivery system as the vaccine compositions.

In certain embodiments, the composition comprises a tag. For example, in some embodiments, the composition comprises a His tag. The present invention is not limited to a His tag and includes other tags such as those known to one of ordinary skill in the art, such as a fluorescent tag (e.g., GFP, YFP, etc.), etc.

### Antigen Delivery System

The present invention also features vaccine compositions in the form of an antigen delivery system. Any appropriate antigen delivery system may be considered for delivery of the antigens described herein. The present invention is not limited to the antigen delivery systems described herein.

In certain embodiments, the antigen delivery system is for targeted delivery of the vaccine composition, e.g., for targeting to the tissues of the body where the virus replicates.

In certain embodiments, the antigen delivery system comprises an adeno-associated virus vector-based antigen delivery system, such as but not limited to the adeno-associated virus vector type 9 (AAV9 serotype), AAV type 8 (AAV8 serotype), etc. (see, for example, FIG. 21, FIG. 22, FIG. 23, and FIG. 24). In certain embodiments, the adeno-associated virus vectors used are tropic, e.g., tropic to lungs, brain, heart and kidney, e.g.. the tissues of the body that express ACE2 receptors (see FIG. 3A)). For example, AAV9 is known to be neurotropic, which would help the vaccine composition to be expressed in the brain.

The present invention is not limited to adeno-associated virus vector-based antigen delivery systems. Examples of other antigen delivery systems include adenoviruses such as but not limited to Ad5, Ad26, Ad35, etc., as well as carriers such as lipid nanoparticles, polymers, peptides, etc. In other embodiments, the antigen delivery system comprises a vesicular stomatitis virus (VSV) vector.

In the antigen delivery system, the antigen or antigens (e.g., epitopes) are operatively linked to a promoter. In certain embodiments, the antigen or antigens (e.g., epitopes) are operatively linked to a generic promoter. For example, in certain embodiments, the antigen or antigens (e.g., epitopes) are operatively linked to a CMV promoter. In certain embodiments, the antigen or antigens (e.g., epitopes) are operatively linked to a CAG, EFIA, EFS, CBh, SFFV, MSCV, mPGK, hPGK, SV40, UBC, or other appropriate promoter.

In some embodiments, the antigen or antigens (e.g., epitopes) are operatively linked to a tissue-specific promoter (e.g., a lung-specific promoter). For example, the antigen or antigens (e.g, epitopes) may be operatively linked to a SpB promoter or a CD144 promoter.

As discussed, in certain embodiments, the vaccine composition comprises a molecular adjuvant. In certain embodiments, the molecular adjuvant is operatively linked to a generic promoter, e.g., as described above. In certain embodiments, the molecular adjuvant is operatively linked to a tissue-specific promoter, e.g., a lung-specific promoter, e.g., SpB or CD144 (see FIG. 21, FIG. 22).

As discussed, in certain embodiments, the vaccine composition comprises a T cell attracting chemokine. In certain embodiments, the T cell attracting chemokine is operatively linked to a generic promoter, e.g., as described above. In certain embodiments, the T cell attracting chemokine is operatively linked to a tissue-specific promoter, e.g., a lung-specific promoter, e.g., SpB or CD144 (eg, see FIG. 21).

As discussed, in certain embodiments, the vaccine composition comprises a composition for promoting T cell proliferation. In certain embodiments, the composition for promoting T cell proliferation is operatively linked to a generic promoter, e.g., as described above. In certain embodiments, the composition for promoting T cell proliferation is operatively linked to a tissue-specific promoter, e.g., a lung-specific promoter, e.g., SpB or CD144 (e.g., see FIG. 23).

Table 11 shows non-limiting examples of promoters that may be used to create a vaccine composition described herein.

TABLE 11

| Promoter | Sequence | SEQ ID NO: |
|---|---|---|
| CAG | CTCGACATTGATTATTGACTAGTTATTAATAGTAATCAATTACGGGGTCATTAGTTCATAGCCCATATATGGAGTTCCGCGTTACATAACTTACGGTAAATGGCCCGCCTGGCTGACCGCCCAACGACCCCCGCCCATTGACGTCAATAATGACGTATGTTCCCATAGTAACGCCAATAGGGACTTTCCATTGACGTCAATGGGTGGAGTATTTACGGTAAACTGCCCACTTGGCAGTACATCAAGTGTATCATATGCCAAGTACGCCCCCTATTGACGTCAATGACGGTAAATGGCCCGCCTGGCATTATGCCCAGTACATGACCTTATGGGACTTTCCTACTTGGCAGTACATCTACGTATTAGTCATCGCTATTACCATGGTCGAGGTGAGCCCCACGTTCTGCTTCACTCTCCCCATCTCCCCCCCCTCCCCACCCCCAATTTTGTATTTATTTATTTTTTAATTATTTTGTGCAGCGATGGGGGCGGGGGGGGGGGGGGGGCGCGCGCCAGGCGGGGCGGGGCGGGGCGAGGGGCGGGGCGGGGCGAGGCGGAGAGGTGCGGCGGCAGCCAATCAGAGCGGCGCGCTCCGAAAGTTTCCTTTTATGGCGAGGCGGCGGCGGCGGCGGCCCTATAAAAAGCGAAGCGCGCGGCGGGCGGGAGTCGCTGCGCGCTGCCTTCGCCCCGTGCCCCGCTCCGCCGCCGCCTCGCGCCGCCCGCCCCGGCTCTGACTGACCGCGTTACTCCCACAGGTGAGCGGGCGGGACGGCCCTTCTCCTCCGGGCTGTAATTAGCGCTTGGTTTAATGACGGCTTGTTTCTTTTCTGTGGCTGCGTGAAAGCCTTGAGGGGCTCCGGGAGGGCCCTTTGTGCGGGGGGAGCGGCTCGGGGGGTGCGTGCGTGTGTGTGTGCGTGGGGAGCGCCGCGTGCGGCTCCGCGCTGCCCGGCGGCTGTGAGCGCTGCGGGCGCGGCGCGGGGCTTTGTGCGCTCCGCAGTGTGCGCGAGGGGAGCGCGGCCGGGGGCGGTGCCCCGCGGTGCGGGGGGGGCTGCGAGGGGAACAAAGGCTGCGTGCGGGGTGTGTGCGTGGGGGGGTGAGCAGGGGGTGTGGGCGCGTCGGTCGGGCTGCAACCCCCCCTGCACCCCCCTCCCCGAGTTGCTGAGCACGGCCCGGCTTCGGGTGCGGGGCTCCGTACGGGGCGTGGCGCGGGGCTCGCCGTGCCGGGCGGGGGGTGGCGGCAGGTGGGGGTGCCGGGCGGGGCGGGGCCGCCTCGGGCCGGGGAGGGCTCGGGGGAGGGGCGCGGCGGCCCCCGGAGCGCCGGCGGCTGTCGAGGCGCGGCGAGCCGCAGCCATTGCCTTTTATGGTAATCGTGCGAGAGGGCGCAGGGACTTCCTTTGTCCCAAATCTGTGCGGAGCCGAAATCTGGGAGGCGCCGCCGCACCCCCTCTAGCGGGCGCGGGGCGAAGCGGTGCGGCGCCGGCAGGAAGGAAATGGGCGGGGAGGGCCTTCGTGCGTCGCCGCGCCGCCGTCCCCTTCTCCCTCTCCAGCCTCGGGGCTGTCCGCGGGGGGACGGCTGCCTTCGGGGGGGACGGGGCAGGGCGGGGTTCGGCTTCTGGCGTGTGACCGGCGGCTCTAGAGCCTCTGCTAACCATGTTCATGCCTTCTTCTTTTTCCTACAGCTCCTGGGCAACGTGCTGGTTATTGTGCTGTCTCATCATTTTGGCAAAGAATTG | 167 |
| CMV | TAGTTATTAATAGTAATCAATTACGGGGTCATTAGTTCATAGCCCATATATGGAGTTCCGCGTTACATAACTTACGGTAAATGGCCCGCCTGGCTGACCGCCCAACGACCCCCGCCCATTGACGTCAATAATGACGTATGTTCCCATAGTAACGCCAATAGGGACTTTCCATTGACGTCAATGGGTGGAGTATTTACGGTAAACTGCCCACTTGGCAGTACATCAAGTGTATCATATGCCAAGTACGCCCCCTATTGACGTCAATGACGGTAAATGGCCCGCCTGGCATTATGCCCAGTACATGACCTTATGGGACTTTCCTACTTGGCAGTACATCTACGTATTAGTCATCGCTATTACCATGGTGATGCGGTTTTGGCAGTACATCAATGGCGTGGATAGCGGTTTGACTCACGGGGATTTCCAAGTCTCCACCCCATTGACGTCAATGGGAGTTTGTTTTGGCACCAAAATCAACGGGACTTTCCAAAATGTCGTAACAACTCCGCCCCATTGACGCAAATGGGCGGTAGGCGTGTACGGTGGGAGGTCTATATAAGCAGAGCTGGTTTAGTGAACCGTCAGATC | 168 |
| SP-B | GTATAGGGCTGTCTGGGAGCCACTCCAGGGCCACAGAAATCTTGTCTCTGACTCAGGGTATTTTGTTTTCTGTTTTGTGTAAATGCTCTTCTGACTAATGCAAACCATGTGTCCATAGAACCAGAAGATTTTTCCAGGGGAAAAGGTAAGGAGGTGGTGAGAGTGTCCTGGGTCTGCCCTTCCAGGGCTTGCCCTGGGTTAAGAGCCAGGCAGGAAGCTCTCAAGAGCATTGCTCAAGAGTAGAGGGGGCCTGGGAGGCCCAGGGAGGGGATGGGAGGGGAACACCCAGGCTGCCCCCAACCAGATGCCCTCCACCCTCCTCAACCTCCCTCCCACGGCCTGGAGAGGTGGGACCAGGTATGGAGGCTTGAGAGCCCCTGGTTGGAGGAAGCCACAAGTCCAGGAACATGGGAGTCTGGGCAGGGGGCAAAGGAGGCAGGAACAGGCCATCAGCCAGGACAGGTGGTAAGGCAGGCAGGAGTGTTCCTGCTGGGAAAAGGTGGGATCAAGCACCTGGAGGGCTCTTCAGAGCAAAGACAAACACTGAGGTCGCTGCCACTCCTACAGAGCCCCCACGCCCCGCCCAGCTATAAGGGGCCATGCACCAAGCAGGGTACCCAGGCTGCAGAGGTGCC | 169 |
| CD144 | CATCCATGCCCATGGCCTCAGATGCCAGCCATAAGCTGTTGGGTTCCAAACCTCGACTCCAGGCTGGACTCACCCCTGTCTCCCCCACCAGCCTGACACCTCCACCTGGGTATCTAACGAGCATCTCAAACTCAACCTGCCTGAGACAGAGGAATCACTATCCCCTCCTCCTCCAAAAATATCCTTCCATCACACTCCCCATCTTGTGCTCTGATTTACTAAACGGCCCTGGGCCCTCTCTTTCTCAGGGTCTCTGCTTGCCCAGCTATATAATAAAACAAGTTTGGGACTTCCCAACCATTCACCCATGGAAAAACAGAAGCAACTCTTCAAAGGACAGATTCCCAGGATCTGCCCTGGGAGATTCCAAATCAGTTGATCTGGGGTGAGCCCAGTCCTCTGTAGTTTTTAGAAGCTCCTCCTATGTCTCTCCTGGTCAGCAGAATCTTGGCCCCTCCCTTCCCCCCAGCCTCTTGGTTCTTCTGGGCTCTGATCCAGCCTCAGCGTCACTGTCTTCCACGCCCCTCTTTGATTCTCGTTTATGTCAAAAGCCTTGTGAGGATGAGGCTGTGATTATCCCCATTTTACAGATGAGGAAACTGTGGCTCCAGGATGACACAACTGGCCAGAGGTCACATCAGAAGCAGAGCTGGGTCACTTGACTCCACCCAATATCCCTAAATGCAAACATCCCCTACAGACCGAGGCTGGCACCTTAGAGCTGGAGTCCATGCCCGCTCTGACCAGGAGAAGCCAACCTGGTCCTCCAGAGCCAAGAGCTTCTGTCCCTTTCCCATCTCCTGAAGCCTCCCTGTCACCTTTAAAGTCCATTCCCACAAAGACATCATGGGATCACCACAGAAAATCAAGCTCTGGGGCTAGGCTGACCCCAGCTAGATTTTTGGCTCTTTTATACCCCAGCTGGGTGGACAAGCACCTTAAACCCGCTGAGCCTCAGCTTCCCGGGCTATAAAATGGGGGTGATGACACCTGCCTGTAGCATTCCAAGGAGGGTTAAATGTGATGCTGCAGCCAAGGGTCCCCACAGCCAGGCTCTTTGCAGGTGCTGGGTTCAGAGTCCCAGAGCTGAGGCCGGGAGTAGGGGTTCAAGTGGGGTGCCCCAGGCAGGGTCCAGTGCCAGCCCTCTGTGGAGACAGCCATCCGGGGCCGAGGCAGCCGCCCACCGCAGGGCCTGCCTATCTGCAGCCAGCCCAGCCCTCACAAAGGAACAATAACAGGAAACCATCCCAGGGGGAAGTGGGCCAGGGCCAGCTGGAAAACCTGAAGGGGAGGCAGCCAGGCCTCCCTCGCCAGCGGGGTGTGGCTCCCCTCCAAAGACGGTCGGCTGACAGGCTCCACAGAGCTCCACTCACGCTCAGCCCTGGACGGACAGGCAGTCCAACGGAACAGAAACATCCCTCAGCCCACAGGCACGGTGAGTGGGGGCTCCCACACTCCCCTCCACCCCAAACCCGCCACCCTGCGCCCAAGATGGGAGGGTCCTCAGCTTCCCCATCTGTAGAATGGGCATCGTCCCACTCCCATGACAGAGAGGCTCC | 170 |
| wild type native leader sequence | ATGTTCGTGTTCCTGGTGCTGCTGCCCCTGGTGAGCAGC | 171 |

In certain embodiments, the T cell attracting chemokine and the composition that promotes T cell proliferation are driven by the same promoter (e.g., the T cell attracting chemokine and the composition that promotes T cell proliferation are synthesized as a peptide). In certain embodiments, the T cell attracting chemokine and the composition that promotes T cell proliferation are driven by different promoters. In certain embodiments, the antigen, the T cell attracting chemokine, and the composition that promotes T cell proliferation are driven by the same promoter. In certain embodiments, the antigen or antigens, the T cell attracting chemokine, and the composition that promotes T cell proliferation are driven by the different promoters. In certain embodiments, the T cell attracting chemokine and the composition that promotes T cell proliferation are driven by the same promoter, and the antigen or antigens are driven by a different promoter.

In some embodiments, the antigen delivery system comprises one or more linkers between the T cell attracting chemokine and the composition that promotes T cell proliferation. In certain embodiments, linkers are used between one or more of the epitopes. The linkers may allow for cleavage of the separate molecules (e.g.,. chemokine). For example, in some embodiments, a linker is positioned between IL-7 (or IL-2) and CCL5, CXCL9, CXCL10, CXCL11, CCL25, CCL28, CXCL14, CXCL17, etc. In some embodiments, a linker is positioned between IL-15 (or IL-2) and CCL5, CXCL9, CXCL10, CXCL11, CCL25, CCL28, CXCL14, CXCL17, etc. In some embodiments, a linker is positioned between the antigen and another composition, e.g., IL-15, IL-7, IL-2, CCL5, CXCL9, CXCL10, CXCL11, CCL25, CCL28, CXCL14, CXCL17, etc. A non-limiting example of a linker is T2A, E2A, P2A (see Table 12), or the like (e.g., see FIG. 24). The composition may feature a different linker between each open reading frame.

TABLE 12

| | SEQUENCE | SEQ ID NO: |
|---|---|---|
| T2A Linker | GGAAGCGGAGAGGGCAGGGGAAGTCTTCTAACATGCGGGGACGTGG AGGAAAATCCCGGCCCC | 172 |
| E2A Linker | GGAAGCGGACAGTGTACTAATTATGCTCTCTTGAAATTGGCTGGAGATGTTGAGAGCAACCCAGGTCCC | 173 |
| P2A Linker | GGAAGCGGAGCCACGAACTTCTCTCTGTTAAAGCAAGCAGGAGATGT TGAAGAAAACCCCGGGCCT | 174 |
| 6-His Tag | CATCACCATCACCATCAC | 175 |

The present invention includes mRNA sequences encoding any of the vaccine compositions or portions thereof herein. The present invention also includes modified mRNA sequences encoding any of the vaccine compositions or portions thereof herein. The present invention also includes DNA sequence encoding any of the vaccine compositions or portions thereof herein.

In certain embodiments, nucleic acids of a vaccine composition herein are chemically modified. In some embodiments, the nucleic acids of a vaccine composition therein are unmodified In some embodiments, all or a portion of the uracil in the open reading frame has a chemical modification. In some embodiments, a chemical modification is in the 5-position of the uracil. In some embodiments, a chemical modification is a N1-methyl pseudouridine. In some embodiments, all or a portion of the uracil in the open reading frame has a N1-methyl pseudouridine in the 5-position of the uracil.

In certain embodiments, an open reading frame of a vaccine composition herein encodes one antigen or epitopes. In some embodiments, an open reading frame of a vaccine composition herein encodes two or more antigens or epitopes. In some embodiments, an open reading frame of a vaccine composition herein encodes five or more antigens or epitopes. In some embodiments, an open reading frame of a vaccine composition herein encodes ten or more antigens or epitopes. In some embodiments, an open reading frame of a vaccine composition herein encodes 50 or more antigens or epitopes.

### Epitope Arrangements

The target epitopes of the compositions described may be arranged in various configurations (see, for example, FIG. 25, FIG. 20). In some embodiments, the target epitopes may be arranged such that one or more CD8+ T cell epitopes are followed by one or more CD4+ T cell epitopes followed by one or more B cell epitopes. In some embodiments, the target epitopes may be arranged such that one or more CD8+ T cell epitopes are followed by one or more B cell epitopes followed by one or more CD4+ T cell epitopes. In other embodiments, the target epitopes may be arranged such that one or more CD4+ T cell epitopes are followed by one or more CD8+ T cell epitopes followed by one or more B cell epitopes. In other embodiments, the target epitopes may be arranged such that one or more CD4+ T cell epitopes are followed by one or more B cell epitopes followed by one or more CD8+ T cell epitopes. In some embodiments, the target epitopes may be arranged such that one or more B cell epitopes are followed by one or more CD4+ T cell epitopes followed by one or more CD8+ T cell epitopes. In other embodiments, the target epitopes may be arranged such that one or more B cell epitopes are followed by one or more CD8+ T cell epitopes followed by one or more CD4+ T cell epitopes.

In some embodiments, the target epitopes may be arranged such that one or more pairs of CD4+-CD8+ T cell epitopes are followed by one or more pairs of CD4+ T cell -B cell epitopes. In other embodiments, the target epitopes may be arranged such that CD8+ T cell, CD4+ T cell, and B cell epitopes are repeated one or more times.

In other embodiments, the target epitopes may be arranged such that one or more CD4+ T cell epitopes are followed by one or more CD8+ T cell epitopes. In embodiments, the target epitopes may be arranged such that one or more CD8+ T cell epitopes are followed by one or more CD4+ T cell epitopes. In some embodiments, the target epitopes may be arranged such that one or more CD4+ T cell epitopes are followed by one or more B cell target epitopes. In some embodiments, the target epitopes may be arranged such that one or more CD8+ T cell epitopes are followed by one or more B cell target epitopes. In other embodiments, the target epitopes may be arranged such that one or more B cell epitopes are followed by one or more CD4+ T cell target epitopes. In some embodiments, the target epitopes may be arranged such that one or more B cell epitopes are followed by one or more CD8+ T cell target epitopes.

Likewise, the other components of the vaccine composition may be arranged in various configurations. For example, in certain embodiments, the T cell attracting chemokine is followed by the composition for promoting T cell proliferation. In certain embodiments, the composition for promoting T cell proliferation is followed by the T cell attracting chemokine.

### Methods

The present invention also features methods for designing and/or producing a multi-epitope, pan-coronavirus composition Briefly, the method may comprise determining target epitopes, selecting desired target epitopes (e.g, two or more, etc.), and synthesizing an antigen comprising the selected target epitopes. The method may comprise determining target epitopes, selecting desired target epitopes, and synthesizing a nucleotide composition (e.g., DNA, modified DNA, mRNA, modified mRNA, antigen delivery system, etc.) encoding the antigen comprising the selected target epitopes. In some embodiments, the method further comprises creating a vaccine composition comprising the antigen, nucleotide compositions, and/or antigen delivery system and a pharmaceutical carrier.

The methods herein may also include the steps of designing the antigen delivery system. For example, the methods may comprise inserting molecular adjuvants, chemokines, linkers, tags, etc. into the antigen delivery system. In some embodiments, one or more components is inserted into a different antigen delivery system from the antigen or antigens (e.g., the epitopes). For example, the present invention provides embodiments wherein the antigen or antigens (e.g., the epitopes) are within a first antigen delivery system and one or more additional components (e.g., chemokine, etc.) are within a second delivery system. In some embodiments, the antigen or antigens (e.g., the epitopes) and one or more additional components are within a first delivery system, and one or more additional components are within a second delivery system. In some embodiments, the antigen or antigens (e.g., the epitopes) and one or more additional components are within a first delivery system, and the antigen or antigens (e.g., the epitopes) and one or more additional components are within a second delivery system.

In some embodiments, the method comprises determining target epitopes from at least two of the following 1. coronavirus B-cell epitopes, 2. coronavirus CD4+ T cell epitopes, and/or 3. coronavirus CD8+ T cell epitopes. In some embodiments, each of the target epitopes are conserved epitopes, e.g., as described herein. For example, the target epitopes may be conserved among two or a combination of: at least one SARS-CoV-2 human strains in current circulation, at least one coronavirus that has caused a previous human outbreak, at least one coronavirus isolated from bats, at least one coronavirus isolated from pangolin, at least one coronavirus isolated from civet cats, at least one coronavirus strain isolated from mink, and at least one coronavirus strain isolated from camels or any other animal that is receptive to coronavirus. In some embodiments, the composition comprises at least two of the following: one or more coronavirus B-cell target epitopes, one or more coronavirus CD4⁺ T cell target epitopes, and/or one or more coronavirus CD8⁺ T cell target epitopes.

In certain embodiments, the method comprises selecting at least one epitope from at least two of: one or more conserved coronavirus B-cell epitopes; one or more conserved coronavirus CD4+ T cell epitopes; and one or more conserved coronavirus CD8+ T cell epitopes; and synthesizing an antigen comprising the selected epitopes. In certain embodiments, the method comprises selecting at least one epitope from at least two of: one or more conserved coronavirus B-cell epitopes; one or more conserved coronavirus CD4+ T cell epitopes; and one or more conserved coronavirus CD8+ T cell epitopes; and synthesizing an antigen delivery system that encodes an antigen comprising the selected epitopes.

In some embodiments, the method comprises determining one or more conserved large sequences that are derived from coronavirus sequences (e.g., SARS-CoV-2, variants, common cold coronaviruses, previously known coronavirus strains, animal coronaviruses, etc.). The method may comprise selecting at least one large conserved sequence and synthesizing an antigen comprising the selected large conserved sequence(s). The method may comprise synthesizing a nucleotide composition (e.g., DNA, modified DNA, mRNA, modified mRNA, antigen delivery system, etc.) encoding the antigen comprising the selected large conserved sequence(s). In some embodiments, the method further comprises creating a vaccine composition comprising the antigen, nucleotide compositions, and/or antigen delivery system and a pharmaceutical carrier. In some embodiments, the large sequences comprise one or more conserved epitopes described herein, e.g., one or more conserved B-cell target epitopes and/or one or more conservedCD4+ T cell target epitopes and/or one or more conservedCD8+ T cell target epitopes.

In some embodiments, each of the large sequences are conserved among two or a combination of: at least two SARS-CoV-2 human strains in current circulation, at least one coronavirus that has caused a previous human outbreak, at least one coronavirus isolated from bats, at least one coronavirus isolated from pangolin, at least one coronavirus isolated from civet cats, at least one coronavirus strain isolated from mink, and at least one coronavirus strain isolated from camels or any other animal that is receptive to coronavirus.

As previously discussed, the compositions described herein, e.g., the epitopes, the vaccine compositions, the antigen delivery systems, the chemokines, the adjuvants, etc. may be used to prevent a coronavirus disease in a subject. In some embodiments, the compositions described herein, e.g., the epitopes, the vaccine compositions, the antigen delivery systems, the chemokines, the adjuvants, etc. may be used to prevent a coronavirus infection prophylactically in a subject. In some embodiments, the compositions described herein, e.g., the epitopes, the vaccine compositions, the antigen delivery systems, the chemokines, the adjuvants, etc. may elicit an immune response in a subject. In some embodiments, the compositions described herein, e.g., the epitopes, the vaccine compositions, the antigen delivery systems, the chemokines, the adjuvants, etc. may prolong an immune response induced by the multi-epitope pan-coronavirus recombinant vaccine composition and increases T-cell migration to the lungs.

Methods for preventing a coronavirus disease in a subject may comprise administering to the subject a therapeutically effective amount of a multi-epitope, pan-coronavirus recombinant vaccine composition according to the present invention. In some embodiments, the composition elicits an immune response in the subject. In some embodiments, the composition induces memory B and T cells. In some embodiments, the composition induces resident memory T cells (Tᵣₘ) In some embodiments, the composition prevents virus replication, e.g., in the areas where the virus normally replicates such as lungs, brain, heart, and kidney. In some embodiments, the composition prevents a cytokine storm, e.g., in the areas where the virus normally replicates such as lungs, brain, heart, and kidney. In some embodiments, the composition prevents inflammation or an inflammatory response, e.g., in the areas where the virus normally replicates such as lungs, brain, heart, and kidney. In some embodiments, the composition improves homing and retention of T cells, e.g., in the areas where the virus normally replicates such as lungs, brain, heart, and kidney.

Methods for preventing a coronavirus infection prophylactically in a subject may comprise administering to the subject a prophylactically effective amount of a multi-epitope, pan-coronavirus recombinant vaccine composition according to the present invention. In some embodiments, the composition elicits an immune response in the subject. In some embodiments, the composition induces memory B and T cells. In some embodiments, the composition induces resident memory T cells (Trm). In some embodiments, the composition prevents virus replication, e.g., in the areas where the virus normally replicates such as lungs, brain, heart, and kidney. In some embodiments, the composition prevents a cytokine storm, e.g., in the areas where the virus normally replicates such as lungs, brain, heart, and kidney. In some embodiments, the composition prevents inflammation or an inflammatory response, e.g., in the areas where the virus normally replicates such as lungs, brain, heart, and kidney. In some embodiments, the composition improves homing and retention of T cells, e.g., in the areas where the virus normally replicates such as lungs, brain, heart, and kidney.

Methods for eliciting an immune response in a subject may comprise administering to the subject a vaccine composition according to the present invention, wherein the composition elicits an immune response in the subject. In some embodiments, the composition induces memory B and T cells. In some embodiments, the composition induces resident memory T cells (Trm). In some embodiments, the composition prevents virus replication, e.g., in the areas where the virus normally replicates such as lungs, brain, heart, and kidney. In some embodiments, the composition prevents a cytokine storm, e.g., in the areas where the virus normally replicates such as lungs, brain, heart, and kidney. In some embodiments, the composition prevents inflammation or an inflammatory response, e.g., in the areas where the virus normally replicates such as lungs, brain, heart, and kidney. In some embodiments, the composition improves homing and retention of T cells, e.g., in the areas where the virus normally replicates such as lungs, brain, heart, and kidney.

Methods for prolonging an immune response induced by a vaccine composition of the present invention and increasing T cell migration to particular tissues (e.g., lung, brain, heart, kidney, etc.) may comprise co-expressing a T-cell attracting chemokine, a composition that promotes T cell proliferation, and a vaccine composition (e.g., antigen) according to the present invention.

Methods for prolonging the retention of memory T-cell into the lungs induced by a vaccine composition of the present invention and increasing virus-specific tissue resident memory T-cells (T_{RM} cells) may comprise co-expressing a T-cell attracting chemokine, a composition that promotes T cell proliferation, and a vaccine composition (e.g., antigen) according to the present invention.

The vaccine composition may be administered through standard means, e.g., through an intravenous route (i.v.), an intranasal route (i.n.), or a sublingual route (s.l.) route.

In certain embodiments, the method comprises administering to the subject a second (e.g., booster) dose. The second dose may comprise the same vaccine composition or a different vaccine composition. Additional doses of one or more vaccine compositions may be administered.

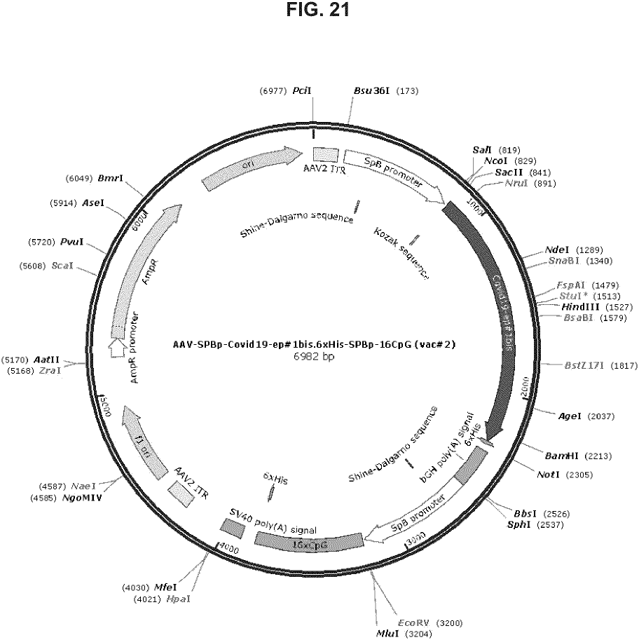

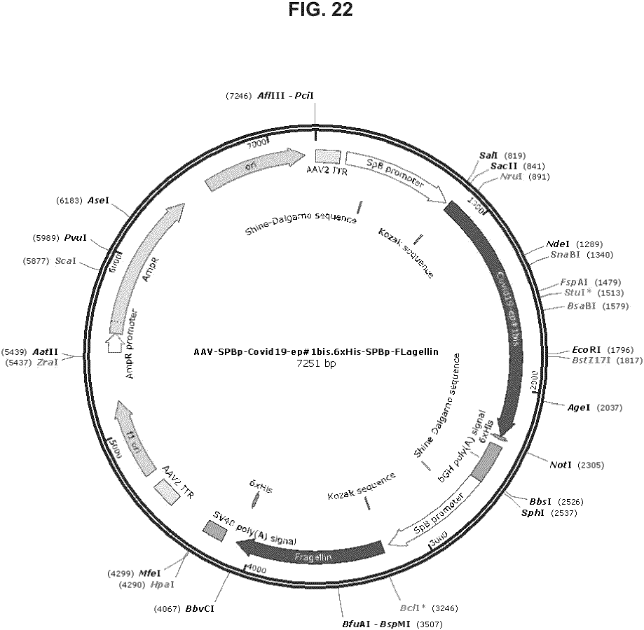

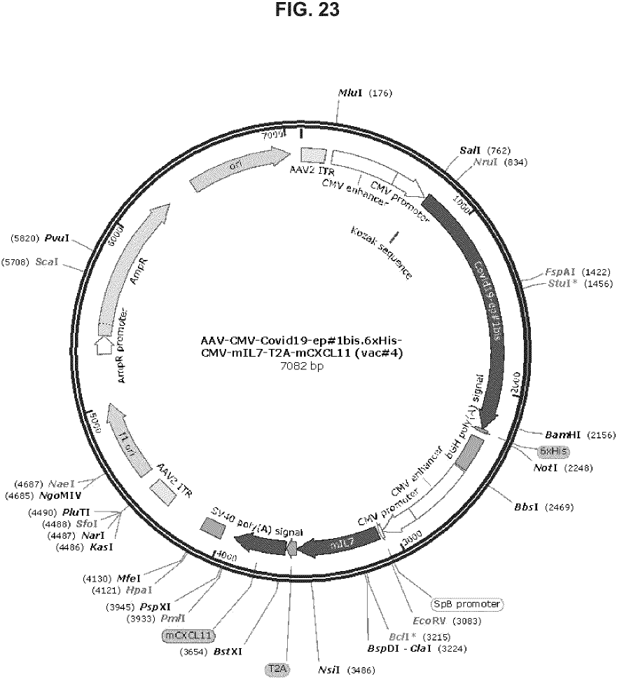

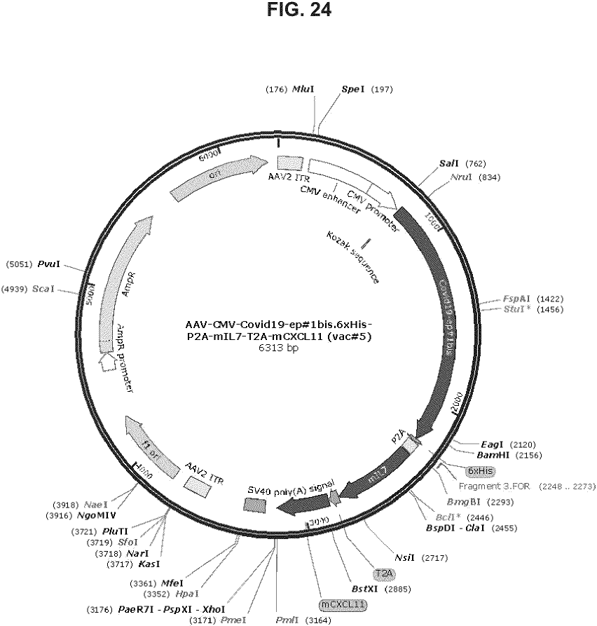

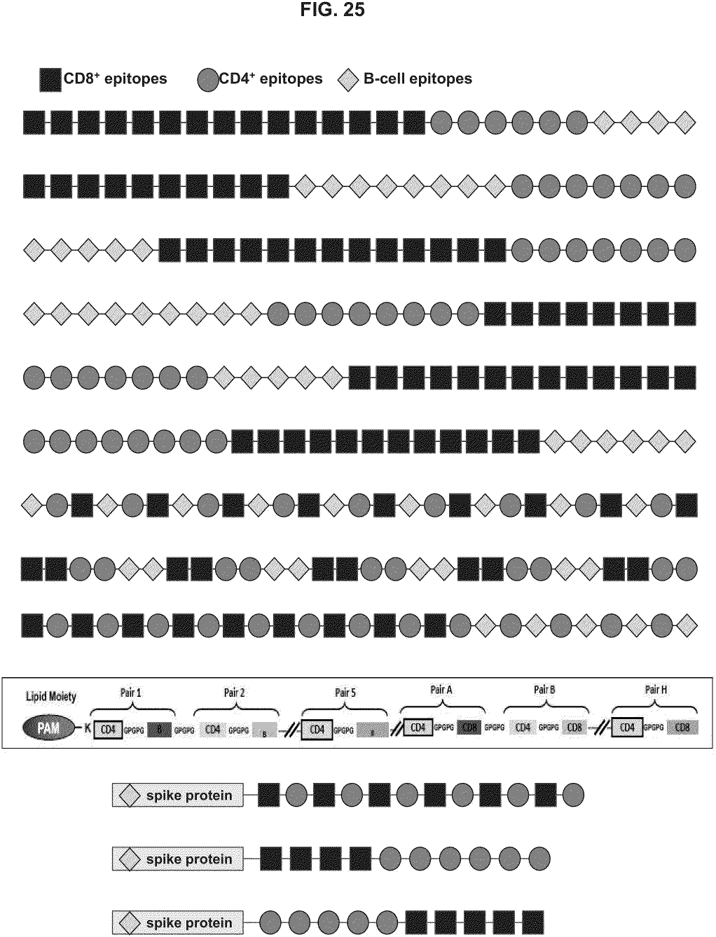

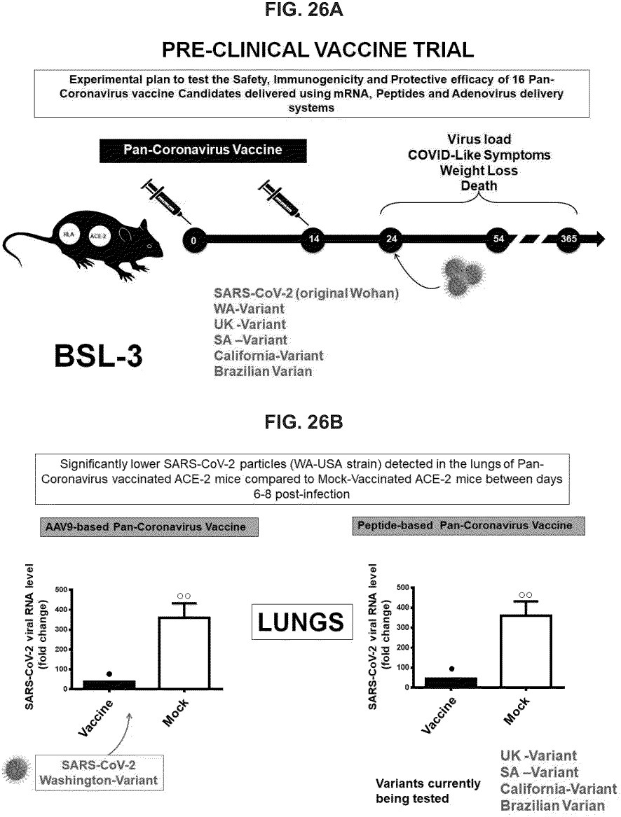

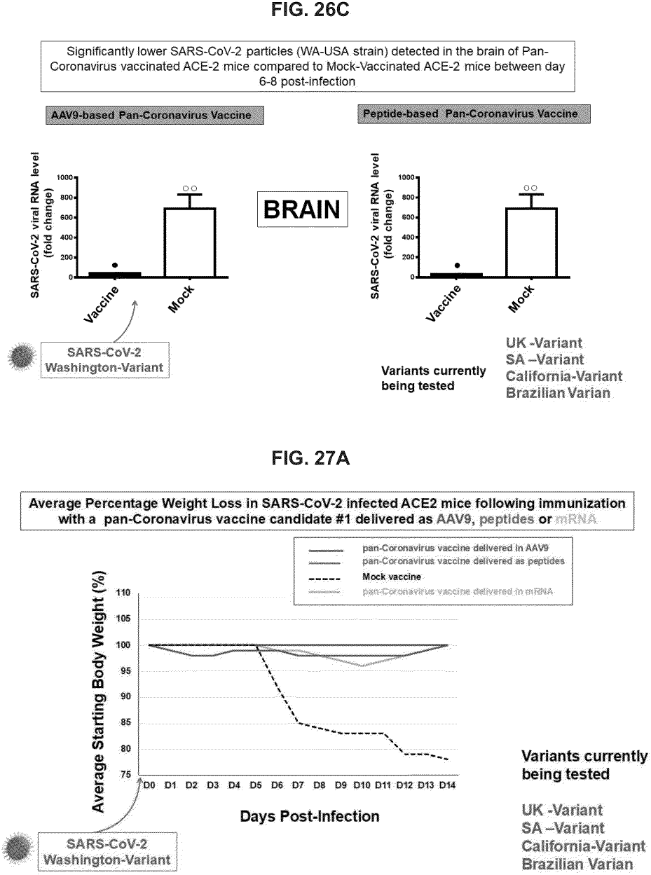

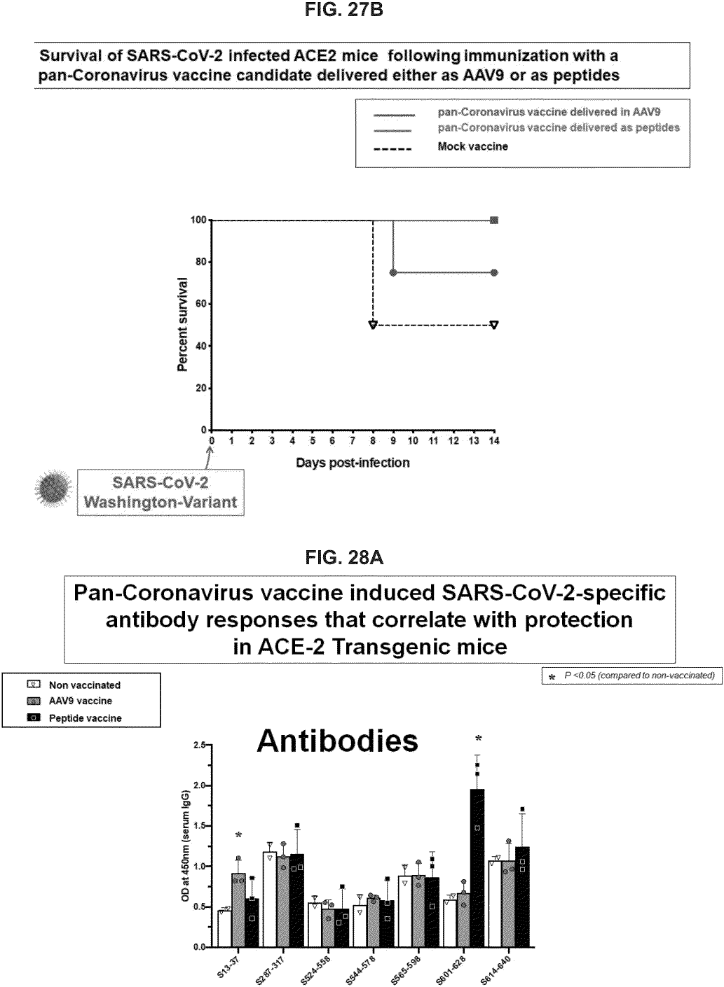

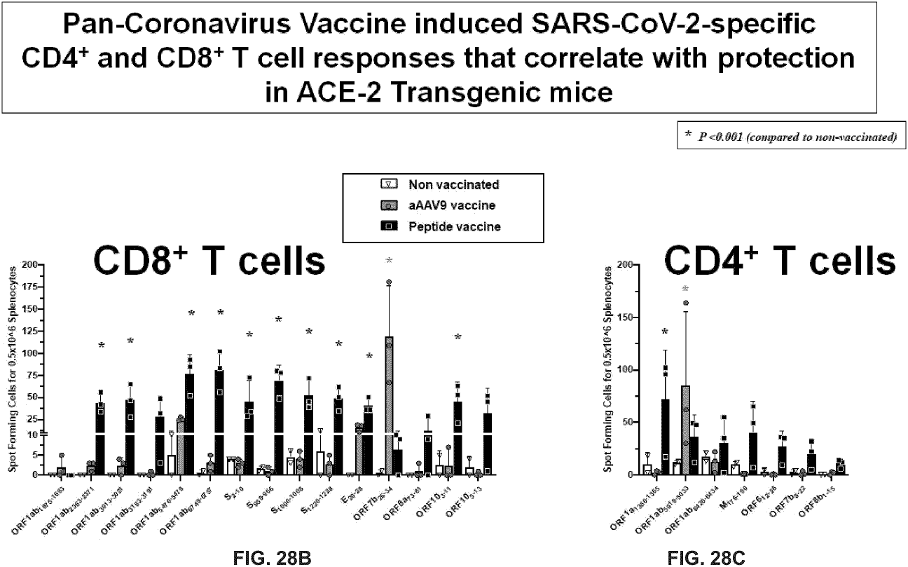

The vaccine composition (SEQ ID NO: 139) of the present invention has been tested in pre-clinical trials using “humanized” HLA double transgenic mice (FIG. 26A). FIG. 26B and FIG. 26C shows that vaccinated mice had significantly lower SARS-CoV-2 particles detected in the lungs (FIG. 26B) and in the brain (FIG. 26C) when compared to mock vaccinated mice 6-8 days after infection. Additionally, there is no difference between how the vaccine was delivered (peptide or adeno-associated virus (AAV9)) and the effectiveness of the vaccine (FIG. 26B and FIG. 26C) and the survival of the mice (FIG. 27A and FIG. 27B). Furthermore, FIGS. 28C and 28D show that both the peptide or adeno-associated virus vaccine are able to induce a SARS-CoV-2-specific CD 4+ and CD 8+ T cell response.

The vaccine compositions of the present invention decrease inflammation and increase T cells lining alveoli epithelial cells. For example, FIGS. 29A and 29B show a decrease in inflammation, and an increase in T cells lining alveoli epithelial cells.

### Sequential Vaccine Delivery Methodology

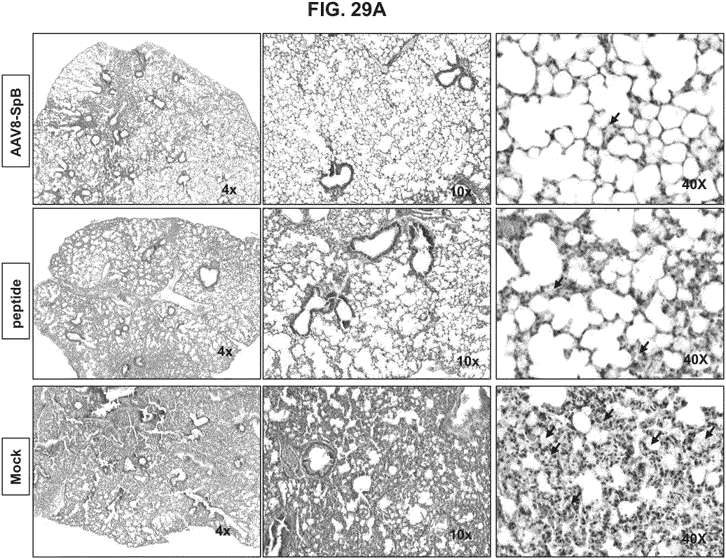

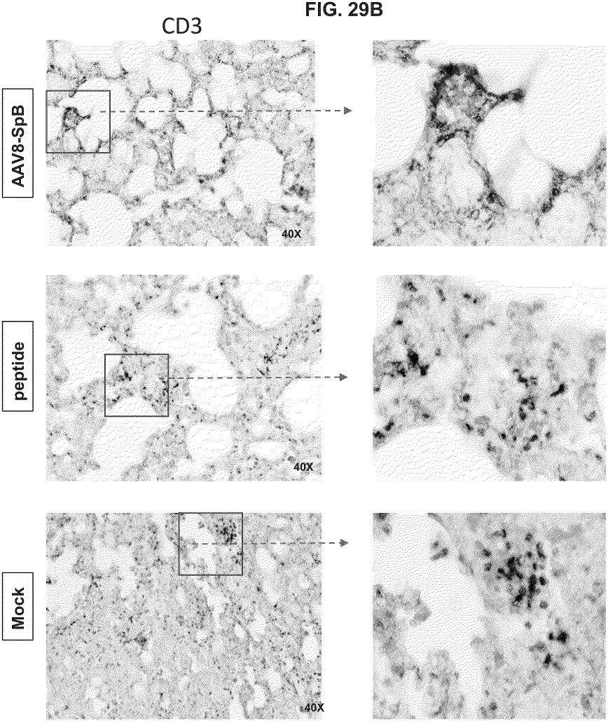

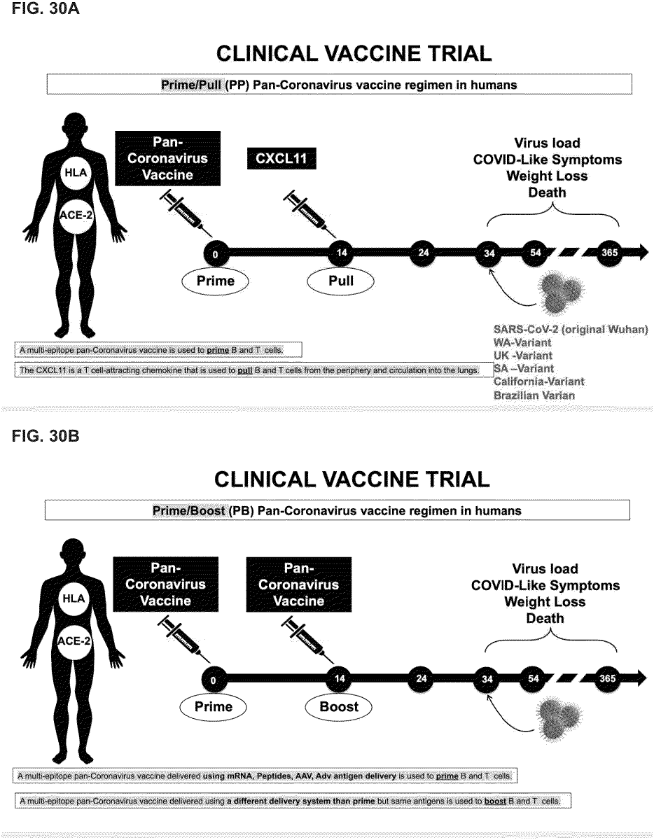

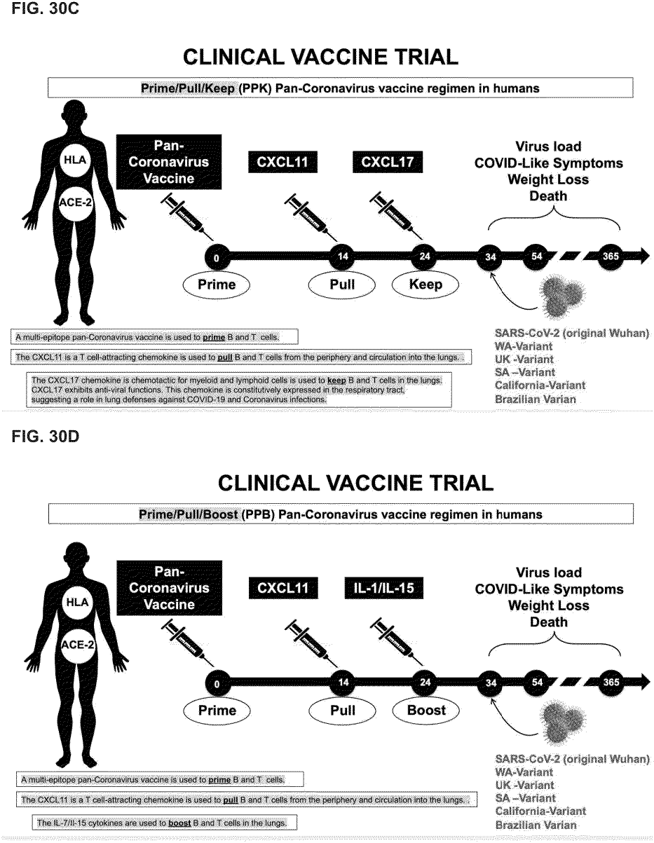

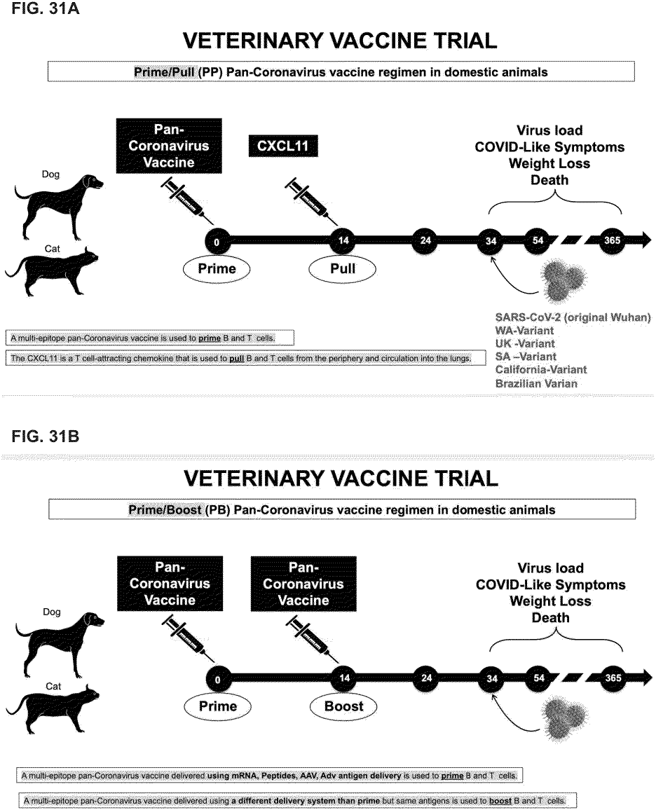

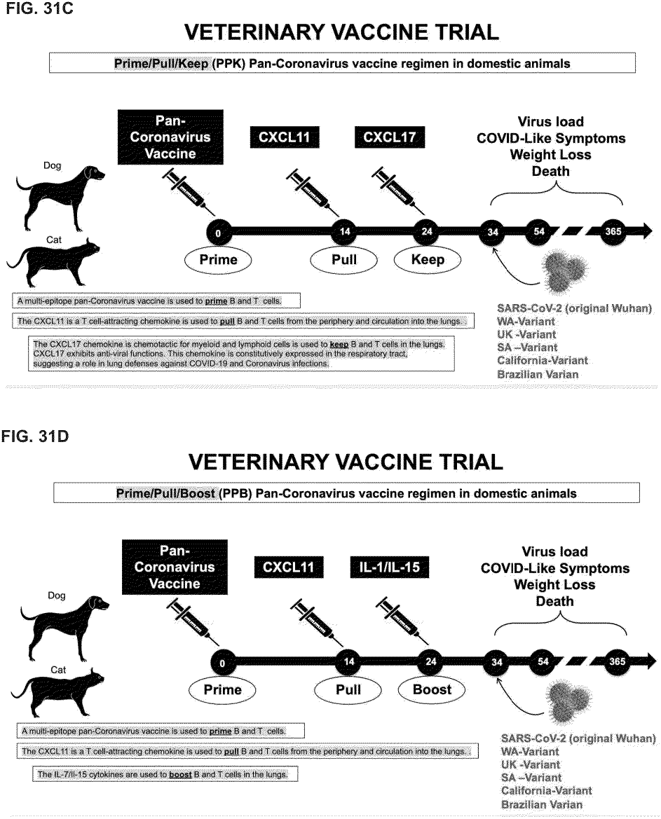

In some embodiments, the present invention features a method of delivering the vaccine to induce heterologous immunity in a subject (e.g., prime/boost, see FIG. 30B and FIG. 31B). In some embodiments, the method comprises administering a first composition, e.g., a first pan-coronavirus recombinant vaccine composition dose using a first delivery system and further administering a second composition, e.g., a second vaccine composition dose using a second delivery system. In other embodiments, the first delivery system and the second delivery system are different. In some embodiments, the second composition is administered 8 days after administration of the first composition. In some embodiments, the second composition is administered 9 days after administration of the first composition. In some embodiments, the second composition is administered 10 days after administration of the first composition. In some embodiments, the second composition is administered 11 days after administration of the first composition. In some embodiments, the second composition is administered 12 days after administration of the first composition. In some embodiments, the second composition is administered 13 days after administration of the first composition. In some embodiments, the second composition is administered 14 days after administration of the first composition. In some embodiments, the second composition is administered from 14 to 30 days after administration of the first composition. In some embodiments, the second composition is administered from 30 to 60 days after administration of the first composition.

In some embodiments, the first delivery system or the second delivery system comprises an mRNA, a modified mRNA or a peptide vector In other embodiments, the peptide vector comprises adenovirus or an adeno-associated virus vector.

In some embodiments, the present invention features a method of delivering the vaccine to induce heterologous immunity in a subject (e.g., prime/pull, see FIG. 30A and FIG. 31A). In some embodiments, the method comprises administering a pan-coronavirus recombinant vaccine composition and further administering at least one T-cell attracting chemokine after administering the pan-coronavirus recombinant vaccine composition. In some embodiments, the T-cell attracting chemokine is administered 8 days after the vaccine composition is administered. In some embodiments, the T-cell attracting chemokine is administered 9 days after the vaccine composition is administered. In some embodiments, the T-cell attracting chemokine is administered 10 days after the vaccine composition is administered. In some embodiments, the T-cell attracting chemokine is administered 11 days after the vaccine composition is administered. In some embodiments, the T-cell attracting chemokine is administered 12 days after the vaccine composition is administered. In some embodiments, the T-cell attracting chemokine is administered 13 days after the vaccine composition is administered. In some embodiments, the T-cell attracting chemokine is administered 14 days after the vaccine composition is administered. In some embodiments, the T-cell attracting chemokine is administered from 14 to 30 days after administration of the vaccine composition. In some embodiments, the T-cell attracting chemokine is administered from 30 to 60 days after administration of the vaccine composition.

The present invention also features a novel “prime, pull, and boost” strategy. In other embodiments, the present invention features a method to increase the size and maintenance of lung-resident B-cells, CD4+ T cells and CD8+ T cells to protect against SARS-CoV-2 (FIG. 30D and FIG. 31D). In some embodiments, the method comprises administering a pan-coronavirus recombinant vaccine composition and administering at least one T-cell attracting chemokine after administering the pan-coronavirus recombinant vaccine composition. In some embodiments, the method further comprises administering at least one cytokine after administering the T-cell attracting chemokine. In some embodiments, the T-cell attracting chemokine is administered 8 days after the vaccine composition is administered. In some embodiments, the T-cell attracting chemokine is administered 9 days after the vaccine composition is administered. In some embodiments, the T-cell attracting chemokine is administered 10 days after the vaccine composition is administered. In some embodiments, the T-cell attracting chemokine is administered 11 days after the vaccine composition is administered. In some embodiments, the T-cell attracting chemokine is administered 12 days after the vaccine composition is administered. In some embodiments, the T-cell attracting chemokine is administered 13 days after the vaccine composition is administered. In some embodiments, the T-cell attracting chemokine is administered 14 days after the vaccine composition is administered. In some embodiments, the T-cell attracting chemokine is administered from 14 to 30 days after administration of the vaccine composition. In some embodiments, the T-cell attracting chemokine is administered from 30 to 60 days after administration of the vaccine composition. In some embodiments, the cytokine is administered 8 days after administering the T-cell attracting chemokine. In some embodiments, the cytokine is administered 9 days after administering the T-cell attracting chemokine. In some embodiments, the cytokine is administered 10 days after administering the T-cell attracting chemokine. In some embodiments, the cytokine is administered 11 days after administering the T-cell attracting chemokine. In some embodiments, the cytokine is administered 12 days after administering the T-cell attracting chemokine. In some embodiments, the cytokine is administered 13 days after administering the T-cell attracting chemokine. In some embodiments, the cytokine is administered 14 days after administering the T-cell attracting chemokine. In some embodiments, the cytokine is administered from 14 to 30 days after administering the T-cell attracting chemokine, In some embodiments, the cytokine is administered from 30 to 60 days after administering the T-cell attracting chemokine.

The present invention further features a novel “prime, pull, and keep” strategy (FIG. 30C and FIG. 31C). For example, the present invention features a method to increase the size and maintenance of lung-resident B-cells, CD4+ T cells and CD8+ T cells to protect against SARS-CoV-2. In some embodiments, the method comprises administering a pan-coronavirus recombinant vaccine composition and administering at least one T-cell attracting chemokine after administering the pan-coronavirus recombinant vaccine composition. In some embodiments, the method further comprises administering at least one mucosal chemokine after administering the T-cell attracting chemokine. In some embodiments, the T-cell attracting chemokine is administered 8 days after the vaccine composition is administered. In some embodiments, the T-cell attracting chemokine is administered 9 days after the vaccine composition is administered. In some embodiments, the T-cell attracting chemokine is administered 10 days after the vaccine composition is administered. In some embodiments, the T-cell attracting chemokine is administered 11 days after the vaccine composition is administered. In some embodiments, the T-cell attracting chemokine is administered 12 days after the vaccine composition is administered. In some embodiments, the T-cell attracting chemokine is administered 13 days after the vaccine composition is administered. In some embodiments, the T-cell attracting chemokine is administered 14 days after the vaccine composition is administered. In some embodiments, the T-cell attracting chemokine is administered from 14 to 30 days after administration of the vaccine composition. In some embodiments, the T-cell attracting chemokine is administered from 30 to 60 days after administration of the vaccine composition. In some embodiments, the mucosal chemokine is administered 8 days after administering the T-cell attracting chemokine. In some embodiments, the mucosal chemokine is administered 9 days after administering the T-cell attracting chemokine. In some embodiments, the mucosal chemokine is administered 10 days after administering the T-cell attracting chemokine. In some embodiments, the mucosal chemokine is administered 11 days after administering the T-cell attracting chemokine. In some embodiments, the mucosal chemokine is administered 12 days after administering the T-cell attracting chemokine. In some embodiments, the mucosal chemokine is administered 13 days after administering the T-cell attracting chemokine. In some embodiments, the mucosal chemokine is administered 14 days after administering the T-cell attracting chemokine. In some embodiments, the mucosal chemokine is administered from 14 to 30 days after administering the T-cell attracting chemokine. In some embodiments, the mucosal chemokine is administered from 30 to 60 days after administering the T-cell attracting chemokine.

In some embodiments, the mucosal chemokines may comprise CCL25, CCL28,CXCL14, CXCL17, or a combination thereof. In some embodiments, the T-cell attracting chemokines may comprise CCL5, CXCL9, CXCL10, CXCL11, or a combination thereof. In some embodiments, the cytokines may comprise IL-15, IL-7, IL-2, or a combination thereof.

In some embodiments, the efficacy (or effectiveness) of a vaccine composition herein is greater than 60%. In some embodiments, the efficacy (or effectiveness) of a vaccine composition herein is greater than 70%. In some embodiments, the efficacy (or effectiveness) of a vaccine composition herein is greater than 80%. In some embodiments, the efficacy (or effectiveness) of a vaccine composition herein is greater than 90%. In some embodiments, the efficacy (or effectiveness) of a vaccine composition herein is greater than 95%.

Vaccine efficacy may be assessed using standard analyses (see, e.g., Weinberg et al, J Infect Dis. 2010 Jun. 1; 201(11):1607-10). For example, vaccine efficacy may be measured by double-blind, randomized, clinical controlled trials. Vaccine efficacy may be expressed as a proportionate reduction in disease attack rate (AR) between the unvaccinated (ARU) and vaccinated (ARV) study cohorts and can be calculated from the relative risk (RR) of disease among the vaccinated group with use of the following formulas: Efficacy=(ARU-ARV)/ARU×100; and Efficacy=(1-RR)×100

Likewise, vaccine effectiveness may be assessed using standard analyses (see, e.g., Weinberg et al., J Infect Dis. 2010 Jun. 1; 201(11):1607-10). Vaccine effectiveness is an assessment of how a vaccine (which may have already proven to have high vaccine efficacy) reduces disease in a population. This measure can assess the net balance of benefits and adverse effects of a vaccination program, not just the vaccine itself, under natural field conditions rather than in a controlled clinical trial. Vaccine effectiveness is proportional to vaccine efficacy (potency) but is also affected by how well target groups in the population are immunized, as well as by other non-vaccine-related factors that influence the ‘real-world’ outcomes of hospitalizations, ambulatory visits, or costs. For example, a retrospective case control analysis may be used, in which the rates of vaccination among a set of infected cases and appropriate controls are compared. Vaccine effectiveness may be expressed as a rate difference, with use of the odds ratio (OR) for developing infection despite vaccination: Effectiveness=(1-OR)×100.

In some embodiments, the vaccine immunizes the subject against a coronavirus for up to 1 year. In some embodiments, the vaccine immunizes the subject against a coronavirus for up to 2 years. In some embodiments, the vaccine immunizes the subject against a coronavirus for more than 1 year, more than 2 years, more than 3 years, more than 4 years, or for 5-10 years.

In some embodiments, the subject is a young adult between the ages of about 20 years and about 50 years (e.g., about 20, 25, 30, 35, 40, 45 or 50 years old).

In some embodiments, the subject is an elderly subject about 60 years old, about 70 years old, or older (e.g., about 60, 65, 70, 75, 80, 85 or 90 years old).

In some embodiments, the subject is about 5 years old or younger. For example, the subject may be between the ages of about 1 year and about 5 years (e.g., about 1, 2, 3, 5 or 5 years), or between the ages of about 6 months and about 1 year (e.g., about 6. 7, 8, 9, 10, 11 or 12 months). In some embodiments, the subject is about 12 months or younger (e.g., 12, 11, 10, 9, 8, 7, 6, 5, 4, 3, 2 months or 1 month). In some embodiments, the subject is about 6 months or younger.

In some embodiments, the subject was bom full term (e.g., about 37-42 weeks). In some embodiments, the subject was bom prematurely, for example, at about 36 weeks of gestation or earlier (e.g., about 36, 35, 34, 33, 32, 31, 30, 29, 28, 27, 26 or 25 weeks). For example, the subject may have been bom at about 32 weeks of gestation or earlier. In some embodiments, the subject was born prematurely between about 32 weeks and about 36 weeks of gestation. In such subjects, a vaccine may be administered later in life, for example, at the age of about 6 months to about 5 years, or older.

In some embodiments, the subject is pregnant (e.g., in the first, second or third trimester) when administered a vaccine.

In some embodiments, the subject has a chronic pulmonary disease (e.g., chronic obstructive pulmonary disease (COPD) or asthma) or is at risk thereof. Two forms of COPD include chronic bronchitis, which involves a long-term cough with mucus, and emphysema, which involves damage to the lungs over time. Thus, a subject administered a vaccine may have chronic bronchitis or emphysema.

In some embodiments, the subject has been exposed to a coronavirus.. In some embodiments, the subject is infected with a coronavirus. In some embodiments, the subject is at risk of infection by a coronavirus.

In some embodiments, the subject is immunocompromised (has an impaired immune system, e.g., has an immune disorder or autoimmune disorder).

### Pharmaceutical Carriers

In certain embodiments, the vaccine composition further comprises a pharmaceutical carrier. Pharmaceutical carriers are well known to one of ordinary skill in the art. For example, in certain embodiments, the pharmaceutical carrier is selected from the group consisting of water, an alcohol, a natural or hardened oil, a natural or hardened wax, a calcium carbonate, a sodium carbonate, a calcium phosphate, kaolin, talc, lactose and combinations thereof. In some embodiments, the pharmaceutical carrier may comprise a lipid nanoparticle, an adenovirus vector, or an adeno-associated virus vector. In some embodiments, the vaccine composition is constructed using an adeno-associated virus vectors-based antigen delivery system.

Also provided herein is vaccine of any one of the foregoing paragraphs, formulated in a nanoparticle (e.g., a lipid nanoparticle). In some embodiments, the nanoparticle has a mean diameter of 50-200 nm In some embodiments, the nanoparticle is a lipid nanoparticle. In some embodiments, the lipid nanoparticle comprises a cationic lipid, a PEG-modified lipid, a sterol and a non-cationic lipid. In some embodiments, the lipid nanoparticle comprises a molar ratio of about 20-60% cationic lipid, 0.5-15% PEG-modified lipid, 25-55% sterol, and 25% non-cationic lipid. In some embodiments, the cationic lipid is an ionizable cationic lipid, and the non-cationic lipid is a neutral lipid, and the sterol is a cholesterol. In some embodiments, the cationic lipid is selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319).

### Hybrid Vaccine Candidates

Referring now to FIGS. 30, 31, 32A, and 32B, the present invention may further feature a pan-coronavirus-influenza recombinant vaccine composition. The composition comprises at least a portion of a coronavirus spike (S) protein and at least a portion of an influenza hemagglutinin (HA) protein.

In some embodiments, the portion of an influenza hemagglutinin (HA) protein is highly conserved among human influenza viruses. The portion of an influenza hemagglutinin (HA) protein may be derived from one or more of: H1N1 virus strain, H3N2 virus strain, influenza B virus strains, or variants thereof.

In some embodiments, the H1N1 virus strains or variants are selected from 28566 available complete genome sequences in NCBI for hemagglutinin (HA) gene. Some of the prominent strains are: OK384178.1, OM642156.1. OM654386.1, OL840606.1, OK625377.1, OM865246.1, OM935941.1, OM642158.1, OM935953.1, MW840068.1, MW839847.1, MW839825.1, MW930730.1, MT227010.1, LC638096.1, LC638077.1, LC637999.1, and LC645067.1. In some embodiments, the H3N2 virus strains or variants are selected from 33620 available complete genome sequences in NCBI for hemagglutinin (HA) gene. Some of the prominent strains are: MZ005227.1, MW849238.1, MZ203409.1, MZ198318.1, MZ198312.1, MZ198295.1, MZ198289.1, MZ198265.1, MW789449.1, MW798370.1, MW790182.1, MW789645.1, MW789778.1, MW789685.1, MW789659.1, and MW790001.1. In some embodiments, the influenza B virus strains or variants are selected from 16596 available complete genome sequences in NCBI for hemagglutinin (HA) gene. Some of the prominent strains are: M10298.1, MT738525.1, MT808088.1, MT056751.1, MT314641.1, MT874090.1, MT242979.1, MT315665.1, MT1055640.1, MT057563.1, MT056955.1, MT243019.1, MT306916.1, MT057571.1, MT314793.1, MT343026.1, MT874109.1, MT243795.1, MT315769.1, and KX885875.1.

TABLE 13

Shows non-limiting examples of a portion of an influenza hemagglutinin (HA) protein that may be used in accordance with the present invention.

| | Sequence | SEQ ID NO: |
|---|---|---|
| HA (nucleotide) | TTCGGAGCTATTGCTGGTTTCTTGGAAGGAGGATGGGAAGGAATGA TTGCAGGTTGGCACGGATACACATCTCATGGAGCACATGGAGTAGC AGTGGCAGCAGACCTTAAGAGTACCCA | 285 |
| HA (amino acid) | FGAIAGFLEGGWEGMIAGWHGYTSHGAHGVAVAADLKSTX | 286 |
| HA-H1N1 | ATGAAGGCAATACTAGTAGTTCTGCTATATACATTTGCAACCGCAAATGCAGACACATTATGTATAGGTTATCATGCGAACAACTCAACAGACACTGTAGACACAGTACTAGAAAAGAATGTAACAGTAACACACTCTGTTAACCTTCTAGAAGACAAGCATAACGGGAAACTATGCAAACTAAGAGGGGTAGCCCCATTGCATTTGGGTAAATGTAACATTGCTGGCTGGATCCTGGGAAATCCAGAGTGTGAATCACTCTCCACAGCAAGCTCATGGTCCTACATTGTGGAAACATCTAGTTCAGACAATGGAACGTGTTACCCAGGAGATTTCATCGATTATGAGGAGCTAAGAGAGCAATTGAGCTCAGTGTCATCATTTGAAAGGTTTGAGATATTCCCCAAGACAAGTTCATGGCCCAATCATGACTCGAACAAAGGTGTAACGACAGCATGTCCTCATGCTGGAGCAAAAAGCTTCTACAAAAATTTAATATGGCTAGTTAAAAAAGGAAATTCATACCCAAAGCTCAGCAAATCCTACATTAATGATAAAGGGAAAGAAGTCCTCGTGCTATGGGGCATTCACCATCCACCTACTAGTGCTGACCAACAAAGTCTCTATCAGAATGCAGATGCATATGTTTTTGTGGGGACATCAAGATACAGCAAGAAGTTCAAGCCGGAAATAGCAATAAGGCCCAAAGTGAGGGATCAAGAAGGGAGAATGAACTATTACTGGACACTAGTAGAGCCGGGAGACAAAATAACATTCGAAGCAACTGGAAATCTAGTGGTACCGAGATATGCATTCGCAATGGAAAGAAATGCTGGATCTGGTATTATCATTTCAGATACACCAGTCCACGATTGCAATACAACTTTCAGACACCCAAGGGTGCTATAAACACCAGCCTCCCATTTCAGAACATACATCCGATCACAATTGGAAAATGTCCAAAATATGTAAAAAGCACAAAATTGAGACTGGCCACAGGATTGAGGAATGTCCCGTCCATTCAATCTAGAGGCCTATTTGGGGCCATTGCCGGTTTCATTGAAGGGGGGTGGACAGGGATGGTAGATGGATGGTACGGTTATCACCATCAAAATGAGCAGGGGTCAGGATATGCAGCCGACCTGAAGAGCACACAGAATGCCATTGACGAGATTACTAACAAAGTAAACTCTGTTATTGAAAAAATGAATACACAGTTCACAGCAGTAGGTAAAGAGTTCAACCACCTGGAAAAAAGAATAGAGAATTTAAATAAAAAAGTTGATGATGGTTTCCTGGACATTTGGACTTACAATGCCGAACTGTTGGTTCTATTGGAAAATGAAAGAACTTTGGACTACCACGATTCAAAGGTGAAGAACTTATATGAAAAGGTAAGAAGCCAGTTAAAAAACAATGCCAAGGAAATTGGAAACGGCTGCTTTGAATTTTACCACAAATGTGATAACACGTGCATGGAAAGTGTCAAAAATGGGACTTATGACTACCCAAAATACTCAGAGGAAGCAAAATTAAACAGAGAAGAAATAGATGGGGTAAAGCTGGAATCAACAAGGATTTACCAGATTTTGGCGATCTATTCAACCGTCGCCAGTTCATTGGTACTGGTAGTCTCCCTGGGGGCAATCAGTTTCTGGATGTGCTCTAATGGGTCTCTACAGTGTAGAATATGTATTTAA | 287 |
| HA H3N2 | AGCAAAAGCAGGGGATAATTCTATTAACCATGAAGACTATCATTGCTTTGAGCTACATTCTATGTCTGGTTTTCGCTCAAAAACTTCCTGGAAATGACAATAGCACTGCAACGCTGTGCCTTGGGCACCATGCAGTACCAAACGGAACGATAGTGAAAACAATCACGAATGACCAAATTGAAGTTACTAATGCTACTGAGCTGGTTCAGAATTCCTCAATAGGTGAAATATGCGACAGTCCTCATCAGATCCTTGATGGAGAAAACTGCACACTAATAGATGCTCTATTGGGAGACCCTCAGTGTGATGGCTTTCAAAATAAGAAATGGGACCTTTTTGTTGAACGAAGCAAAGCCTACAGCAACTGTTACCCTTATGATGTGCCGGATTATGCCTCCCTTAGGTCACTAGTTGCCTCATCCGGCACACTGGAGTTTAACAATGAAAGCTTCAATTGGGCTGGAGTCACTCAAAACGGAACAAGTTCTGCTTGCATAAGGGGATCTAATAGTAGTTTCTTTAGTAGATTAAATTGGTTGACCCACTTAAACTTCAAGTACCCAGCATTGAACGTGACTATGCCAAACAATGAACAATTTGACAAATTGTACATTTGGGGGGTTCACCACCCGGGTACGGACAAGGACCAAATCTTCCTGTATGCTCAATCATCAGGAAGAATCACAGTATCTACCAAAAGAAGCCAACAAGCTGTAATCCCGAATATCGGATCTAGACCCAGAATAAGGAATATCCCTAGCAGAATAAGCATCTATTGGACAATAGTAAAACCGGGAGACATACTTTTGATTAACAGCACAGGGAATCTAATTGCTCCTAGGGGTTACTTCAAAATACGAAGTGGGAAAAGCTCAATAATGAGATCAGATGCACCCATTGGCAAATGCAAGTCTGAATGCATCACTCCAAATGGAAGCATTCCCAATGACAAACCATTCCAAAATGTAAACAGGATCACATACGGGGCCTGTCCCAGATATGTTAAGCAAAGCACTCTGAAATTGGCAACAGGAATGCGAAATGTACCAGAGAAACAAACTAGAGGCATATTTGGCGCAATAGCGGGTTTCATAGAAAATGGTTGGGAGGGAATGGTGGATGGTTGGTACGGTTTCAGGCATCAAAATTCTGAGGGAAGAGGACAAGCAGCAGATCTCAAAAGCACTCAAGCAGCAATCGATCAAATCAATGGGAAGCTGAATCGATTGATCGGGAAAACCAACGAGAAATTCCATCAGATTGAGAAAGAATTCTCAGAAGTAGAAGGGAGAATTCAGGACCTTGAGAAATATGTTGAGGACACAAAAATAGATCTCTGGTCATACAACGCAGAGCTTCTTGTTGCCCTGGAAAACCAACATACAATTGATCTAACTGACTCAGAAATGAACAAACTGTTTGAAAAAACAAAGAAGCAACTGAGGGAAAATGCTGAGGATATGGGCAATGGTTGTTTCAAAATATACCACAAATGTGACAATGCCTGCATAGGATCAATCAGAAATGGAACTTATGACCACGATGTATACAGGGATGAAGCATTAAACAACCGGTTCCAGATCAAGGGAGTTGAGCTGAAGTCAGGGTACAAAGATTGGATCCTATGGATTTCCTTTGCCATATCATGIIIIIIGCTTTGTGTTGCTTTGTTGGGGTTCATCATGTGGGCCTGCCAAAAGGGCAACATTAGGTGCAACATTTGCATTTGAGTGCATTAATTAAAAACACCCTTGTTTCTACT | 288 |
| HA Influenza B | ATTTTCTAATATCCACAAAATGAAGGCAATAATTGTACTACTCATGGTAGTAACATCCAATGCAGATCGAATCTGCACTGGGATAACATCGTCAAACTCACCACATGTCGTCAAAACTGCTACTCAAGGGGAGGTCAACGTGACCGGTGTAATACCACTGACAACAACACCCACCAAATCTCATTTTGCAAATCTCAAAGGAACAGAAACCAGGGGGAAACTATGCCCAAAATGCCTCAACTGCACAGATCTGGATGTAGCCTTGGGCAGACCAAAATGCACAGGGAAAATACCCTCTGCAAGGGTTTCAATACTCCATGAAGTCAGACCTGTTACATCTGGGTGCTTTCCTATAATGCACGATAGAACAAAAATTAGACAGCTGCCTAACCTTCTCCGAGGATACGAACATGTCAGGTTATCAACTCACAACGTTATCAATGCAAAAGATGCACCAGGAAGACCCTACGAAATTGGAACCTCAGGGTCTTGCCCTAACATTACCAATGGAAACGGATTCTTCGCAACAATGGCTTGGGCCGTCCCAAAAAACAAAACAGCAACAAATCCATTAACAATAGAAGTACCATACATTTGTACAGAAGGAGAAGACCAAATTACCGTTTGGGGGTTCCACTCTGACAACGAGACCCAAATGGCAAAGCTCTATGGGGACTCAAAGCCCCAGAAGTTCACCTCATCTGCCAACGGAGTGACCACACATTACGTTTCACAGATTGGTGGCTTCCCAAATCAAACAGAAGACGGAGGACTACCACAAAGTGGCAGAATTGTTGTTGATTACATGGTGCAGAAATCTGGAAAAACAGGAACAATTACCTATCAAAGAGGTATTTTATTGCCTCAAAAAGTGTGGTGCGCAAGTGGCAGGAGCAAGGTAATAAAAGGATCCTTGCCCTTAATTGGAGAAGCAGATTGCCTCCATGAAAAATACGGTGGATTAAACAAAAGCAAGCCTTACTACACAGGGGAACATGCAAAGGCCATAGGAAATTGCCCAATATGGGTGAAAACACCCTTGAAGCTGGCCAATGGAACCAAATATAGACCCCCTGCAAAACTATTAAAGGAAAGAGGTTTCTTCGGAGCCATTGCTGGTTTCTTAGAGGGAGGATGGGAAGGAATGATTGCAGGTTGGCACGGATACACATCCCATGGGGCACATGGAGTAGCGGTGGCAGCTGACCTTAAGAGCACTCAAGAGGCCATAAACAAGATAACAAAAAATCTCAACTCTTTGAGTGAGCTGGAAGTAAAGAATCTTCAAAGACTAAGCGGTGCCATGGATGAACTCCACAACGAAATACTAGAACTAGATGAGAAAGTGGATGATCTCAGAGCTGACACAATAAGCTCACAAATAGAACTCGCAGTCCTGCTTTCCAATGAAGGAATAATAAACAGTGAAGATGAACATCTCTTGGCGCTTGAAAGAAAGCTGAAGAAAATGCTGGGCCCCTCTGCTGTAGAGATAGGGAATGGATGCTTTGAAACCAAACACAAGTGCAACCAGACCTGCCTCGACAGAATAGCTGCTGGTACCTTTGATGCAGGAGAATTTTCTCTCCCCACCTTTGATTCACTGAATATTACTGCTGCATCTTTAAATGACGACGGATTGGACAATCATACTATACTGCTTTACTACTCAACTGCTGCCTCCAGTTTGGCTGTAACACTGATGATAGCTATCTTTGTTGTTTATATGGTCTCCAGAGACAATGTTTCTTGCTCCATTTGTCTATAAGGAAAGTTAAGCCCTGTATTTTCCTTTATTGTAGTGCTTGTTTGCTTGTTGTCATTACAAAGAAACGTTATTGAAAAAT | 289 |

### EXAMPLE

The following is a non-limiting example of the present invention. It is to be understood that said example is not intended to limit the present invention in any way. Equivalents or substitutes are within the scope of the present invention.

### Evolutionary Convergence of Human Sars-Cov-2 Into Bat and Pangolin-Derived Sars-Like Coronaviruses: Understanding The Animal Origins of Sars-Cov-2 Is Critical For The Development of a Pre-Emptive Pan-Coronavirus Vaccine to Protect From Future Human Outbreaks and Deter Future Zoonosis.

Sequence comparison among SARS-CoV-2 and previous Coronavirus strains: We retrieved 81,963 human SARS-CoV-2 genome sequences from GISAID database representing countries from North America, South America, Central America, Europe, Asia, Oceania, and Africa. Furthermore, the full-length sequences of SARS-CoV strains (SARS-CoV-2-Wuhan-Hu-1 (MN908947.3), SARS-CoV-Urbani (AY278741.1), HKU1-Genotype B (AY884001), CoV-OC43 (KF923903), CoV-NL63 (NC_005831), CoV-229E (KY983587)) and MERS (NC_019843)) found in the human host were obtained from the NCBI GenBank SARS-CoV-2 genome sequences from bat (RATG13 (MN996532.2), ZXC21 (MG772934.1), YN01 (EPI_ISL_412976), YN02(EPI_ISL_412977)), and pangolin (GX-P2V (MT072864.1), GX-P5E (MT040336.1), GX-P5L (MT0403351), GX-P1E (MT040334.1), GX-P4L (MT040333.1), GX-P3B (MT072865.1), MP789 (MT121216.1), Guangdong-P2S (EPI_ISL_410544)) were obtained from NCBI (www.ncbi.nlm.nih.gov/nuccore) and GSAID (www.gisaid.org). More so, the SARS-CoV strains from bat (WIV16 (KT444582.1), WIV1 (KF367457.1), YNLF_31C (KP886808.1), Rs672 (FJ588686.1), recombinant strain (FJ211859.1), camel (KT368891.1, MN514967.1, KF917527.1, NC_028752.1), and civet (Civet007, A022, B039)) were also retrieved from the NCBI GenBank. The sequences were aligned using the ClustalW algorithm in MEGA X.

Sequence conservation analysis of SARS-CoV-2: The SARS-CoV-2-Wuhan-Hu-1 (MN908947.3) protein sequence was compared with SARS-CoV and MERS-CoV specific protein sequences obtained from human, bat, pangolin, civet and camel. The Sequence Variation Analysis was performed on the consensus aligned protein sequences from each virus strain. This Sequence Homology Analysis identified consensus protein sequences from the SARS-CoV and MERS-CoV and predicted the Epitope Sequence Analysis.

First, the present invention screened for an evolutionary relationship among human SARS-CoV-2 and SARS-CoV/MERS-CoV strains from previous outbreaks (i.e., Urbani, MERS-CoV, OC43, NL63, 229E, HKU1-genotype-B) along with 25 SARS-like Coronaviruses genome sequence (SL-CoVs) obtained from different animal species: Bats (Rhinolophus affinis and Rhinolophus malayanus), civet cats (Paguma larvata) and pangolins (Manis javanica), and MERS-CoVs from camels (Camelus dromedarius and Camelus bactrianus). These sequence alignments revealed similarity of the original human-SARS-CoV-2 strain found in Wuhan, China to four bat SL-CoV strains: hCoV-19-bat-Yunnan-RmYN02, bat-CoV-19-ZXC21, and hCoV-19-bat-Yunnan-RaTG13 obtained from the Yunnan and Zhejiang provinces of China (FIG. 2A). With further genetic distance analysis, it was discovered that the least evolutionary divergence between SARS-CoV-2 isolate Wuhan-Hu-1 and the above mentioned three SL-CoVs isolates from bats, namely: (1) Bat-CoV-RaTG13 (0.1), (2) bat-CoV-19-ZXC21 (0.1) and (3) Bat-CoV-YN02 (0.2). Moreover, the phylogenetic analysis performed among the whole genome sequences of a total of 81.963 SARS-CoV-2 strains for which sequences have been reported in circulation in 190 countries suggest an evolutionary convergence of bat and pangolin SL-CoVs into the human SARS-CoV-2 strains (FIG. 2B). Furthermore, through a complete genome tree derived from the 81,963 SARS-CoV-2 genome sequences submitted from Asian, African, North American, South American, European, and Oceanian regions, it was confirmed that the least evolutionary divergence for SARS-CoV-2 strains is in SL-CoVs isolated from bats and pangolins (FIG. 2B).

Altogether, the phylogenetic analysis and genetic distance suggest that the highly contagious and deadly human-SARS-CoV-2 strain originated from bats, most likely from either the Bat-CoV-19-ZXC21 or Bat-CoV-RaTG13 strains, that spilled over into humans after further mutations and/or recombination. These mutations and/or recombination(s) possibly contributed to the rapid global expansion of the highly contagious and deadly SARS-CoV-2.

### Genome-Wide Identification of SARS-CoV-2 CD8⁺ T Cell epitopes That are Highly Conserved Between Human and Bat/Pangolin Coronaviruses.

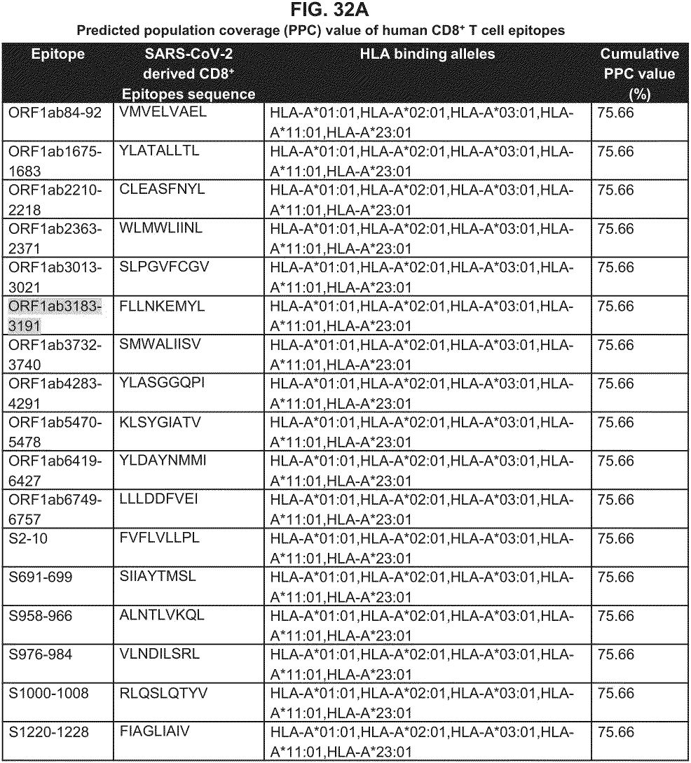

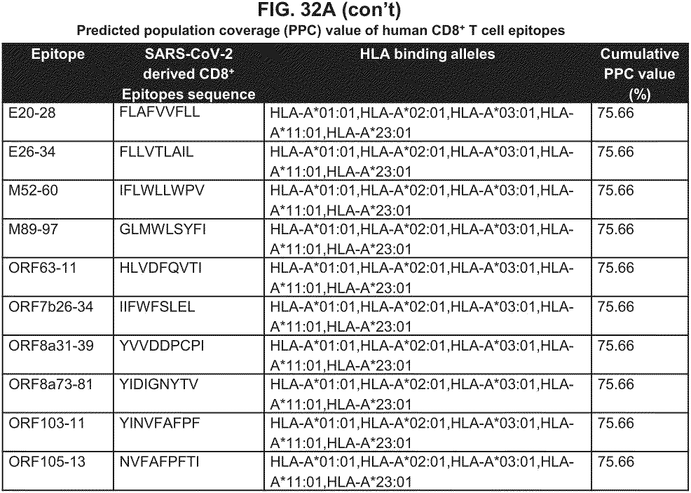

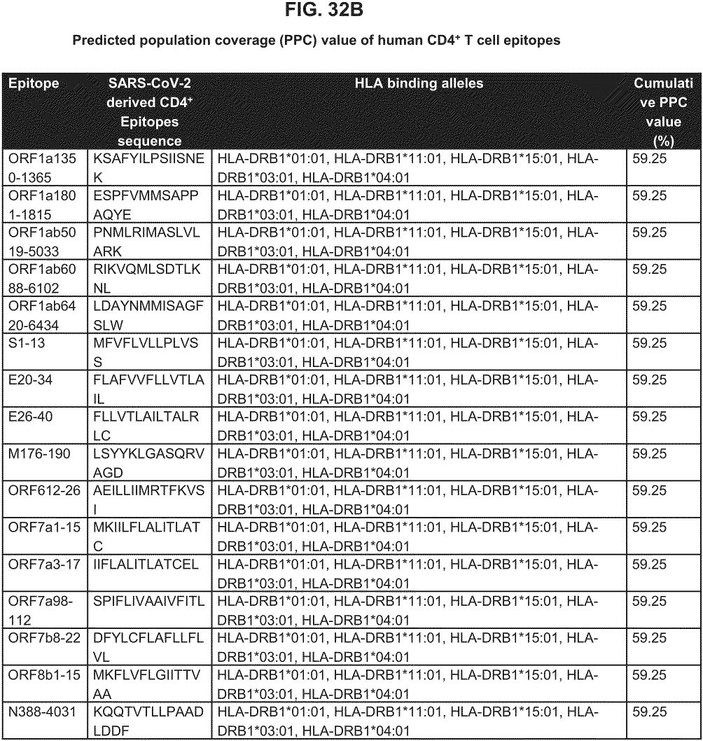

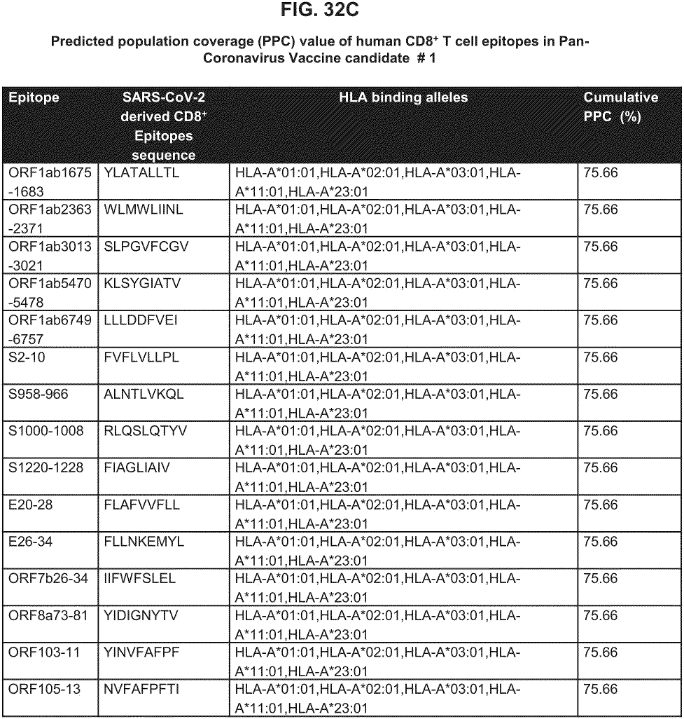

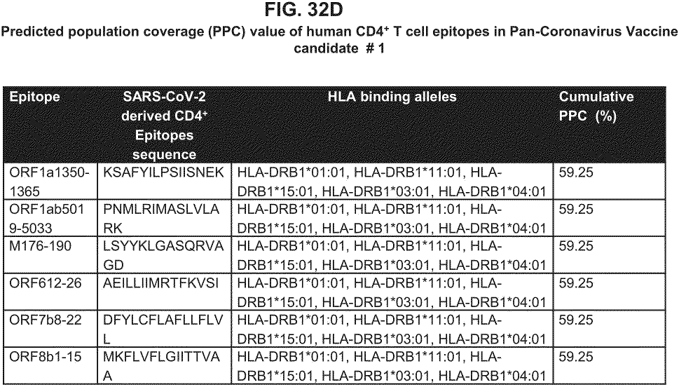

SARS-CoV-2 CD8 and CD4 T Cell Epitope Prediction: Epitope prediction was carried out using the twelve proteins predicted for the reference SARS-CoV-2 isolate, Wuhan-Hu-1. The corresponding SARS-CoV-2 protein accession identification numbers are: YP_009724389.1 (ORF1ab), YP_009725295.1 (ORF1a), YP_009724390.1 (surface glycoprotein), YP_009724391.1 (ORF3a), YP_009724392.1 (envelope protein), YP_009724393.1 (membrane glycoprotein), YP_009724394.1 (ORF6), YP_009724395.1 (ORF7a), YP_009725318.1 (ORF7b), YP_009724396.1 (ORF8), YP_009724397.2 (nucleocapsid phosphoprotein), YP_009725255.1 (ORF10). The tools used for CD8⁺ T cell-based epitope prediction were SYFPEITHI, MHC-I binding predictions, and Class I Immunogenicity. Of these, the latter two were hosted on the IEDB platform. For the prediction of CD4⁺ T cell epitopes, we used multiple databases and algorithms, namely SYFPEITHI, MHC-II Binding Predictions, Tepitool, and TEPITOPEpan. For CD8⁺ T cell epitope prediction, we selected the 5 most frequent HLA-A class I alleles (HLA-A*01:01, HLA-A*02:01, HLA-A*03:01, HLA-A*11:01, HLA-A*23:01) with large coverage of the world population, regardless of race and ethnicity (FIG. 32A, FIG. 32C) using a phenotypic frequency cutoff ≥ 6%. Similarly, for CD4 T cell epitope prediction, selected HLA-DRB1 *01:01, HLA-DRB1*11:01, HLA-DRB1*15:01, HLA-DRB1*03:01, HLA-DRB1*04:01 alleles with large population coverage (FIG. 32B, FIG. 32D). Subsequently, using NetMHC we analyzed the SARS-CoV-2 protein sequence against all the aforementioned MHC-I and MHC-II alleles. Epitopes with 9mer length for MHC-I and 15mer length for MHC-II were predicted. Subsequently, the peptides were analyzed for binding stability to the respective HLA allotype. The stringent epitope selection criteria were based on picking the top 1% epitopes focused on prediction percentile scores.

Potential CD8⁺ T cell epitopes were first predicted from the entire genome sequence of the first SARS-CoV-2-Wuhan-Hu-1 strain (NCBI GenBank accession number MN908947.3). For this, multiple databases and algorithms were used including the SYFPEITHI, MHC-I processing predictions, MHC-I binding predictions, MHC-I immunogenicity and Immune Epitope Database (IEDB). Epitopes restricted to the five most frequent human leukocyte antigen (HLA) class I alleles with large coverage in worldwide human populations, regardless of race and ethnicity (i.e., HLA-A*01:01, HLA-A*02:01, HLA-A*03:01, HLA-A*11:01. HLA-A*23:01) were focused on (FIG. 32A, FIG. 32C).

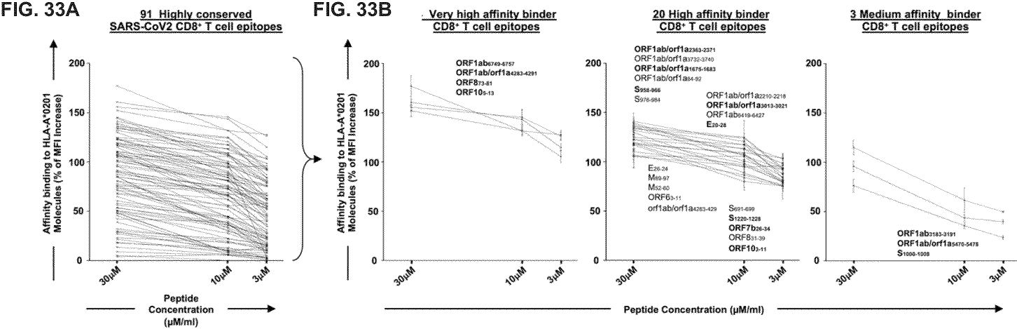

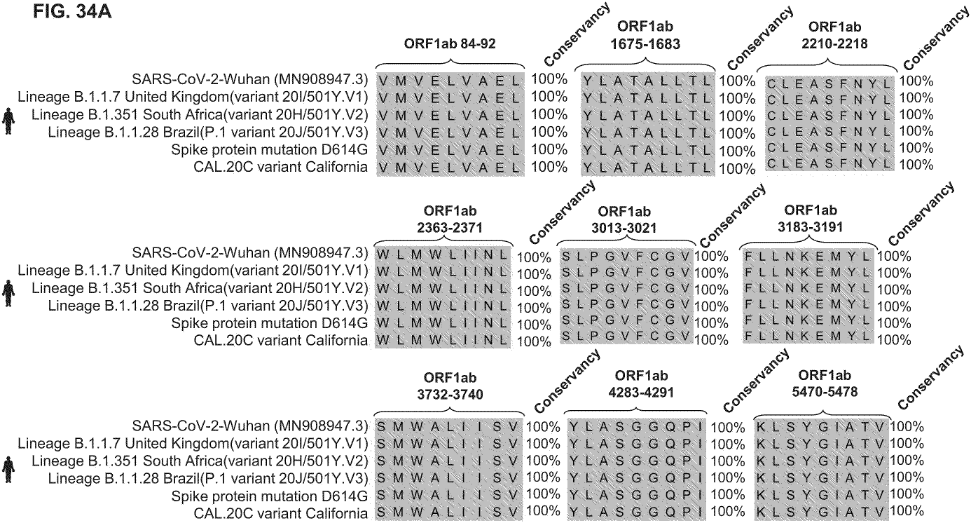

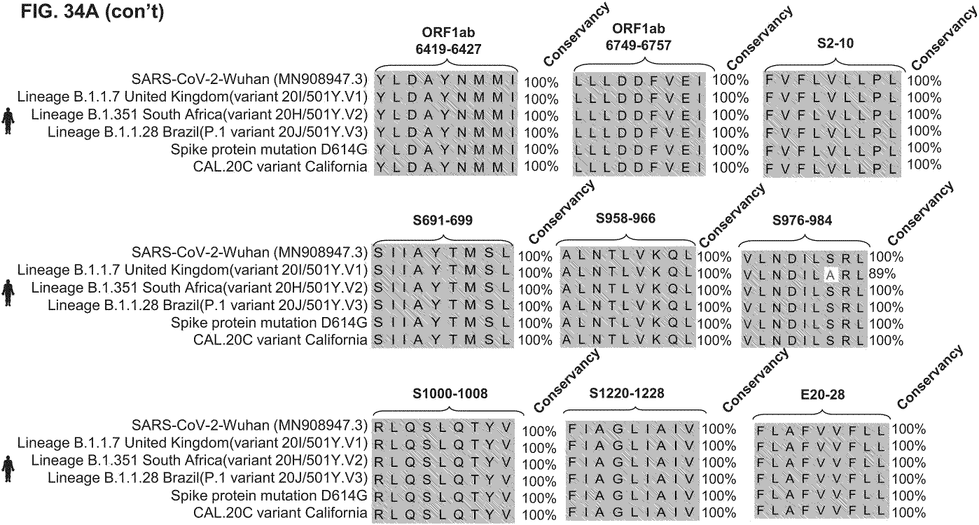

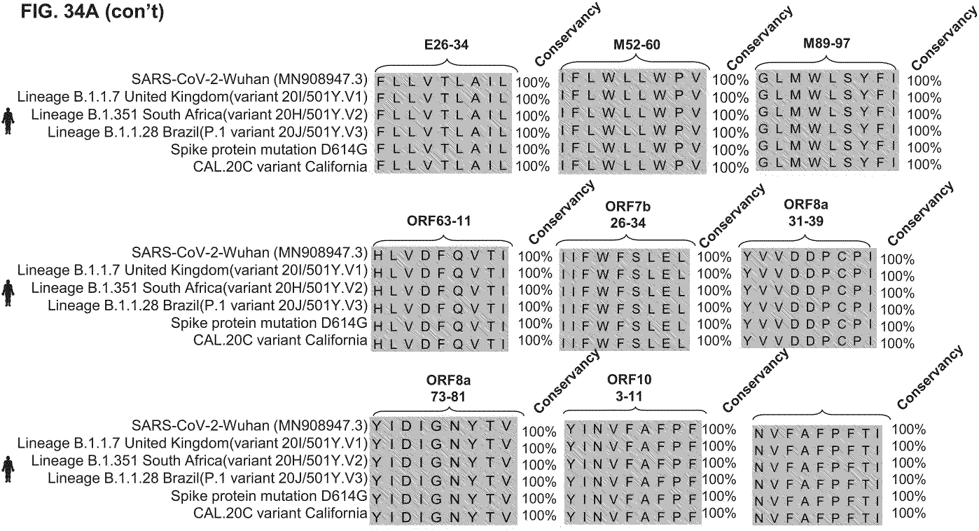

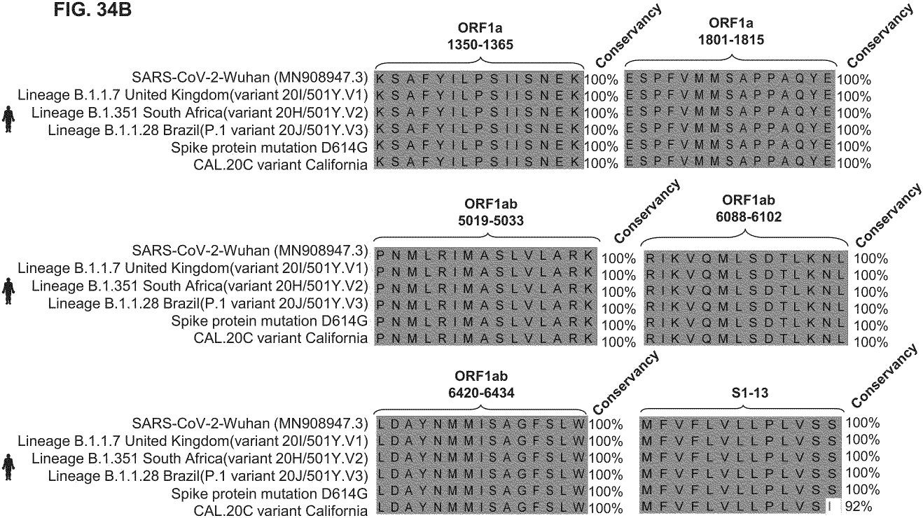

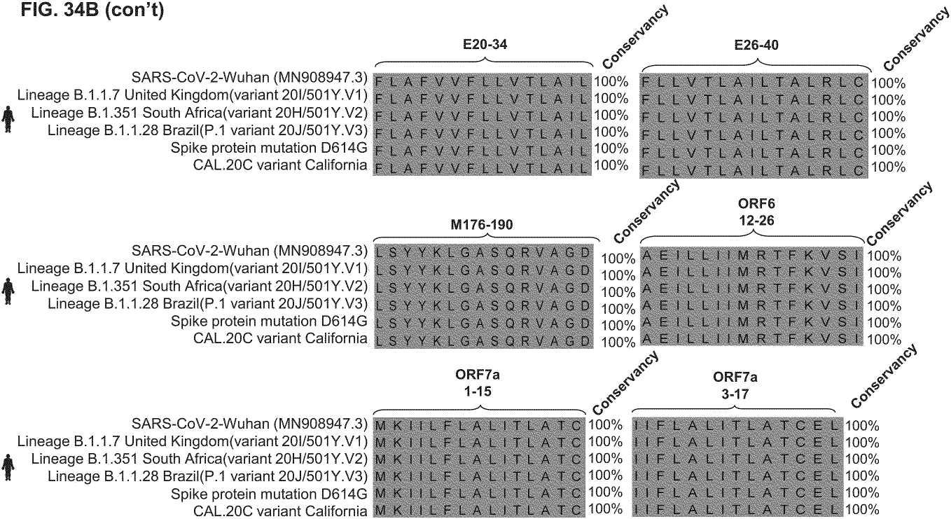

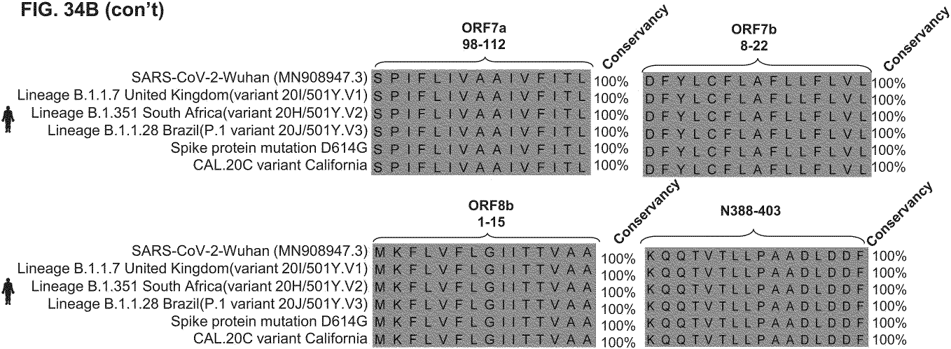

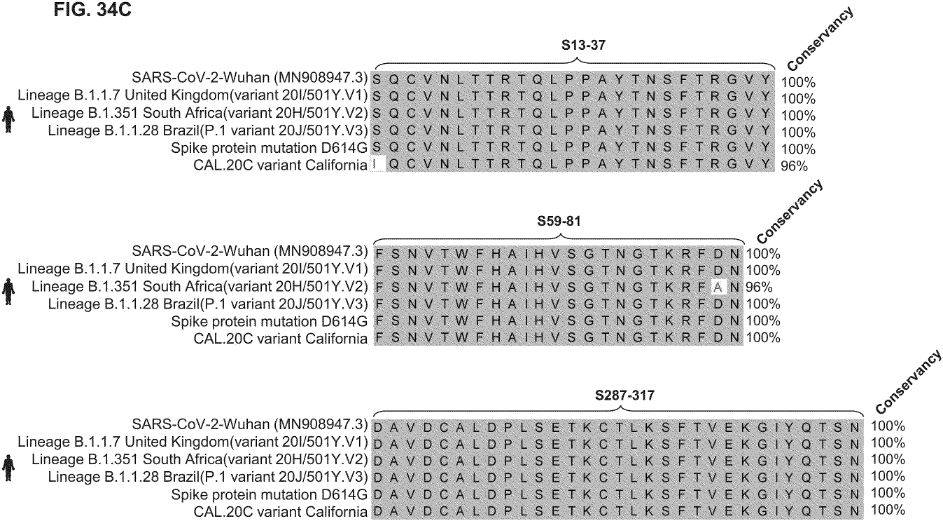

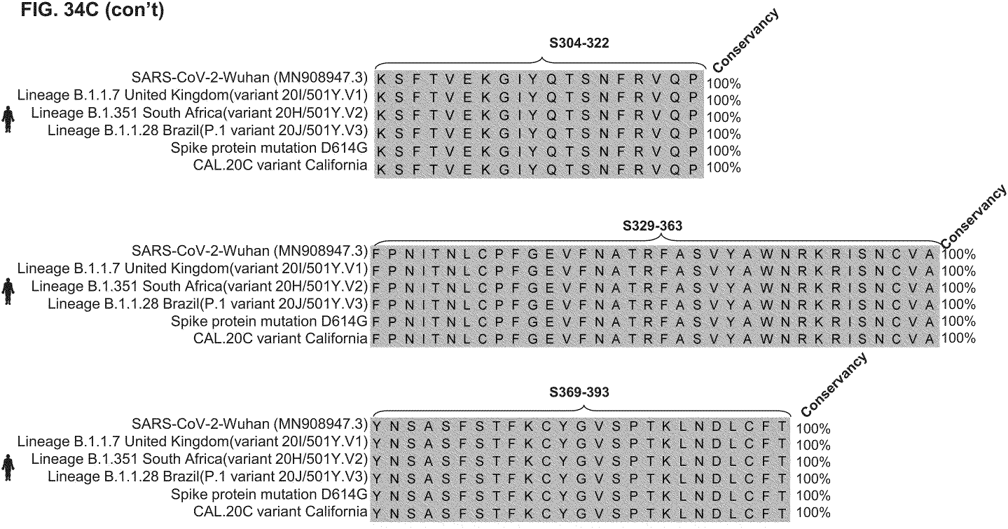

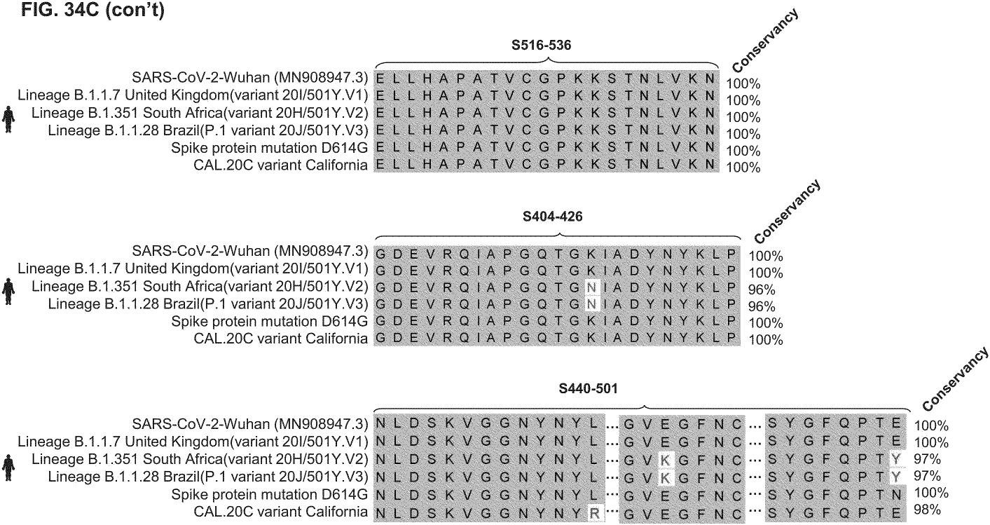

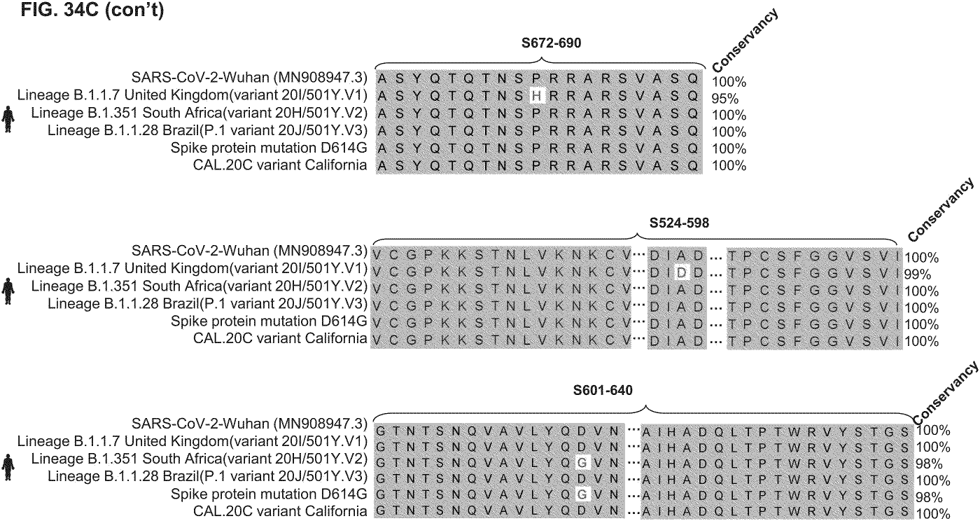

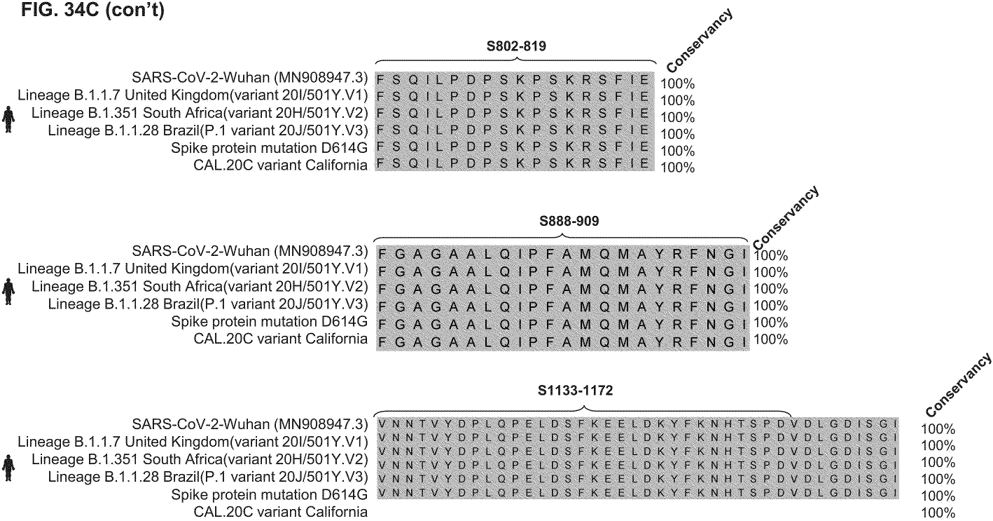

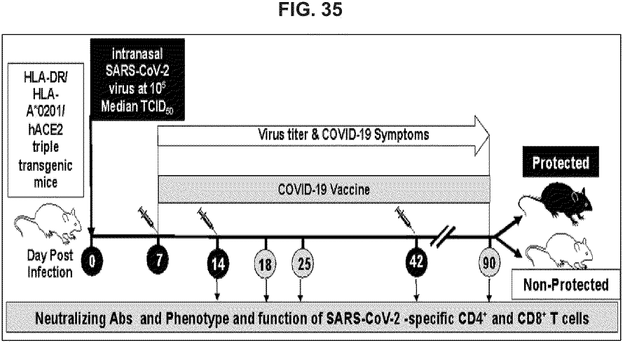

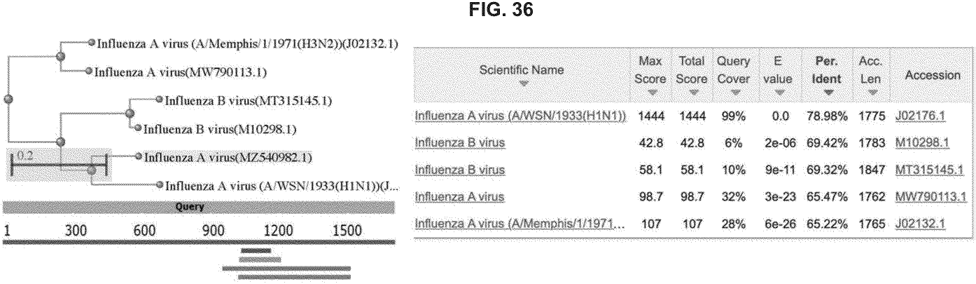

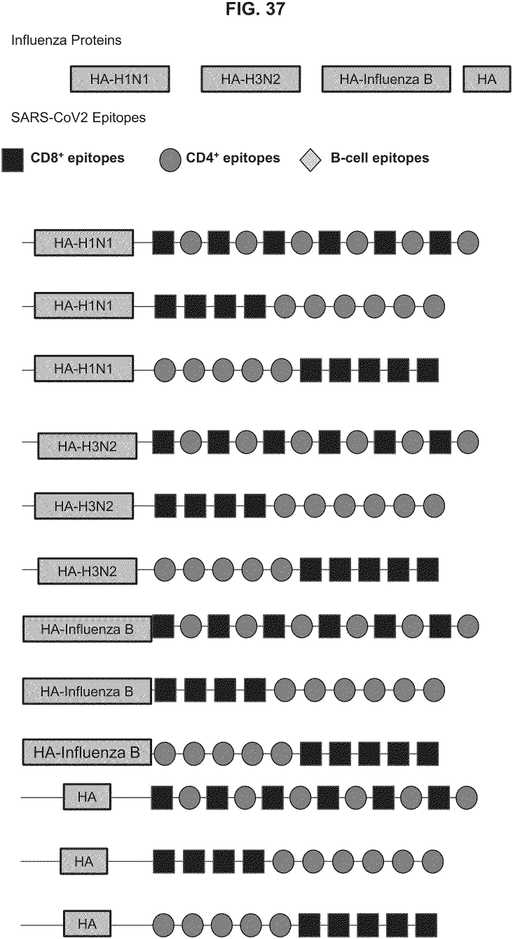

Using the aforementioned criteria, a total of 9,660 potential CD8⁺ T cell epitopes were originally identified derived from 12 structural proteins (surface glycoprotein, membrane glycoprotein, nucleocapsid phosphoprotein) and open-reading-frames (ORFs) of SARS-CoV-2-Wuhan-Hu-1 strain. Subsequently, this large pool of epitopes was narrowed down to 91 epitopes, that are highly conserved among: (i) over 81,000 SARS-CoV-2 strains (that currently circulate in 190 countries on 6 continents); (ii) the 4 major “common cold” Coronaviruses that caused previous outbreaks (i.e., hCoV-OC43, hCoV-229E, hCoV-HKU1 genotype B, and hCoV-NL63); and (iii) the SL-CoVs that are isolated from bats, civet cats, pangolins and camels (FIG. 33). While the highest degree of similarity (expressed as % of resemblance) was identified among 81,963 SARS-CoV-2 strains, 6 strains of previous human SARS-CoVs and 18 animal SL-CoVs strains isolated from bats and pangolins, only a small percentage of similarity was found between the SARS-CoV-2 and MERS-CoV strains. However, a significantly lower degree of similarity was recorded amongst the SARS-CoV-2 and the SL-CoVs strains isolated from civet cats’ and camels’ CoVs.

Twenty-seven SARS-CoV-2 human CD8⁺ T cell epitopes were further identified, out of the 91 epitopes, that bound with high affinity with HLA-A*02:01 molecules, using in vitro peptide-HLA binding assay. Four epitopes were found to be very high affinity binders. The 27 epitopes with high binding affinity were later confirmed in silico using molecular docking models across 5 major HLA-A*01:01, HLA-A*02:01, HLA-A*03:01, HLA-A*1 1:01, HLA-A*23:01 haplotypes (FIG. 6A, FIG. 6B). The highest binding affinity to HLA-A*02:01 molecules, with the highest interaction similarity (Sᵢₙₜₑᵣ) scores (blue squares), were recorded for ORF1ab₆₇₄₉₋₆₇₅₇, S₂₋₁₀, S₉₅₈₋₉₆₆, S₁₂₂₀₋₁₂₂₈, E₂₆₋₃₄. ORF8₈₃₋₉₁, ORF10₃₋₁₁ and ORF10₅₋₁₃ whereas minimum Sᵢₙₜₑᵣ score was observed for ORF1 ab₃₇₃₂₋₃₇₄₀, S₆₉₁₋₆₉₉ and M₈₉₋₉₇. Other CD8⁺ T cell epitopes like ORF1 ab₁₆₇₅₋ ₁₆₈₃, ORF1ab₂₃₆₃₋₂₃₇₁, ORF1ab₃₀₁₃₋₃₀₂₁ and ORF7b₂₆₋₃₄ were also found with intermediate Sᵢₙₜₑᵣ scores (FIG. 6A, FIG. 6B). While the identified highly conserved CD8⁺ T cell epitopes were distributed within 8 of the 12 structural and non-structural ORFs (i.e., ORF1ab, S, E, M, ORF6, ORF7b, ORF8, and ORF10), the highest numbers of epitopes were localized in the replicase polyprotein 1ab/1a (ORF1ab) (9 epitopes) followed by the spike glycoprotein (S) (5 epitopes).

Altogether, these results identified 27 highly conserved potential human CD8⁺ T cell epitopes from the sequence of SARS-CoV-2 that are highly conserved among 81,963 SARS-CoV-2 strains, the 4 major “common cold” Coronaviruses (i.e., hCoV-OC43, hCoV-229E, hCoV-HKU1 genotype B, and hCoV-NL63), newly found highly transmissible variants and several SL-CoV strains that are isolated from bats and pangolins. These results suggest that both the structural and the non-structural proteins are immunodominant antigens that are targeted by human CD8⁺ T cells from both COVID-19 patients and “common cold” Coronaviruses infected healthy individuals.

### In Silico Screening of Potential Promiscuous Sars-Cov-2 Cd4⁺ T Cell Epitopes That are Highly Conserved Between Human and Bat/Pangolin Coronaviruses.

Subsequently a total of 9,594 potential HLA-DR-restricted CD4⁺ T cell epitopes were identified from the whole genome sequence of SARS-CoV-2-Wuhan-Hu-1 strain (MN908947.3) using multiple databases and algorithms including the SYFPEITHI, MHC-II Binding Predictions, Tepitool and TEPITOPEpan). These potential promiscuous CD4⁺ T cell epitopes were screened in silico against the five most frequent HLA-DR alleles with large coverage in the human population, regardless of race or ethnicity: HLA-DRB1*01:01, HLA-DRB1*11:01, HLA-DRB1*15:01, HLA-DRB1*03:01, HLA-DRB1*04:01. The number of potential CD4⁺ T cell epitopes was later narrowed down to 16 epitopes based on: (i) the epitope sequences that are highly conserved among 81,963 SARS-CoV-2 strains, the 4 major “common cold” and 25 SL-CoV strains isolated from bats, civet cats, pangolins and camels (FIG. 10); and (ii) their high binding affinity to HLA-DR molecules using in silico molecular docking models (FIG. 11A, FIG. 11B). The sequences of most of the 16 CD4⁺ T cell epitopes are 100% conserved and common among 81,963 SARS-CoV-2 strains currently circulating in 6 continents. A high degree of sequence similarities was also identified in the sequences of most 16 CD4⁺ T cell epitopes among the SARS-CoV-2 strains and the six strains of previous human SARS-CoVs (e.g., up to 100% sequence identity for epitopes ORF1ab₅₀₁₉₋₅₀₃₃, ORF1ab₆₀₈₈₋₆₁₀₂, ORF1ab ₆₄₂₀₋₆₄₃₄. E₂₀₋₃₄, E₂₆₋₄₀ and M₁₇₆₋₁₉₀). Moreover, a high degree of sequence similarities was also identified among the SARS-CoV-2 and the SLx-CoV strains isolated from bats and pangolins. In contrast, a lower sequence similarity was identified among CD4⁺ T cell epitopes from SARS-CoV-2 strains and the SL-CoV strains isolated from civet cats followed by MERS-like CoV strains isolated from camels.

The 16 highly conserved CD4⁺ T cell epitopes are distributed within 9 out of the 12 structural and non-structural ORFs (i.e., ORF1ab, S, E, M, ORF6, ORF7a, ORF7b, ORF8 and N). The highest numbers of epitopes were localized in the replicase polyprotein ORF1ab/1a (5 epitopes) followed by ORF7a (3 epitopes). Unlike the human CD8* T cell epitopes, the human CD4⁺ T cell epitopes are found to be expressed in each of the structural S, E, M. and N proteins. Two epitopes are from the envelope protein (E), 1 epitope from the membrane protein (M), 1 epitope from the nucleoprotein (N) protein, and 1 epitope from the spike protein (S). The remaining CD4⁺ T cell epitopes are distributed among the ORF6, ORF7a, ORF7b and ORF8 proteins.

Altogether, these results identified 16 potential CD4⁺ T cell epitopes from the whole sequence of SARS-CoV-2 that cross-react and have high sequence similarity among 81,963 SARS-CoV-2 strains, the main 4 major “common cold” Coronaviruses and the SL-CoV strains isolated from bats and pangolins. Similar to CD8⁺ T cell epitopes, the replicase polyprotein ORF1ab appeared to be the most immunodominant antigen with a high number of conserved epitopes that may possibly be targeted by human CD4⁺ T cells.

### Cross-Reactive Human and Animal Coronavirus-Derived Epitopes, Spanning the Whole Virus Proteome, are Targeted by Memory Cd4⁺ And Cd8⁺ T Cells From Sars-Cov-2 Patients and Unexposed Healthy Individuals.

Human study population: Sixty-three COVID-19 patients and ten unexposed healthy individuals, who had never been exposed to SARS-CoV-2 or COVID-19 patients, were enrolled in this study. Seventy-eight percent were non-White (African, Asian, Hispanic and others) and 22% were white. Forty-four percent were females, and 56% were males with an age range of 26-95 (median 62). None of the symptomatic patients were on antiviral or anti-inflammatory drug treatments at the time of blood sample collections. The COVID-19 patients (n = 63) were divided into 4 groups depending on the severity of the symptoms: Group 1 that comprised of SARS-CoV-2 infected patients that never developed any symptoms or any viral diseases (i.e., asymptomatic patients) (n = 11); Group 2 with mild symptoms (i.e., Inpatient only, n = 32); Group 3 with moderate symptoms (i.e., ICU admission, n = 11) and Group 4 with severe symptoms (i.e., ICU admission +/- Intubation or death, n = 9). As expected, compared to the asymptomatic group, all of the 3 symptomatic groups (i.e., mild, moderate and severe) had higher percentages of comorbidities, including diabetes (22% to 64%), hypertension (64% to 78%),cardiovascular disease (11% to 18%) and obesity (9% to 50%). The final Group 5 was comprised of unexposed healthy individuals (controls), with no history of COVID-19 or contact with COVID-19 patients (n = 10) collected prior to 2019. All subjects were enrolled at the University of California Irvine under Institutional Review Board-approved protocols (IRB # 2020-5779). A written informed consent was received from all participants prior to inclusion in this study.

Whether the potential SARS-CoV-2 CD4⁺ and CD8⁺ T cell epitopes that are highly conserved between human and animal Coronaviruses would recall memory CDS⁺ T cells from COVID-19 patients as well as from healthy individuals, who have never been exposed to SARS-CoV-2 or to COVID-19 patients were assessed (i.e., from healthy individuals blood samples that were collected from 2014 to 2018)

Blood-derived peripheral blood mononuclear cells (PBMCs) from COVID-19 patients and healthy individuals were analyzed by ELISpot for frequencies in SARS-CoV-2 epitopes-specific IFN-Y-producing CD8⁺ T cells. As shown in FIG. 7B, significant numbers of SARS-CoV-2 epitopes-specific memory CD8⁺ T cells producing IFN-_{Y} were detected in PBMCs of COVID-19 patients. Out of the 27 highly conserved cross-reactive SARS-CoV-2 CD8⁺ T cell epitopes (FIG. 5) selected for their binding affinity with HLA-A*02:01 molecules (FIG. 33B), strong T cell responses (mean SFCs > 50 per 0.5×10⁶ PBMCs fixed as threshold) were detected in COVID-19 patients against 10 epitopes derived from: (i) structural proteins like Spike (i.e., S₉₅₈₋₉₆₆, S₉₇₆₋₉₈₄, S₁₀₀₀₋₁₀₀₈ and S₁₂₂₀₋₁₂₂₈) or the Envelope proteins (i.e., E₂₆₋₃₄) and (ii) non-structural proteins (i.e., ORF1ab₁₆₇₅₋₁₆₈₃, ORF1ab₂₂₁₀₋₂₂₁₈, ORF1ab₆₇₄₉₋₆₇₅₇, ORF6₃₋₁₁, ORF10₃₋₁₁) (FIG. 2B). In addition, 12 other SARS-CoV-2 CD8⁺ T cell epitopes from structural of non-structural SARS-CoV-2 proteins induced an intermediate response (with a mean SFCs between 25 and 50 per 0.5×10⁶ PBMCs) in COVID-19 patients: ORF1ab₈₄₋₉₂, ORF1ab₃₀₁₃₋₃₀₂₁, ORF1ab₃₁₈₃₋₃₁₉₁. ORF1ab₃₇₃₂₋₃₇₄₀, ORF1ab₄₂₈₃₋₄₂₉₁, ORF1a6₈₄₁₉₋₆₄₂₇, S₂₋₁₀, S₆₉₁₋₆₉₉, E₂₀₋₂₈, M₅₂₋₆₀, M₈₉₋₉₇ and ORF10₅₋₁₃.

Moreover, among the 27 SARS-CoV-2 epitopes, 7 epitopes recalled a strong memory CD8⁺ T cells response (mean SFCs > 50) from unexposed healthy individuals (i.e., ORF1ab₁₆₇₅₋₁₆₈₃, ORF1ab₃₇₃₂₋ ₃₇₄₀, ORF1ab₄₂₈₃₋₄₂₉₀, ORF1ab₅₄₇₀₋₅₄₇₈, ORF1ab₆₇₄₉₋₈₇₅₇, S₉₇₆₋₉₈₄ and S₁₀₀₀₋₁₀₀₈, S₁₂₂₀₋₁₂₂₈) and 5 epitopes recalled a memory CD8⁺ T cells response that was intermediate (ORF1ab₆₄₁₉₋₆₄₂₇, S₂₋₁₀, E₂₆₋₃₄, ORF10₃₋₁₁ and ORF10₅₋₁₃) (FIG. 7B). However, the unexposed healthy individuals exhibited a different pattern of CD8⁺ T cell immunodominance as compared to COVID-19 patients. The epitopes-specificity and function of memory CD8⁺ T cells in HLA-*A02:01-positive COVID-19 patients and healthy individuals were then compared using flow cytometry (FIG. 7C). For a better comparison, a similar FACS gating strategy was applied to PBMCs-derived T cells from both COVID-19 and healthy donors (data not shown). The COVID-19 patients appeared to have a higher frequency of CD8⁺ T cells compared to healthy donors (FIG. 7C). Tetramer staining showed that many of SARS-CoV-2 epitope-specific CD8⁺ T cells are multifunctional producing IFN-y, TNF-α and expressing CD69 and CD107^{a/b} markers of activation and cytotoxicity in COVID-19 patients.

Similar to SARS-CoV-2 memory CD8⁺ T cells, memory CD4⁺ T cells specific to several highly conserved SARS-CoV-2 epitopes were detected in both COVID-19-recovered patients and unexposed healthy individuals (FIG. 12A, FIG. 12B. FIG. 12C). Out of the 16 highly conserved cross-reactive SARS-CoV-2 CD4⁺ T cell epitopes (FIG. 10), strong T cell responses (mean SFCs > 50 per 0.5×10⁶ PBMCs fixed as a threshold) were detected in COVID-19 patients against 2 epitopes, one derived from the structural protein M (M₁₇₆₋₁₉₀) and one from the non-structural protein ORF1a (ORF1a₁₃₅₀₋₁₃₆₅) (FIG. 12A. FIG. 12B). Moreover, 6 additional SARS-CoV-2 CD8⁺ T cell epitopes from non-structural SARS-CoV-2 proteins (i.e., ORF1a₁₈₀₁₋₁₈₁₅, ORF1₆₀₈₈₋₆₁₀₂. ORF1a₆₄₂₀₋₆₄₃₄, ORF6₁₂₋₂₆, ORF7a₃₋₁₇ and ORF8b₁₋₁₅) and two more epitopes from structural proteins (i.e., S₁₋₁₃ and N₃₈₈₋₄₀₃) induced an intermediate CD4⁺ T cell response (mean SFCs between 25 and 50 per 0.5×10⁶ PBMCs) in COVID-19 patients (Similar to SARS-CoV-2 memory CD8⁺ T cells, memory CD4⁺ T cells specific to several highly conserved SARS-CoV-2 epitopes were detected in both COVID-19-recovered patients and unexposed healthy individuals. Out of the 16 highly conserved cross-reactive SARS-CoV-2 CD4⁺ T cell epitopes , strong T cell responses (mean SFCs > 50 per 0.5×10⁶ PBMCs fixed as a threshold) were detected in COVID-19 patients against 2 epitopes, one derived from the structural protein M (M₁₇₆₋₁₉₀) and one from the non-structural protein ORF1a (ORF1a₁₃₅₀₋₁₃₆₅). Moreover, 6 additional SARS-CoV-2 CD8⁺ T cell epitopes from non-structural SARS-CoV-2 proteins (i.e.. ORF1a₁₈₀₁₋ ₁₈₁₅, ORF1a₆₀₈₈₋₆₁₀₂, ORF1a₆₄₂₀₋₆₄₃₄. ORF6₁₂₋₂₆, ORF7a₃₋₁₇ and ORF8b₁₋₁₅) and two more epitopes from structural proteins (i.e., S₁₋₁₃ and N₃₈₈₋₄₀₃) induced an intermediate CD4⁺ T cell response (mean SFCs between 25 and 50 per 0.5×10⁶ PBMCs) in COVID-19 patients.

Besides, among the 16 SARS-CoV-2 epitopes, 2 epitopes recalled a strong memory CD4⁺ T cells response (mean SFCs > 50) from unexposed healthy individuals with no history of COVID-19 (i.e., ORF1a₁₃₅₀₋₁₃₆₅ and ORF6₁₂₋₂₆) (FIG. 12B and FIG. 12C). Furthermore, 5 additional epitopes recalled an intermediate CD4⁺ T cells response in these unexposed healthy individuals (i.e., ORF1a₁₈₀₁₋₁₈₁₅, S₁₋₁₃, M₁₇₆₋ ₁₉₀, ORF8b₁₋₁₅ and N₃₈₈₋₄₀₃). Unlike for CD8⁺ T cell responses, the unexposed healthy individuals exhibited a similar pattern of CD4⁺ T cell immunodominance as compared to COVID-19 patients, with few differences in the magnitude of the responses only. Multifunctional SARS-CoV-2 epitopes-specific CD4⁺ T cells, expressing CD69, CD107^{a/b} and TNF-a, were detected using specific tetramers in PBMCs of HLA-DR1 positive COVID-19 patients and healthy individuals (FIG. 12C) with a trend showing higher percentage of these cells in COVID-19 patients, although not significantly higher.

The immunogenicity of the identified SARS-CoV-2 human CD4⁺ and CD8⁺ T cell epitopes was assessed in “humanized” HLA-DR1/HLA-A*02:01 double transgenic mice (FIG. 8A and FIG. 13A). A mixture of peptides incorporating CD4⁺ T-cell or CD8⁺ T-cell epitopes were delivered with CpG and Alum, as shown in FIG. 8A and FIG. 13A and detailed herein. As a negative control, mice received adjuvant alone. The induced SARS-CoV-2 epitope-specific CD4⁺ and CD8⁺ T cell responses were determined in the spleen using multiple immunological assays, including IFN-y ELISpot, FACS surface markers of activation, markers of cytotoxic degranulation and intracellular cytokine staining. The gating strategy used for mice is shown in FIG. 8B and FIG. 13B Two weeks after the second immunization with the mixture of CD8⁺ T-cell peptides, 10 out of 27 highly conserved SARS-CoV-2 human CD8⁺ T cell epitope peptides were immunogenic in “humanized” HLA-DR1/HLA-A*02:01 double transgenic mice (FIG. 8A). The remaining 17 CD8⁺ T cell epitopes presented moderate/low immunogenicity levels in HLA-DR1/HLA-A*02:01 double transgenic mice. The immunogenic epitopes were derived from both structural Spike protein (S₂₋₁₀, S₉₅₈₋₉₆₆, S₁₀₀₀₋₁₀₀₈ and S₁₂₂₀₋₁₂₂₈) and Envelope protein (E₂₀₋₂₈) and from non-structural proteins (i.e., ORF1ab₂₃₆₃₋₂₃₇₁, ORF1ab₃₇₃₂₋ ₃₇₄₀, ORF1ab₅₄₇₀₋₅₄₇₈, ORF8₇₃₋₈₁, and ORF10₅₋₁₃). Moreover, 7 out of 16 SARS-CoV-2 peptides induced significant CD4⁺ T-cell responses in “humanized” HLA-DR1/HLA-A*02:01 double transgenic mice (FIG. 13C, FIG. 13D). The immunogenic epitopes were derived from both structural Spike protein (S₁₋₁₃) and membrane protein (M₁₇₆₋₁₉₀) and from non-structural proteins (ORF1a₁₃₅₀₋₁₃₆₅, ORF1a₅₀₁₉₋₅₀₃₃, ORF1a₆₄₂₀₋ ₆₄₃₄, ORF6₁₂₋₂₆, ORF7b₈₋₂₂ and ORF8b₁₋₁₅). The remaining 9 CD4⁺ T cell epitopes presented moderate/low level of immunogenicity in HLA-DR1/HLA-A*02:01 double transgenic mice.

Altogether, these results indicate that pre-existing memory CD4⁺ T and CD8⁺ T cells specific to both structural and non-structural protein antigens and epitopes are present in COVID-19 patients and unexposed healthy individuals. While SARS-CoV-2-specific CD4⁺ and CD8⁺ T cells in COVID-19 patients and healthy donors target epitopes from the whole virus proteome, most T cell epitopes are concentrated in the non-structural proteins, with ORF1a/b being the most targeted antigens. These memory T cells recognized highly conserved SARS-CoV-2 epitopes that cross-react with the human and animal Coronaviruses. It is likely that infection with a “common cold” Coronavirus and/or human exposition with animal and pet related coronaviruses induced long-lasting memory CD4⁺ and CD8⁺ T cells specific to the structural and non-structural SARS-CoV-2 epitopes in healthy unexposed individuals. Heterologous immunity and heterologous immunopathology orchestrated by these cross-reactive epitope-specific memory CD4⁺ and CD8⁺ T cells, following previous multiple exposures to “common cold” Coronaviruses, may have shaped protection versus susceptibility to SARS-CoV-2 infection and disease, with a yet-to-be determined mechanism(s).

### Identification of B-cell Epitopes From SARS-CoV-2 Spike Protein That are Highly Conserved Between Human and Animal Coronaviruses, That are Antigenic in Humans and Immunogenic in “Humanized” HLA Transgenic Mice

SARS-CoV-2 B Cell Epitope Prediction: Linear B cell epitope predictions were carried out on the surface glycoprotein (S), the primary target of B cell immune responses for SARS-CoV. We used the BepiPred 2.0 algorithm embedded in the B cell prediction analysis tool hosted on the IEDB platform. For each protein, the epitope probability score for each amino acid and the probability of exposure was retrieved. Potential B cell epitopes were predicted using a cutoff of 0.55 (corresponding to a specificity greater than 0.81 and sensitivity below 0.3) and considering sequences having more than 5 amino acid residues. This screening process resulted in 28 B-cell peptides. From this pool, we selected 10 B-cell epitopes with 19 to 62 amino acid lengths. Three B-cell epitopes were observed to possess receptor binding domain (RBD) region specific amino acids. Structure-based antibody prediction was performed by using Discotope 2.0, and a positivity cutoff greater than -2.5 was applied (corresponding to specificity greater than or equal to 0.80 and sensitivity below 0.39), using the SARS-CoV-2 spike glycoprotein structure (PDB ID: 6M1D).

Protein-peptide molecular docking: Computational peptide docking of B cell peptides into the ACE2 Complex (binding protein) was performed using the GalaxyPepDock under GalaxyWEB. To retrieve the ACE2 structure, we used the X-ray crystallographic structure ACE2-B0AT1 complex-6M1D available on the Protein Data Bank. The 6M1D with a structural weight of 334.09 kDa, possesses 2 unique protein chains, 2,706 residues, and 21,776 atoms. In this study, flexible target docking based on an energy-optimization algorithm was carried out on the ligand-binding domain containing ACE2 within the 4GBX structure. Similarity scores were calculated for protein-peptide interaction pairs for each residue. The prediction accuracy is estimated from a linear model as the relationship between the fraction of correctly predicted binding site residues and the template-target similarity measured by the protein structure similarity score and interaction similarity score (S_{Inter}) obtained by linear regression. S_{Inter} shows the similarity of amino acids of the B-cell peptides aligned to the contacting residues in the amino acids of the ACE2 template structure. Higher S_{Inter} score represents a more significant binding affinity among the ACE2 molecule and B-cell peptides. Subsequently, molecular docking models were built based on distance restraints for protein-peptide pairs using GalaxyPepDock. Based on the optimized energy scores, docking models were ranked. While performing the protein-peptide docking analysis for CD8⁺ T cell epitope peptides, we used the X-ray Crystal structure of HLA-A*02:01 in complex-4UQ3 available on the Protein Data Bank and for CD4 peptides X-ray crystallographic structure HLA-DM-HLA-DRB1 Complex-4GBX.

Next, potential linear B-cell (antibody) epitopes were predicted on Spike protein sequence of the first SARS-CoV-2-Wuhan-Hu-1 strain (NCBI GenBank accession number MN908947.3) using BepiPred 2.0, with a cutoff of 0.55 (corresponding to a specificity greater than 0.81 and sensitivity below 0.30) and considering sequences having more than 5 amino acid residues. This stringent screening process initially resulted in the identification of 15 linear B-cell epitopes. From this pool of 28 potential epitopes, 15 B-cell epitopes were later selected, (19 to 62 amino acids in length), based on: (i) their sequences being highly conserved between SARS-CoV-2, the main 4 major “common cold” Coronaviruses (CoV-OC43 (KF923903), CoV-229E (KY983587), CoV-HKU1 (AY884001), and CoV-NL63 (NC_005831)) (68), and the SARS-like SL-CoVs that are isolated from bats, civet cats, pangolins and camels; and (ii) the probability of exposure each linear epitope to the surface of infected target cells (FIG. 14). The Spike epitope sequences highlighted in blue indicate a high degree of homology among the currently circulating 81,963 SARS-CoV-2 strains and at least a 50% conservancy among two or more human SARS-CoV strains from previous outbreaks, and the SL-CoV strains isolated from bats, civet cats, pangolins and camels (FIG. 14). Two of the 15 B-cell epitopes namely S₃₆₉₋₃₉₃, and S₄₄₀₋₅₀₁ overlap with the Spike’s receptor binding domain (RBD) region that bind to the ACE2 receptor (designated as RBD-1 and RBD-2 in FIG. 15A, FIG. 15B). Higher interaction similarity scores were observed for RBD-derived epitopes S₃₆₉₋₃₉₃ and S₄₇₁₋₅₀₁ when molecular docking was performed against the ACE2 receptor. Upon screening for the glycosylation regions, B-cell epitopes S₁₃₋₃₇, S₅₉₋₈₁, S₃₂₉₋₃₆₃, S₆₀₁₋₆₄₀, S₁₁₃₃₋₁₁₇₂ with Asparagines were predicted to be N-glycosylated. In contrast, B-cell epitopes S₅₁₆₋₅₃₆, S₅₂₄₋₅₉₈, S₈₀₂₋₈₁₉ were observed to be the O-glycosylated. The remaining B-cell epitopes S₂₈₇₋₃₁₇, S₃₀₄₋₃₂₂, S₃₆₉₋₃₉₃, S₄₀₄₋₅₀₁, S₄₄₀₋₅₀₁, S₆₇₂₋₆₉₀, and S₈₈₈₋₉₀₉ were found to possess no glycosylation.

The ability of each of the 15 B-cell epitopes selected from the Spike protein, that showed a high conservancy between human and animal Coronaviruses, to induce SARS-CoV-2 epitope-specific antibody-producing plasma B cells and IgG antibodies in B6 mice was later determined (FIG. 16A). Synthetic peptides corresponding to each linear B cell epitope were produced. Since 4 epitopes were too long to synthetize (e.g., 62 amino acids), they were divided into 2 or 3 short fragments resulting in a total of 22 B-cell epitope peptides. As illustrated in FIG. 16A, groups of five B6 mice each received two subcutaneous (s.c.) injections with mixtures of 3 to 4 B-cell epitope peptides, mixed with CpG and Alum adjuvants. Negative control mice received adjuvant alone, without Ags. The frequency of antibody-producing plasma B cells and the level of IgG antibodies specific to each SARS-CoV-2 B cell epitope were determined in the spleen and in the serum using FACS staining of CD138 and B220 surface markers and IgG-ELISpot and ELISA assays, respectively. The gating strategy used to determine the frequencies of plasma B-cells in the spleen is shown in FIG. 16B. Out of the 22 Spike B-cell epitopes, 7 epitopes (S₁₃₋₃₇, S₂₈₇₋₃₁₇, S₅₂₄₋₅₅₈, S₅₄₄₋₅₇₈, S₅₆₅₋₅₉₈, S₆₀₁₋₆₂₈, and S₆₁₄₋₆₄₀) induced high frequencies of CD138⁺B220⁺ plasma B cells in the spleen of B6 mice (FIG. 16C). The IgG ELISpot assay confirmed that 7 out of the 22 Spike B-cell epitopes induced significant numbers of IgG-producing B cells in the spleen (FIG. 16D). Moreover, significant amounts of IgG were detected in the serum of the immunized B6 mice. These IgG antibodies were specific to 6 out of the 22 Spike B-cell peptide epitopes (S₁₃₋₃₇, S₅₉₋₈₁, S₂₈₇₋₃₁₇, S₅₆₅₋₅₉₈, S₆₀₁₋₆₂₈, and S₆₁₄₋₆₄₀) (FIG. 16E). As expected, non-immunized animals or those that received adjuvant alone did not develop detectable IgG responses. Of these 6 highly immunogenic B cell peptides, 5 peptides (S₁₃₋₃₇, S₅₉₋₈₁, S₂₈₇₋₃₁₇, S₆₀₁₋₆₂₈, and S₆₁₄₋₆₄₀) were highly antigenic as they were recognized by serum IgG from COVID-19 patients, confirming the presence of at least one native linear B cell epitope in each peptide (FIG. 16F). In summary, we identified five highly conserved immunogenic and antigenic human B-cell target epitopes from the Spike SARS-CoV-2 virus that recall IgG antibodies from COVID-19 patients. This study further discovered five highly conserved B-cell epitopes from SARS-CoV-2: S₁₃₋₃₇, S₂₈₇₋₃₁₇, S₃₃₈₋₃₆₃, S₆₁₄₋₆₄₀, and S₁₁₃₃₋₁₁₆₀ that are recognized by IgG antibodies from healthy individuals who were never exposed to COVID-19. suggesting B-cell epitopes cross-reactivity to other human Coronaviruses (FIG. 16G). Besides their application in vaccines; some of these epitopes can be used in diagnostics of all Coronaviruses infections because of the conservancy of these epitopes. This applies to both B and T cell epitopes.

### Additional Materials and Methods

Epitope conservancy analysis: The Epitope Conservancy Analysis tool was used to compute the degree of the conservancy of CD8⁺ T cell, CD4⁺ T cell, and B-cell epitopes within a given protein sequence of SARS-CoV-2 set at 100% identity level. The fraction of protein sequences that contain the regions similar to epitopes were evaluated on the degree of similarity or correspondence among two sequences. The CD8⁺ T cell, and CD4⁺ T cell epitopes were screened against all the twelve structural and non-structural proteins of SARS-CoV-2 namely YP_009724389.1 (ORF1ab), YP_009725295.1 (ORF1a), YP_009724390.1 (surface glycoprotein), YP_009724391.1 (ORF3a). YP_009724392.1 (envelope protein), YP_009724393.1 (membrane glycoprotein), YP_009724394.1 (ORF6), YP_009724395.1 (ORF7a), YP_009725318.1 (ORF7b), YP_009724396.1 (ORF8), YP_009724397.2 (nucleocapsid phosphoprotein), YP_009725255.1 (ORF10). B-cell epitopes were screened for their conservancy against surface glycoprotein (YP_009724390.1) of SARS-CoV-2. Epitope linear sequence conservancy approach was used for linear epitope sequences with a sequence identity threshold set at ≥ 50%. This analysis resulted in (i) the calculated degree of conservancy (percent of protein sequence matches a specified identity level) and (ii) the matching minimum/maximum identity levels within the protein sequence set. The CD8⁺ and CD4⁺ T cell epitopes that showed ≥ 50% conservancy in at-least two human SARS-CoV strains, and two SARS-CoV strains (from bat/civet/pangolin/camel) were selected as candidate epitopes. N and O glycosylation sites were screened using NetNGlyc 1.0 and NetOGlyc 4.0 prediction servers, respectively.

Population-Coverage-Based T Cell Epitope Selection: For a robust epitope screening, we evaluated the conservancy of CD8⁺ T cell, CD4⁺ T cell, and B cell epitopes within Human-SARS-CoV-2 genome sequences representing North America, South America, Africa, Europe, Asia, and Australia. As of August 27^{th}, 2020, the NextStrain database recorded 81,963 human-SARS-CoV-2 genome sequences and the number of genome sequences continues growing daily. In the present analysis, 81,963 human-SARS-CoV-2 genome sequences were extrapolated from the GISAID and NCBI GenBank databases. We therefore considered all the 81,963 SARS-CoV-2 genome sequences representing six continents for subsequent conservancy analysis. We set a threshold for a candidate CD8⁺ T cell, CD4⁺ T cell, and B-cell epitope if the epitope showed 100% sequence conservancy in ≥ 95 human-SARS-CoV-2 genome sequences. Furthermore, population coverage calculation (PPC) was carried out using the Population Coverage software hosted on IEDB platform (47). PPC was performed to evaluate the distribution of screened CD8⁺ and CD4⁺ T cell epitopes in world population at large in combination with HLA-I (HLA-A*01:01,HLA-A*02:01,HLA-A*03:01,HLA-A*11:01,HLA-A*23:01), and HLA-II (HLA-DRB1*01:01, HLA-DRB1*11:01, HLA-DRB1*15:01, HLA-DRB1*03:01, HLA-DRB1*04:01) alleles.

Peptide synthesis: Potential peptide epitopes (9-mer long for CD8* T cell epitopes and 15-mer long for CD4⁺ T cell epitopes) identified from twelve human-SARS-CoV-2 proteins namely ORF1ab. ORF1a, surface glycoprotein, ORF3a, envelope protein, membrane glycoprotein. ORF6, ORF7a, ORF7b, ORF8, nucleocapsid phosphoprotein, and ORF10 were synthesized using solid-phase peptide synthesis and standard 9-fluorenylmethoxycarbonyl technology (21^{st} Century Biochemicals, Inc. Marlborough, MA). The purity of peptides was over 90%, as determined by reversed-phase high-performance liquid chromatography (Vydac C18) and mass spectroscopy (VOYAGER MALDI-TOF System). Stock solutions were made at 1 mg/mL in 10% DMSO in PBS. Similar method of synthesis was used for B cell peptide epitopes from the spike protein of SARS-CoV-2.

Cell Lines: T₂ (174 × CEM.T2) mutant hybrid cell line derived from the T-lymphoblast cell line CEM was obtained from the ATCC (www.atcc.org). The T₂ cell line was maintained in IMDM (ATCC, Manassas, VA) supplemented with 10% heat-inactivated fetal calf serum (FCS) and 100 U of penicillin/mL, 100 U of streptomycin/mL (Sigma-Aldrich, St. Louis, MO). T₂ cells lack the functional transporter associated with antigen processing (TAP) heterodimer and failed to express normal amounts of HLA-A*02:01 on the cell surface. HLA-A*02:01 surface expression is stabilized following the binding of exogenous peptides to these MHC class I molecules.

Stabilization of HLA-A*02:01 on class-I-HLA-transfected B × T hybrid cell lines: To determine whether synthetic peptides could stabilize HLA-A*02:01 molecule expression on the T₂ cell surface, peptide-inducing HLA-A*02:01 up-regulation on T₂ cells was examined according to a previously described protocol). T₂ cells (3 × 10⁵/well) were incubated with different concentrations (30, 10 and 3 µM) of 91 individual CD8⁺ T cell specific peptides in 48-well plates for 18 hours at 26° C. Cells were then incubated at 37° C. for 3 hours in the presence of 0.7 µL/mL BD GolgiStop™ to block cell surface expression of newly synthesized HLA-A*02:01 molecules, and human β-2 microglobulin (1 µg/mL). The cells were subsequently washed with FACS buffer (1% BSA and 0.1% sodium azide in phosphate-buffered saline) and stained with anti-HLA-A2 specific monoclonal antibody (clone BB7.2) (BD-Pharmingen, San Diego, CA) at 4° C. for 30 minutes. After incubation, the cells were washed with FACS buffer, fixed with 2% paraformaldehyde in phosphate-buffered saline, and analyzed by flow cytometry using a Fortessa (Becton Dickinson) flow cytometer equipped with a BD High Throughput Sampler for rapid analysis of samples prepared in plate format. The acquired data were analyzed with FlowJo software (BD Biosciences, San Jose, CA) and expression was measured by mean fluorescence intensity (MFI). Percent MFI increase was calculated as follows: Percent MFI increase = (MFI with the given peptide - MFI without peptide) / (MFI without peptide) × 100. Each experiment was performed 3 times, and means + SD values were calculated.

HLA-A*02:01 and HLA-DR1 double transgenic mice: A colony of human leukocyte antigens (HLA) class I and class II double transgenic (Tg) mice was maintained at the University of California Irvine (50) vivarium and treated in accordance with the AAALAC (Association for Assessment and Accreditation of Laboratory Animal Care) according to Institutional Animal Care and Use Committee-approved animal protocols (IACUC # 2020-19-111), and NIH (National Institutes of Health) guidelines. The HLA Tg mice retain their endogenous mouse major histocompatibility complex (MHC) locus and express human HLA-A*02:01 and HLA-DRB*01 under the control of its normal promoter. Prior to this study, the expression of HLA-A*02:01 and DR1 molecules on the PBMCs of each HLA-Tg mouse were confirmed by fluorescence-activated cell sorting (FACS).

Immunization of mice: Groups of age-matched HLA transgenic mice/B6 mice (n = 3) were immunized subcutaneously, on days 0 and 14, with a mixture of four SARS-CoV-2-derived human CD4⁺ T/ CD8⁺ T /B cell peptide epitopes delivered in alum and CpG₁₈₂₆ adjuvants. As a negative control, mice received adjuvants alone (mock-immunized).

Splenocytes isolation: Spleens were harvested from mice in two weeks post second immunization. Spleens were placed in 10 ml of cold PBS with 10% fetal bovine serum (FBS) and 2X antibiotic-antimycotic (Life Technologies, Carlsbad, CA). Spleens were minced finely and sequentially passed through a 100 µm screen and a 70 µm screen (BD Biosciences, San Jose. CA). Cells were then pelleted by centrifugation at 400 × g for 10 minutes at 4° C. Red blood cells were lysed using a lysis buffer (ammonium chloride) and washed again. Isolated splenocytes were diluted to 1 × 10⁶ viable cells per ml in RPMI media with 10% (v/v) FBS and 2 × antibiotic-antimycotic. Viability was determined by trypan blue staining.

Flow cytometry analysis: PBMCs/Splenocytes were analyzed by flow cytometry. The following antibodies were used: CD8, CD4. CD62L, CD107^{a/b}, CD44, CD69, TNF-α and IFN-β). For surface staining, mAbs against various cell markers were added to a total of 1×10⁶ cells in phosphate-buffered saline containing 1% FBS and 0.1% sodium azide (fluorescence-activated cell sorter [FACS] buffer) and left for 45 minutes at 4° C. At the end of the incubation period, the cells were washed twice with FACS buffer. A total of 100,000 events were acquired by LSRII (Becton Dickinson, Mountain View, CA) followed by analysis using FlowJo software (TreeStar, Ashland, OR).

ELISpot assay: All reagents used were filtered through a 0.22 µm filter. Wells of 96-well Multiscreen HTS Plates (Millipore, Billerica, MA) were pre-wet with 30% ethanol and then coated with 100 µl primary anti-IFN-gamma antibody solution (10 µg/ml of 1-D1K coating antibody from Mabtech in PBS, pH 7.4, V-E4) OVN at 4° C. After washing, nonspecific binding was blocked with 200 µl of RPMI media with 10% (v/v) FBS for 2 hours at room temperature. Following the blockade, 0.5 × 10⁶ cells from patients PBMCs (or from mouse splenocytes) in 100 µl of RPMI were mixed with 10 µg individual peptides (with DMSO for no stimulation or with individual peptide at a final concentration of 10 µg/ml). After incubation in humidified 5% CO₂ at 37° C. for 72 hours (samples from COVID-19 patients) or 5 days (for healthy donor samples, to recall their T-cell memory), cells were removed by washing (using PBS and PBS-Tween 0.02% solution) and 100 µl of biotinylated secondary anti-IFN-y antibody (clone 7-B6-1. Mabtech) in blocking buffer (PBS 0.5% FBS) was added to each well. Following a 2-hour incubation and washing, HRP-conjugated streptavidin was diluted 1:1000, and wells were incubated with 100 µl for 1 hour at room temperature. Following washing, wells were incubated for 1 hour at room temperature with 100 µl of TMB detection reagent and spots counted with an automated EliSpot Reader System (ImmunoSpot reader, Cellular Technology, Shaker Heights, OH).

ELISA based assay to access the efficacy of receptor-binding domain region towards inducing specific antibodies against B-cell epitopes in HLA-A2 treated mice: The efficacy of our B-cell peptide-epitopes towards inducing specific antibodies was measured in the HLADR1/A*02:01 immunized mice by ELISA. ELISA plates (Cat. M5785, Sigma Aldrich) were first coated overnight at 4° C. with 10 µg/ml of each B cell peptide epitope. Subsequently, plates were washed five times with PBS-Tween 0.01% before starting the blocking by adding PBS 1% BSA for 3 hours at room temperature, followed by a second wash. Sera of C57BL/6 mice immunized either with pool B cell peptides alum/CpG or adjuvant alone (control) were added into the wells at various dilutions (⅕, 1/25, 1/125, and 1/625 or PBS only, in triplicates). Plates were incubated at 4° C. overnight with the sera, then washed with PBS-Tween 0.01% before adding anti-mouse IgG antibody (Mabtech - 1/500 dilution). After the last washing, Streptavidin-HRP (Mabtech - 1/1000 dilution) was added for 30 minutes at room temperature. Finally, we added 100µl of filtered TMB substrate for 15 minutes and blocked the reaction with H₂S0₄ before the read-out (OD measurement was done at 450 nm on the Bio-Rad iMark microplate reader). The same procedure was followed to measure the titers of antibodies specific against our 15 screened B-cell epitopes in the sera of COVID-19 patients (n = 40) and healthy donors (n = 10), using anti-human IgG antibody as the secondary antibody (Mabtech - 1/500 dilution).

Constructing the Phylogenetic Tree: Phylogenetic analyses were conducted in MEGA X. The evolutionary history was performed, and phylogenetic tree was constructed using the Maximum Likelihood method and Tamura-Nei model. The Maximum Likelihood method assumes that each locus evolves independently by pure genetic drift. The tree with the highest log likelihood was selected. Initial tree(s) for the heuristic search were obtained by applying Neighbor-Join and BioNJ algorithms to a matrix of pairwise genetic distances estimated using the Tamura-Nei model, and then selecting the topology with superior log likelihood value. This analysis involved available nucleotide sequences of SARS-CoV-2 from humans (Homo Sapiens), bat (Rhinolophus affinis, Rhinolophus malayanus), and pangolin (Manis javanica). In addition, genome sequences from previous outbreaks of SARS-CoV in human, bat, civet, and camel were taken into consideration while performing the evolutionary analyses.

Data and Code Availability: The human specific SARS-CoV-2 complete genome sequences were retrieved from the GISAID database, whereas the SARS-CoV-2 sequences for pangolin (Manis javanica), and bat (Rhinolophus affinis, Rhinolophus malayanus) were retrieved from NCBI Genome sequences of previous strains of SARS-CoV for human, bat, civet, and camel were retrieved from the NCBI GenBank.

Statistical analyses: Data for each differentially expressed marker among blockade-treated, and mock-treated groups of HLA Tg mice were compared by analysis of variance (ANOVA) and Student’s t-test using GraphPad Prism version 6 (La Jolla, CA). Statistical differences observed in the measured CD8-, CD4- T cells and antibody responses between healthy donors and COVID-19 patients were calculated using ANOVA and multiple t-test comparison procedures in GraphPad Prism. Data are expressed as the mean ± SD. Results were considered statistically significant at P ≤ 0.05.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of or “consisting of”, and as such the written description requirement for claiming one or more embodiments of the present invention using the phrase “consisting essentially of or “consisting of” is met.

What is claimed is:

1. A universal multi-epitope, pan-coronavirus recombinant vaccine composition, the composition comprising at least two of:
a) one or more conserved coronavirus B-cell target epitopes;
b) one or more conserved coronavirus CD4⁺ T cell target epitopes;
c) one or more conserved coronavirus CD8⁺ T cell target epitopes; wherein at least one epitope is derived from a non-spike protein.

2. The composition of claim 1, wherein the non-spike proteins are encoded by ORF1 ab, ORF3a, ORF6, ORF7a, ORF7b, ORF8, or ORF10, or derived from an Envelope protein, a Membrane protein, or a Nucleocapsid protein.

3. The composition of claim 1, wherein the one or more conserved epitopes are derived from one or more of: one or more SARS-CoV-2 human strains or variants in current circulation; one or more coronaviruses that has caused a previous human outbreak; one or more coronaviruses isolated from animals selected from a group consisting of bats, pangolins, civet cats, minks, camels, and other animal receptive to coronaviruses; or one or more coronaviruses that cause the common cold.

4. The composition of claim 3, wherein the one or more SARS-CoV-2 human strains or variants in current circulation are selected from: variant B.1.177; variant B.1.160, variant B.1.1.7 (UK), variant P.1 (Japan/Brazil), variant B.1.351 (South Africa), variant B.1.427 (California), variant B.1.429 (California), variant B.1.258; variant B.1.221; variant B.1.367; variant B.1.1.277; variant B.1.1.302; variant B.1.525; variant B.1.526, variant S:677H; variant S:677P; B.1.617.2-Delta, variant B.1.1.529-Omicron (BA.1); sub-variant Omicron (BA.1); sub-variant Omicron (BA.2); sub-variant Omicron (BA.3); sub-variant Omicron (BA.4); sub-variant Omicron (BA.5).

5. The composition of claim 3, wherein the one or more coronaviruses that cause the common cold are selected from: 229E alpha coronavirus, NL63 alpha coronavirus, OC43 beta coronavirus, and HKU1 beta coronavirus.

6. The composition of claim 1, wherein target epitopes are derived from a SARS-CoV-2 protein selected from a group consisting of: ORF1ab protein, Spike glycoprotein, ORF3a protein, Envelope protein, Membrane glycoprotein, ORF6 protein, ORF7a protein, ORF7b protein, ORF8 protein, Nucleocapsid protein and ORF10 protein.

7. The composition of claim 1, wherein the one or more conserved coronavirus CD8⁺ T cell target epitopes are selected from SEQ ID NO: 2-29, SEQ ID NO: 30-57, SEQ ID NO: 184-203, SEQ ID NO: 204-224, or a combination thereof; wherein the one or more conserved coronavirus CD4⁺ T cell target epitopes are selected from SEQ ID NO: 58-73, SEQ ID NO: 74-105, SEQ ID NO: 225-243, SEQ ID NO: 244-262, or a combination thereof; wherein the one or more coronavirus B cell target epitopes are selected from SEQ ID NO: 106-116, SEQ ID NO: 117-138, SEQ ID NO: 263-270, SEQ ID NO: 271-284, or a combination thereof.

8. The composition of claim 1 further comprising a T cell attracting chemokine, wherein the T cell attracting chemokine is CCL5, CXCL9, CXCL10, CXCL11, or a combination thereof.

9. The composition of claim 1 further comprising a composition that promotes T cell proliferation, wherein the composition that promotes T cell proliferation is IL-7 or IL-15.

10. The composition of claim 1, wherein the vaccine composition protects against disease caused by one or more coronavirus variants or coronavirus subvariants.

11. The composition of claim 10, wherein the coronavirus variants or coronavirus subvariants comprise past or currently circulating coronavirus variants or coronavirus subvariants wherein the coronavirus variants comprise alpha, beta, gamma, delta, and omicron.

12. The composition of claim 10, wherein the coronavirus variants or coronavirus subvariants comprise future variants or future subvariants of human and animal coronavirus.

13. The composition of claim 1, wherein the vaccine composition protects against infection and reinfection of coronavirus variants or coronavirus subvariants.

14. The composition of claim 13, wherein the coronavirus variants or coronavirus subvariants comprise past or currently circulating coronavirus variants or coronavirus subvariants, wherein the coronavirus variants comprise alpha, beta, gamma, delta, and omicron.

15. The composition of claim 13, wherein the coronavirus variants or coronavirus subvariants comprise future variants or future subvariants of human and animal coronavirus.

16. The composition of claim 13, wherein the vaccine composition protects against infection or reinfection of one or more coronavirus variant or coronavirus subvariant.

17. The composition of claim 16, wherein the vaccine composition protects against infection or reinfection of multiple coronavirus variants or coronavirus subvariants.

18. The composition of claim 16, wherein the vaccine composition protects against infection or reinfection caused by one coronavirus variants or coronavirus subvariants.

19. The composition of claim 1, wherein the vaccine composition induces strong and long-lasting protection mediated by antibodies (Abs), CD4+ T helper (Th1) cells, and/or CD8+ cytotoxic T-cells (CTL).

20. The composition of claim 1, wherein the composition protects against Sarbecoviruses, wherein sarbecoviruses comprise SARS-CoV1 or SARS-CoV2.

21. A multi-epitope, pan-coronavirus recombinant vaccine composition, the composition comprising at least two of:
a) one or more conserved coronavirus B-cell target epitopes selected from SEQ ID NO: 106-116, SEQ ID NO: 117-138, SEQ ID NO: 263-270, SEQ ID NO: 271-284,or a combination thereof;
b) one or more conserved coronavirus CD4⁺ T cell target epitopes selected from SEQ ID NO: 58-73, SEQ ID NO: 74-105, SEQ ID NO: 225-243, SEQ ID NO: 244-262, or a combination thereof;
c) one or more conserved coronavirus CD8⁺ T cell target epitopes selected from SEQ ID NO: 2-29, SEQ ID NO: 30-57, SEQ ID NO: 184-203, SEQ ID NO: 204-224, or a combination thereof;
wherein at least one epitope is derived from a non-spike protein, wherein the composition induces immunity to only the epitopes.

22. A multi-epitope, pan-coronavirus recombinant vaccine composition comprising one of SEQ ID NO: 139-155.
